SEQUENCE LISTING (1923 sequences)

SEQ ID NO: 1 - protein, 6 aa, synthetic construct
  REGION 1..6: note = Synthetic
HHHHHH

SEQ ID NO: 2 - protein, 5 aa, synthetic construct
  REGION 1..5: note = Synthetic
DDDDK

SEQ ID NO: 3 - protein, 6 aa, synthetic construct
  REGION 1..6: note = Synthetic
ADDDDK

SEQ ID NO: 4 - protein, 6 aa, Homo sapiens
DPQFQK

SEQ ID NO: 5 - protein, 6 aa, Homo sapiens
LQQWYR

SEQ ID NO: 6 - protein, 6 aa, Homo sapiens
SELNLR

SEQ ID NO: 7 - protein, 21 aa, Homo sapiens
FNHFSLTLNTNHGHILVDYSK

SEQ ID NO: 8 - protein, 7 aa, Homo sapiens
INYTEGR

SEQ ID NO: 9 - protein, 8 aa, Homo sapiens
AVLHVALR

SEQ ID NO: 10 - protein, 10 aa, Homo sapiens
SNTPILVDGK

SEQ ID NO: 11 - protein, 14 aa, Homo sapiens
VWYVSNIDGTHIAK

SEQ ID NO: 12 - protein, 17 aa, Homo sapiens
TLAQLNPESSLFIIASK

SEQ ID NO: 13 - protein, 15 aa, Homo sapiens
TFTTQETITNAETAK

SEQ ID NO: 14 - protein, 8 aa, Homo sapiens
EWFLQAAK

SEQ ID NO: 15 - protein, 7 aa, Homo sapiens
DPSAVAK

SEQ ID NO: 16 - protein, 11 aa, Homo sapiens
HFVALSTNTTK

SEQ ID NO: 17 - protein, 7 aa, Homo sapiens
ELQAAGK

SEQ ID NO: 18 - protein, 15 aa, Homo sapiens
VFEGNRPTNSIVFTK

SEQ ID NO: 19 - protein, 22 aa, Homo sapiens
IFVQGIIWDINSFDQWGVELGK

SEQ ID NO: 20 - protein, 25 aa, Homo sapiens
LWVLVVLGTSWVGWGSQGTEAAQLR

SEQ ID NO: 21 - protein, 7 aa, Homo sapiens
EYEPYFK

SEQ ID NO: 22 - protein, 24 aa, Homo sapiens
EKPQSTISGLLGPTLYAEVGDIIK

SEQ ID NO: 23 - protein, 13 aa, Homo sapiens
ADKPLSIHPQGIR

SEQ ID NO: 24 - protein, 16 aa, Homo sapiens
LSEGASYLDHTFPAEK

SEQ ID NO: 25 - protein, 10 aa, Homo sapiens
GTLTEGGTQK

SEQ ID NO: 26 - protein, 13 aa, Homo sapiens
QIVLLFAVFDESK

SEQ ID NO: 27 - protein, 9 aa, Homo sapiens
WIISSLTPK

SEQ ID NO: 28 - protein, 13 aa, Homo sapiens
SQHLDNFSNQIGK

SEQ ID NO: 29 - protein, 10 aa, Homo sapiens
EDGILGPIIR

SEQ ID NO: 30 - protein, 11 aa, Homo sapiens
AVQPGETYTYK

SEQ ID NO: 31 - protein, 17 aa, Homo sapiens
AADIEQQAVFAVFDENK

SEQ ID NO: 32 - protein, 10 aa, Homo sapiens
SWYLEDNINK

SEQ ID NO: 33 - protein, 17 aa, Homo sapiens
LEPEDEESDADYDYQNR

SEQ ID NO: 34 - protein, 8 aa, Homo sapiens
LAAALGIR

SEQ ID NO: 35 - protein, 11 aa, Homo sapiens
FTVNNLAEPQK

SEQ ID NO: 36 - protein, 15 aa, Homo sapiens
APSHQQATTAGSPLR

SEQ ID NO: 37 - protein, 10 aa, Homo sapiens
LLSLGAGEFK

SEQ ID NO: 38 - protein, 6 aa, Homo sapiens
SQEHAK

SEQ ID NO: 39 - protein, 10 aa, Homo sapiens
DPPSDLLLLK

SEQ ID NO: 40 - protein, 6 aa, Homo sapiens
QSNSSK

SEQ ID NO: 41 - protein, 7 aa, Homo sapiens
WHLASEK

SEQ ID NO: 42 - protein, 14 aa, Homo sapiens
AWGESTPLANKPGK

SEQ ID NO: 43 - protein, 6 aa, Homo sapiens
QSGHPK

SEQ ID NO: 44 - protein, 6 aa, Homo sapiens
SQFLIK

SEQ ID NO: 45 - protein, 10 aa, Homo sapiens
HTHHAPLSPR

SEQ ID NO: 46 - protein, 6 aa, Homo sapiens
TFHPLR

SEQ ID NO: 47 - protein, 10 aa, Homo sapiens
SEAYNTFSER

SEQ ID NO: 48 - protein, 7 aa, Homo sapiens
HSLVLHK

SEQ ID NO: 49 - protein, 12 aa, Homo sapiens
EFNPLVIVGLSK

SEQ ID NO: 50 - protein, 11 aa, Homo sapiens
DGTDYIEIIPK

SEQ ID NO: 51 - protein, 23 aa, Homo sapiens
EEVQSSEDDYAEIDYVPYDDPYK

SEQ ID NO: 52 - protein, 7 aa, Homo sapiens
TNINSSR

SEQ ID NO: 53 - protein, 11 aa, Homo sapiens
DPDNIAAWYLR

SEQ ID NO: 54 - protein, 6 aa, Homo sapiens
SNNGNR

SEQ ID NO: 55 - protein, 19 aa, Homo sapiens
NYYIAAEEISWDYSEFVQR

SEQ ID NO: 56 - protein, 17 aa, Homo sapiens
ETDIEDSDDIPEDTTYK

SEQ ID NO: 57 - protein, 8 aa, Homo sapiens
YLDSTFTK

SEQ ID NO: 58 - protein, 15 aa, Homo sapiens
GEYEEHLGILGPIIR

SEQ ID NO: 59 - protein, 10 aa, Homo sapiens
AEVDDVIQVR

SEQ ID NO: 60 - protein, 18 aa, Homo sapiens
NLASRPYSLHAHGLSYEK

SEQ ID NO: 61 - protein, 11 aa, Homo sapiens
TYEDDSPEWFK

SEQ ID NO: 62 - protein, 20 aa, Homo sapiens
EDNAVQPNSSYTYVWHATER

SEQ ID NO: 63 - protein, 12 aa, Homo sapiens
AWAYYSAVNPEK

SEQ ID NO: 64 - protein, 6 aa, Homo sapiens
SWYYEK

SEQ ID NO: 65 - protein, 15 aa, Homo sapiens
QHQLGVWPLLPGSFK

SEQ ID NO: 66 - protein, 18 aa, Homo sapiens
ASKPGWWLLNTEVGENQR

SEQ ID NO: 67 - protein, 11 aa, Homo sapiens
ASEFLGYWEPR

SEQ ID NO: 68 - protein, 14 aa, Homo sapiens
LNNGGSYNAWSVEK

SEQ ID NO: 69 - protein, 13 aa, Homo sapiens
EVIITGIQTQGAK

SEQ ID NO: 70 - protein, 11 aa, Homo sapiens
ENQFDPPIVAR

SEQ ID NO: 71 - protein, 8 aa, Homo sapiens
AYNRPTLR

SEQ ID NO: 72 - protein, 8 aa, Homo sapiens
QITASSFK

SEQ ID NO: 73 - protein, 11 aa, Homo sapiens
SWWGDYWEPFR

SEQ ID NO: 74 - protein, 7 aa, Homo sapiens
VNAWQAK

SEQ ID NO: 75 - protein, 9 aa, Homo sapiens
QWLEIDLLK

SEQ ID NO: 76 - protein, 17 aa, Homo sapiens
SYTIHYSEQGVEWKPYR

SEQ ID NO: 77 - protein, 9 aa, Homo sapiens
IFEGNTNTK

SEQ ID NO: 78 - protein, 10 aa, Homo sapiens
NFFNPPIISR

SEQ ID NO: 79 - protein, 9 aa, Homo sapiens
TWNQSIALR

SEQ ID NO: 80 - protein, 13 aa, Homo sapiens
EVWYFGLHYVDNK

SEQ ID NO: 81 - protein, 7 aa, Homo sapiens
GFPTWLK

SEQ ID NO: 82 - protein, 7 aa, Homo sapiens
VSAQEVR

SEQ ID NO: 83 - protein, 17 aa, Homo sapiens
FYPEDVAEELIQDITQK

SEQ ID NO: 84 - protein, 6 aa, Homo sapiens
FGDYNK

SEQ ID NO: 85 - protein, 8 aa, Homo sapiens
SGYLSSER

SEQ ID NO: 86 - protein, 6 aa, Homo sapiens
DQWEDR

SEQ ID NO: 87 - protein, 9 aa, Homo sapiens
IQVWHAEHR

SEQ ID NO: 88 - protein, 18 aa, Homo sapiens
GTDLWLGVDALGLNIYEK

SEQ ID NO: 89 - protein, 7 aa, Homo sapiens
QQLETEK

SEQ ID NO: 90 - protein, 7 aa, Homo sapiens
LQDYEEK

SEQ ID NO: 91 - protein, 8 aa, Homo sapiens
ELSEQIQR

SEQ ID NO: 92 - protein, 8 aa, Homo sapiens
ALQLEEER

SEQ ID NO: 93 - protein, 7 aa, Homo sapiens
QAVDQIK

SEQ ID NO: 94 - protein, 15 aa, Homo sapiens
SQEQLAAELAEYTAK

SEQ ID NO: 95 - protein, 8 aa, Homo sapiens
IALLEEAR

SEQ ID NO: 96 - protein, 10 aa, Homo sapiens
EDEVEEWQHR

SEQ ID NO: 97 - protein, 8 aa, Homo sapiens
EAQDDLVK

SEQ ID NO: 98 - protein, 13 aa, Homo sapiens
QLLTLSSELSQAR

SEQ ID NO: 99 - protein, 7 aa, Homo sapiens
IDEFEAL

SEQ ID NO: 100 - protein, 11 aa, Homo sapiens
VVSVDENFRPR

SEQ ID NO: 101 - protein, 13 aa, Homo sapiens
NELIPLIYLENPR

SEQ ID NO: 102 - protein, 8 aa, Homo sapiens
IAQWQSLK

SEQ ID NO: 103 - protein, 22 aa, Homo sapiens
LEAGINQLSFPLSSEPIQGSYR

SEQ ID NO: 104 - protein, 10 aa, Homo sapiens
VVVQTESGGR

SEQ ID NO: 105 - protein, 14 aa, Homo sapiens
IQHPFTVEEFVLPK

SEQ ID NO: 106 - protein, 12 aa, Homo sapiens
EEGTDLEVTANR

SEQ ID NO: 107 - protein, 10 aa, Homo sapiens
ISEITNIVSK

SEQ ID NO: 108 - protein, 15 aa, Homo sapiens
QGIPFFAQVLLVDGK

SEQ ID NO: 109 - protein, 18 aa, Homo sapiens
GSFALSFPVESDVAPIAR

SEQ ID NO: 110 - protein, 6 aa, Homo sapiens
VFTNSK

SEQ ID NO: 111 - protein, 7 aa, Homo sapiens
GEVFTLK

SEQ ID NO: 112 - protein, 7 aa, Homo sapiens
LPSNVVK

SEQ ID NO: 113 - protein, 11 aa, Homo sapiens
AVGYLITGYQR

SEQ ID NO: 114 - protein, 12 aa, Homo sapiens
HQDGSYSTFGER

SEQ ID NO: 115 - protein, 11 aa, Homo sapiens
EGTHGSHVYTK

SEQ ID NO: 116 - protein, 12 aa, Homo sapiens
ALLAYAFSLLGK

SEQ ID NO: 117 - protein, 8 aa, Homo sapiens
EILNSLDK

SEQ ID NO: 118 - protein, 14 aa, Homo sapiens
EDNLVHWERPQRPK

SEQ ID NO: 119 - protein, 23 aa, Homo sapiens
QQNAQGGFSSTQDTVVALHALSR

SEQ ID NO: 120 - protein, 8 aa, Homo sapiens
EDSPFALK

SEQ ID NO: 121 - protein, 10 aa, Homo sapiens
SLFTDLVAEK

SEQ ID NO: 122 - protein, 15 aa, Homo sapiens
ISASSEVAFLSIQIK

SEQ ID NO: 123 - protein, 7 aa, Homo sapiens
GPTQDFR

SEQ ID NO: 124 - protein, 6 aa, Homo sapiens
VSVQLK

SEQ ID NO: 125 - protein, 12 aa, Homo sapiens
ASPAFLASQNTK

SEQ ID NO: 126 - protein, 10 aa, Homo sapiens
QTLSWTVTPK

SEQ ID NO: 127 - protein, 13 aa, Homo sapiens
TLLVEAEGIEQEK

SEQ ID NO: 128 - protein, 7 aa, Homo sapiens
ASSPSGK

SEQ ID NO: 129 - protein, 8 aa, Homo sapiens
EGSGDGGR

SEQ ID NO: 130 - protein, 13 aa, Homo sapiens
AELNPWPEYIYTR

SEQ ID NO: 131 - protein, 10 aa, Homo sapiens
AEHDSILAEK

SEQ ID NO: 132 - protein, 6 aa, Homo sapiens
DSKPIK

SEQ ID NO: 133 - protein, 7 aa, Homo sapiens
VTLPDGK

SEQ ID NO: 134 - protein, 8 aa, Homo sapiens
QVDAESWK

SEQ ID NO: 135 - protein, 10 aa, Homo sapiens
VNTPTTTVYR

SEQ ID NO: 136 - protein, 9 aa, Homo sapiens
NSSTYWEGK

SEQ ID NO: 137 - protein, 10 aa, Homo sapiens
IYGISFPDPK

SEQ ID NO: 138 - protein, 13 aa, Homo sapiens
GAYIYNALIEFIR

SEQ ID NO: 139 - protein, 14 aa, Homo sapiens
GFQEVVTPNIFNSR

SEQ ID NO: 140 - protein, 9 aa, Homo sapiens
LADFGVLHR

SEQ ID NO: 141 - protein, 12 aa, Homo sapiens
NELSGALTGLTR

SEQ ID NO: 142 - protein, 11 aa, Homo sapiens
TVYSVFGFSFK

SEQ ID NO: 143 - protein, 9 aa, Homo sapiens
LNLSTRPEK

SEQ ID NO: 144 - protein, 13 aa, Homo sapiens
FLGDIEVWDQAEK

SEQ ID NO: 145 - protein, 12 aa, Homo sapiens
QLENSLNEFGEK

SEQ ID NO: 146 - protein, 14 aa, Homo sapiens
WELNSGDGAFYGPK

SEQ ID NO: 147 - protein, 6 aa, Homo sapiens
IDIQIK

SEQ ID NO: 148 - protein, 13 aa, Homo sapiens
FNLTYVSHDGDDK

SEQ ID NO: 149 - protein, 7 aa, Homo sapiens
RPVIVHR

SEQ ID NO: 150 - protein, 7 aa, Homo sapiens
QQFHDAK

SEQ ID NO: 151 - protein, 8 aa, Homo sapiens
ISGTVNIR

SEQ ID NO: 152 - protein, 8 aa, Homo sapiens
TISETIER

SEQ ID NO: 153 - protein, 7 aa, Homo sapiens
ILLEAAR

SEQ ID NO: 154 - protein, 6 aa, Homo sapiens
SIFEAR

SEQ ID NO: 155 - protein, 12 aa, Homo sapiens
GIKPVTLELGGK

SEQ ID NO: 156 - protein, 7 aa, Homo sapiens
FTEEVVK

SEQ ID NO: 157 - protein, 10 aa, Homo sapiens
IGDPLLEDTR

SEQ ID NO: 158 - protein, 6 aa, Homo sapiens
VLGFVK

SEQ ID NO: 159 - protein, 15 aa, Homo sapiens
ANDTTFGLAAGVFTR

SEQ ID NO: 160 - protein, 10 aa, Homo sapiens
VTIEYYSQLK

SEQ ID NO: 161 - protein, 11 aa, Homo sapiens
VEPADASGTEK

SEQ ID NO: 162 - protein, 8 aa, Homo sapiens
AFEPATGR

SEQ ID NO: 163 - protein, 10 aa, Homo sapiens
EVNLAVQNAK

SEQ ID NO: 164 - protein, 8 aa, Homo sapiens
GQLLPVSK

SEQ ID NO: 165 - protein, 12 aa, Homo sapiens
ALQQGLVGLELK

SEQ ID NO: 166 - protein, 13 aa, Homo sapiens
ATTGYPDPYGGEK

SEQ ID NO: 167 - protein, 9 aa, Homo sapiens
LALFQAIGK

SEQ ID NO: 168 - protein, 12 aa, Homo sapiens
LSELEPGTGDLR

SEQ ID NO: 169 - protein, 9 aa, Homo sapiens
FLNPNTLER

SEQ ID NO: 170 - protein, 9 aa, Homo sapiens
LTYHQLLER

SEQ ID NO: 171 - protein, 12 aa, Homo sapiens
APGSGLALLPLK

SEQ ID NO: 172 - protein, 9 aa, Homo sapiens
LAAVDVSAR

SEQ ID NO: 173 - protein, 19 aa, Homo sapiens
YLEGTGSVAGVVLLPEGHK

SEQ ID NO: 174 - protein, 8 aa, Homo sapiens
LWVDEAVR

SEQ ID NO: 175 - protein, 10 aa, Homo sapiens
GLVDRPLALR

SEQ ID NO: 176 - protein, 8 aa, Homo sapiens
LPLEAALR

SEQ ID NO: 177 - protein, 17 aa, Homo sapiens
QLSQAGSFSDGTHGGLR

SEQ ID NO: 178 - protein, 11 aa, Homo sapiens
GGEPQGPPFIK

SEQ ID NO: 179 - protein, 14 aa, Homo sapiens
QALSTATATVSVGK

SEQ ID NO: 180 - protein, 20 aa, Homo sapiens
GRPVSLWELLFSEAISSEQR

SEQ ID NO: 181 - protein, 17 aa, Homo sapiens
LAAELSATLEQAAATAR

SEQ ID NO: 182 - protein, 7 aa, Homo sapiens
VTFSGLR

SEQ ID NO: 183 - protein, 10 aa, Homo sapiens
DTVTPGELLK

SEQ ID NO: 184 - protein, 11 aa, Homo sapiens
AEIIDQDLYER

SEQ ID NO: 185 - protein, 9 aa, Homo sapiens
LEHGQATAK

SEQ ID NO: 186 - protein, 10 aa, Homo sapiens
DVGSLASAQR

SEQ ID NO: 187 - protein, 7 aa, Homo sapiens
LSIYEAR

SEQ ID NO: 188 - protein, 25 aa, Homo sapiens
GLLRPGTALILLEAQAATGFIIDPK

SEQ ID NO: 189 - protein, 9 aa, Homo sapiens
GHSVEEALR

SEQ ID NO: 190 - protein, 11 aa, Homo sapiens
AAVIGPDVFAK

SEQ ID NO: 191 - protein, 16 aa, Homo sapiens
VSAWELINSEYFSEGR

SEQ ID NO: 192 - protein, 9 aa, Homo sapiens
EVTLGQVAK

SEQ ID NO: 193 - protein, 8 aa, Homo sapiens
LLEAETQR

SEQ ID NO: 194 - protein, 10 aa, Homo sapiens
GVVGPELYGR

SEQ ID NO: 195 - protein, 9 aa, Homo sapiens
AEGAIAGFR

SEQ ID NO: 196 - protein, 10 aa, Homo sapiens
GLIPWEQAAR

SEQ ID NO: 197 - protein, 9 aa, Homo sapiens
SLQAVPGAK

SEQ ID NO: 198 - protein, 10 aa, Homo sapiens
GLLEDVQEGR

SEQ ID NO: 199 - protein, 12 aa, Homo sapiens
TTVPQLLASVQR

SEQ ID NO: 200 - protein, 8 aa, Homo sapiens
ASIAQAVR

SEQ ID NO: 201 - protein, 11 aa, Homo sapiens
DGLLPTGLGQR

SEQ ID NO: 202 - protein, 19 aa, Homo sapiens
LLEAQVASGFLVDPLNNQR

SEQ ID NO: 203 - protein, 8 aa, Homo sapiens
LSVEDAVK

SEQ ID NO: 204 - protein, 6 aa, Homo sapiens
VGLVGR

SEQ ID NO: 205 - protein, 11 aa, Homo sapiens
ELSEQLGQAER

SEQ ID NO: 206 - protein, 11 aa, Homo sapiens
AAAGYPDPYSR

SEQ ID NO: 207 - protein, 17 aa, Homo sapiens
LGLLDTQTSQVLTAVDK

SEQ ID NO: 208 - protein, 8 aa, Homo sapiens
FFFDPSAR

SEQ ID NO: 209 - protein, 9 aa, Homo sapiens
DQVTYQQLR

SEQ ID NO: 210 - protein, 7 aa, Homo sapiens
VPVSTGR

SEQ ID NO: 211 - protein, 15 aa, Homo sapiens
QVVSAVTALVEAAER

SEQ ID NO: 212 - protein, 8 aa, Homo sapiens
QPLQATFR

SEQ ID NO: 213 - protein, 13 aa, Homo sapiens
TLDELSQGTTTVK

SEQ ID NO: 214 - protein, 19 aa, Homo sapiens
SLEGGNFIAGVLIQGTQER

SEQ ID NO: 215 - protein, 8 aa, Homo sapiens
LTVEEAFK

SEQ ID NO: 216 - protein, 10 aa, Homo sapiens
ILADPSDDTK

SEQ ID NO: 217 - protein, 12 aa, Homo sapiens
GENYVYINEATR

SEQ ID NO: 218 - protein, 21 aa, Homo sapiens
FADQVVSFWDLLSSPYFTEDR

SEQ ID NO: 219 - protein, 15 aa, Homo sapiens
ELIQEYGAQSGGLEK

SEQ ID NO: 220 - protein, 20 aa, Homo sapiens
LLEIITTTIEETETQNQGIK

SEQ ID NO: 221 - protein, 12 aa, Homo sapiens
GEVTAADLFNSR

SEQ ID NO: 222 - protein, 6 aa, Homo sapiens
TLHTLR

SEQ ID NO: 223 - protein, 17 aa, Homo sapiens
LSVDEAVDVGLVNEELR

SEQ ID NO: 224 - protein, 6 aa, Homo sapiens
AATGYR

SEQ ID NO: 225 - protein, 19 aa, Homo sapiens
LLEVQVATGGVIDPQHHHR

SEQ ID NO: 226 - protein, 8 aa, Homo sapiens
LPLETAYR

SEQ ID NO: 227 - protein, 10 aa, Homo sapiens
DIYALISDQK

SEQ ID NO: 228 - protein, 9 aa, Homo sapiens
FVDPNTQEK

SEQ ID NO: 229 - protein, 10 aa, Homo sapiens
DSEHIDDETR

SEQ ID NO: 230 - protein, 13 aa, Homo sapiens
ALEAEQVEITVGR

SEQ ID NO: 231 - protein, 19 aa, Homo sapiens
GQKPTLWALLNSEYVTEEK

SEQ ID NO: 232 - protein, 15 aa, Homo sapiens
ALQTVAQLILELIEK

SEQ ID NO: 233 - protein, 6 aa, Homo sapiens
QETSNK

SEQ ID NO: 234 - protein, 8 aa, Homo sapiens
HLWFQGIR

SEQ ID NO: 235 - protein, 19 aa, Homo sapiens
LSVEEPVPAGVVGSEIQEK

SEQ ID NO: 236 - protein, 18 aa, Homo sapiens
GFFDPNTHENLTYVQLLR

SEQ ID NO: 237 - protein, 12 aa, Homo sapiens
GSAVHQLSEELR

SEQ ID NO: 238 - protein, 22 aa, Homo sapiens
VTPGSGALQGQSVSVWELLFYR

SEQ ID NO: 239 - protein, 6 aa, Homo sapiens
EVSEDR

SEQ ID NO: 240 - protein, 25 aa, Homo sapiens
AGTLTVEELGATLTSLLAQAQAQAR

SEQ ID NO: 241 - protein, 13 aa, Homo sapiens
AEAEAGSPRPDPR

SEQ ID NO: 242 - protein, 15 aa, Homo sapiens
AVPVWDVLASGYVSR

SEQ ID NO: 243 - protein, 19 aa, Homo sapiens
EELLAEFGSGTLDLPALTR

SEQ ID NO: 244 - protein, 22 aa, Homo sapiens
LTAIIEEAEEAPGARPQLQDAR

SEQ ID NO: 245 - protein, 7 aa, Homo sapiens
EPGPAGR

SEQ ID NO: 246 - protein, 7 aa, Homo sapiens
GDGDSGR

SEQ ID NO: 247 - protein, 19 aa, Homo sapiens
EGQGEGETQEAAAAAAAAR

SEQ ID NO: 248 - protein, 6 aa, Homo sapiens
QEQTLR

SEQ ID NO: 249 - protein, 23 aa, Homo sapiens
GQFQGRPVSVWDVLFSSYLSEAR

SEQ ID NO: 250 - protein, 23 aa, Homo sapiens
DELLAQHAAGALGLPDLVAVLTR

SEQ ID NO: 251 - protein, 8 aa, Homo sapiens
VIEETEER

SEQ ID NO: 252 - protein, 19 aa, Homo sapiens
QVSASELHTSGILGPETLR

SEQ ID NO: 253 - protein, 7 aa, Homo sapiens
DLAQGTK

SEQ ID NO: 254 - protein, 19 aa, Homo sapiens
LSVEEAVAAGVVGGEIQEK

SEQ ID NO: 255 - protein, 18 aa, Homo sapiens
AVPVWDVLASGYVSGAAR

SEQ ID NO: 256 - protein, 23 aa, Homo sapiens
LTAIIEEAEEAPGARPQLQDAWR

SEQ ID NO: 257 - protein, 25 aa, Homo sapiens
ASTLTVEELGATLTSLLAQAQAQAR

SEQ ID NO: 258 - protein, 19 aa, Homo sapiens
EGQGEGETQEAAAATAAAR

SEQ ID NO: 259 - protein, 19 aa, Homo sapiens
GFFDPNTHENLTYLQLLQR

SEQ ID NO: 260 - protein, 17 aa, Homo sapiens
ATLDPETGLLFLSLSLQ

SEQ ID NO: 261 - protein, 10 aa, Homo sapiens
SSVLESLVGR

SEQ ID NO: 262 - protein, 7 aa, Homo sapiens
GTGIVTR

SEQ ID NO: 263 - protein, 15 aa, Homo sapiens
RPLILQLVHVSQEDK

SEQ ID NO: 264 - protein, 15 aa, Homo sapiens
TTGEENGVEAEEWGK

SEQ ID NO: 265 - protein, 9 aa, Homo sapiens
LYTDFDEIR

SEQ ID NO: 266 - protein, 9 aa, Homo sapiens
QEIENETER

SEQ ID NO: 267 - protein, 6 aa, Homo sapiens
ISGNNK

SEQ ID NO: 268 - protein, 10 aa, Homo sapiens
GVSPEPIHLK

SEQ ID NO: 269 - protein, 8 aa, Homo sapiens
VPVGDQPK

SEQ ID NO: 270 - protein, 7 aa, Homo sapiens
DIELQIR

SEQ ID NO: 271 - protein, 7 aa, Homo sapiens
EVDPDGR

SEQ ID NO: 272 - protein, 7 aa, Homo sapiens
TLAVITK

SEQ ID NO: 273 - protein, 9 aa, Homo sapiens
LGIIGVVNR

SEQ ID NO: 274 - protein, 8 aa, Homo sapiens
SQLDINNK

SEQ ID NO: 275 - protein, 7 aa, Homo sapiens
SVTDSIR

SEQ ID NO: 276 - protein, 8 aa, Homo sapiens
DEYAFLQK

SEQ ID NO: 277 - protein, 7 aa, Homo sapiens
YPSLANR

SEQ ID NO: 278 - protein, 22 aa, Homo sapiens
INVLAAQYQSLLNSYGEPVDDK

SEQ ID NO: 279 - protein, 10 aa, Homo sapiens
SATLLQLITK

SEQ ID NO: 280 - protein, 21 aa, Homo sapiens
TLESVDPLGGLNTIDILTAIR

SEQ ID NO: 281 - protein, 21 aa, Homo sapiens
NATGPRPALFVPEVSFELLVK

SEQ ID NO: 282 - protein, 7 aa, Homo sapiens
LEEPSLR

SEQ ID NO: 283 - protein, 8 aa, Homo sapiens
ELPSAVSR

SEQ ID NO: 284 - protein, 23 aa, Homo sapiens
VPSALAPASQEPSPAASAEADGK

SEQ ID NO: 285 - protein, 6 aa, Homo sapiens
LIQDSR

SEQ ID NO: 286 - protein, 22 aa, Homo sapiens
NVASGGGGVGDGVQEPTTGNWR

SEQ ID NO: 287 - protein, 9 aa, Homo sapiens
AEELLAEEK

SEQ ID NO: 288 - protein, 15 aa, Homo sapiens
GHAVNLLDVPVPVAR

SEQ ID NO: 289 - protein, 7 aa, Homo sapiens
SYFLIVR

SEQ ID NO: 290 - protein, 8 aa, Homo sapiens
NIQDSVPK

SEQ ID NO: 291 - protein, 13 aa, Homo sapiens
DTLQSELVGQLYK

SEQ ID NO: 292 - protein, 13 aa, Homo sapiens
ALQGASQIIAEIR

SEQ ID NO: 293 - protein, 14 aa, Homo sapiens
YPPHSVQYTFPNTR

SEQ ID NO: 294 - protein, 11 aa, Homo sapiens
HQQEFAVPDYR

SEQ ID NO: 295 - protein, 22 aa, Homo sapiens
SSHLEVSQASQLLQQQQQQQLR

SEQ ID NO: 296 - protein, 18 aa, Homo sapiens
RPSLLSEFHPGSDRPQER

SEQ ID NO: 297 - protein, 21 aa, Homo sapiens
TSYEPFHPGPSPVDHDSLESK

SEQ ID NO: 298 - protein, 11 aa, Homo sapiens
LEQVSDSHFQR

SEQ ID NO: 299 - protein, 17 aa, Homo sapiens
VSAAVLPLVHPLPEGLR

SEQ ID NO: 300 - protein, 6 aa, Homo sapiens
ASADAK

SEQ ID NO: 301 - protein, 11 aa, Homo sapiens
SIVQIIYDENR

SEQ ID NO: 302 - protein, 8 aa, Homo sapiens
IFEGLGPK

SEQ ID NO: 303 - protein, 13 aa, Homo sapiens
VELPLYNQPSDTK

SEQ ID NO: 304 - protein, 7 aa, Homo sapiens
VYHENIK

SEQ ID NO: 305 - protein, 6 aa, Homo sapiens
LILFFK

SEQ ID NO: 306 - protein, 10 aa, Homo sapiens
GAGLSATIAR

SEQ ID NO: 307 - protein, 20 aa, Homo sapiens
SEHEISEIIDGLSEQENNEK

SEQ ID NO: 308 - protein, 6 aa, Homo sapiens
FIQHPK

SEQ ID NO: 309 - protein, 11 aa, Homo sapiens
NFGLIASYLER

SEQ ID NO: 310 - protein, 12 aa, Homo sapiens
NQQIARPSQEEK

SEQ ID NO: 311 - protein, 11 aa, Homo sapiens
IDGTAEETEER

SEQ ID NO: 312 - protein, 6 aa, Homo sapiens
EQATPR

SEQ ID NO: 313 - protein, 7 aa, Homo sapiens
GLVEHGR

SEQ ID NO: 314 - protein, 7 aa, Homo sapiens
NWAAIAK

SEQ ID NO: 315 - protein, 11 aa, Homo sapiens
HNLDNLLQQHK

SEQ ID NO: 316 - protein, 9 aa, Homo sapiens
ADSVDVEVR

SEQ ID NO: 317 - protein, 8 aa, Homo sapiens
VPENHASK

SEQ ID NO: 318 - protein, 7 aa, Homo sapiens
VEGDNTK

SEQ ID NO: 319 - protein, 21 aa, Homo sapiens
EWEVLQPAPHQVITNLPEGVR

SEQ ID NO: 320 - protein, 18 aa, Homo sapiens
LPTTRPTRPPPPLIPSSK

SEQ ID NO: 321 - protein, 6 aa, Homo sapiens
QQESAK

SEQ ID NO: 322 - protein, 8 aa, Homo sapiens
SATLPYIK

SEQ ID NO: 323 - protein, 7 aa, Homo sapiens
QEEFSPR

SEQ ID NO: 324 - protein, 12 aa, Homo sapiens
SQNSQPEGLLVR

SEQ ID NO: 325 - protein, 8 aa, Homo sapiens
AQHEGVVR

SEQ ID NO: 326 - protein, 13 aa, Homo sapiens
GTAGAIQEGSITR

SEQ ID NO: 327 - protein, 10 aa, Homo sapiens
ISVESIPSLR

SEQ ID NO: 328 - protein, 22 aa, Homo sapiens
GSITQGTPALPQTGIPTEALVK

SEQ ID NO: 329 - protein, 11 aa, Homo sapiens
SGHILSYDNIK

SEQ ID NO: 330 - protein, 8 aa, Homo sapiens
TAHEISLK

SEQ ID NO: 331 - protein, 10 aa, Homo sapiens
SYESVEGNIK

SEQ ID NO: 332 - protein, 8 aa, Homo sapiens
GSPHSDLK

SEQ ID NO: 333 - protein, 11 aa, Homo sapiens
ATTESFEDGLK

SEQ ID NO: 334 - protein, 6 aa, Homo sapiens
ESPPIR

SEQ ID NO: 335 - protein, 8 aa, Homo sapiens
AFEGAITK

SEQ ID NO: 336 - protein, 11 aa, Homo sapiens
GKPYDGITTIK

SEQ ID NO: 337 - protein, 9 aa, Homo sapiens
QDILTQESR

SEQ ID NO: 338 - protein, 23 aa, Homo sapiens
TPEVVQSTRPIIEGSISQGTPIK

SEQ ID NO: 339 - protein, 11 aa, Homo sapiens
FDNNSGQSAIK

SEQ ID NO: 340 - protein, 8 aa, Homo sapiens
SLITGPSK

SEQ ID NO: 341 - protein, 6 aa, Homo sapiens
AGETVR

SEQ ID NO: 342 - protein, 13 aa, Homo sapiens
HTSVVSSGPSVLR

SEQ ID NO: 343 - protein, 8 aa, Homo sapiens
STLHEAPK

SEQ ID NO: 344 - protein, 14 aa, Homo sapiens
AQLSPGIYDDTSAR

SEQ ID NO: 345 - protein, 10 aa, Homo sapiens
TSDVTISSNK

SEQ ID NO: 346 - protein, 6 aa, Homo sapiens
STNHER

SEQ ID NO: 347 - protein, 8 aa, Homo sapiens
STLTPTQR

SEQ ID NO: 348 - protein, 6 aa, Homo sapiens
ESIPAK

SEQ ID NO: 349 - protein, 20 aa, Homo sapiens
SPVPGVDPVVSHSPFDPHHR

SEQ ID NO: 350 - protein, 9 aa, Homo sapiens
GSTAGEVYR

SEQ ID NO: 351 - protein, 13 aa, Homo sapiens
ALDPAAAAYLFQR

SEQ ID NO: 352 - protein, 12 aa, Homo sapiens
EQPLGLPYPATR

SEQ ID NO: 353 - protein, 23 aa, Homo sapiens
YETPSDAIEVISPASSPAPPQEK

SEQ ID NO: 354 - protein, 10 aa, Homo sapiens
LQTYQPEVVK

SEQ ID NO: 355 - protein, 10 aa, Homo sapiens
ANQAENDPTR

SEQ ID NO: 356 - protein, 18 aa, Homo sapiens
YSPESQAQSVHHQRPGSR

SEQ ID NO: 357 - protein, 9 aa, Homo sapiens
VSPENLVDK

SEQ ID NO: 358 - protein, 22 aa, Homo sapiens
SHVSSEPYEPISPPQVPVVHEK

SEQ ID NO: 359 - protein, 10 aa, Homo sapiens
QDSLLLLSQR

SEQ ID NO: 360 - protein, 8 aa, Homo sapiens
GAEPAEQR

SEQ ID NO: 361 - protein, 14 aa, Homo sapiens
SPGSISYLPSFFTK

SEQ ID NO: 362 - protein, 18 aa, Homo sapiens
GHSFADPASNLGLEDIIR

SEQ ID NO: 363 - protein, 18 aa, Homo sapiens
EPAPLLSAQYETLSDSDD

SEQ ID NO: 364 - protein, 13 aa, Homo sapiens
SAAVSEQQQLEQK

SEQ ID NO: 365 - protein, 6 aa, Homo sapiens
TLEVEK

SEQ ID NO: 366 - protein, 15 aa, Homo sapiens
TTITAANFIDVIITR

SEQ ID NO: 367 - protein, 6 aa, Homo sapiens
QIASDK

SEQ ID NO: 368 - protein, 15 aa, Homo sapiens
GSQSSDSSSSLSSHR

SEQ ID NO: 369 - protein, 6 aa, Homo sapiens
GFGSDK

SEQ ID NO: 370 - protein, 10 aa, Homo sapiens
EAILDIITSR

SEQ ID NO: 371 - protein, 7 aa, Homo sapiens
DLIADLK

SEQ ID NO: 372 - protein, 6 aa, Homo sapiens
YELTGK

SEQ ID NO: 373 - protein, 11 aa, Homo sapiens
DAISGIGTDEK

SEQ ID NO: 374 - protein, 16 aa, Homo sapiens
DLEADIIGDTSGHFQK

SEQ ID NO: 375 - protein, 25 aa, Homo sapiens
EEDDVVSEDLVQQDVQDLYEAGELK

SEQ ID NO: 376 - protein, 15 aa, Homo sapiens
WGTDEAQFIYILGNR

SEQ ID NO: 377 - protein, 8 aa, Homo sapiens
LVFDEYLK

SEQ ID NO: 378 - protein, 11 aa, Homo sapiens
TTGKPIEASIR

SEQ ID NO: 379 - protein, 9 aa, Homo sapiens
GELSGDFEK

SEQ ID NO: 380 - protein, 9 aa, Homo sapiens
STPEYFAER

SEQ ID NO: 381 - protein, 8 aa, Homo sapiens
NDTSGEYK

SEQ ID NO: 382 - protein, 16 aa, Homo sapiens
GTVRPANDFNPDADAK

SEQ ID NO: 383 - protein, 16 aa, Homo sapiens
GLGTDEDTIIDIITHR

SEQ ID NO: 384 - protein, 9 aa, Homo sapiens
SEISGDLAR

SEQ ID NO: 385 - protein, 9 aa, Homo sapiens
ALIEILATR

SEQ ID NO: 386 - protein, 6 aa, Homo sapiens
TNAEIR

SEQ ID NO: 387 - protein, 7 aa, Homo sapiens
AINEAYK

SEQ ID NO: 388 - protein, 15 aa, Homo sapiens
SLEDALSSDTSGHFR

SEQ ID NO: 389 - protein, 10 aa, Homo sapiens
ILISLATGHR

SEQ ID NO: 390 - protein, 11 aa, Homo sapiens
EEGGENLDQAR

SEQ ID NO: 391 - protein, 20 aa, Homo sapiens
EDAQVAAEILEIADTPSGDK

SEQ ID NO: 392 - protein, 6 aa, Homo sapiens
SYPHLR

SEQ ID NO: 393 - protein, 7 aa, Homo sapiens
VFQEFIK

SEQ ID NO: 394 - protein, 11 aa, Homo sapiens
DAFVAIVQSVK

SEQ ID NO: 395 - protein, 9 aa, Homo sapiens
NKPLFFADK

SEQ ID NO: 396 - protein, 7 aa, Homo sapiens
GAGTDEK

SEQ ID NO: 397 - protein, 9 aa, Homo sapiens
SEIDLLNIR

SEQ ID NO: 398 - protein, 16 aa, Homo sapiens
SLHQAIEGDTSGDFLK

SEQ ID NO: 399 - protein, 14 aa, Homo sapiens
GFSSGSAVVSGGSR

SEQ ID NO: 400 - protein, 16 aa, Homo sapiens
HGGGGGGFGGGGFGSR

SEQ ID NO: 401 - protein, 9 aa, Homo sapiens
SLVGLGGTK

SEQ ID NO: 402 - protein, 22 aa, Homo sapiens
SISISVAGGGGGFGAAGGFGGR

SEQ ID NO: 403 - protein, 9 aa, Homo sapiens
VDPEIQNVK

SEQ ID NO: 404 - protein, 9 aa, Homo sapiens
YLDGLTAER

SEQ ID NO: 405 - protein, 11 aa, Homo sapiens
TAAENDFVTLK

SEQ ID NO: 406 - protein, 6 aa, Homo sapiens
VELQSK

SEQ ID NO: 407 - protein, 12 aa, Homo sapiens
VDLLNQEIEFLK

SEQ ID NO: 408 - protein, 10 aa, Homo sapiens
EEAEALYHSK

SEQ ID NO: 409 - protein, 10 aa, Homo sapiens
YEELQVTVGR

SEQ ID NO: 410 - protein, 9 aa, Homo sapiens
LQGEIAHVK

SEQ ID NO: 411 - protein, 12 aa, Homo sapiens
NVQDAIADAEQR

SEQ ID NO: 412 - protein, 6 aa, Homo sapiens
GEHALK

SEQ ID NO: 413 - protein, 12 aa, Homo sapiens
LNDLEEALQQAK

SEQ ID NO: 414 - protein, 9 aa, Homo sapiens
AAFGGSGGR

SEQ ID NO: 415 - protein, 20 aa, Homo sapiens
GSSSGGGYSSGSSSYGSGGR

SEQ ID NO: 416 - protein, 23 aa, Homo sapiens
GGSGGGGSISGGGYGSGGGSGGR

SEQ ID NO: 417 - protein, 8 aa, Homo sapiens
YGSGGGSK

SEQ ID NO: 418 - protein, 15 aa, Homo sapiens
GGSISGGGYGSGGGK

SEQ ID NO: 419 - protein, 8 aa, Homo sapiens
HSSGGGSR

SEQ ID NO: 420 - protein, 20 aa, Homo sapiens
GGSSSGGGYGSGGGGSSSVK

SEQ ID NO: 421 - protein, 16 aa, Homo sapiens
GSSGEAFGSSVTFSFR

SEQ ID NO: 422 - protein, 15 aa, Homo sapiens
VGAHAGEYGAEALER

SEQ ID NO: 423 - protein, 16 aa, Homo sapiens
TYFPHFDLSHGSAQVK

SEQ ID NO: 424 - protein, 12 aa, Homo sapiens
FLASVSTVLTSK

SEQ ID NO: 425 - protein, 20 aa, Homo sapiens
ELLLPNWQGSGSHGLTIAQR

SEQ ID NO: 426 - protein, 17 aa, Homo sapiens
DDGVFVQEVTQNSPAAR

SEQ ID NO: 427 - protein, 10 aa, Homo sapiens
SPEPGQTWTR

SEQ ID NO: 428 - protein, 15 aa, Homo sapiens
SEDGVEGDLGETQSR

SEQ ID NO: 429 - protein, 6 aa, Homo sapiens
TITVTR

SEQ ID NO: 430 - protein, 11 aa, Homo sapiens
VTAYTVDVTGR

SEQ ID NO: 431 - protein, 10 aa, Homo sapiens
DIDISSPEFK

SEQ ID NO: 432 - protein, 17 aa, Homo sapiens
HELTEISNVDVETQSGK

SEQ ID NO: 433 - protein, 18 aa, Homo sapiens
LPSGSGAASPTGSAVDIR

SEQ ID NO: 434 - protein, 18 aa, Homo sapiens
AGAISASGPELQGAGHSK

SEQ ID NO: 435 - protein, 11 aa, Homo sapiens
VGGSGVNVNAK

SEQ ID NO: 436 - protein, 7 aa, Homo sapiens
GLDLGGR

SEQ ID NO: 437 - protein, 17 aa, Homo sapiens
GGVQVPAVDISSSLGGR

SEQ ID NO: 438 - protein, 16 aa, Homo sapiens
AVEVQGPSLESGDHGK

SEQ ID NO: 439 - protein, 7 aa, Homo sapiens
FGVSTGR

SEQ ID NO: 440 - protein, 6 aa, Homo sapiens
EGQTPK

SEQ ID NO: 441 - protein, 11 aa, Homo sapiens
VSAPEVSVGHK

SEQ ID NO: 442 - protein, 16 aa, Homo sapiens
GPQITGPSLEGDLGLK

SEQ ID NO: 443 - protein, 7 aa, Homo sapiens
FSVSGAK

SEQ ID NO: 444 - protein, 11 aa, Homo sapiens
VSAPGVQGDVK

SEQ ID NO: 445 - protein, 7 aa, Homo sapiens
GPQVALK

SEQ ID NO: 446 - protein, 16 aa, Homo sapiens
VDIETPNLEGTLTGPR

SEQ ID NO: 447 - protein, 7 aa, Homo sapiens
LGSPSGK

SEQ ID NO: 448 - protein, 9 aa, Homo sapiens
GGVDVTLPR

SEQ ID NO: 449 - protein, 7 aa, Homo sapiens
VPEVDVR

SEQ ID NO: 450 - protein, 18 aa, Homo sapiens
VDVSAPDVEAHGPEWNLK

SEQ ID NO: 451 - protein, 9 aa, Homo sapiens
GDISISGPK

SEQ ID NO: 452 - protein, 16 aa, Homo sapiens
VNVEAPDVNLEGLGGK

SEQ ID NO: 453 - protein, 9 aa, Homo sapiens
GEYDVTVPK

SEQ ID NO: 454 - protein, 18 aa, Homo sapiens
VDIDAPDVDVHGPDWHLK

SEQ ID NO: 455 - protein, 7 aa, Homo sapiens
FSVPGFK

SEQ ID NO: 456 - protein, 12 aa, Homo sapiens
AEGPEVDVNLPK

SEQ ID NO: 457 - protein, 9 aa, Homo sapiens
ADVDISGPK

SEQ ID NO: 458 - protein, 16 aa, Homo sapiens
IDVTAPDVSIEEPEGK

SEQ ID NO: 459 - protein, 7 aa, Homo sapiens
VPDVELK

SEQ ID NO: 460 - protein, 16 aa, Homo sapiens
VGVEVPDVNIEGPEGK

SEQ ID NO: 461 - protein, 12 aa, Homo sapiens
GEGPEFDVNLSK

SEQ ID NO: 462 - protein, 9 aa, Homo sapiens
ANVDISAPK

SEQ ID NO: 463 - protein, 16 aa, Homo sapiens
VDTNAPDLSLEGPEGK

SEQ ID NO: 464 - protein, 9 aa, Homo sapiens
GNVDISAPK

SEQ ID NO: 465 - protein, 14 aa, Homo sapiens
APDVEGQGLDWSLK

SEQ ID NO: 466 - protein, 12 aa, Homo sapiens
GEGPEVDVNLPK

SEQ ID NO: 467 - protein, 9 aa, Homo sapiens
ADVVVSGPK

SEQ ID NO: 468 - protein, 16 aa, Homo sapiens
VDIEAPDVSLEGPEGK

SEQ ID NO: 469 - protein, 9 aa, Homo sapiens
GDVDVSVPK

SEQ ID NO: 470 - protein, 7 aa, Homo sapiens
VPDVEIK

SEQ ID NO: 471 - protein, 8 aa, Homo sapiens
EVDVNLPK

SEQ ID NO: 472 - protein, 9 aa, Homo sapiens
ADIDVSGPK

SEQ ID NO: 473 - protein, 16 aa, Homo sapiens
VDVEVPDVSLEGPEGK

SEQ ID NO: 474 - protein, 18 aa, Homo sapiens
VDISAPDVDVHGPDWHLK

SEQ ID NO: 475 - protein, 9 aa, Homo sapiens
GEGPEVDVK

SEQ ID NO: 476 - protein, 9 aa, Homo sapiens
ADVDVSGPK

SEQ ID NO: 477 - protein, 11 aa, Homo sapiens
ISIPDVGLHLK

SEQ ID NO: 478 - protein, 9 aa, Homo sapiens
GDYDVTVPK

SEQ ID NO: 479 - protein, 6 aa, Homo sapiens
VEGEIK

SEQ ID NO: 480 - protein, 7 aa, Homo sapiens
APDVDIK

SEQ ID NO: 481 - protein, 18 aa, Homo sapiens
VDINAPDVEVHGPDWHLK

SEQ ID NO: 482 - protein, 9 aa, Homo sapiens
ADLGVSGPK

SEQ ID NO: 483 - protein, 16 aa, Homo sapiens
VDIDVPDVNLEAPEGK

SEQ ID NO: 484 - protein, 9 aa, Homo sapiens
TDVDVSLPK

SEQ ID NO: 485 - protein, 6 aa, Homo sapiens
VEGDLK

SEQ ID NO: 486 - protein, 7 aa, Homo sapiens
GPEIDVK

SEQ ID NO: 487 - protein, 18 aa, Homo sapiens
VDIDAPDVEVHDPDWHLK

SEQ ID NO: 488 - protein, 25 aa, Homo sapiens
ADIDVSGPSVDTDAPDLDIEGPEGK

SEQ ID NO: 489 - protein, 15 aa, Homo sapiens
GEIDASVPELEGDLR

SEQ ID NO: 490 - protein, 9 aa, Homo sapiens
GDADVSVPK

SEQ ID NO: 491 - protein, 9 aa, Homo sapiens
GDVDVSLPK

SEQ ID NO: 492 - protein, 7 aa, Homo sapiens
VPDVDIR

SEQ ID NO: 493 - protein, 9 aa, Homo sapiens
GDVDVSAPK

SEQ ID NO: 494 - protein, 7 aa, Homo sapiens
GPELDVK

SEQ ID NO: 495 - protein, 9 aa, Homo sapiens
GEVDVDVPK

SEQ ID NO: 496 - protein, 19 aa, Homo sapiens
GPHVDVSGPDIDIEGPEGK

SEQ ID NO: 497 - protein, 13 aa, Homo sapiens
GDVDVSVPEVEGK

SEQ ID NO: 498 - protein, 16 aa, Homo sapiens
VDVNAPDVQAPDWHLK

SEQ ID NO: 499 - protein, 16 aa, Homo sapiens
VDIEGPDVNIEGPEGK

SEQ ID NO: 500 - protein, 7 aa, Homo sapiens
GPEVDIK

SEQ ID NO: 501 - protein, 18 aa, Homo sapiens
VDINAPDVGVQGPDWHLK

SEQ ID NO: 502 - protein, 9 aa, Homo sapiens
GEGPDGDVK

SEQ ID NO: 503 - protein, 12 aa, Homo sapiens
GEGPDVDVNLPK

SEQ ID NO: 504 - protein, 9 aa, Homo sapiens
GDVDVTGPK

SEQ ID NO: 505 - protein, 7 aa, Homo sapiens
GPEVDLK

SEQ ID NO: 506 - protein, 18 aa, Homo sapiens
VDIDVPDVNVQGPDWHLK

SEQ ID NO: 507 - protein, 16 aa, Homo sapiens
VDVEGPDVNIEGPEGK

SEQ ID NO: 508 - protein, 9 aa, Homo sapiens
GDVDISLPK

SEQ ID NO: 509 - protein, 7 aa, Homo sapiens
GPEVDIR

SEQ ID NO: 510 - protein, 21 aa, Homo sapiens
GPQVDIDVPDVGVQGPDWHLK

SEQ ID NO: 511 - protein, 9 aa, Homo sapiens
ADLDVSGPK

SEQ ID NO: 512 - protein, 16 aa, Homo sapiens
VDIDVPDVNIEGPEGK

SEQ ID NO: 513 - protein, 18 aa, Homo sapiens
VDINAPDVDVQGPDWHLK

SEQ ID NO: 514 - protein, 16 aa, Homo sapiens
VDVDVPDVNIEGPDAK

SEQ ID NO: 515 - protein, 15 aa, Homo sapiens
GEVDVSLANVEGDLK

SEQ ID NO: 516 - protein, 7 aa, Homo sapiens
GPALDIK

SEQ ID NO: 517 - protein, 16 aa, Homo sapiens
IDVDAPDIDIHGPDAK

SEQ ID NO: 518 - protein, 9 aa, Homo sapiens
GDVDVSGPK

SEQ ID NO: 519 - protein, 7 aa, Homo sapiens
APSLDIK

SEQ ID NO: 520 - protein, 10 aa, Homo sapiens
GPEVDVSGPK

SEQ ID NO: 521 - protein, 6 aa, Homo sapiens
LNIEGK

SEQ ID NO: 522 - protein, 7 aa, Homo sapiens
FNFSGSK

SEQ ID NO: 523 - protein, 9 aa, Homo sapiens
VQTPEVDVK

SEQ ID NO: 524 - protein, 10 aa, Homo sapiens
KPDIDITGPK

SEQ ID NO: 525 - protein, 13 aa, Homo sapiens
VDINAPDVEVQGK

SEQ ID NO: 526 - protein, 15 aa, Homo sapiens
GDLDIAGPNLEGDFK

SEQ ID NO: 527 - protein, 21 aa, Homo sapiens
APEVNLNAPDVDVHGPDWNLK

SEQ ID NO: 528 - protein, 12 aa, Homo sapiens
AEGPDVAVDLPK

SEQ ID NO: 529 - protein, 18 aa, Homo sapiens
VDINAPDVDVHGPDWHLK

SEQ ID NO: 530 - protein, 12 aa, Homo sapiens
GEGPEVDVTLPK

SEQ ID NO: 531 - protein, 25 aa, Homo sapiens
ADIDISGPNVDVDVPDVNIEGPDAK

SEQ ID NO: 532 - protein, 9 aa, Homo sapiens
GDVVVSLPK

SEQ ID NO: 533 - protein, 16 aa, Homo sapiens
VDIDTPDINIEGSEGK

SEQ ID NO: 534 - protein, 11 aa, Homo sapiens
VDINAPDVDVR

SEQ ID NO: 535 - protein, 7 aa, Homo sapiens
GPDWHLK

SEQ ID NO: 536 - protein, 7 aa, Homo sapiens
APEVDIK

SEQ ID NO: 537 - protein, 16 aa, Homo sapiens
VDIDTPDIDIHGPEGK

SEQ ID NO: 538 - protein, 18 aa, Homo sapiens
VDIDVPDVDVQGPDWHLK

SEQ ID NO: 539 - protein, 16 aa, Homo sapiens
VDIDVPDVNIEGPDAK

SEQ ID NO: 540 - protein, 12 aa, Homo sapiens
GEGPDVDVTLPK

SEQ ID NO: 541 - protein, 9 aa, Homo sapiens
ADIEISGPK

SEQ ID NO: 542 - protein, 16 aa, Homo sapiens
VDIDAPDVSIEGPDAK

SEQ ID NO: 543 - protein, 19 aa, Homo sapiens
GPSLDIDTPDVNIEGPEGK

SEQ ID NO: 544 - protein, 12 aa, Homo sapiens
GPEVDIEGPEGK

SEQ ID NO: 545 - protein, 16 aa, Homo sapiens
VGIDTPDIDIHGPEGK

SEQ ID NO: 546 - protein, 9 aa, Homo sapiens
GDVDVTLPK

SEQ ID NO: 547 - protein, 7 aa, Homo sapiens
GPEADIK

SEQ ID NO: 548 - protein, 18 aa, Homo sapiens
VDINTPDVDVHGPDWHLK

SEQ ID NO: 549 - protein, 12 aa, Homo sapiens
GEGPDVDVSLPK

SEQ ID NO: 550 - protein, 16 aa, Homo sapiens
VDVDIPDVNIEGPDAK

SEQ ID NO: 551 - protein, 11 aa, Homo sapiens
ISIPDVDLDLK

SEQ ID NO: 552 - protein, 9 aa, Homo sapiens
GDFDVSVPK

SEQ ID NO: 553 - protein, 6 aa, Homo sapiens
VEGTLK

SEQ ID NO: 554 - protein, 9 aa, Homo sapiens
LDFEGPDAK

SEQ ID NO: 555 - protein, 7 aa, Homo sapiens
LSGPSLK

SEQ ID NO: 556 - protein, 11 aa, Homo sapiens
VTAPDVDLHLK

SEQ ID NO: 557 - protein, 7 aa, Homo sapiens
IGFSGPK

SEQ ID NO: 558 - protein, 9 aa, Homo sapiens
LEGGEVDLK

SEQ ID NO: 559 - protein, 6 aa, Homo sapiens
FGFGAK

SEQ ID NO: 560 - protein, 25 aa, Homo sapiens
SPSLDVTVPEAELNLETPEISVGGK

SEQ ID NO: 561 - protein, 17 aa, Homo sapiens
QGFDLNVPGGEIDASLK

SEQ ID NO: 562 - protein, 16 aa, Homo sapiens
APDVDVNIAGPDAALK

SEQ ID NO: 563 - protein, 9 aa, Homo sapiens
AEAPLPSPK

SEQ ID NO: 564 - protein, 25 aa, Homo sapiens
LEGELQAPDLELSLPAIHVEGLDIK

SEQ ID NO: 565 - protein, 6 aa, Homo sapiens
IEGDLK

SEQ ID NO: 566 - protein, 18 aa, Homo sapiens
VQANLGAPDINIEGLDAK

SEQ ID NO: 567 - protein, 21 aa, Homo sapiens
TPSFGISAPQVSIPDVNVNLK

SEQ ID NO: 568 - protein, 21 aa, Homo sapiens
GDVPSVGLEGPDVDLQGPEAK

SEQ ID NO: 569 - protein, 7 aa, Homo sapiens
IGIPGVK

SEQ ID NO: 570 - protein, 9 aa, Homo sapiens
LEGPDVSLK

SEQ ID NO: 571 - protein, 11 aa, Homo sapiens
VSGPDLDLNLK

SEQ ID NO: 572 - protein, 14 aa, Homo sapiens
VHAPGLNLSGVGGK

SEQ ID NO: 573 - protein, 9 aa, Homo sapiens
VPGIDATTK

SEQ ID NO: 574 - protein, 11 aa, Homo sapiens
LNVGAPDVTLR

SEQ ID NO: 575 - protein, 15 aa, Homo sapiens
GPSLQGDLAVSGDIK

SEQ ID NO: 576 - protein, 18 aa, Homo sapiens
VSVGAPDLSLEASEGSIK

SEQ ID NO: 577 - protein, 18 aa, Homo sapiens
LPQFGISTPGSDLHVNAK

SEQ ID NO: 578 - protein, 9 aa, Homo sapiens
GPQVSGELK

SEQ ID NO: 579 - protein, 9 aa, Homo sapiens
GPGVDVNLK

SEQ ID NO: 580 - protein, 14 aa, Homo sapiens
ISAPNVDFNLEGPK

SEQ ID NO: 581 - protein, 10 aa, Homo sapiens
GSLGATGEIK

SEQ ID NO: 582 - protein, 12 aa, Homo sapiens
LPTGQISGPEIK

SEQ ID NO: 583 - protein, 16 aa, Homo sapiens
GSEVGFHGAAPDISVK

SEQ ID NO: 584 - protein, 24 aa, Homo sapiens
GGADVSGGVSAPDISLGEGHLSVK

SEQ ID NO: 585 - protein, 7 aa, Homo sapiens
GSGGEWK

SEQ ID NO: 586 - protein, 14 aa, Homo sapiens
GPQVSSALNLDTSK

SEQ ID NO: 587 - protein, 11 aa, Homo sapiens
FAGGLHFSGPK

SEQ ID NO: 588 - protein, 6 aa, Homo sapiens
VEGGVK

SEQ ID NO: 589 - protein, 6 aa, Homo sapiens
FTFSGR

SEQ ID NO: 590 - protein, 20 aa, Homo sapiens
AEASIQAGAGDGEWEESEVK

SEQ ID NO: 591 - protein, 7 aa, Homo sapiens
FNFSKPK

SEQ ID NO: 592 - protein, 15 aa, Homo sapiens
GGVTGSPEASISGSK

SEQ ID NO: 593 - protein, 18 aa, Homo sapiens
ASLGSLEGEAEAEASSPK

SEQ ID NO: 594 - protein, 8 aa, Homo sapiens
SNSFSDER

SEQ ID NO: 595 - protein, 25 aa, Homo sapiens
EFSGPSTPTGTLEFEGGEVSLEGGK

SEQ ID NO: 596 - protein, 10 aa, Homo sapiens
FGTFGGLGSK

SEQ ID NO: 597 - protein, 14 aa, Homo sapiens
GHYEVTGSDDETGK

SEQ ID NO: 598 - protein, 11 aa, Homo sapiens
LQGSGVSLASK

SEQ ID NO: 599 - protein, 12 aa, Homo sapiens
LSSSSSNDSGNK

SEQ ID NO: 600 - protein, 15 aa, Homo sapiens
VGIQLPEVELSVSTK

SEQ ID NO: 601 - protein, 9 aa, Homo sapiens
VDGSVDFYR

SEQ ID NO: 602 - protein, 6 aa, Homo sapiens
DWATYK

SEQ ID NO: 603 - protein, 23 aa, Homo sapiens
LGEFWLGNDNIHALTAQGTSELR

SEQ ID NO: 604 - protein, 14 aa, Homo sapiens
VDLVDFEDNYQFAK

SEQ ID NO: 605 - protein, 13 aa, Homo sapiens
GTHGSFANGINWK

SEQ ID NO: 606 - protein, 7 aa, Homo sapiens
GYNYSYK

SEQ ID NO: 607 - protein, 8 aa, Homo sapiens
GEAGTNGK

SEQ ID NO: 608 - protein, 9 aa, Homo sapiens
LVASSHISK

SEQ ID NO: 609 - protein, 9 aa, Homo sapiens
TSLSVDPSR

SEQ ID NO: 610 - protein, 11 aa, Homo sapiens
GYPEPQVTWHR

SEQ ID NO: 611 - protein, 10 aa, Homo sapiens
NGQPITSGGR

SEQ ID NO: 612 - protein, 15 aa, Homo sapiens
GTFSLVIHAVHEEDR

SEQ ID NO: 613 - protein, 14 aa, Homo sapiens
QVTVELTVEGSFAK

SEQ ID NO: 614 - protein, 9 aa, Homo sapiens
QLGQPVVSK

SEQ ID NO: 615 - protein, 13 aa, Homo sapiens
ITGRPQPQVTWLK

SEQ ID NO: 616 - protein, 10 aa, Homo sapiens
GNVPLQPSAR

SEQ ID NO: 617 - protein, 6 aa, Homo sapiens
VSVSEK

SEQ ID NO: 618 - protein, 7 aa, Homo sapiens
ATNSDVR

SEQ ID NO: 619 - protein, 8 aa, Homo sapiens
EVTNVISK

SEQ ID NO: 620 - protein, 9 aa, Homo sapiens
LDSLEAAAK

SEQ ID NO: 621 - protein, 16 aa, Homo sapiens
GGSPPWAANSQPQPPR

SEQ ID NO: 622 - protein, 10 aa, Homo sapiens
TAPQTPVLQK

SEQ ID NO: 623 - protein, 11 aa, Homo sapiens
TSSSITLQAAR

SEQ ID NO: 624 - protein, 6 aa, Homo sapiens
VQPEPR

SEQ ID NO: 625 - protein, 14 aa, Homo sapiens
APGLGVLSPSGEER

SEQ ID NO: 626 - protein, 13 aa, Homo sapiens
RPAPPRPATFPTR

SEQ ID NO: 627 - protein, 12 aa, Homo sapiens
QPGLGSQDVVSK

SEQ ID NO: 628 - protein, 6 aa, Homo sapiens
DSAFPK

SEQ ID NO: 629 - protein, 11 aa, Homo sapiens
FESKPQSQEVK

SEQ ID NO: 630 - protein, 6 aa, Homo sapiens
ENQTVK

SEQ ID NO: 631 - protein, 14 aa, Homo sapiens
ITWLLNGQPIQYAR

SEQ ID NO: 632 - protein, 25 aa, Homo sapiens
SEYLLPVAPSKPTAPIFLQGLSDLK

SEQ ID NO: 633 - protein, 23 aa, Homo sapiens
TQAVLTVQEPHDGTQPWFISKPR

SEQ ID NO: 634 - protein, 19 aa, Homo sapiens
DTGHFEVLQNEDVFTLVLK

SEQ ID NO: 635 - protein, 14 aa, Homo sapiens
VQPWHAGQYEILLK

SEQ ID NO: 636 - protein, 11 aa, Homo sapiens
YGSLRPGWPAR

SEQ ID NO: 637 - protein, 14 aa, Homo sapiens
GQGWLEEEDGEDVR

SEQ ID NO: 638 - protein, 8 aa, Homo sapiens
QHTEEAIR

SEQ ID NO: 639 - protein, 10 aa, Homo sapiens
QQEVEQLDFR

SEQ ID NO: 640 - protein, 8 aa, Homo sapiens
TLSEDDLK

SEQ ID NO: 641 - protein, 7 aa, Homo sapiens
TVSEEER

SEQ ID NO: 642 - protein, 10 aa, Homo sapiens
VHSPQQVDFR

SEQ ID NO: 643 - protein, 12 aa, Homo sapiens
VPPPKPATPDFR

SEQ ID NO: 644 - protein, 16 aa, Homo sapiens
LPAENGSSSAETLNAK

SEQ ID NO: 645 - protein, 10 aa, Homo sapiens
GHAGTTDNEK

SEQ ID NO: 646 - protein, 11 aa, Homo sapiens
SESQGTAPAFK

SEQ ID NO: 647 - protein, 10 aa, Homo sapiens
LQDVHVAEGK

SEQ ID NO: 648 - protein, 6 aa, Homo sapiens
ALPEDR

SEQ ID NO: 649 - protein, 16 aa, Homo sapiens
SSLPPVLGTESDATVK

SEQ ID NO: 650 - protein, 10 aa, Homo sapiens
AGESVELFGK

SEQ ID NO: 651 - protein, 9 aa, Homo sapiens
VENSENGSK

SEQ ID NO: 652 - protein, 7 aa, Homo sapiens
LTILAAR

SEQ ID NO: 653 - protein, 16 aa, Homo sapiens
STSFNVQDLLPDHEYK

SEQ ID NO: 654 - protein, 11 aa, Homo sapiens
DEVEVSDDDEK

SEQ ID NO: 655 - protein, 7 aa, Homo sapiens
EPEVDYR

SEQ ID NO: 656 - protein, 9 aa, Homo sapiens
TVTINTEQK

SEQ ID NO: 657 - protein, 10 aa, Homo sapiens
VSDFYDIEER

SEQ ID NO: 658 - protein, 11 aa, Homo sapiens
IIDEDFELTER

SEQ ID NO: 659 - protein, 11 aa, Homo sapiens
QISEGVEYIHK

SEQ ID NO: 660 - protein, 8 aa, Homo sapiens
LIDFGLAR

SEQ ID NO: 661 - protein, 8 aa, Homo sapiens
LENAGSLK

SEQ ID NO: 662 - protein, 8 aa, Homo sapiens
DFISNLLK

SEQ ID NO: 663 - protein, 6 aa, Homo sapiens
TGNAVR

SEQ ID NO: 664 - protein, 14 aa, Homo sapiens
SSTGSPTSPLNAEK

SEQ ID NO: 665 - protein, 11 aa, Homo sapiens
DLEVVEGSAAR

SEQ ID NO: 666 - protein, 13 aa, Homo sapiens
IEGYPDPEVVWFK

SEQ ID NO: 667 - protein, 6 aa, Homo sapiens
DDQSIR

SEQ ID NO: 668 - protein, 7 aa, Homo sapiens
VIEGINR

SEQ ID NO: 669 - protein, 7 aa, Homo sapiens
GLSNAER

SEQ ID NO: 670 - protein, 14 aa, Homo sapiens
ALDGINSGITHAGR

SEQ ID NO: 671 - protein, 14 aa, Homo sapiens
VAHEINHGIGQAGK

SEQ ID NO: 672 - protein, 14 aa, Homo sapiens
AVQGFHTGVHQAGK

SEQ ID NO: 673 - protein, 14 aa, Homo sapiens
LGQGVNHAADQAGK

SEQ ID NO: 674 - protein, 14 aa, Homo sapiens
LGQGAHHAAGQAGK

SEQ ID NO: 675 - protein, 14 aa, Homo sapiens
ELQNAHNGVNQASK

SEQ ID NO: 676 - protein, 14 aa, Homo sapiens
LGHGVNNAAGQVGK

SEQ ID NO: 677 - protein, 19 aa, Homo sapiens
LIHHGVHHGANQAGSEAGK

SEQ ID NO: 678 - protein, 18 aa, Homo sapiens
FGQGVDNAAGQAGNEAGR

SEQ ID NO: 679 - protein, 18 aa, Homo sapiens
FGQGVHHAAGQAGNEAGR

SEQ ID NO: 680 - protein, 14 aa, Homo sapiens
FGQGAHHGLSEGWK

SEQ ID NO: 681 - protein, 14 aa, Homo sapiens
FGQGIHHAAGQVGK

SEQ ID NO: 682 - protein, 18 aa, Homo sapiens
FGQGAHHAAGQAGNEAGR

SEQ ID NO: 683 - protein, 14 aa, Homo sapiens
FGQGVHHGLSEGWK

SEQ ID NO: 684 - protein, 14 aa, Homo sapiens
FGQGVHHTAGQVGK

SEQ ID NO: 685 - protein, 14 aa, Homo sapiens
FGQGVHHAASQFGK

SEQ ID NO: 686 - protein, 14 aa, Homo sapiens
LGHGVHHGVNEAWK

SEQ ID NO: 687 - protein, 14 aa, Homo sapiens
FGQGVHHAASQVGK

SEQ ID NO: 688 - protein, 14 aa, Homo sapiens
VVQGLHHGVSQAGR

SEQ ID NO: 689 - protein, 18 aa, Homo sapiens
EAGQFGHDIHHTAGQAGK

SEQ ID NO: 690 - protein, 18 aa, Homo sapiens
EGDIAVHGVQPGVHEAGK

SEQ ID NO: 691 - protein, 18 aa, Homo sapiens
EAGQFGQGVHHTLEQAGK

SEQ ID NO: 692 - protein, 10 aa, Homo sapiens
INAANYASVK

SEQ ID NO: 693 - protein, 6 aa, Homo sapiens
QLPNHR

SEQ ID NO: 694 - protein, 14 aa, Homo sapiens
EGTIQVQGQALFFR

SEQ ID NO: 695 - protein, 10 aa, Homo sapiens
EALPGSGQAR

SEQ ID NO: 696 - protein, 10 aa, Homo sapiens
FSVLLLHGIR

SEQ ID NO: 697 - protein, 14 aa, Homo sapiens
FSSETWQNLGTLHR

SEQ ID NO: 698 - protein, 13 aa, Homo sapiens
AVAIDLPGLGHSK

SEQ ID NO: 699 - protein, 6 aa, Homo sapiens
QAPGQR

SEQ ID NO: 700 - protein, 8 aa, Homo sapiens
WPEPVFGR

SEQ ID NO: 701 - protein, 16 aa, Homo sapiens
LASPGFPGEYANDQER

SEQ ID NO: 702 - protein, 10 aa, Homo sapiens
WTLTAPPGYR

SEQ ID NO: 703 - protein, 15 aa, Homo sapiens
DTFYSLGSSLDITFR

SEQ ID NO: 704 - protein, 7 aa, Homo sapiens
AGYVLHR

SEQ ID NO: 705 - protein, 15 aa, Homo sapiens
SGELSSPEYPRPYPK

SEQ ID NO: 706 - protein, 20 aa, Homo sapiens
SNTVTITFVTDESGDHTGWK

SEQ ID NO: 707 - protein, 13 aa, Homo sapiens
VEYITGPGVTTYK

SEQ ID NO: 708 - protein, 6 aa, Homo sapiens
IYGGQK

SEQ ID NO: 709 - protein, 9 aa, Homo sapiens
HDASALDIR

SEQ ID NO: 710 - protein, 15 aa, Homo sapiens
TDDIGTASGWGLTQR

SEQ ID NO: 711 - protein, 16 aa, Homo sapiens
GDSGGALVFLDSETER

SEQ ID NO: 712 - protein, 15 aa, Homo sapiens
VINYIPWIENIISDF

SEQ ID NO: 713 - protein, 14 aa, Homo sapiens
SPDELPSAGGDGGK

SEQ ID NO: 714 - protein, 8 aa, Homo sapiens
EKPNSAHR

SEQ ID NO: 715 - protein, 10 aa, Homo sapiens
EETAGSYDSR

SEQ ID NO: 716 - protein, 9 aa, Homo sapiens
FQPLLDGLK

SEQ ID NO: 717 - protein, 8 aa, Homo sapiens
SGTTIALK

SEQ ID NO: 718 - protein, 11 aa, Homo sapiens
LGLHQVLQDLR

SEQ ID NO: 719 - protein, 18 aa, Homo sapiens
VQLNVFDEQGEEDSYDLK

SEQ ID NO: 720 - protein, 16 aa, Homo sapiens
AEPHFLSILQHLLLVR

SEQ ID NO: 721 - protein, 11 aa, Homo sapiens
NDYEARPQYYK

SEQ ID NO: 722 - protein, 8 aa, Homo sapiens
NGADPDFK

SEQ ID NO: 723 - protein, 8 aa, Homo sapiens
LQDLQGEK

SEQ ID NO: 724 - protein, 7 aa, Homo sapiens
DALHSEK

SEQ ID NO: 725 - protein, 7 aa, Homo sapiens
QQIATEK

SEQ ID NO: 726 - protein, 16 aa, Homo sapiens
QDLEAEVSQLTGEVAK

SEQ ID NO: 727 - protein, 9 aa, Homo sapiens
LYKPEVQLR

SEQ ID NO: 728 - protein, 6 aa, Homo sapiens
RPNWSK

SEQ ID NO: 729 - protein, 9 aa, Homo sapiens
FENNELFAK

SEQ ID NO: 730 - protein, 10 aa, Homo sapiens
LTLTFSAQTK

SEQ ID NO: 731 - protein, 8 aa, Homo sapiens
DQEGGEEK

SEQ ID NO: 732 - protein, 13 aa, Homo sapiens
TAQNLSIFLGSFR

SEQ ID NO: 733 - protein, 7 aa, Homo sapiens
LNAILFK

SEQ ID NO: 734 - protein, 10 aa, Homo sapiens
FPDELAHVEK

SEQ ID NO: 735 - protein, 8 aa, Homo sapiens
VSAENLQK

SEQ ID NO: 736 - protein, 7 aa, Homo sapiens
QISDVER

SEQ ID NO: 737 - protein, 12 aa, Homo sapiens
DVQNFPAATDEK

SEQ ID NO: 738 - protein, 8 aa, Homo sapiens
DAQEQYNK

SEQ ID NO: 739 - protein, 11 aa, Homo sapiens
ELGEYFLFDPK

SEQ ID NO: 740 - protein, 13 aa, Homo sapiens
NSETFPTILEEAK

SEQ ID NO: 741 - protein, 17 aa, Homo sapiens
VPAQLLGLLLLWLPGAK

SEQ ID NO: 742 - protein, 18 aa, Homo sapiens
ASQSISSWLAWYQQKPGK

SEQ ID NO: 743 - protein, 17 aa, Homo sapiens
EVNTSGFAPARPPPQPR

SEQ ID NO: 744 - protein, 11 aa, Homo sapiens
DDVSISVEEGK

SEQ ID NO: 745 - protein, 20 aa, Homo sapiens
ENILHVSENVIFTDVNSILR

SEQ ID NO: 746 - protein, 10 aa, Homo sapiens
GNAAWQEQLK

SEQ ID NO: 747 - protein, 6 aa, Homo sapiens
APVHVK

SEQ ID NO: 748 - protein, 17 aa, Homo sapiens
WFGFLEAQQAFQSVGTK

SEQ ID NO: 749 - protein, 7 aa, Homo sapiens
WDVSTTK

SEQ ID NO: 750 - protein, 15 aa, Homo sapiens
FPPEASGYLHIGHAK

SEQ ID NO: 751 - protein, 13 aa, Homo sapiens
AALLNQHYQVNFK

SEQ ID NO: 752 - protein, 6 aa, Homo sapiens
IQPHPR

SEQ ID NO: 753 - protein, 7 aa, Homo sapiens
TTEYHDR

SEQ ID NO: 754 - protein, 14 aa, Homo sapiens
DEQFYWIIEALGIR

SEQ ID NO: 755 - protein, 9 aa, Homo sapiens
KPYIWEYSR

SEQ ID NO: 756 - protein, 10 aa, Homo sapiens
LNLNNTVLSK

SEQ ID NO: 757 - protein, 17 aa, Homo sapiens
LTWFVNEGLVDGWDDPR

SEQ ID NO: 758 - protein, 10 aa, Homo sapiens
QFIAAQGSSR

SEQ ID NO: 759 - protein, 6 aa, Homo sapiens
IWAFNK

SEQ ID NO: 760 - protein, 8 aa, Homo sapiens
VIDPVAPR

SEQ ID NO: 761 - protein, 6 aa, Homo sapiens
YVALLK

SEQ ID NO: 762 - protein, 14 aa, Homo sapiens
NPEVGLKPVWYSPK

SEQ ID NO: 763 - protein, 7 aa, Homo sapiens
IISLDAK

SEQ ID NO: 764 - protein, 6 aa, Homo sapiens
LNLENK

SEQ ID NO: 765 - protein, 8 aa, Homo sapiens
GDIIQLQR

SEQ ID NO: 766 - protein, 8 aa, Homo sapiens
NETSAPFK

SEQ ID NO: 767 - protein, 9 aa, Homo sapiens
VAVQGDVVR

SEQ ID NO: 768 - protein, 8 aa, Homo sapiens
EDVDAAVK

SEQ ID NO: 769 - protein, 6 aa, Homo sapiens
QLLSLK

SEQ ID NO: 770 - protein, 14 aa, Homo sapiens
SLYDEVAAQGEVVR

SEQ ID NO: 771 - protein, 19 aa, Homo sapiens
EYIPGQPPLSQSSDSSPTR

SEQ ID NO: 772 - protein, 13 aa, Homo sapiens
NSEPAGLETPEAK

SEQ ID NO: 773 - protein, 9 aa, Homo sapiens
VASQGEVVR

SEQ ID NO: 774 - protein, 14 aa, Homo sapiens
DQVDIAVQELLQLK

SEQ ID NO: 775 - protein, 18 aa, Homo sapiens
SLIGVEYKPVSATGAEDK

SEQ ID NO: 776 - protein, 6 aa, Homo sapiens
QNKPQK

SEQ ID NO: 777 - protein, 6 aa, Homo sapiens
QNDGQR

SEQ ID NO: 778 - protein, 18 aa, Homo sapiens
NQGGGLSSSGAGEGQGPK

SEQ ID NO: 779 - protein, 14 aa, Homo sapiens
EENLADWYSQVITK

SEQ ID NO: 780 - protein, 8 aa, Homo sapiens
DFFDAEIK

SEQ ID NO: 781 - protein, 15 aa, Homo sapiens
THVADFAPEVAWVTR

SEQ ID NO: 782 - protein, 6 aa, Homo sapiens
WVQSHR

SEQ ID NO: 783 - protein, 7 aa, Homo sapiens
HPQPFLR

SEQ ID NO: 784 - protein, 19 aa, Homo sapiens
FAGGDYTTTIEAFISASGR

SEQ ID NO: 785 - protein, 16 aa, Homo sapiens
AIQGGTSHHLGQNFSK

SEQ ID NO: 786 - protein, 13 aa, Homo sapiens
QFAYQNSWGLTTR

SEQ ID NO: 787 - protein, 6 aa, Homo sapiens
EALIAK

SEQ ID NO: 788 - protein, 7 aa, Homo sapiens
LLSVNIR

SEQ ID NO: 789 - protein, 8 aa, Homo sapiens
DNYSPGWK

SEQ ID NO: 790 - protein, 7 aa, Homo sapiens
FNHWELK

SEQ ID NO: 791 - protein, 11 aa, Homo sapiens
LTVAENEAETK

SEQ ID NO: 792 - protein, 15 aa, Homo sapiens
LQAILEDIQVTLFTR

SEQ ID NO: 793 - protein, 7 aa, Homo sapiens
YYTLFGR

SEQ ID NO: 794 - protein, 8 aa, Homo sapiens
GVFVVAAK

SEQ ID NO: 795 - protein, 11 aa, Homo sapiens
TPFGAYGGLLK

SEQ ID NO: 796 - protein, 13 aa, Homo sapiens
DFTATDLSEFAAK

SEQ ID NO: 797 - protein, 9 aa, Homo sapiens
ETPALTINR

SEQ ID NO: 798 - protein, 8 aa, Homo sapiens
YALQSQQR

SEQ ID NO: 799 - protein, 8 aa, Homo sapiens
HNFTPLAR

SEQ ID NO: 800 - protein, 8 aa, Homo sapiens
SLDLDISK

SEQ ID NO: 801 - protein, 20 aa, Homo sapiens
TNVNGGAIALGHPLGGSGSR

SEQ ID NO: 802 - protein, 10 aa, Homo sapiens
ITAHLVHELR

SEQ ID NO: 803 - protein, 7 aa, Homo sapiens
LETADLK

SEQ ID NO: 804 - protein, 25 aa, Homo sapiens
WVTFPQVDGQWEELSGLDEEQHSVR

SEQ ID NO: 805 - protein, 9 aa, Homo sapiens
APGQAHWLR

SEQ ID NO: 806 - protein, 6 aa, Homo sapiens
TGWVPR

SEQ ID NO: 807 - protein, 10 aa, Homo sapiens
GAVHVYATLR

SEQ ID NO: 808 - protein, 11 aa, Homo sapiens
VDTVAAEHLTR

SEQ ID NO: 809 - protein, 9 aa, Homo sapiens
RPGAEATGK

SEQ ID NO: 810 - protein, 6 aa, Homo sapiens
LGPLSK

SEQ ID NO: 811 - protein, 7 aa, Homo sapiens
FPETVPR

SEQ ID NO: 812 - protein, 19 aa, Homo sapiens
LNGSSLHLEWSAPLESGGR

SEQ ID NO: 813 - protein, 8 aa, Homo sapiens
EDLTYALR

SEQ ID NO: 814 - protein, 10 aa, Homo sapiens
DLVEPWVVVR

SEQ ID NO: 815 - protein, 10 aa, Homo sapiens
EVPPAVSDIR

SEQ ID NO: 816 - protein, 14 aa, Homo sapiens
SSPSSLSLAWAVPR

SEQ ID NO: 817 - protein, 12 aa, Homo sapiens
APSGAVLDYEVK

SEQ ID NO: 818 - protein, 9 aa, Homo sapiens
GAEGPSSVR

SEQ ID NO: 819 - protein, 9 aa, Homo sapiens
GASYLVQVR

SEQ ID NO: 820 - protein, 25 aa, Homo sapiens
SEAGYGPFGQEHHSQTQLDESEGWR

SEQ ID NO: 821 - protein, 7 aa, Homo sapiens
EAEYSDK

SEQ ID NO: 822 - protein, 11 aa, Homo sapiens
HGQYLIGHGTK

SEQ ID NO: 823 - protein, 8 aa, Homo sapiens
EIDVSYVK

SEQ ID NO: 824 - protein, 6 aa, Homo sapiens
GGYTER

SEQ ID NO: 825 - protein, 18 aa, Homo sapiens
FLEENSSDPTYTSSLGGK

SEQ ID NO: 826 - protein, 10 aa, Homo sapiens
FPQVVSALDK

SEQ ID NO: 827 - protein, 13 aa, Homo sapiens
ENGGASHPLLDQR

SEQ ID NO: 828 - protein, 15 aa, Homo sapiens
QPHYSAFGSVGEWLR

SEQ ID NO: 829 - protein, 18 aa, Homo sapiens
SQAKPGTPGGTGGPAPQY

SEQ ID NO: 830 - protein, 9 aa, Homo sapiens
NVKPDQWVK

SEQ ID NO: 831 - protein, 10 aa, Homo sapiens
LNLGTVGFYR

SEQ ID NO: 832 - protein, 9 aa, Homo sapiens
DLSLPPVDR

SEQ ID NO: 833 - protein, 12 aa, Homo sapiens
LGLQNDLFSLAR

SEQ ID NO: 834 - protein, 11 aa, Homo sapiens
AGIISTVEVLK

SEQ ID NO: 835 - protein, 9 aa, Homo sapiens
DVFSPIGER

SEQ ID NO: 836 - protein, 17 aa, Homo sapiens
LGWDPKPGEGHLDALLR

SEQ ID NO: 837 - protein, 6 aa, Homo sapiens
GLVLGK

SEQ ID NO: 838 - protein, 7 aa, Homo sapiens
ATLEEAR

SEQ ID NO: 839 - protein, 6 aa, Homo sapiens
DHVEGK

SEQ ID NO: 840 - protein, 8 aa, Homo sapiens
QILSADLR

SEQ ID NO: 841 - protein, 9 aa, Homo sapiens
SPVYLTVLK

SEQ ID NO: 842 - protein, 13 aa, Homo sapiens
VLGATLLPDLIQK

SEQ ID NO: 843 - protein, 9 aa, Homo sapiens
DNWEELYNR

SEQ ID NO: 844 - protein, 9 aa, Homo sapiens
YQGGFLISR

SEQ ID NO: 845 - protein, 10 aa, Homo sapiens
LSVEGFAVDK

SEQ ID NO: 846 - protein, 13 aa, Homo sapiens
AFFESHPAPSAER

SEQ ID NO: 847 - protein, 12 aa, Homo sapiens
DAESIHQYLLQR

SEQ ID NO: 848 - protein, 7 aa, Homo sapiens
DIAAHIK

SEQ ID NO: 849 - protein, 6 aa, Homo sapiens
ETEGFR

SEQ ID NO: 850 - protein, 8 aa, Homo sapiens
SAQFFNYK

SEQ ID NO: 851 - protein, 14 aa, Homo sapiens
IQALGLGEDWNVEK

SEQ ID NO: 852 - protein, 9 aa, Homo sapiens
GTSAGGGQK

SEQ ID NO: 853 - protein, 20 aa, Homo sapiens
EDLVILFADSYDVLFASGPR

SEQ ID NO: 854 - protein, 15 aa, Homo sapiens
SQVVFSAEELIYPDR

SEQ ID NO: 855 - protein, 8 aa, Homo sapiens
YPVVSDGK

SEQ ID NO: 856 - protein, 16 aa, Homo sapiens
FLGSGGFIGYAPNLSK

SEQ ID NO: 857 - protein, 19 aa, Homo sapiens
LVAEWEGQDSDSDQLFYTK

SEQ ID NO: 858 - protein, 7 aa, Homo sapiens
IFLDPEK

SEQ ID NO: 859 - protein, 10 aa, Homo sapiens
EQINITLDHR

SEQ ID NO: 860 - protein, 15 aa, Homo sapiens
IFQNLDGALDEVVLK

SEQ ID NO: 861 - protein, 18 aa, Homo sapiens
NLAYDTLPVLIHGNGPTK

SEQ ID NO: 862 - protein, 12 aa, Homo sapiens
LQLNYLGNYIPR

SEQ ID NO: 863 - protein, 6 aa, Homo sapiens
LHYPQK

SEQ ID NO: 864 - protein, 11 aa, Homo sapiens
LFIHNHEQHHK

SEQ ID NO: 865 - protein, 18 aa, Homo sapiens
AQVEEFLAQHGSEYQSVK

SEQ ID NO: 866 - protein, 7 aa, Homo sapiens
LLIQQNK

SEQ ID NO: 867 - protein, 17 aa, Homo sapiens
LWSNFWGALSADGYYAR

SEQ ID NO: 868 - protein, 11 aa, Homo sapiens
SEDYVDIVQGR

SEQ ID NO: 869 - protein, 16 aa, Homo sapiens
VGVWNVPYISNIYLIK

SEQ ID NO: 870 - protein, 13 aa, Homo sapiens
GELQSSDLFHHSK

SEQ ID NO: 871 - protein, 13 aa, Homo sapiens
HTLGHLLSLDSYR

SEQ ID NO: 872 - protein, 19 aa, Homo sapiens
TTHLHNDLWEVFSNPEDWK

SEQ ID NO: 873 - protein, 8 aa, Homo sapiens
YIHQNYTK

SEQ ID NO: 874 - protein, 8 aa, Homo sapiens
LYPGYYTR

SEQ ID NO: 875 - protein, 10 aa, Homo sapiens
AQFDLAFVVR

SEQ ID NO: 876 - protein, 12 aa, Homo sapiens
LTHYHEGLPTTR

SEQ ID NO: 877 - protein, 9 aa, Homo sapiens
YIAVSFVDP

SEQ ID NO: 878 - protein, 17 aa, Homo sapiens
GDAKPEDNLLVLTVATK

SEQ ID NO: 879 - protein, 6 aa, Homo sapiens
NNFVIR

SEQ ID NO: 880 - protein, 7 aa, Homo sapiens
NPADPQR

SEQ ID NO: 881 - protein, 8 aa, Homo sapiens
IPSNPSHR

SEQ ID NO: 882 - protein, 9 aa, Homo sapiens
ELPQSIVYK

SEQ ID NO: 883 - protein, 23 aa, Homo sapiens
SVPSDIFQIQATTIYANTINTFR

SEQ ID NO: 884 - protein, 11 aa, Homo sapiens
SGNENGEFYLR

SEQ ID NO: 885 - protein, 10 aa, Homo sapiens
LTIIVGPFSF

SEQ ID NO: 886 - protein, 12 aa, Homo sapiens
EAAIVDPVQPQK

SEQ ID NO: 887 - protein, 19 aa, Homo sapiens
LTTVLTTHHHWDHAGGNEK

SEQ ID NO: 888 - protein, 6 aa, Homo sapiens
LESGLK

SEQ ID NO: 889 - protein, 7 aa, Homo sapiens
VYGGDDR

SEQ ID NO: 890 - protein, 7 aa, Homo sapiens
IGALTHK

SEQ ID NO: 891 - protein, 15 aa, Homo sapiens
ITHLSTLQVGSLNVK

SEQ ID NO: 892 - protein, 8 aa, Homo sapiens
ALLEVLGR

SEQ ID NO: 893 - protein, 10 aa, Homo sapiens
HVEPGNAAIR

SEQ ID NO: 894 - protein, 7 aa, Homo sapiens
LDDDSER

SEQ ID NO: 895 - protein, 8 aa, Homo sapiens
DISSIGLK

SEQ ID NO: 896 - protein, 7 aa, Homo sapiens
LTSAIAK

SEQ ID NO: 897 - protein, 7 aa, Homo sapiens
IGEYLEK

SEQ ID NO: 898 - protein, 9 aa, Homo sapiens
TVSPALISR

SEQ ID NO: 899 - protein, 12 aa, Homo sapiens
ADVFHAYLSLLK

SEQ ID NO: 900 - protein, 14 aa, Homo sapiens
ITSEALLVTQQLVK

SEQ ID NO: 901 - protein, 11 aa, Homo sapiens
ALTLIAGSPLK

SEQ ID NO: 902 - protein, 19 aa, Homo sapiens
IDLRPVLGEGVPILASFLR

SEQ ID NO: 903 - protein, 12 aa, Homo sapiens
LGTLSALDILIK

SEQ ID NO: 904 - protein, 8 aa, Homo sapiens
VYPSSLSK

SEQ ID NO: 905 - protein, 14 aa, Homo sapiens
ISGSILNELIGLVR

SEQ ID NO: 906 - protein, 8 aa, Homo sapiens
QSYYSIAK

SEQ ID NO: 907 - protein, 14 aa, Homo sapiens
EGPAVVGQFIQDVK

SEQ ID NO: 908 - protein, 6 aa, Homo sapiens
STDSIR

SEQ ID NO: 909 - protein, 23 aa, Homo sapiens
LLALLSLGEVGHHIDLSGQLELK

SEQ ID NO: 910 - protein, 15 aa, Homo sapiens
SVILEAFSSPSEEVK

SEQ ID NO: 911 - protein, 9 aa, Homo sapiens
QYLLLHSLK

SEQ ID NO: 912 - protein, 24 aa, Homo sapiens
EIISSASVVGLKPYVENIWALLLK

SEQ ID NO: 913 - protein, 12 aa, Homo sapiens
LTLIDPETLLPR

SEQ ID NO: 914 - protein, 11 aa, Homo sapiens
GYLISGSSYAR

SEQ ID NO: 915 - protein, 8 aa, Homo sapiens
SSVVTAVK

SEQ ID NO: 916 - protein, 15 aa, Homo sapiens
FTISDHPQPIDPLLK

SEQ ID NO: 917 - protein, 10 aa, Homo sapiens
TLEDPDLNVR

SEQ ID NO: 918 - protein, 18 aa, Homo sapiens
VALVTFNSAAHNKPSLIR

SEQ ID NO: 919 - protein, 15 aa, Homo sapiens
DLLDTVLPHLYNETK

SEQ ID NO: 920 - protein, 10 aa, Homo sapiens
HTVDDGLDIR

SEQ ID NO: 921 - protein, 15 aa, Homo sapiens
LDIFEFLNHVEDGLK

SEQ ID NO: 922 - protein, 6 aa, Homo sapiens
DHYDIK

SEQ ID NO: 923 - protein, 6 aa, Homo sapiens
LVEPLR

SEQ ID NO: 924 - protein, 13 aa, Homo sapiens
AVAALLTIPEAEK

SEQ ID NO: 925 - protein, 18 aa, Homo sapiens
VIRPLDQPSSFDATPYIK

SEQ ID NO: 926 - protein, 9 aa, Homo sapiens
AADIDQEVK

SEQ ID NO: 927 - protein, 15 aa, Homo sapiens
SQGVNDNEEGFFSAR

SEQ ID NO: 928 - protein, 7 aa, Homo sapiens
GHRPLDK

SEQ ID NO: 929 - protein, 19 aa, Homo sapiens
EEAPSLRPAPPPISGGGYR

SEQ ID NO: 930 - protein, 15 aa, Homo sapiens
DNENVVNEYSSELEK

SEQ ID NO: 931 - protein, 18 aa, Homo sapiens
HQLYIDETVNSNIPTNLR

SEQ ID NO: 932 - protein, 7 aa, Homo sapiens
SILENLR

SEQ ID NO: 933 - protein, 9 aa, Homo sapiens
QDGSVDFGR

SEQ ID NO: 934 - protein, 13 aa, Homo sapiens
QGFGNVATNTDGK

SEQ ID NO: 935 - protein, 14 aa, Homo sapiens
AHYGGFTVQNEANK

SEQ ID NO: 936 - protein, 7 aa, Homo sapiens
YQISVNK

SEQ ID NO: 937 - protein, 11 aa, Homo sapiens
DNDGWLTSDPR

SEQ ID NO: 938 - protein, 10 aa, Homo sapiens
EDGGGWWYNR

SEQ ID NO: 939 - protein, 8 aa, Homo sapiens
IRPFFPQQ

SEQ ID NO: 940 - protein, 6 aa, Homo sapiens
VFVFPR

SEQ ID NO: 941 - protein, 7 aa, Homo sapiens
AYSDLSR

SEQ ID NO: 942 - protein, 12 aa, Homo sapiens
AYSLFSYNTQGR

SEQ ID NO: 943 - protein, 8 aa, Homo sapiens
DNELLVYK

SEQ ID NO: 944 - protein, 10 aa, Homo sapiens
VGEYSLYIGR

SEQ ID NO: 945 - protein, 10 aa, Homo sapiens
QGYFVEAQPK

SEQ ID NO: 946 - protein, 13 aa, Homo sapiens
IVLGQEQDSYGGK

SEQ ID NO: 947 - protein, 11 aa, Homo sapiens
GYVIIKPLVWV

SEQ ID NO: 948 - protein, 14 aa, Homo sapiens
EDPQTFYYAVAVVK

SEQ ID NO: 949 - protein, 10 aa, Homo sapiens
DGAGDVAFVK

SEQ ID NO: 950 - protein, 11 aa, Homo sapiens
HSTIFENLANK

SEQ ID NO: 951 - protein, 7 aa, Homo sapiens
KPVDEYK

SEQ ID NO: 952 - protein, 17 aa, Homo sapiens
EDLIWELLNQAQEHFGK

SEQ ID NO: 953 - protein, 11 aa, Homo sapiens
EFQLFSSPHGK

SEQ ID NO: 954 - protein, 8 aa, Homo sapiens
DSAHGFLK

SEQ ID NO: 955 - protein, 11 aa, Homo sapiens
SASDLTWDNLK

SEQ ID NO: 956 - protein, 11 aa, Homo sapiens
EGYYGYTGAFR

SEQ ID NO: 957 - protein, 11 aa, Homo sapiens
HQTVPQNTGGK

SEQ ID NO: 958 - protein, 7 aa, Homo sapiens
NPDPWAK

SEQ ID NO: 959 - protein, 9 aa, Homo sapiens
APNHAVVTR

SEQ ID NO: 960 - protein, 8 aa, Homo sapiens
YLGEEYVK

SEQ ID NO: 961 - protein, 6 aa, Homo sapiens
AVGNLR

SEQ ID NO: 962 - protein, 14 aa, Homo sapiens
AVTEQGHELSNEER

SEQ ID NO: 963 - protein, 12 aa, Homo sapiens
YLIPNATQPESK

SEQ ID NO: 964 - protein, 11 aa, Homo sapiens
YLSEVASGDNK

SEQ ID NO: 965 - protein, 19 aa, Homo sapiens
QTTVSNSQQAYQEAFEISK

SEQ ID NO: 966 - protein, 19 aa, Homo sapiens
TAFDEAIAELDTLNEESYK

SEQ ID NO: 967 - protein, 22 aa, Homo sapiens
DNLTLWTSENQGDEGDAGEGEN

SEQ ID NO: 968 - protein, 12 aa, Homo sapiens
EGDVLTLLESER

SEQ ID NO: 969 - protein, 6 aa, Homo sapiens
QAPGQR

SEQ ID NO: 970 - protein, 17 aa, Homo sapiens
EVNTSGFAPARPPPQPR

SEQ ID NO: 971 - protein, 9 aa, Homo sapiens
NVKPDQWVK

SEQ ID NO: 972 - protein, 10 aa, Homo sapiens
LNLGTVGFYR

SEQ ID NO: 973 - protein, 9 aa, Homo sapiens
DLSLPPVDR

SEQ ID NO: 974 - protein, 12 aa, Homo sapiens
LGLQNDLFSLAR

SEQ ID NO: 975 - protein, 11 aa, Homo sapiens
AGIISTVEVLK

SEQ ID NO: 976 - protein, 9 aa, Homo sapiens
DVFSPIGER

SEQ ID NO: 977 - protein, 17 aa, Homo sapiens
LGWDPKPGEGHLDALLR

SEQ ID NO: 978 - protein, 6 aa, Homo sapiens
GLVLGK

SEQ ID NO: 979 - protein, 7 aa, Homo sapiens
ATLEEAR

SEQ ID NO: 980 - protein, 6 aa, Homo sapiens
DHVEGK

SEQ ID NO: 981 - protein, 8 aa, Homo sapiens
QILSADLR

SEQ ID NO: 982 - protein, 9 aa, Homo sapiens
SPVYLTVLK

SEQ ID NO: 983 - protein, 13 aa, Homo sapiens
VLGATLLPDLIQK

SEQ ID NO: 984 - protein, 9 aa, Homo sapiens
DNWEELYNR

SEQ ID NO: 985 - protein, 9 aa, Homo sapiens
YQGGFLISR

SEQ ID NO: 986 - protein, 10 aa, Homo sapiens
LSVEGFAVDK

SEQ ID NO: 987 - protein, 13 aa, Homo sapiens
AFFESHPAPSAER

SEQ ID NO: 988 - protein, 12 aa, Homo sapiens
DAESIHQYLLQR

SEQ ID NO: 989 - protein, 7 aa, Homo sapiens
DIAAHIK

SEQ ID NO: 990 - protein, 6 aa, Homo sapiens
NNFVIR

SEQ ID NO: 991 - protein, 7 aa, Homo sapiens
NPADPQR

SEQ ID NO: 992 - protein, 8 aa, Homo sapiens
IPSNPSHR

SEQ ID NO: 993 - protein, 9 aa, Homo sapiens
ELPQSIVYK

SEQ ID NO: 994 - protein, 23 aa, Homo sapiens
SVPSDIFQIQATTIYANTINTFR

SEQ ID NO: 995 - protein, 11 aa, Homo sapiens
SGNENGEFYLR

SEQ ID NO: 996 - protein, 10 aa, Homo sapiens
LTIIVGPFSF

SEQ ID NO: 997 - protein, 15 aa, Homo sapiens
VGAHAGEYGAEALER

SEQ ID NO: 998 - protein, 16 aa, Homo sapiens
TYFPHFDLSHGSAQVK

SEQ ID NO: 999 - protein, 12 aa, Homo sapiens
FLASVSTVLTSK

SEQ ID NO: 1000 - protein, 20 aa, Homo sapiens
ELLLPNWQGSGSHGLTIAQR

SEQ ID NO: 1001 - protein, 17 aa, Homo sapiens
DDGVFVQEVTQNSPAAR

SEQ ID NO: 1002 - protein, 10 aa, Homo sapiens
SPEPGQTWTR

SEQ ID NO: 1003 - protein, 15 aa, Homo sapiens
SEDGVEGDLGETQSR

SEQ ID NO: 1004 - protein, 6 aa, Homo sapiens
TITVTR

SEQ ID NO: 1005 - protein, 11 aa, Homo sapiens
VTAYTVDVTGR

SEQ ID NO: 1006 - protein, 10 aa, Homo sapiens
DIDISSPEFK

SEQ ID NO: 1007 - protein, 17 aa, Homo sapiens
HELTEISNVDVETQSGK

SEQ ID NO: 1008 - protein, 18 aa, Homo sapiens
LPSGSGAASPTGSAVDIR

SEQ ID NO: 1009 - protein, 18 aa, Homo sapiens
AGAISASGPELQGAGHSK

SEQ ID NO: 1010 - protein, 11 aa, Homo sapiens
VGGSGVNVNAK

SEQ ID NO: 1011 - protein, 7 aa, Homo sapiens
GLDLGGR

SEQ ID NO: 1012 - protein, 17 aa, Homo sapiens
GGVQVPAVDISSSLGGR

SEQ ID NO: 1013 - protein, 16 aa, Homo sapiens
AVEVQGPSLESGDHGK

SEQ ID NO: 1014 - protein, 7 aa, Homo sapiens
FGVSTGR

SEQ ID NO: 1015 - protein, 6 aa, Homo sapiens
EGQTPK

SEQ ID NO: 1016 - protein, 11 aa, Homo sapiens
VSAPEVSVGHK

SEQ ID NO: 1017 - protein, 16 aa, Homo sapiens
GPQITGPSLEGDLGLK

SEQ ID NO: 1018 - protein, 7 aa, Homo sapiens
FSVSGAK

SEQ ID NO: 1019 - protein, 11 aa, Homo sapiens
VSAPGVQGDVK

SEQ ID NO: 1020 - protein, 7 aa, Homo sapiens
GPQVALK

SEQ ID NO: 1021 - protein, 16 aa, Homo sapiens
VDIETPNLEGTLTGPR

SEQ ID NO: 1022 - protein, 7 aa, Homo sapiens
LGSPSGK

SEQ ID NO: 1023 - protein, 9 aa, Homo sapiens
GGVDVTLPR

SEQ ID NO: 1024 - protein, 7 aa, Homo sapiens
VPEVDVR

SEQ ID NO: 1025 - protein, 18 aa, Homo sapiens
VDVSAPDVEAHGPEWNLK

SEQ ID NO: 1026 - protein, 9 aa, Homo sapiens
GDISISGPK

SEQ ID NO: 1027 - protein, 16 aa, Homo sapiens
VNVEAPDVNLEGLGGK

SEQ ID NO: 1028 - protein, 9 aa, Homo sapiens
GEYDVTVPK

SEQ ID NO: 1029 - protein, 18 aa, Homo sapiens
VDIDAPDVDVHGPDWHLK

SEQ ID NO: 1030 - protein, 7 aa, Homo sapiens
FSVPGFK

SEQ ID NO: 1031 - protein, 12 aa, Homo sapiens
AEGPEVDVNLPK

SEQ ID NO: 1032 - protein, 9 aa, Homo sapiens
ADVDISGPK

SEQ ID NO: 1033 - protein, 16 aa, Homo sapiens
IDVTAPDVSIEEPEGK

SEQ ID NO: 1034 - protein, 7 aa, Homo sapiens
VPDVELK

SEQ ID NO: 1035 - protein, 16 aa, Homo sapiens
VGVEVPDVNIEGPEGK

SEQ ID NO: 1036 - protein, 12 aa, Homo sapiens
GEGPEFDVNLSK

SEQ ID NO: 1037 - protein, 9 aa, Homo sapiens
ANVDISAPK

SEQ ID NO: 1038 - protein, 16 aa, Homo sapiens
VDTNAPDLSLEGPEGK

SEQ ID NO: 1039 - protein, 9 aa, Homo sapiens
GNVDISAPK

SEQ ID NO: 1040 - protein, 14 aa, Homo sapiens
APDVEGQGLDWSLK

SEQ ID NO: 1041 - protein, 12 aa, Homo sapiens
GEGPEVDVNLPK

SEQ ID NO: 1042 - protein, 9 aa, Homo sapiens
ADVVVSGPK

SEQ ID NO: 1043 - protein, 16 aa, Homo sapiens
VDIEAPDVSLEGPEGK

SEQ ID NO: 1044 - protein, 9 aa, Homo sapiens
GDVDVSVPK

SEQ ID NO: 1045 - protein, 7 aa, Homo sapiens
VPDVEIK

SEQ ID NO: 1046 - protein, 8 aa, Homo sapiens
EVDVNLPK

SEQ ID NO: 1047 - protein, 9 aa, Homo sapiens
ADIDVSGPK

SEQ ID NO: 1048 - protein, 16 aa, Homo sapiens
VDVEVPDVSLEGPEGK

SEQ ID NO: 1049 - protein, 18 aa, Homo sapiens
VDISAPDVDVHGPDWHLK

SEQ ID NO: 1050 - protein, 9 aa, Homo sapiens
GEGPEVDVK

SEQ ID NO: 1051 - protein, 9 aa, Homo sapiens
ADVDVSGPK

SEQ ID NO: 1052 - protein, 11 aa, Homo sapiens
ISIPDVGLHLK

SEQ ID NO: 1053 - protein, 9 aa, Homo sapiens
GDYDVTVPK

SEQ ID NO: 1054 - protein, 6 aa, Homo sapiens
VEGEIK

SEQ ID NO: 1055 - protein, 7 aa, Homo sapiens
APDVDIK

SEQ ID NO: 1056 - protein, 18 aa, Homo sapiens
VDINAPDVEVHGPDWHLK

SEQ ID NO: 1057 - protein, 9 aa, Homo sapiens
ADLGVSGPK

SEQ ID NO: 1058 - protein, 16 aa, Homo sapiens
VDIDVPDVNLEAPEGK

SEQ ID NO: 1059 - protein, 9 aa, Homo sapiens
TDVDVSLPK

SEQ ID NO: 1060 - protein, 6 aa, Homo sapiens
VEGDLK

SEQ ID NO: 1061 - protein, 7 aa, Homo sapiens
GPEIDVK

SEQ ID NO: 1062 - protein, 18 aa, Homo sapiens
VDIDAPDVEVHDPDWHLK

SEQ ID NO: 1063 - protein, 25 aa, Homo sapiens
ADIDVSGPSVDTDAPDLDIEGPEGK

SEQ ID NO: 1064 - protein, 15 aa, Homo sapiens
GEIDASVPELEGDLR

SEQ ID NO: 1065 - protein, 9 aa, Homo sapiens
GDADVSVPK

SEQ ID NO: 1066 - protein, 9 aa, Homo sapiens
GDVDVSLPK

SEQ ID NO: 1067 - protein, 7 aa, Homo sapiens
VPDVDIR

SEQ ID NO: 1068 - protein, 9 aa, Homo sapiens
GDVDVSAPK

SEQ ID NO: 1069 - protein, 7 aa, Homo sapiens
GPELDVK

SEQ ID NO: 1070 - protein, 9 aa, Homo sapiens
GEVDVDVPK

SEQ ID NO: 1071 - protein, 19 aa, Homo sapiens
GPHVDVSGPDIDIEGPEGK

SEQ ID NO: 1072 - protein, 13 aa, Homo sapiens
GDVDVSVPEVEGK

SEQ ID NO: 1073 - protein, 16 aa, Homo sapiens
VDVNAPDVQAPDWHLK

SEQ ID NO: 1074 - protein, 16 aa, Homo sapiens
VDIEGPDVNIEGPEGK

SEQ ID NO: 1075 - protein, 7 aa, Homo sapiens
GPEVDIK

SEQ ID NO: 1076 - protein, 18 aa, Homo sapiens
VDINAPDVGVQGPDWHLK

SEQ ID NO: 1077 - protein, 9 aa, Homo sapiens
GEGPDGDVK

SEQ ID NO: 1078 - protein, 12 aa, Homo sapiens
GEGPDVDVNLPK

SEQ ID NO: 1079 - protein, 9 aa, Homo sapiens
GDVDVTGPK

SEQ ID NO: 1080 - protein, 7 aa, Homo sapiens
GPEVDLK

SEQ ID NO: 1081 - protein, 18 aa, Homo sapiens
VDIDVPDVNVQGPDWHLK

SEQ ID NO: 1082 - protein, 16 aa, Homo sapiens
VDVEGPDVNIEGPEGK

SEQ ID NO: 1083 - protein, 9 aa, Homo sapiens
GDVDISLPK

SEQ ID NO: 1084 - protein, 7 aa, Homo sapiens
GPEVDIR

SEQ ID NO: 1085 - protein, 21 aa, Homo sapiens
GPQVDIDVPDVGVQGPDWHLK

SEQ ID NO: 1086 - protein, 9 aa, Homo sapiens
ADLDVSGPK

SEQ ID NO: 1087 - protein, 16 aa, Homo sapiens
VDIDVPDVNIEGPEGK

SEQ ID NO: 1088 - protein, 18 aa, Homo sapiens
VDINAPDVDVQGPDWHLK

SEQ ID NO: 1089 - protein, 16 aa, Homo sapiens
VDVDVPDVNIEGPDAK

SEQ ID NO: 1090 - protein, 15 aa, Homo sapiens
GEVDVSLANVEGDLK

SEQ ID NO: 1091 - protein, 7 aa, Homo sapiens
GPALDIK

SEQ ID NO: 1092 - protein, 16 aa, Homo sapiens
IDVDAPDIDIHGPDAK

SEQ ID NO: 1093 - protein, 9 aa, Homo sapiens
GDVDVSGPK

SEQ ID NO: 1094 - protein, 7 aa, Homo sapiens
APSLDIK

SEQ ID NO: 1095 - protein, 10 aa, Homo sapiens
GPEVDVSGPK

SEQ ID NO: 1096 - protein, 6 aa, Homo sapiens
LNIEGK

SEQ ID NO: 1097 - protein, 7 aa, Homo sapiens
FNFSGSK

SEQ ID NO: 1098 - protein, 9 aa, Homo sapiens
VQTPEVDVK

SEQ ID NO: 1099 - protein, 10 aa, Homo sapiens
KPDIDITGPK

SEQ ID NO: 1100 - protein, 13 aa, Homo sapiens
VDINAPDVEVQGK

SEQ ID NO: 1101 - protein, 15 aa, Homo sapiens
GDLDIAGPNLEGDFK

SEQ ID NO: 1102 - protein, 21 aa, Homo sapiens
APEVNLNAPDVDVHGPDWNLK

SEQ ID NO: 1103 - protein, 12 aa, Homo sapiens
AEGPDVAVDLPK

SEQ ID NO: 1104 - protein, 18 aa, Homo sapiens
VDINAPDVDVHGPDWHLK

SEQ ID NO: 1105 - protein, 12 aa, Homo sapiens
GEGPEVDVTLPK

SEQ ID NO: 1106 - protein, 25 aa, Homo sapiens
ADIDISGPNVDVDVPDVNIEGPDAK

SEQ ID NO: 1107 - protein, 9 aa, Homo sapiens
GDVVVSLPK

SEQ ID NO: 1108 - protein, 16 aa, Homo sapiens
VDIDTPDINIEGSEGK

SEQ ID NO: 1109 - protein, 11 aa, Homo sapiens
VDINAPDVDVR

SEQ ID NO: 1110 - protein, 7 aa, Homo sapiens
GPDWHLK

SEQ ID NO: 1111 - protein, 7 aa, Homo sapiens
APEVDIK

SEQ ID NO: 1112 - protein, 16 aa, Homo sapiens
VDIDTPDIDIHGPEGK

SEQ ID NO: 1113 - protein, 18 aa, Homo sapiens
VDIDVPDVDVQGPDWHLK

SEQ ID NO: 1114 - protein, 16 aa, Homo sapiens
VDIDVPDVNIEGPDAK

SEQ ID NO: 1115 - protein, 12 aa, Homo sapiens
GEGPDVDVTLPK

SEQ ID NO: 1116 - protein, 9 aa, Homo sapiens
ADIEISGPK

SEQ ID NO: 1117 - protein, 16 aa, Homo sapiens
VDIDAPDVSIEGPDAK

SEQ ID NO: 1118 - protein, 19 aa, Homo sapiens
GPSLDIDTPDVNIEGPEGK

SEQ ID NO: 1119 - protein, 12 aa, Homo sapiens
GPEVDIEGPEGK

SEQ ID NO: 1120 - protein, 16 aa, Homo sapiens
VGIDTPDIDIHGPEGK

SEQ ID NO: 1121 - protein, 9 aa, Homo sapiens
GDVDVTLPK

SEQ ID NO: 1122 - protein, 7 aa, Homo sapiens
GPEADIK

SEQ ID NO: 1123 - protein, 18 aa, Homo sapiens
VDINTPDVDVHGPDWHLK

SEQ ID NO: 1124 - protein, 12 aa, Homo sapiens
GEGPDVDVSLPK

SEQ ID NO: 1125 - protein, 16 aa, Homo sapiens
VDVDIPDVNIEGPDAK

SEQ ID NO: 1126 - protein, 11 aa, Homo sapiens
ISIPDVDLDLK

SEQ ID NO: 1127 - protein, 9 aa, Homo sapiens
GDFDVSVPK

SEQ ID NO: 1128 - protein, 6 aa, Homo sapiens
VEGTLK

SEQ ID NO: 1129 - protein, 9 aa, Homo sapiens
LDFEGPDAK

SEQ ID NO: 1130 - protein, 7 aa, Homo sapiens
LSGPSLK

SEQ ID NO: 1131 - protein, 11 aa, Homo sapiens
VTAPDVDLHLK

SEQ ID NO: 1132 - protein, 7 aa, Homo sapiens
IGFSGPK

SEQ ID NO: 1133 - protein, 9 aa, Homo sapiens
LEGGEVDLK

SEQ ID NO: 1134 - protein, 6 aa, Homo sapiens
FGFGAK

SEQ ID NO: 1135 - protein, 25 aa, Homo sapiens
SPSLDVTVPEAELNLETPEISVGGK

SEQ ID NO: 1136 - protein, 17 aa, Homo sapiens
QGFDLNVPGGEIDASLK

SEQ ID NO: 1137 - protein, 16 aa, Homo sapiens
APDVDVNIAGPDAALK

SEQ ID NO: 1138 - protein, 9 aa, Homo sapiens
AEAPLPSPK

SEQ ID NO: 1139 - protein, 25 aa, Homo sapiens
LEGELQAPDLELSLPAIHVEGLDIK

SEQ ID NO: 1140 - protein, 6 aa, Homo sapiens
IEGDLK

SEQ ID NO: 1141 - protein, 18 aa, Homo sapiens
VQANLGAPDINIEGLDAK

SEQ ID NO: 1142 - protein, 21 aa, Homo sapiens
TPSFGISAPQVSIPDVNVNLK

SEQ ID NO: 1143 - protein, 21 aa, Homo sapiens
GDVPSVGLEGPDVDLQGPEAK

SEQ ID NO: 1144 - protein, 7 aa, Homo sapiens
IGIPGVK

SEQ ID NO: 1145 - protein, 9 aa, Homo sapiens
LEGPDVSLK

SEQ ID NO: 1146 - protein, 11 aa, Homo sapiens
VSGPDLDLNLK

SEQ ID NO: 1147 - protein, 14 aa, Homo sapiens
VHAPGLNLSGVGGK

SEQ ID NO: 1148 - protein, 9 aa, Homo sapiens
VPGIDATTK

SEQ ID NO: 1149 - protein, 11 aa, Homo sapiens
LNVGAPDVTLR

SEQ ID NO: 1150 - protein, 15 aa, Homo sapiens
GPSLQGDLAVSGDIK

SEQ ID NO: 1151 - protein, 18 aa, Homo sapiens
VSVGAPDLSLEASEGSIK

SEQ ID NO: 1152 - protein, 18 aa, Homo sapiens
LPQFGISTPGSDLHVNAK

SEQ ID NO: 1153 - protein, 9 aa, Homo sapiens
GPQVSGELK

SEQ ID NO: 1154 - protein, 9 aa, Homo sapiens
GPGVDVNLK

SEQ ID NO: 1155 - protein, 14 aa, Homo sapiens
ISAPNVDFNLEGPK

SEQ ID NO: 1156 - protein, 10 aa, Homo sapiens
GSLGATGEIK

SEQ ID NO: 1157 - protein, 12 aa, Homo sapiens
LPTGQISGPEIK

SEQ ID NO: 1158 - protein, 16 aa, Homo sapiens
GSEVGFHGAAPDISVK

SEQ ID NO: 1159 - protein, 24 aa, Homo sapiens
GGADVSGGVSAPDISLGEGHLSVK

SEQ ID NO: 1160 - protein, 7 aa, Homo sapiens
GSGGEWK

SEQ ID NO: 1161 - protein, 14 aa, Homo sapiens
GPQVSSALNLDTSK

SEQ ID NO: 1162 - protein, 11 aa, Homo sapiens
FAGGLHFSGPK

SEQ ID NO: 1163 - protein, 6 aa, Homo sapiens
VEGGVK

SEQ ID NO: 1164 - protein, 6 aa, Homo sapiens
FTFSGR

SEQ ID NO: 1165 - protein, 20 aa, Homo sapiens
AEASIQAGAGDGEWEESEVK

SEQ ID NO: 1166 - protein, 7 aa, Homo sapiens
FNFSKPK

SEQ ID NO: 1167 - protein, 15 aa, Homo sapiens
GGVTGSPEASISGSK

SEQ ID NO: 1168 - protein, 18 aa, Homo sapiens
ASLGSLEGEAEAEASSPK

SEQ ID NO: 1169 - protein, 8 aa, Homo sapiens
SNSFSDER

SEQ ID NO: 1170 - protein, 25 aa, Homo sapiens
EFSGPSTPTGTLEFEGGEVSLEGGK

SEQ ID NO: 1171 - protein, 10 aa, Homo sapiens
FGTFGGLGSK

SEQ ID NO: 1172 - protein, 14 aa, Homo sapiens
GHYEVTGSDDETGK

SEQ ID NO: 1173 - protein, 11 aa, Homo sapiens
LQGSGVSLASK

SEQ ID NO: 1174 - protein, 12 aa, Homo sapiens
LSSSSSNDSGNK

SEQ ID NO: 1175 - protein, 15 aa, Homo sapiens
VGIQLPEVELSVSTK

SEQ ID NO: 1176 - protein, 9 aa, Homo sapiens
VDGSVDFYR

SEQ ID NO: 1177 - protein, 6 aa, Homo sapiens
DWATYK

SEQ ID NO: 1178 - protein, 23 aa, Homo sapiens
LGEFWLGNDNIHALTAQGTSELR

SEQ ID NO: 1179 - protein, 14 aa, Homo sapiens
VDLVDFEDNYQFAK

SEQ ID NO: 1180 - protein, 13 aa, Homo sapiens
GTHGSFANGINWK

SEQ ID NO: 1181 - protein, 7 aa, Homo sapiens
GYNYSYK

SEQ ID NO: 1182 - protein, 8 aa, Homo sapiens
GEAGTNGK

SEQ ID NO: 1183 - protein, 6 aa, Homo sapiens
QAPGQR

SEQ ID NO: 1184 - protein, 8 aa, Homo sapiens
WPEPVFGR

SEQ ID NO: 1185 - protein, 16 aa, Homo sapiens
LASPGFPGEYANDQER

SEQ ID NO: 1186 - protein, 10 aa, Homo sapiens
WTLTAPPGYR

SEQ ID NO: 1187 - protein, 15 aa, Homo sapiens
DTFYSLGSSLDITFR

SEQ ID NO: 1188 - protein, 7 aa, Homo sapiens
AGYVLHR

SEQ ID NO: 1189 - protein, 15 aa, Homo sapiens
SGELSSPEYPRPYPK

SEQ ID NO: 1190 - protein, 20 aa, Homo sapiens
SNTVTITFVTDESGDHTGWK

SEQ ID NO: 1191 - protein, 13 aa, Homo sapiens
VEYITGPGVTTYK

SEQ ID NO: 1192 - protein, 6 aa, Homo sapiens
IYGGQK

SEQ ID NO: 1193 - protein, 9 aa, Homo sapiens
HDASALDIR

SEQ ID NO: 1194 - protein, 15 aa, Homo sapiens
TDDIGTASGWGLTQR

SEQ ID NO: 1195 - protein, 16 aa, Homo sapiens
GDSGGALVFLDSETER

SEQ ID NO: 1196 - protein, 15 aa, Homo sapiens
VINYIPWIENIISDF

SEQ ID NO: 1197 - protein, 14 aa, Homo sapiens
SPDELPSAGGDGGK

SEQ ID NO: 1198 - protein, 8 aa, Homo sapiens
EKPNSAHR

SEQ ID NO: 1199 - protein, 10 aa, Homo sapiens
EETAGSYDSR

SEQ ID NO: 1200 - protein, 9 aa, Homo sapiens
FQPLLDGLK

SEQ ID NO: 1201 - protein, 8 aa, Homo sapiens
SGTTIALK

SEQ ID NO: 1202 - protein, 11 aa, Homo sapiens
LGLHQVLQDLR

SEQ ID NO: 1203 - protein, 18 aa, Homo sapiens
VQLNVFDEQGEEDSYDLK

SEQ ID NO: 1204 - protein, 16 aa, Homo sapiens
AEPHFLSILQHLLLVR

SEQ ID NO: 1205 - protein, 11 aa, Homo sapiens
NDYEARPQYYK

SEQ ID NO: 1206 - protein, 8 aa, Homo sapiens
NGADPDFK

SEQ ID NO: 1207 - protein, 8 aa, Homo sapiens
LQDLQGEK

SEQ ID NO: 1208 - protein, 7 aa, Homo sapiens
DALHSEK

SEQ ID NO: 1209 - protein, 7 aa, Homo sapiens
QQIATEK

SEQ ID NO: 1210 - protein, 16 aa, Homo sapiens
QDLEAEVSQLTGEVAK

SEQ ID NO: 1211 - protein, 9 aa, Homo sapiens
LYKPEVQLR

SEQ ID NO: 1212 - protein, 6 aa, Homo sapiens
RPNWSK

SEQ ID NO: 1213 - protein, 9 aa, Homo sapiens
FENNELFAK

SEQ ID NO: 1214 - protein, 10 aa, Homo sapiens
LTLTFSAQTK

SEQ ID NO: 1215 - protein, 8 aa, Homo sapiens
DQEGGEEK

SEQ ID NO: 1216 - protein, 13 aa, Homo sapiens
TAQNLSIFLGSFR

SEQ ID NO: 1217 - protein, 7 aa, Homo sapiens
LNAILFK

SEQ ID NO: 1218 - protein, 10 aa, Homo sapiens
FPDELAHVEK

SEQ ID NO: 1219 - protein, 8 aa, Homo sapiens
VSAENLQK

SEQ ID NO: 1220 - protein, 7 aa, Homo sapiens
QISDVER

SEQ ID NO: 1221 - protein, 12 aa, Homo sapiens
DVQNFPAATDEK

SEQ ID NO: 1222 - protein, 8 aa, Homo sapiens
DAQEQYNK

SEQ ID NO: 1223 - protein, 11 aa, Homo sapiens
ELGEYFLFDPK

SEQ ID NO: 1224 - protein, 13 aa, Homo sapiens
NSETFPTILEEAK

SEQ ID NO: 1225 - protein, 17 aa, Homo sapiens
VPAQLLGLLLLWLPGAK

SEQ ID NO: 1226 - protein, 18 aa, Homo sapiens
ASQSISSWLAWYQQKPGK

SEQ ID NO: 1227 - protein, 17 aa, Homo sapiens
EVNTSGFAPARPPPQPR

SEQ ID NO: 1228 - protein, 8 aa, Homo sapiens
GVFVVAAK

SEQ ID NO: 1229 - protein, 11 aa, Homo sapiens
TPFGAYGGLLK

SEQ ID NO: 1230 - protein, 13 aa, Homo sapiens
DFTATDLSEFAAK

SEQ ID NO: 1231 - protein, 9 aa, Homo sapiens
ETPALTINR

SEQ ID NO: 1232 - protein, 8 aa, Homo sapiens
YALQSQQR

SEQ ID NO: 1233 - protein, 8 aa, Homo sapiens
HNFTPLAR

SEQ ID NO: 1234 - protein, 8 aa, Homo sapiens
SLDLDISK

SEQ ID NO: 1235 - protein, 20 aa, Homo sapiens
TNVNGGAIALGHPLGGSGSR

SEQ ID NO: 1236 - protein, 10 aa, Homo sapiens
ITAHLVHELR

SEQ ID NO: 1237 - protein, 7 aa, Homo sapiens
LETADLK

SEQ ID NO: 1238 - protein, 25 aa, Homo sapiens
WVTFPQVDGQWEELSGLDEEQHSVR

SEQ ID NO: 1239 - protein, 9 aa, Homo sapiens
APGQAHWLR

SEQ ID NO: 1240 - protein, 6 aa, Homo sapiens
TGWVPR

SEQ ID NO: 1241 - protein, 10 aa, Homo sapiens
GAVHVYATLR

SEQ ID NO: 1242 - protein, 11 aa, Homo sapiens
VDTVAAEHLTR

SEQ ID NO: 1243 - protein, 9 aa, Homo sapiens
RPGAEATGK

SEQ ID NO: 1244 - protein, 6 aa, Homo sapiens
LGPLSK

SEQ ID NO: 1245 - protein, 7 aa, Homo sapiens
FPETVPR

SEQ ID NO: 1246 - protein, 19 aa, Homo sapiens
LNGSSLHLEWSAPLESGGR

SEQ ID NO: 1247 - protein, 8 aa, Homo sapiens
EDLTYALR

SEQ ID NO: 1248 - protein, 10 aa, Homo sapiens
DLVEPWVVVR

SEQ ID NO: 1249 - protein, 10 aa, Homo sapiens
EVPPAVSDIR

SEQ ID NO: 1250 - protein, 14 aa, Homo sapiens
SSPSSLSLAWAVPR

SEQ ID NO: 1251 - protein, 12 aa, Homo sapiens
APSGAVLDYEVK

SEQ ID NO: 1252 - protein, 9 aa, Homo sapiens
GAEGPSSVR

SEQ ID NO: 1253 - protein, 9 aa, Homo sapiens
GASYLVQVR

SEQ ID NO: 1254 - protein, 25 aa, Homo sapiens
SEAGYGPFGQEHHSQTQLDESEGWR

SEQ ID NO: 1255 - protein, 7 aa, Homo sapiens
EAEYSDK

SEQ ID NO: 1256 - protein, 11 aa, Homo sapiens
HGQYLIGHGTK

SEQ ID NO: 1257 - protein, 8 aa, Homo sapiens
EIDVSYVK

SEQ ID NO: 1258 - protein, 6 aa, Homo sapiens
GGYTER

SEQ ID NO: 1259 - protein, 18 aa, Homo sapiens
FLEENSSDPTYTSSLGGK

SEQ ID NO: 1260 - protein, 10 aa, Homo sapiens
FPQVVSALDK

SEQ ID NO: 1261 - protein, 13 aa, Homo sapiens
ENGGASHPLLDQR

SEQ ID NO: 1262 - protein, 15 aa, Homo sapiens
QPHYSAFGSVGEWLR

SEQ ID NO: 1263 - protein, 18 aa, Homo sapiens
SQAKPGTPGGTGGPAPQY

SEQ ID NO: 1264 - protein, 9 aa, Homo sapiens
NVKPDQWVK

SEQ ID NO: 1265 - protein, 10 aa, Homo sapiens
LNLGTVGFYR

SEQ ID NO: 1266 - protein, 9 aa, Homo sapiens
DLSLPPVDR

SEQ ID NO: 1267 - protein, 12 aa, Homo sapiens
LGLQNDLFSLAR

SEQ ID NO: 1268 - protein, 11 aa, Homo sapiens
AGIISTVEVLK

SEQ ID NO: 1269 - protein, 9 aa, Homo sapiens
DVFSPIGER

SEQ ID NO: 1270 - protein, 17 aa, Homo sapiens
LGWDPKPGEGHLDALLR

SEQ ID NO: 1271 - protein, 6 aa, Homo sapiens
GLVLGK

SEQ ID NO: 1272 - protein, 7 aa, Homo sapiens
ATLEEAR

SEQ ID NO: 1273 - protein, 6 aa, Homo sapiens
DHVEGK

SEQ ID NO: 1274 - protein, 8 aa, Homo sapiens
QILSADLR

SEQ ID NO: 1275 - protein, 9 aa, Homo sapiens
SPVYLTVLK

SEQ ID NO: 1276 - protein, 13 aa, Homo sapiens
VLGATLLPDLIQK

SEQ ID NO: 1277 - protein, 9 aa, Homo sapiens
DNWEELYNR

SEQ ID NO: 1278 - protein, 9 aa, Homo sapiens
YQGGFLISR

SEQ ID NO: 1279 - protein, 10 aa, Homo sapiens
LSVEGFAVDK

SEQ ID NO: 1280 - protein, 13 aa, Homo sapiens
AFFESHPAPSAER

SEQ ID NO: 1281 - protein, 12 aa, Homo sapiens
DAESIHQYLLQR

SEQ ID NO: 1282 - protein, 7 aa, Homo sapiens
DIAAHIK

SEQ ID NO: 1283 - protein, 6 aa, Homo sapiens
ETEGFR

SEQ ID NO: 1284 - protein, 8 aa, Homo sapiens
SAQFFNYK

SEQ ID NO: 1285 - protein, 14 aa, Homo sapiens
IQALGLGEDWNVEK

SEQ ID NO: 1286 - protein, 9 aa, Homo sapiens
GTSAGGGQK

SEQ ID NO: 1287 - protein, 20 aa, Homo sapiens
EDLVILFADSYDVLFASGPR

SEQ ID NO: 1288 - protein, 15 aa, Homo sapiens
SQVVFSAEELIYPDR

SEQ ID NO: 1289 - protein, 8 aa, Homo sapiens
YPVVSDGK

SEQ ID NO: 1290 - protein, 16 aa, Homo sapiens
FLGSGGFIGYAPNLSK

SEQ ID NO: 1291 - protein, 19 aa, Homo sapiens
LVAEWEGQDSDSDQLFYTK

SEQ ID NO: 1292 - protein, 7 aa, Homo sapiens
IFLDPEK

SEQ ID NO: 1293 - protein, 10 aa, Homo sapiens
EQINITLDHR

SEQ ID NO: 1294 - protein, 15 aa, Homo sapiens
IFQNLDGALDEVVLK

SEQ ID NO: 1295 - protein, 18 aa, Homo sapiens
NLAYDTLPVLIHGNGPTK

SEQ ID NO: 1296 - protein, 12 aa, Homo sapiens
LQLNYLGNYIPR

SEQ ID NO: 1297 - protein, 6 aa, Homo sapiens
LHYPQK

SEQ ID NO: 1298 - protein, 11 aa, Homo sapiens
LFIHNHEQHHK

SEQ ID NO: 1299 - protein, 18 aa, Homo sapiens
AQVEEFLAQHGSEYQSVK

SEQ ID NO: 1300 - protein, 7 aa, Homo sapiens
LLIQQNK

SEQ ID NO: 1301 - protein, 17 aa, Homo sapiens
LWSNFWGALSADGYYAR

SEQ ID NO: 1302 - protein, 11 aa, Homo sapiens
SEDYVDIVQGR

SEQ ID NO: 1303 - protein, 16 aa, Homo sapiens
VGVWNVPYISNIYLIK

SEQ ID NO: 1304 - protein, 13 aa, Homo sapiens
GELQSSDLFHHSK

SEQ ID NO: 1305 - protein, 13 aa, Homo sapiens
HTLGHLLSLDSYR

SEQ ID NO: 1306 - protein, 19 aa, Homo sapiens
TTHLHNDLWEVFSNPEDWK

SEQ ID NO: 1307 - protein, 8 aa, Homo sapiens
YIHQNYTK

SEQ ID NO: 1308 - protein, 8 aa, Homo sapiens
LYPGYYTR

SEQ ID NO: 1309 - protein, 10 aa, Homo sapiens
AQFDLAFVVR

SEQ ID NO: 1310 - protein, 12 aa, Homo sapiens
LTHYHEGLPTTR

SEQ ID NO: 1311 - protein, 9 aa, Homo sapiens
YIAVSFVDP

SEQ ID NO: 1312 - protein, 17 aa, Homo sapiens
GDAKPEDNLLVLTVATK

SEQ ID NO: 1313 - protein, 6 aa, Homo sapiens
NNFVIR

SEQ ID NO: 1314 - protein, 7 aa, Homo sapiens
NPADPQR

SEQ ID NO: 1315 - protein, 8 aa, Homo sapiens
IPSNPSHR

SEQ ID NO: 1316 - protein, 9 aa, Homo sapiens
ELPQSIVYK

SEQ ID NO: 1317 - protein, 23 aa, Homo sapiens
SVPSDIFQIQATTIYANTINTFR

SEQ ID NO: 1318 - protein, 11 aa, Homo sapiens
SGNENGEFYLR

SEQ ID NO: 1319 - protein, 10 aa, Homo sapiens
LTIIVGPFSF

SEQ ID NO: 1320 - protein, 19 aa, Homo sapiens
AGVAGLSAWTLQPQWIQVR

SEQ ID NO: 1321 - protein, 12 aa, Homo sapiens
IYGLGSLALYEK

SEQ ID NO: 1322 - protein, 10 aa, Homo sapiens
HLLIGVSSDR

SEQ ID NO: 1323 - protein, 11 aa, Homo sapiens
SEVATLTAAGK

SEQ ID NO: 1324 - protein, 11 aa, Homo sapiens
THSDQFLVAFK

SEQ ID NO: 1325 - protein, 6 aa, Homo sapiens
SVISYK

SEQ ID NO: 1326 - protein, 10 aa, Homo sapiens
ESTTSEQSAR

SEQ ID NO: 1327 - protein, 7 aa, Homo sapiens
LTLTFNR

SEQ ID NO: 1328 - protein, 6 aa, Homo sapiens
QAVITK

SEQ ID NO: 1329 - protein, 13 aa, Homo sapiens
ELIEIISGAAALD

SEQ ID NO: 1330 - protein, 6 aa, Homo sapiens
LHLITR

SEQ ID NO: 1331 - protein, 10 aa, Homo sapiens
NLQEVLGEEK

SEQ ID NO: 1332 - protein, 6 aa, Homo sapiens
IADFLK

SEQ ID NO: 1333 - protein, 9 aa, Homo sapiens
APWELLELR

SEQ ID NO: 1334 - protein, 9 aa, Homo sapiens
VSYYENVIK

SEQ ID NO: 1335 - protein, 8 aa, Homo sapiens
GTDYQLSK

SEQ ID NO: 1336 - protein, 8 aa, Homo sapiens
EYTLDVYR

SEQ ID NO: 1337 - protein, 11 aa, Homo sapiens
LSSVVTQHDSK

SEQ ID NO: 1338 - protein, 7 aa, Homo sapiens
AGAEVVK

SEQ ID NO: 1339 - protein, 24 aa, Homo sapiens
QVEHPLLSGLLYPGLQALDEEYLK

SEQ ID NO: 1340 - protein, 11 aa, Homo sapiens
VDAQFGGIDQR

SEQ ID NO: 1341 - protein, 7 aa, Homo sapiens
IFTFAEK

SEQ ID NO: 1342 - protein, 9 aa, Homo sapiens
YLPALGYSK

SEQ ID NO: 1343 - protein, 7 aa, Homo sapiens
HVLFPLK

SEQ ID NO: 1344 - protein, 7 aa, Homo sapiens
SEFVILR

SEQ ID NO: 1345 - protein, 10 aa, Homo sapiens
TYTAYVDLEK

SEQ ID NO: 1346 - protein, 13 aa, Homo sapiens
DFAAEVVHPGDLK

SEQ ID NO: 1347 - protein, 9 aa, Homo sapiens
NSVEVALNK

SEQ ID NO: 1348 - protein, 6 aa, Homo sapiens
LLDPIR

SEQ ID NO: 1349 - protein, 7 aa, Homo sapiens
FNTPALK

SEQ ID NO: 1350 - protein, 11 aa, Homo sapiens
LASAAYPDPSK

SEQ ID NO: 1351 - protein, 11 aa, Homo sapiens
NSEPEEVIPSR

SEQ ID NO: 1352 - protein, 6 aa, Homo sapiens
IITVEK

SEQ ID NO: 1353 - protein, 11 aa, Homo sapiens
HPDADSLYVEK

SEQ ID NO: 1354 - protein, 9 aa, Homo sapiens
IDVGEAEPR

SEQ ID NO: 1355 - protein, 12 aa, Homo sapiens
TVVSGLVQFVPK

SEQ ID NO: 1356 - protein, 20 aa, Homo sapiens
QVEPLDPPAGSAPGEHVFVK

SEQ ID NO: 1357 - protein, 10 aa, Homo sapiens
GQPDEELKPK

SEQ ID NO: 1358 - protein, 6 aa, Homo sapiens
LQADFK

SEQ ID NO: 1359 - protein, 12 aa, Homo sapiens
EAAIVDPVQPQK

SEQ ID NO: 1360 - protein, 19 aa, Homo sapiens
LTTVLTTHHHWDHAGGNEK

SEQ ID NO: 1361 - protein, 6 aa, Homo sapiens
LESGLK

SEQ ID NO: 1362 - protein, 7 aa, Homo sapiens
VYGGDDR

SEQ ID NO: 1363 - protein, 7 aa, Homo sapiens
IGALTHK

SEQ ID NO: 1364 - protein, 15 aa, Homo sapiens
ITHLSTLQVGSLNVK

SEQ ID NO: 1365 - protein, 8 aa, Homo sapiens
ALLEVLGR

SEQ ID NO: 1366 - protein, 10 aa, Homo sapiens
HVEPGNAAIR

SEQ ID NO: 1367 - protein, 7 aa, Homo sapiens
LDDDSER

SEQ ID NO: 1368 - protein, 8 aa, Homo sapiens
DISSIGLK

SEQ ID NO: 1369 - protein, 7 aa, Homo sapiens
LTSAIAK

SEQ ID NO: 1370 - protein, 7 aa, Homo sapiens
IGEYLEK

SEQ ID NO: 1371 - protein, 9 aa, Homo sapiens
TVSPALISR

SEQ ID NO: 1372 - protein, 12 aa, Homo sapiens
ADVFHAYLSLLK

SEQ ID NO: 1373 - protein, 14 aa, Homo sapiens
ITSEALLVTQQLVK

SEQ ID NO: 1374 - protein, 11 aa, Homo sapiens
ALTLIAGSPLK

SEQ ID NO: 1375 - protein, 19 aa, Homo sapiens
IDLRPVLGEGVPILASFLR

SEQ ID NO: 1376 - protein, 12 aa, Homo sapiens
LGTLSALDILIK

SEQ ID NO: 1377 - protein, 8 aa, Homo sapiens
VYPSSLSK

SEQ ID NO: 1378 - protein, 14 aa, Homo sapiens
ISGSILNELIGLVR

SEQ ID NO: 1379 - protein, 8 aa, Homo sapiens
QSYYSIAK

SEQ ID NO: 1380 - protein, 14 aa, Homo sapiens
EGPAVVGQFIQDVK

SEQ ID NO: 1381 - protein, 6 aa, Homo sapiens
STDSIR

SEQ ID NO: 1382 - protein, 23 aa, Homo sapiens
LLALLSLGEVGHHIDLSGQLELK

SEQ ID NO: 1383 - protein, 15 aa, Homo sapiens
SVILEAFSSPSEEVK

SEQ ID NO: 1384 - protein, 9 aa, Homo sapiens
QYLLLHSLK

SEQ ID NO: 1385 - protein, 24 aa, Homo sapiens
EIISSASVVGLKPYVENIWALLLK

SEQ ID NO: 1386 - protein, 12 aa, Homo sapiens
LTLIDPETLLPR

SEQ ID NO: 1387 - protein, 11 aa, Homo sapiens
GYLISGSSYAR

SEQ ID NO: 1388 - protein, 8 aa, Homo sapiens
SSVVTAVK

SEQ ID NO: 1389 - protein, 15 aa, Homo sapiens
FTISDHPQPIDPLLK

SEQ ID NO: 1390 - protein, 10 aa, Homo sapiens
TLEDPDLNVR

SEQ ID NO: 1391 - protein, 18 aa, Homo sapiens
VALVTFNSAAHNKPSLIR

SEQ ID NO: 1392 - protein, 15 aa, Homo sapiens
DLLDTVLPHLYNETK

SEQ ID NO: 1393 - protein, 10 aa, Homo sapiens
HTVDDGLDIR

SEQ ID NO: 1394 - protein, 15 aa, Homo sapiens
LDIFEFLNHVEDGLK

SEQ ID NO: 1395 - protein, 6 aa, Homo sapiens
DHYDIK

SEQ ID NO: 1396 - protein, 6 aa, Homo sapiens
LVEPLR

SEQ ID NO: 1397 - protein, 13 aa, Homo sapiens
AVAALLTIPEAEK

SEQ ID NO: 1398 - protein, 18 aa, Homo sapiens
VIRPLDQPSSFDATPYIK

SEQ ID NO: 1399 - protein, 9 aa, Homo sapiens
AADIDQEVK

SEQ ID NO: 1400 - protein, 10 aa, Homo sapiens
SDGTPFPWNK

SEQ ID NO: 1401 - protein, 23 aa, Homo sapiens
VEITASQPTSTIILHSHHLQISR

SEQ ID NO: 1402 - protein, 13 aa, Homo sapiens
LSEEPLQVLEHPR

SEQ ID NO: 1403 - protein, 13 aa, Homo sapiens
ILASTQFEPTAAR

SEQ ID NO: 1404 - protein, 6 aa, Homo sapiens
ASFSIK

SEQ ID NO: 1405 - protein, 18 aa, Homo sapiens
SVTVAEGLIEDHFDVTVK

SEQ ID NO: 1406 - protein, 9 aa, Homo sapiens
VSVYAVPDK

SEQ ID NO: 1407 - protein, 10 aa, Homo sapiens
ESALLFDAEK

SEQ ID NO: 1408 - protein, 8 aa, Homo sapiens
VGDYFFGK

SEQ ID NO: 1409 - protein, 9 aa, Homo sapiens
EYLSADAFK

SEQ ID NO: 1410 - protein, 9 aa, Homo sapiens
SGIVQYLQK

SEQ ID NO: 1411 - protein, 18 aa, Homo sapiens
SQHSSSSSHWHQEGVDVK

SEQ ID NO: 1412 - protein, 10 aa, Homo sapiens
GFPLITITVR

SEQ ID NO: 1413 - protein, 22 aa, Homo sapiens
GSDGAPDTGYLWHVPLTFITSK

SEQ ID NO: 1414 - protein, 14 aa, Homo sapiens
TDVLILPEEVEWIK

SEQ ID NO: 1415 - protein, 11 aa, Homo sapiens
GTHTAVSSNDR

SEQ ID NO: 1416 - protein, 15 aa, Homo sapiens
ASLINNAFQLVSIGK

SEQ ID NO: 1417 - protein, 9 aa, Homo sapiens
ALDLSLYLK

SEQ ID NO: 1418 - protein, 12 aa, Homo sapiens
QTWTDEGSVSER

SEQ ID NO: 1419 - protein, 10 aa, Homo sapiens
YQFSLSSTEK

SEQ ID NO: 1420 - protein, 10 aa, Homo sapiens
LQWLLDESFK

SEQ ID NO: 1421 - protein, 13 aa, Homo sapiens
TQEFPQILTLIGR

SEQ ID NO: 1422 - protein, 13 aa, Homo sapiens
NPVGYPLAWQFLR

SEQ ID NO: 1423 - protein, 7 aa, Homo sapiens
GFFSSLK

SEQ ID NO: 1424 - protein, 7 aa, Homo sapiens
ENGSQLR

SEQ ID NO: 1425 - protein, 7 aa, Homo sapiens
VWLQSEK

SEQ ID NO: 1426 - protein, 24 aa, Homo sapiens
GSVLLAQELPQQLTSPGYPEPYGK

SEQ ID NO: 1427 - protein, 9 aa, Homo sapiens
GQESSTDIK

SEQ ID NO: 1428 - protein, 8 aa, Homo sapiens
APEGFAVR

SEQ ID NO: 1429 - protein, 7 aa, Homo sapiens
EFVSSGR

SEQ ID NO: 1430 - protein, 8 aa, Homo sapiens
TQPSSENK

SEQ ID NO: 1431 - protein, 6 aa, Homo sapiens
TAHLHK

SEQ ID NO: 1432 - protein, 22 aa, Homo sapiens
GFLALYQTVAVNYSQPISEASR

SEQ ID NO: 1433 - protein, 14 aa, Homo sapiens
GSEAINAPGDNPAK

SEQ ID NO: 1434 - protein, 15 aa, Homo sapiens
LGNFPWQAFTSIHGR

SEQ ID NO: 1435 - protein, 12 aa, Homo sapiens
WILTAAHTIYPK

SEQ ID NO: 1436 - protein, 8 aa, Homo sapiens
LGNHPVHR

SEQ ID NO: 1437 - protein, 8 aa, Homo sapiens
VVVHPDYR

SEQ ID NO: 1438 - protein, 9 aa, Homo sapiens
VLSYVDWIK

SEQ ID NO: 1439 - protein, 13 aa, Homo sapiens
TFIIDYYFEVVQK

SEQ ID NO: 1440 - skipped

SEQ ID NO: 1441 - protein, 9 aa, Homo sapiens
FGVYDIDNK

SEQ ID NO: 1442 - protein, 7 aa, Homo sapiens
TGRPAGK

SEQ ID NO: 1443 - protein, 11 aa, Homo sapiens
GSITISAEEIK

SEQ ID NO: 1444 - protein, 9 aa, Homo sapiens
SDPYLEFHK

SEQ ID NO: 1445 - protein, 11 aa, Homo sapiens
NNLNPVWRPFK

SEQ ID NO: 1446 - protein, 8 aa, Homo sapiens
NSGVISVK

SEQ ID NO: 1447 - protein, 16 aa, Homo sapiens
LYGPTNFSPIINHVAR

SEQ ID NO: 1448 - protein, 8 aa, Homo sapiens
QAIVNASR

SEQ ID NO: 1449 - protein, 9 aa, Homo sapiens
SPLGEVAIR

SEQ ID NO: 1450 - protein, 7 aa, Homo sapiens
QFQNAPK

SEQ ID NO: 1451 - protein, 11 aa, Homo sapiens
NTAEWLLSHTK

SEQ ID NO: 1452 - protein, 17 aa, Homo sapiens
LTQAQIFDYGEIPNFPR

SEQ ID NO: 1453 - protein, 9 aa, Homo sapiens
STVPGHAGR

SEQ ID NO: 1454 - protein, 9 aa, Homo sapiens
LVFGFLNGR

SEQ ID NO: 1455 - protein, 22 aa, Homo sapiens
VFHLLGVDTLVVTNAAGGLNPK

SEQ ID NO: 1456 - protein, 15 aa, Homo sapiens
DHINLPGFSGQNPLR

SEQ ID NO: 1457 - protein, 6 aa, Homo sapiens
GPNDER

SEQ ID NO: 1458 - protein, 6 aa, Homo sapiens
ALSTWK

SEQ ID NO: 1459 - protein, 10 aa, Homo sapiens
VFGFSLITNK

SEQ ID NO: 1460 - protein, 11 aa, Homo sapiens
ANHEEVLAAGK

SEQ ID NO: 1461 - protein, 6 aa, Homo sapiens
ILNRPK

SEQ ID NO: 1462 - protein, 11 aa, Homo sapiens
LEEFVQGNLER

SEQ ID NO: 1463 - protein, 8 aa, Homo sapiens
EVFENTER

SEQ ID NO: 1464 - protein, 6 aa, Homo sapiens
TTEFWK

SEQ ID NO: 1465 - protein, 6 aa, Homo sapiens
NSADNK

SEQ ID NO: 1466 - protein, 6 aa, Homo sapiens
LAENQK

SEQ ID NO: 1467 - protein, 8 aa, Homo sapiens
VSVSQTSK

SEQ ID NO: 1468 - protein, 21 aa, Homo sapiens
VVGGEDAKPGQFPWQVVLNGK

SEQ ID NO: 1469 - protein, 19 aa, Homo sapiens
ITVVAGEHNIEETEHTEQK

SEQ ID NO: 1470 - protein, 13 aa, Homo sapiens
IIPHHNYNAAINK

SEQ ID NO: 1471 - protein, 8 aa, Homo sapiens
EYTNIFLK

SEQ ID NO: 1472 - protein, 11 aa, Homo sapiens
FGSGYVSGWGR

SEQ ID NO: 1473 - protein, 9 aa, Homo sapiens
SALVLQYLR

SEQ ID NO: 1474 - protein, 6 aa, Homo sapiens
VPLVDR

SEQ ID NO: 1475 - protein, 11 aa, Homo sapiens
ALDFAVGEYNK

SEQ ID NO: 1476 - protein, 16 aa, Homo sapiens
QIVAGVNYFLDVELGR

SEQ ID NO: 1477 - protein, 17 aa, Homo sapiens
VPAQLLGLLLLWLSGAR

SEQ ID NO: 1478 - protein, 16 aa, Homo sapiens
LLIYDASNLETGVPSR

SEQ ID NO: 1479 - protein, 22 aa, Homo sapiens
GLEWVSAISGSGGSTYYADSVK

SEQ ID NO: 1480 - protein, 6 aa, Homo sapiens
AHQVLR

SEQ ID NO: 1481 - protein, 9 aa, Homo sapiens
ANSFLEELR

SEQ ID NO: 1482 - protein, 15 aa, Homo sapiens
EIFQNVDDTLAFWSK

SEQ ID NO: 1483 - protein, 6 aa, Homo sapiens
SGWEGR

SEQ ID NO: 1484 - protein, 12 aa, Homo sapiens
DTEDQEDQVDPR

SEQ ID NO: 1485 - protein, 13 aa, Homo sapiens
GDSPWQVVLLDSK

SEQ ID NO: 1486 - protein, 7 aa, Homo sapiens
LGEYDLR

SEQ ID NO: 1487 - protein, 8 aa, Homo sapiens
WELDLDIK

SEQ ID NO: 1488 - protein, 10 aa, Homo sapiens
EVFVHPNYSK

SEQ ID NO: 1489 - protein, 20 aa, Homo sapiens
ELNQAGQETLVTGWGYHSSR

SEQ ID NO: 1490 - protein, 8 aa, Homo sapiens
TFVLNFIK

SEQ ID NO: 1491 - protein, 10 aa, Homo sapiens
YLDWIHGHIR

SEQ ID NO: 1492 - protein, 13 aa, Homo sapiens
TIPIDGDFFSYTR

SEQ ID NO: 1493 - protein, 17 aa, Homo sapiens
VAEQTPLTALYVANLIK

SEQ ID NO: 1494 - protein, 11 aa, Homo sapiens
VAFTGSTEIGR

SEQ ID NO: 1495 - protein, 12 aa, Homo sapiens
VIQVAAGSSNLK

SEQ ID NO: 1496 - protein, 14 aa, Homo sapiens
TFVQEDIYDEFVER

SEQ ID NO: 1497 - protein, 9 aa, Homo sapiens
VVGNPFDSK

SEQ ID NO: 1498 - protein, 13 aa, Homo sapiens
TEQGPQVDETQFK

SEQ ID NO: 1499 - protein, 9 aa, Homo sapiens
ILGYINTGK

SEQ ID NO: 1500 - protein, 8 aa, Homo sapiens
TIEEVVGR

SEQ ID NO: 1501 - protein, 15 aa, Homo sapiens
ANNSTYGLAAAVFTK

SEQ ID NO: 1502 - protein, 14 aa, Homo sapiens
ELGEYGLQAYTEVK

SEQ ID NO: 1503 - protein, 10 aa, Homo sapiens
AAFQLGSPWR

SEQ ID NO: 1504 - protein, 10 aa, Homo sapiens
LLGIETPLPK

SEQ ID NO: 1505 - protein, 9 aa, Homo sapiens
ELLLPGNNR

SEQ ID NO: 1506 - protein, 11 aa, Homo sapiens
TFLTVYWTPER

SEQ ID NO: 1507 - protein, 14 aa, Homo sapiens
VELAPLPSWQPVGK

SEQ ID NO: 1508 - protein, 9 aa, Homo sapiens
ANLTVVLLR

SEQ ID NO: 1509 - protein, 17 aa, Homo sapiens
EPAVGEPAEVTTTVLVR

SEQ ID NO: 1510 - protein, 15 aa, Homo sapiens
LNPTVTYGNDSFSAK

SEQ ID NO: 1511 - protein, 14 aa, Homo sapiens
ASVSVTAEDEGTQR

SEQ ID NO: 1512 - protein, 14 aa, Homo sapiens
VTLNGVPAQPLGPR

SEQ ID NO: 1513 - protein, 6 aa, Homo sapiens
AQLLLK

SEQ ID NO: 1514 - protein, 8 aa, Homo sapiens
ATPEDNGR

SEQ ID NO: 1515 - protein, 6 aa, Homo sapiens
VLYGPR

SEQ ID NO: 1516 - protein, 16 aa, Homo sapiens
DGTFPLPIGESVTVTR

SEQ ID NO: 1517 - protein, 8 aa, Homo sapiens
STQGEVTR

SEQ ID NO: 1518 - protein, 9 aa, Homo sapiens
VTVNVLSPR

SEQ ID NO: 1519 - protein, 6 aa, Homo sapiens
VVVITK

SEQ ID NO: 1520 - protein, 15 aa, Homo sapiens
NPDDITQEEYGEFYK

SEQ ID NO: 1521 - protein, 7 aa, Homo sapiens
ALLFIPR

SEQ ID NO: 1522 - protein, 7 aa, Homo sapiens
FYEAFSK

SEQ ID NO: 1523 - protein, 10 aa, Homo sapiens
LGIHEDSTNR

SEQ ID NO: 1524 - protein, 15 aa, Homo sapiens
HLEINPDHPIVETLR

SEQ ID NO: 1525 - protein, 12 aa, Homo sapiens
GTVTDFPGFDER

SEQ ID NO: 1526 - protein, 7 aa, Homo sapiens
ADAETLR

SEQ ID NO: 1527 - protein, 16 aa, Homo sapiens
GLGTDEESILTLLTSR

SEQ ID NO: 1528 - protein, 8 aa, Homo sapiens
QEISAAFK

SEQ ID NO: 1529 - protein, 7 aa, Homo sapiens
DLLDDLK

SEQ ID NO: 1530 - protein, 6 aa, Homo sapiens
SELTGK

SEQ ID NO: 1531 - protein, 8 aa, Homo sapiens
LYDAYELK

SEQ ID NO: 1532 - protein, 7 aa, Homo sapiens
GAGTNEK

SEQ ID NO: 1533 - protein, 9 aa, Homo sapiens
VLTEIIASR

SEQ ID NO: 1534 - protein, 25 aa, Homo sapiens
QVYEEEYGSSLEDDVVGDTSGYYQR

SEQ ID NO: 1535 - protein, 25 aa, Homo sapiens
DPDAGIDEAQVEQDAQALFQAGELK

SEQ ID NO: 1536 - protein, 7 aa, Homo sapiens
WGTDEEK

SEQ ID NO: 1537 - protein, 8 aa, Homo sapiens
FITIFGTR

SEQ ID NO: 1538 - protein, 15 aa, Homo sapiens
ETSGNLEQLLLAVVK

SEQ ID NO: 1539 - protein, 11 aa, Homo sapiens
GAGTDDHTLIR

SEQ ID NO: 1540 - protein, 9 aa, Homo sapiens
SEIDLFNIR

SEQ ID NO: 1541 - protein, 8 aa, Homo sapiens
GDTSGDYK

SEQ ID NO: 1542 - protein, 24 aa, Homo sapiens
QSGVNATLPEENQPVVFNHVYNIK

SEQ ID NO: 1543 - protein, 12 aa, Homo sapiens
LEELENLVSSLR

SEQ ID NO: 1544 - protein, 22 aa, Homo sapiens
VPGDQTSTIIQELEPGVEYFIR

SEQ ID NO: 1545 - protein, 8 aa, Homo sapiens
VFAILENK

SEQ ID NO: 1546 - protein, 7 aa, Homo sapiens
SIPVSAR

SEQ ID NO: 1547 - protein, 12 aa, Homo sapiens
VATYLPAPEGLK

SEQ ID NO: 1548 - protein, 23 aa, Homo sapiens
ETSVEVEWDPLDIAFETWEIIFR

SEQ ID NO: 1549 - protein, 8 aa, Homo sapiens
EDEGEITK

SEQ ID NO: 1550 - protein, 7 aa, Homo sapiens
RPETSYR

SEQ ID NO: 1551 - protein, 18 aa, Homo sapiens
QTGLAPGQEYEISLHIVK

SEQ ID NO: 1552 - protein, 10 aa, Homo sapiens
LDAPSQIEVK

SEQ ID NO: 1553 - protein, 11 aa, Homo sapiens
ETFTTGLDAPR

SEQ ID NO: 1554 - protein, 13 aa, Homo sapiens
VSQTDNSITLEWR

SEQ ID NO: 1555 - protein, 7 aa, Homo sapiens
AAIDSYR

SEQ ID NO: 1556 - protein, 16 aa, Homo sapiens
YAPISGGDHAEVDVPK

SEQ ID NO: 1557 - protein, 7 aa, Homo sapiens
SQQATTK

SEQ ID NO: 1558 - protein, 20 aa, Homo sapiens
TTLTGLRPGTEYGIGVSAVK

SEQ ID NO: 1559 - protein, 17 aa, Homo sapiens
ESNPATINAATELDTPK

SEQ ID NO: 1560 - protein, 17 aa, Homo sapiens
DLQVSETAETSLTLLWK

SEQ ID NO: 1561 - protein, 17 aa, Homo sapiens
LNYSLPTGQWVGVQLPR

SEQ ID NO: 1562 - protein, 8 aa, Homo sapiens
NTTSYVLR

SEQ ID NO: 1563 - protein, 16 aa, Homo sapiens
GLEPGQEYNVLLTAEK

SEQ ID NO: 1564 - protein, 23 aa, Homo sapiens
ASTEQAPELENLTVTEVGWDGLR

SEQ ID NO: 1565 - protein, 22 aa, Homo sapiens
LNWTAADQAYEHFIIQVQEANK

SEQ ID NO: 1566 - protein, 9 aa, Homo sapiens
NLTVPGSLR

SEQ ID NO: 1567 - protein, 8 aa, Homo sapiens
AVDIPGLK

SEQ ID NO: 1568 - protein, 17 aa, Homo sapiens
AATPYTVSIYGVIQGYR

SEQ ID NO: 1569 - protein, 8 aa, Homo sapiens
STDLPGLK

SEQ ID NO: 1570 - protein, 10 aa, Homo sapiens
AATHYTITIR

SEQ ID NO: 1571 - protein, 14 aa, Homo sapiens
AGTPYTVTLHGEVR

SEQ ID NO: 1572 - protein, 22 aa, Homo sapiens
LNWTAADNAYEHFVIQVQEVNK

SEQ ID NO: 1573 - protein, 14 aa, Homo sapiens
VEAAQNLTLPGSLR

SEQ ID NO: 1574 - protein, 14 aa, Homo sapiens
AVDIPGLEAATPYR

SEQ ID NO: 1575 - protein, 8 aa, Homo sapiens
VSIYGVIR

SEQ ID NO: 1576 - protein, 12 aa, Homo sapiens
TPVLSAEASTAK

SEQ ID NO: 1577 - protein, 14 aa, Homo sapiens
LLETVEYNISGAER

SEQ ID NO: 1578 - protein, 25 aa, Homo sapiens
TAHISGLPPSTDFIVYLSGLAPSIR

SEQ ID NO: 1579 - protein, 14 aa, Homo sapiens
LLDPQEFTLSGTQR

SEQ ID NO: 1580 - protein, 25 aa, Homo sapiens
AEIVTEAEPEVDNLLVSDATPDGFR

SEQ ID NO: 1581 - protein, 16 aa, Homo sapiens
LSWTADEGVFDNFVLK

SEQ ID NO: 1582 - protein, 14 aa, Homo sapiens
QSEPLEITLLAPER

SEQ ID NO: 1583 - protein, 14 aa, Homo sapiens
EATEYEIELYGISK

SEQ ID NO: 1584 - protein, 17 aa, Homo sapiens
EVIFSDITENSATVSWR

SEQ ID NO: 1585 - protein, 10 aa, Homo sapiens
APTAQVESFR

SEQ ID NO: 1586 - protein, 19 aa, Homo sapiens
WQPAIATVDSYVISYTGEK

SEQ ID NO: 1587 - protein, 6 aa, Homo sapiens
VPEITR

SEQ ID NO: 1588 - protein, 23 aa, Homo sapiens
TVSGNTVEYALTDLEPATEYTLR

SEQ ID NO: 1589 - protein, 7 aa, Homo sapiens
SSTITAK

SEQ ID NO: 1590 - protein, 9 aa, Homo sapiens
FTTDLDSPR

SEQ ID NO: 1591 - protein, 20 aa, Homo sapiens
DLTATEVQSETALLTWRPPR

SEQ ID NO: 1592 - protein, 18 aa, Homo sapiens
ASVTGYLLVYESVDGTVK

SEQ ID NO: 1593 - protein, 25 aa, Homo sapiens
EVIVGPDTTSYSLADLSPSTHYTAK

SEQ ID NO: 1594 - protein, 9 aa, Homo sapiens
IQALNGPLR

SEQ ID NO: 1595 - protein, 8 aa, Homo sapiens
ENFYQNWK

SEQ ID NO: 1596 - protein, 9 aa, Homo sapiens
AYAAGFGDR

SEQ ID NO: 1597 - protein, 12 aa, Homo sapiens
EEFWLGLDNLNK

SEQ ID NO: 1598 - protein, 10 aa, Homo sapiens
ITAQGQYELR

SEQ ID NO: 1599 - protein, 12 aa, Homo sapiens
DHGETAFAVYDK

SEQ ID NO: 1600 - protein, 7 aa, Homo sapiens
FSVGDAK

SEQ ID NO: 1601 - protein, 7 aa, Homo sapiens
SFSTFDK

SEQ ID NO: 1602 - protein, 6 aa, Homo sapiens
GAFWYR

SEQ ID NO: 1603 - protein, 16 aa, Homo sapiens
YGDNNHSQGVNWFHWK

SEQ ID NO: 1604 - protein, 9 aa, Homo sapiens
HGSLGFLPR

SEQ ID NO: 1605 - protein, 16 aa, Homo sapiens
DDPSKPVHLTAFLGYK

SEQ ID NO: 1606 - protein, 8 aa, Homo sapiens
EVDRPGSK

SEQ ID NO: 1607 - protein, 7 aa, Homo sapiens
WQDEDGK

SEQ ID NO: 1608 - protein, 7 aa, Homo sapiens
VAFSVAR

SEQ ID NO: 1609 - protein, 8 aa, Homo sapiens
IGQGYLIK

SEQ ID NO: 1610 - protein, 12 aa, Homo sapiens
NNASTDYDLSDK

SEQ ID NO: 1611 - protein, 7 aa, Homo sapiens
SLLVQTK

SEQ ID NO: 1612 - protein, 8 aa, Homo sapiens
QQLEELAR

SEQ ID NO: 1613 - protein, 9 aa, Homo sapiens
ALAEGVLLR

SEQ ID NO: 1614 - protein, 6 aa, Homo sapiens
NIFDQR

SEQ ID NO: 1615 - protein, 9 aa, Homo sapiens
AIENELLAR

SEQ ID NO: 1616 - protein, 6 aa, Homo sapiens
NIHVIR

SEQ ID NO: 1617 - protein, 8 aa, Homo sapiens
TFEDISEK

SEQ ID NO: 1618 - protein, 7 aa, Homo sapiens
GSLDQDR

SEQ ID NO: 1619 - protein, 14 aa, Homo sapiens
LFVDGQEIAVVYFR

SEQ ID NO: 1620 - protein, 10 aa, Homo sapiens
QYSLQNWEAR

SEQ ID NO: 1621 - protein, 7 aa, Homo sapiens
FVLKPQR

SEQ ID NO: 1622 - protein, 7 aa, Homo sapiens
HVGHLLR

SEQ ID NO: 1623 - protein, 22 aa, Homo sapiens
AIEHADGGVAAGVAVLDNPYPV

SEQ ID NO: 1624 - protein, 7 aa, Homo sapiens
QDDFTAR

SEQ ID NO: 1625 - protein, 13 aa, Homo sapiens
EGIAQTVFLGLNR

SEQ ID NO: 1626 - protein, 9 aa, Homo sapiens
SADGSPALK

SEQ ID NO: 1627 - protein, 17 aa, Homo sapiens
QIEINTISASFGGLASR

SEQ ID NO: 1628 - protein, 6 aa, Homo sapiens
TPAVHR

SEQ ID NO: 1629 - protein, 8 aa, Homo sapiens
HVLSVLSK

SEQ ID NO: 1630 - protein, 8 aa, Homo sapiens
ILSNNPSK

SEQ ID NO: 1631 - protein, 8 aa, Homo sapiens
GLALGIAK

SEQ ID NO: 1632 - protein, 19 aa, Homo sapiens
AWELYGSPNALVLLIAQEK

SEQ ID NO: 1633 - protein, 7 aa, Homo sapiens
DGHSLGR

SEQ ID NO: 1634 - protein, 8 aa, Homo sapiens
AIDGINQR

SEQ ID NO: 1635 - protein, 8 aa, Homo sapiens
SSDANLYR

SEQ ID NO: 1636 - protein, 9 aa, Homo sapiens
FALLGDFFR

SEQ ID NO: 1637 - protein, 7 aa, Homo sapiens
ALLLWGR

SEQ ID NO: 1638 - protein, 8 aa, Homo sapiens
HEFQAETK

SEQ ID NO: 1639 - protein, 7 aa, Homo sapiens
LLDIVAR

SEQ ID NO: 1640 - protein, 6 aa, Homo sapiens
SLYSEK

SEQ ID NO: 1641 - protein, 12 aa, Homo sapiens
ELISNASDALEK

SEQ ID NO: 1642 - protein, 15 aa, Homo sapiens
AFLDALQNQAEASSK

SEQ ID NO: 1643 - protein, 6 aa, Homo sapiens
IIIHLK

SEQ ID NO: 1644 - protein, 7 aa, Homo sapiens
EFSSEAR

SEQ ID NO: 1645 - protein, 14 aa, Homo sapiens
YSNFVSFPLYLNGR

SEQ ID NO: 1646 - protein, 9 aa, Homo sapiens
EWQHEEFYR

SEQ ID NO: 1647 - protein, 10 aa, Homo sapiens
YVAQAHDKPR

SEQ ID NO: 1648 - protein, 6 aa, Homo sapiens
YTLHYK

SEQ ID NO: 1649 - protein, 8 aa, Homo sapiens
TDAPLNIR

SEQ ID NO: 1650 - protein, 11 aa, Homo sapiens
ELGSSVALYSR

SEQ ID NO: 1651 - protein, 6 aa, Homo sapiens
VLIQTK

SEQ ID NO: 1652 - protein, 7 aa, Homo sapiens
ATDILPK

SEQ ID NO: 1653 - protein, 14 aa, Homo sapiens
GVVDSEDIPLNLSR

SEQ ID NO: 1654 - protein, 10 aa, Homo sapiens
ELLQESALIR

SEQ ID NO: 1655 - protein, 7 aa, Homo sapiens
FFIDQSK

SEQ ID NO: 1656 - protein, 12 aa, Homo sapiens
EGIVTATEQEVK

SEQ ID NO: 1657 - protein, 20 aa, Homo sapiens
YESSALPSGQLTSLSEYASR

SEQ ID NO: 1658 - protein, 14 aa, Homo sapiens
LISVETDIVVDHYK

SEQ ID NO: 1659 - protein, 6 aa, Homo sapiens
NVLGSR

SEQ ID NO: 1660 - protein, 13 aa, Homo sapiens
AQLLQPTLEINPR

SEQ ID NO: 1661 - protein, 7 aa, Homo sapiens
LNELLVK

SEQ ID NO: 1662 - protein, 8 aa, Homo sapiens
TTAQLGPR

SEQ ID NO: 1663 - protein, 10 aa, Homo sapiens
NPAWSLQAGR

SEQ ID NO: 1664 - protein, 10 aa, Homo sapiens
LFSTQTAEDK

SEQ ID NO: 1665 - protein, 20 aa, Homo sapiens
EEPLHSIISSTESVQGSTSK

SEQ ID NO: 1666 - protein, 6 aa, Homo sapiens
SITQIK

SEQ ID NO: 1667 - protein, 10 aa, Homo sapiens
EGDLLFTVAK

SEQ ID NO: 1668 - protein, 14 aa, Homo sapiens
DPIVNVWYSVNGER

SEQ ID NO: 1669 - protein, 6 aa, Homo sapiens
TNSAVR

SEQ ID NO: 1670 - protein, 9 aa, Homo sapiens
SGEVLVNVK

SEQ ID NO: 1671 - protein, 9 aa, Homo sapiens
QINDIQLSR

SEQ ID NO: 1672 - protein, 11 aa, Homo sapiens
LFDSTTLEHQK

SEQ ID NO: 1673 - protein, 12 aa, Homo sapiens
FFHLAFEEEFGR

SEQ ID NO: 1674 - protein, 16 aa, Homo sapiens
GHFGPINSVAFHPDGK

SEQ ID NO: 1675 - protein, 12 aa, Homo sapiens
SYSSGGEDGYVR

SEQ ID NO: 1676 - protein, 15 aa, Homo sapiens
IHYFDPQYFEFEFEA

SEQ ID NO: 1677 - protein, 13 aa, Homo sapiens
NEEDEGHSNSSPR

SEQ ID NO: 1678 - protein, 9 aa, Homo sapiens
HSEAATAQR

SEQ ID NO: 1679 - protein, 7 aa, Homo sapiens
LDPITGR

SEQ ID NO: 1680 - protein, 7 aa, Homo sapiens
ESESVDK

SEQ ID NO: 1681 - protein, 14 aa, Homo sapiens
IFVGGLSPDTPEEK

SEQ ID NO: 1682 - protein, 8 aa, Homo sapiens
YHNVGLSK

SEQ ID NO: 1683 - protein, 12 aa, Homo sapiens
EQYQQQQQWGSR

SEQ ID NO: 1684 - protein, 10 aa, Homo sapiens
GGHQNSYKPY

SEQ ID NO: 1685 - protein, 7 aa, Homo sapiens
GHLDYPR

SEQ ID NO: 1686 - protein, 6 aa, Homo sapiens
IDHLER

SEQ ID NO: 1687 - protein, 13 aa, Homo sapiens
LLAENNEIISNIR

SEQ ID NO: 1688 - protein, 15 aa, Homo sapiens
DSVINLSESVEDGPK

SEQ ID NO: 1689 - protein, 7 aa, Homo sapiens
TFNDYFR

SEQ ID NO: 1690 - protein, 11 aa, Homo sapiens
FIWSEISYLSK

SEQ ID NO: 1691 - protein, 9 aa, Homo sapiens
WWDIIDIQK

SEQ ID NO: 1692 - protein, 6 aa, Homo sapiens
VHYAVK

SEQ ID NO: 1693 - protein, 6 aa, Homo sapiens
HFALHK

SEQ ID NO: 1694 - protein, 7 aa, Homo sapiens
TLEFFWR

SEQ ID NO: 1695 - protein, 11 aa, Homo sapiens
VLLAPLGDDFR

SEQ ID NO: 1696 - protein, 15 aa, Homo sapiens
IQFGTLSDFFDALDK

SEQ ID NO: 1697 - protein, 6 aa, Homo sapiens
ADETQR

SEQ ID NO: 1698 - protein, 16 aa, Homo sapiens
DDHYWSGYFTSRPFYK

SEQ ID NO: 1699 - protein, 11 aa, Homo sapiens
AAEILYYFALR

SEQ ID NO: 1700 - protein, 14 aa, Homo sapiens
FLSSSLYTALTEAR

SEQ ID NO: 1701 - protein, 16 aa, Homo sapiens
NLGLFQHHDAITGTAK

SEQ ID NO: 1702 - protein, 10 aa, Homo sapiens
DWVVVDYGTR

SEQ ID NO: 1703 - protein, 12 aa, Homo sapiens
IIGNSAFLLILK

SEQ ID NO: 1704 - protein, 8 aa, Homo sapiens
SQDSLPQK

SEQ ID NO: 1705 - protein, 12 aa, Homo sapiens
YLVVYNPLEQDR

SEQ ID NO: 1706 - protein, 9 aa, Homo sapiens
AHIPPLGLK

SEQ ID NO: 1707 - protein, 18 aa, Homo sapiens
ILESASSNSHLADYVLYK

SEQ ID NO: 1708 - protein, 10 aa, Homo sapiens
VEDSGIFTIK

SEQ ID NO: 1709 - protein, 16 aa, Homo sapiens
HHEVNVQFSWYGTTIK

SEQ ID NO: 1710 - protein, 24 aa, Homo sapiens
SGAYLFLPDGNAKPYVYTTPPFVR

SEQ ID NO: 1711 - protein, 21 aa, Homo sapiens
LYHIQGIEGQSVEVSNIVDIR

SEQ ID NO: 1712 - protein, 6 aa, Homo sapiens
ISSDIK

SEQ ID NO: 1713 - protein, 13 aa, Homo sapiens
FYTDLNGYQIQPR

SEQ ID NO: 1714 - protein, 10 aa, Homo sapiens
GLEQGIQDNK

SEQ ID NO: 1715 - protein, 7 aa, Homo sapiens
ITANLFR

SEQ ID NO: 1716 - protein, 9 aa, Homo sapiens
SAVNTEEEK

SEQ ID NO: 1717 - protein, 15 aa, Homo sapiens
VGNGHSNEAALILHR

SEQ ID NO: 1718 - protein, 10 aa, Homo sapiens
AGFGGDDAPR

SEQ ID NO: 1719 - protein, 11 aa, Homo sapiens
IWYHTFYNELR

SEQ ID NO: 1720 - protein, 18 aa, Homo sapiens
VAPDEHPILLTEAPLNPK

SEQ ID NO: 1721 - protein, 10 aa, Homo sapiens
GYNFTTTAER

SEQ ID NO: 1722 - protein, 9 aa, Homo sapiens
AAASSSPER

SEQ ID NO: 1723 - protein, 13 aa, Homo sapiens
QEYDEAGPPIVHR

SEQ ID NO: 1724 - protein, 8 aa, Homo sapiens
SSFLTEEK

SEQ ID NO: 1725 - protein, 9 aa, Homo sapiens
LYLENGQTK

SEQ ID NO: 1726 - protein, 8 aa, Homo sapiens
FEANTTVK

SEQ ID NO: 1727 - protein, 7 aa, Homo sapiens
DIILTVK

SEQ ID NO: 1728 - protein, 17 aa, Homo sapiens
SIEYFALALEEQYSISR

SEQ ID NO: 1729 - protein, 16 aa, Homo sapiens
LHLLHEEELIQQVVER

SEQ ID NO: 1730 - protein, 7 aa, Homo sapiens
EESHDYR

SEQ ID NO: 1731 - protein, 7 aa, Homo sapiens
DPLDLLK

SEQ ID NO: 1732 - protein, 9 aa, Homo sapiens
LAALHIQER

SEQ ID NO: 1733 - protein, 14 aa, Homo sapiens
DWGIENFISPTLLR

SEQ ID NO: 1734 - protein, 8 aa, Homo sapiens
NQNLLEPR

SEQ ID NO: 1735 - protein, 9 aa, Homo sapiens
QLISAAQLR

SEQ ID NO: 1736 - protein, 11 aa, Homo sapiens
LNYLQILGELK

SEQ ID NO: 1737 - protein, 11 aa, Homo sapiens
ESYIALLVGAK

SEQ ID NO: 1738 - protein, 10 aa, Homo sapiens
YGISQVINSK

SEQ ID NO: 1739 - protein, 9 aa, Homo sapiens
VELTEESEK

SEQ ID NO: 1740 - protein, 7 aa, Homo sapiens
VYLQDVK

SEQ ID NO: 1741 - protein, 12 aa, Homo sapiens
VLTLLLESNSAK

SEQ ID NO: 1742 - protein, 16 aa, Homo sapiens
LLVDPVTSIFLWPGNK

SEQ ID NO: 1743 - protein, 10 aa, Homo sapiens
VSAEEGYESR

SEQ ID NO: 1744 - protein, 15 aa, Homo sapiens
EEQPPGNSPTPEVAR

SEQ ID NO: 1745 - protein, 23 aa, Homo sapiens
STFFHFGSPGLAESIDSDSQEER

SEQ ID NO: 1746 - protein, 22 aa, Homo sapiens
TEFSESAALETFGWAPELSTVR

SEQ ID NO: 1747 - protein, 9 aa, Homo sapiens
VAAADGPAR

SEQ ID NO: 1748 - protein, 10 aa, Homo sapiens
NPTQTLIPVR

SEQ ID NO: 1749 - protein, 6 aa, Homo sapiens
AVDILR

SEQ ID NO: 1750 - protein, 12 aa, Homo sapiens
EAEDSLSITVVR

SEQ ID NO: 1751 - protein, 11 aa, Homo sapiens
LTPPGPPSGPR

SEQ ID NO: 1752 - protein, 15 aa, Homo sapiens
DVSTAEPSATSLQNK

SEQ ID NO: 1753 - protein, 9 aa, Homo sapiens
ALGLLAPLR

SEQ ID NO: 1754 - protein, 7 aa, Homo sapiens
STNPASR

SEQ ID NO: 1755 - protein, 6 aa, Homo sapiens
SVIDSR

SEQ ID NO: 1756 - protein, 8 aa, Homo sapiens
VSSISAIR

SEQ ID NO: 1757 - protein, 6 aa, Homo sapiens
IDPNNK

SEQ ID NO: 1758 - protein, 22 aa, Homo sapiens
EPTIEHGDSSFSLSSGDPNPDR

SEQ ID NO: 1759 - protein, 9 aa, Homo sapiens
YTEPLLSPR

SEQ ID NO: 1760 - protein, 7 aa, Homo sapiens
IASIPTK

SEQ ID NO: 1761 - protein, 11 aa, Homo sapiens
EEPQGQLSLER

SEQ ID NO: 1762 - protein, 11 aa, Homo sapiens
NGTNVFQEESR

SEQ ID NO: 1763 - protein, 25 aa, Homo sapiens
DSGDSPGDVSNNVSQTLDISSPAGK

SEQ ID NO: 1764 - protein, 23 aa, Homo sapiens
IVTSLSLDAPVTGTEQIPPHPPR

SEQ ID NO: 1765 - protein, 7 aa, Homo sapiens
DPQGQSR

SEQ ID NO: 1766 - protein, 15 aa, Homo sapiens
LFVELDLDPDFFLGK

SEQ ID NO: 1767 - protein, 13 aa, Homo sapiens
QTVSPAVPPEGIK

SEQ ID NO: 1768 - protein, 15 aa, Homo sapiens
AEAPNHVTGQDIAPR

SEQ ID NO: 1769 - protein, 11 aa, Homo sapiens
GPQPETEEEDR

SEQ ID NO: 1770 - protein, 7 aa, Homo sapiens
EAAGNLR

SEQ ID NO: 1771 - protein, 14 aa, Homo sapiens
DVVYTYHQFIEAAK

SEQ ID NO: 1772 - protein, 8 aa, Homo sapiens
GYHDLSVK

SEQ ID NO: 1773 - protein, 7 aa, Homo sapiens
VYLPWSR

SEQ ID NO: 1774 - protein, 10 aa, Homo sapiens
VYSTSVTGSR

SEQ ID NO: 1775 - protein, 8 aa, Homo sapiens
SQQSEVTR

SEQ ID NO: 1776 - protein, 15 aa, Homo sapiens
IQYQLVDISQDNALR

SEQ ID NO: 1777 - protein, 9 aa, Homo sapiens
GASALQLER

SEQ ID NO: 1778 - protein, 11 aa, Homo sapiens
ISVYYNEAYGR

SEQ ID NO: 1779 - protein, 23 aa, Homo sapiens
LGALFQPDSFVHGNSGAGNNWAK

SEQ ID NO: 1780 - protein, 18 aa, Homo sapiens
GHYTEGAELIENVLEVVR

SEQ ID NO: 1781 - protein, 12 aa, Homo sapiens
EVDQQLLSVQTR

SEQ ID NO: 1782 - protein, 9 aa, Homo sapiens
LVNIAVDER

SEQ ID NO: 1783 - protein, 8 aa, Homo sapiens
SSPYYALR

SEQ ID NO: 1784 - protein, 21 aa, Homo sapiens
EEIYFLNTNNAFSGVATYTDK

SEQ ID NO: 1785 - protein, 13 aa, Homo sapiens
WEDILSDEVNVAR

SEQ ID NO: 1786 - protein, 9 aa, Homo sapiens
GVASLFAGR

SEQ ID NO: 1787 - protein, 6 aa, Homo sapiens
GILALR

SEQ ID NO: 1788 - protein, 10 aa, Homo sapiens
DVAAEAGVSK

SEQ ID NO: 1789 - protein, 22 aa, Homo sapiens
GDGTFVDAAASAGVDDPHQHGR

SEQ ID NO: 1790 - protein, 9 aa, Homo sapiens
GVALADFNR

SEQ ID NO: 1791 - protein, 13 aa, Homo sapiens
VDIVYGNWNGPHR

SEQ ID NO: 1792 - protein, 6 aa, Homo sapiens
DIASPK

SEQ ID NO: 1793 - protein, 23 aa, Homo sapiens
TVITADFDNDQELEIFFNNIAYR

SEQ ID NO: 1794 - protein, 6 aa, Homo sapiens
SSSANR

SEQ ID NO: 1795 - protein, 21 aa, Homo sapiens
EHGDPLIEELNPGDALEPEGR

SEQ ID NO: 1796 - protein, 11 aa, Homo sapiens
GNQGFNNNWLR

SEQ ID NO: 1797 - protein, 6 aa, Homo sapiens
FGAFAR

SEQ ID NO: 1798 - protein, 6 aa, Homo sapiens
VVLYTK

SEQ ID NO: 1799 - protein, 6 aa, Homo sapiens
SGAHLR

SEQ ID NO: 1800 - protein, 14 aa, Homo sapiens
DEASSVEVTWPDGK

SEQ ID NO: 1801 - protein, 7 aa, Homo sapiens
YGFQSLR

SEQ ID NO: 1802 - protein, 13 aa, Homo sapiens
YQSHDYAFSSVEK

SEQ ID NO: 1803 - protein, 8 aa, Homo sapiens
TLLETLQK

SEQ ID NO: 1804 - protein, 6 aa, Homo sapiens
AGFSEK

SEQ ID NO: 1805 - protein, 7 aa, Homo sapiens
YTGNPTK

SEQ ID NO: 1806 - protein, 15 aa, Homo sapiens
SDFYDIVLVATPLNR

SEQ ID NO: 1807 - protein, 15 aa, Homo sapiens
GELNTSIFSSRPIDK

SEQ ID NO: 1808 - protein, 14 aa, Homo sapiens
EDPEPSTDGTYVWK

SEQ ID NO: 1809 - protein, 9 aa, Homo sapiens
IFSQETLTK

SEQ ID NO: 1810 - protein, 10 aa, Homo sapiens
LFLSYDYAVK

SEQ ID NO: 1811 - protein, 14 aa, Homo sapiens
KPWLAYPHYKPPEK

SEQ ID NO: 1812 - protein, 18 aa, Homo sapiens
IAIIGAGIGGTSAAYYLR

SEQ ID NO: 1813 - protein, 6 aa, Homo sapiens
IDLFER

SEQ ID NO: 1814 - protein, 6 aa, Homo sapiens
EEVGGR

SEQ ID NO: 1815 - protein, 8 aa, Homo sapiens
EAVQTAAK

SEQ ID NO: 1816 - protein, 10 aa, Homo sapiens
LLQAGFSELK

SEQ ID NO: 1817 - protein, 8 aa, Homo sapiens
WNIKPESK

SEQ ID NO: 1818 - protein, 24 aa, Homo sapiens
SQVGFQQVGVETYGGGIWSTWFDR

SEQ ID NO: 1819 - protein, 7 aa, Homo sapiens
DLTLAGR

SEQ ID NO: 1820 - protein, 14 aa, Homo sapiens
LEQQLVHVERPILR

SEQ ID NO: 1821 - protein, 10 aa, Homo sapiens
IPHLAIHLQR

SEQ ID NO: 1822 - protein, 14 aa, Homo sapiens
GTPEPGPLNAVDER

SEQ ID NO: 1823 - protein, 11 aa, Homo sapiens
HEENHRPLFHK

SEQ ID NO: 1824 - protein, 12 aa, Homo sapiens
YASNAVSEALIR

SEQ ID NO: 1825 - protein, 16 aa, Homo sapiens
GFFELFPSLSHNLLVD

SEQ ID NO: 1826 - protein, 10 aa, Homo sapiens
GSGTNGYVQR

SEQ ID NO: 1827 - protein, 6 aa, Homo sapiens
NLSLVR

SEQ ID NO: 1828 - protein, 7 aa, Homo sapiens
GERPDYK

SEQ ID NO: 1829 - protein, 7 aa, Homo sapiens
LEAALVK

SEQ ID NO: 1830 - protein, 11 aa, Homo sapiens
RPNPDILDHER

SEQ ID NO: 1831 - protein, 7 aa, Homo sapiens
DVNPGGK

SEQ ID NO: 1832 - protein, 22 aa, Homo sapiens
EETPGQRPAVTETHQLAELNEK

SEQ ID NO: 1833 - protein, 19 aa, Homo sapiens
AAFGISDSYVDGSSFDPQR

SEQ ID NO: 1834 - protein, 14 aa, Homo sapiens
QPAPEPPKPYSLVR

SEQ ID NO: 1835 - protein, 7 aa, Homo sapiens
STTPAPK

SEQ ID NO: 1836 - protein, 14 aa, Homo sapiens
STSADSASSSDTSR

SEQ ID NO: 1837 - protein, 9 aa, Homo sapiens
THTTALAGR

SEQ ID NO: 1838 - protein, 8 aa, Homo sapiens
SPSPASGR

SEQ ID NO: 1839 - protein, 9 aa, Homo sapiens
QPSSPYEDK

SEQ ID NO: 1840 - protein, 11 aa, Homo sapiens
SATRPSPSPER

SEQ ID NO: 1841 - protein, 17 aa, Homo sapiens
SSTGPEPPAPTPLLAER

SEQ ID NO: 1842 - protein, 18 aa, Homo sapiens
LPQSSSSESSPPSPQPTK

SEQ ID NO: 1843 - protein, 19 aa, Homo sapiens
HASSSPESPKPAPAPGSHR

SEQ ID NO: 1844 - protein, 9 aa, Homo sapiens
EISSSPTSK

SEQ ID NO: 1845 - protein, 8 aa, Homo sapiens
SHSHTPSR

SEQ ID NO: 1846 - protein, 6 aa, Homo sapiens
SPATAK

SEQ ID NO: 1847 - protein, 9 aa, Homo sapiens
SPQRPGWSR

SEQ ID NO: 1848 - protein, 7 aa, Homo sapiens
SGSSQPK

SEQ ID NO: 1849 - protein, 8 aa, Homo sapiens
SSSSPPPK

SEQ ID NO: 1850 - protein, 11 aa, Homo sapiens
QSHSSSSPHPK

SEQ ID NO: 1851 - protein, 6 aa, Homo sapiens
SGTPPR

SEQ ID NO: 1852 - protein, 18 aa, Homo sapiens
QGSITSPQANEQSVTPQR

SEQ ID NO: 1853 - protein, 6 aa, Homo sapiens
AIISPR

SEQ ID NO: 1854 - protein, 12 aa, Homo sapiens
SHSGSSSPSPSR

SEQ ID NO: 1855 - protein, 12 aa, Homo sapiens
YSHSGSSSPDTK

SEQ ID NO: 1856 - protein, 8 aa, Homo sapiens
VKPETPPR

SEQ ID NO: 1857 - protein, 12 aa, Homo sapiens
QSHSGSISPYPK

SEQ ID NO: 1858 - protein, 13 aa, Homo sapiens
AQTPPGPSLSGSK

SEQ ID NO: 1859 - protein, 18 aa, Homo sapiens
SSTPPGESYFGVSSLQLK

SEQ ID NO: 1860 - protein, 10 aa, Homo sapiens
GQSQTSPDHR

SEQ ID NO: 1861 - protein, 9 aa, Homo sapiens
SDTSSPEVR

SEQ ID NO: 1862 - protein, 12 aa, Homo sapiens
QSHSESPSLQSK

SEQ ID NO: 1863 - protein, 6 aa, Homo sapiens
SQTSPK

SEQ ID NO: 1864 - protein, 11 aa, Homo sapiens
SSSPVTELASR

SEQ ID NO: 1865 - protein, 21 aa, Homo sapiens
FQSDSSSYPTVDSNSLLGQSR

SEQ ID NO: 1866 - protein, 7 aa, Homo sapiens
LETAESK

SEQ ID NO: 1867 - protein, 17 aa, Homo sapiens
FSPFPVQDRPESSLVFK

SEQ ID NO: 1868 - protein, 10 aa, Homo sapiens
SGAGSSPETK

SEQ ID NO: 1869 - protein, 17 aa, Homo sapiens
SEEPAGQILSHLSSELK

SEQ ID NO: 1870 - protein, 8 aa, Homo sapiens
ELSNSPLR

SEQ ID NO: 1871 - protein, 11 aa, Homo sapiens
ENSFGSPLEFR

SEQ ID NO: 1872 - protein, 16 aa, Homo sapiens
SSGHSSSELSPDAVEK

SEQ ID NO: 1873 - protein, 8 aa, Homo sapiens
SGSSPGLR

SEQ ID NO: 1874 - protein, 8 aa, Homo sapiens
DGSGTPSR

SEQ ID NO: 1875 - protein, 9 aa, Homo sapiens
ALPQTPRPR

SEQ ID NO: 1876 - protein, 10 aa, Homo sapiens
SPSSPELNNK

SEQ ID NO: 1877 - protein, 10 aa, Homo sapiens
SGSESSVDQK

SEQ ID NO: 1878 - protein, 6 aa, Homo sapiens
TPLGQR

SEQ ID NO: 1879 - protein, 18 aa, Homo sapiens
SGSSQELDVKPSASPQER

SEQ ID NO: 1880 - protein, 10 aa, Homo sapiens
SESDSSPDSK

SEQ ID NO: 1881 - protein, 10 aa, Homo sapiens
SGSSPEVDSK

SEQ ID NO: 1882 - protein, 8 aa, Homo sapiens
SGSSPEVK

SEQ ID NO: 1883 - protein, 12 aa, Homo sapiens
AQSGSDSSPEPK

SEQ ID NO: 1884 - protein, 19 aa, Homo sapiens
GPSPEGSSSTESSPEHPPK

SEQ ID NO: 1885 - protein, 7 aa, Homo sapiens
SSPELTR

SEQ ID NO: 1886 - protein, 9 aa, Homo sapiens
SASSSPETR

SEQ ID NO: 1887 - protein, 12 aa, Homo sapiens
SPSVSSPEPAEK

SEQ ID NO: 1888 - protein, 7 aa, Homo sapiens
SPSPKPR

SEQ ID NO: 1889 - protein, 9 aa, Homo sapiens
SGSSQSTSR

SEQ ID NO: 1890 - protein, 8 aa, Homo sapiens
GGSGYHSR

SEQ ID NO: 1891 - protein, 7 aa, Homo sapiens
TSPAPWK

SEQ ID NO: 1892 - protein, 7 aa, Homo sapiens
ASPATHR

SEQ ID NO: 1893 - protein, 6 aa, Homo sapiens
TPLISR

SEQ ID NO: 1894 - protein, 6 aa, Homo sapiens
TPPVTR

SEQ ID NO: 1895 - protein, 6 aa, Homo sapiens
TSPITR

SEQ ID NO: 1896 - protein, 12 aa, Homo sapiens
SPVPSAFSDQSR

SEQ ID NO: 1897 - protein, 9 aa, Homo sapiens
VPSPTPAPK

SEQ ID NO: 1898 - protein, 11 aa, Homo sapiens
EGRPPEPTPAK

SEQ ID NO: 1899 - protein, 7 aa, Homo sapiens
KPPPGER

SEQ ID NO: 1900 - protein, 7 aa, Homo sapiens
KPIDSLR

SEQ ID NO: 1901 - protein, 9 aa, Homo sapiens
SLSYSPVER

SEQ ID NO: 1902 - protein, 9 aa, Homo sapiens
RPSPQPSPR

SEQ ID NO: 1903 - protein, 9 aa, Homo sapiens
DQQSSSSER

SEQ ID NO: 1904 - protein, 6 aa, Homo sapiens
TPPAIR

SEQ ID NO: 1905 - protein, 6 aa, Homo sapiens
SPLAIR

SEQ ID NO: 1906 - protein, 6 aa, Homo sapiens
SPPAIR

SEQ ID NO: 1907 - protein, 9 aa, Homo sapiens
SASGSSSDR

SEQ ID NO: 1908 - protein, 8 aa, Homo sapiens
SATPPATR

SEQ ID NO: 1909 - protein, 6 aa, Homo sapiens
NHSGSR

SEQ ID NO: 1910 - protein, 10 aa, Homo sapiens
TPPVALNSSR

SEQ ID NO: 1911 - protein, 6 aa, Homo sapiens
IPDHQR

SEQ ID NO: 1912 - protein, 11 aa, Homo sapiens
TSVPENHAQSR

SEQ ID NO: 1913 - protein, 10 aa, Homo sapiens
TAPAANLASR

SEQ ID NO: 1914 - protein, 14 aa, Homo sapiens
TPAIPTAVNLADSR

SEQ ID NO: 1915 - protein, 14 aa, Homo sapiens
TAVAPSAVNLADPR

SEQ ID NO: 1916 - protein, 13 aa, Homo sapiens
TPTAPAVNLAGAR

SEQ ID NO: 1917 - protein, 14 aa, Homo sapiens
TPQAPASANLVGPR

SEQ ID NO: 1918 - protein, 14 aa, Homo sapiens
SAHATAPVNIAGSR

SEQ ID NO: 1919 - protein, 14 aa, Homo sapiens
TAAALAPASLTSAR

SEQ ID NO: 1920 - protein, 8 aa, Homo sapiens
VPLSAYER

SEQ ID NO: 1921 - protein, 8 aa, Homo sapiens
TSPPLLDR

SEQ ID NO: 1922 - protein, 10 aa, Homo sapiens
TPPSAPSQSR

SEQ ID NO: 1923 - protein, 7 aa, Homo sapiens
APSPSSR

(19) United States
(12) Patent Application Publication
(10) Pub. No.: US 2024/0426842 A1
(43) Pub. Date: Dec. 26, 2024

(54) NOVEL BIOMARKERS AND METHODS FOR DIAGNOSING AND EVALUATING TRAUMATIC BRAIN INJURY

(71) Applicants: Abbott Laboratories, Abbott Park, IL (US); Cedars-Sinai Medical Center, Los Angeles, CA (US)

(72) Inventors: Jennifer Van Eyk, Los Angeles, CA (US); Beth McQuiston, Abott Park, IL (US); Saul Datwyler, Abott Park, IL (US); Raj Chandran, Abbott Park, IL (US); Vidya Venkatraman, Los Angeles, CA (US); Shenyan Zhang, Los Angeles, CA (US)

(21) Appl. No.: 18/640,030

(22) Filed: Apr. 19, 2024

Related U.S. Application Data
(63) Continuation of application No. 17/224,685, filed on Apr. 7, 2021, now Pat. No. 11,994,523.
Application No. 17/224,685 is a division of application No. 16/233,895, filed on Dec. 27, 2018, now abandoned.
(60) Provisional application No. 62/630,704, filed on Feb. 14, 2018.
(60) Provisional application No. 62/611,778, filed on Dec. 29, 2017.

(51) Int. Cl. G01N 33/68

(52) U.S. Cl. CPC G01N 33/6896; G01N 2560/00; G01N 2800/28; G01N 2800/50; G01N 2800/52; G01N 2800/60

(57) ABSTRACT

The present disclosure relates to methods for diagnosing and evaluating a subject that has sustained or may have sustained an injury to the head, such as a traumatic brain injury (TBI). In particular, the present disclosure identifies various biomarkers, the detection and/or differential expression of which can be used to assess the presence or absence of a TBI in a subject, and can be used as a basis for diagnosing a subject as having a specific type of TBI (e.g., severe TBI or subclasses of mild TBI). The various TBI biomarkers can be detected individually or in combination and can be used as an important diagnostic, prognostic, and/or TBI risk stratification tool as part of assessing a subject's TBI status.

### CROSS REFERENCE TO RELATED APPLICATIONS

This application is a continuation of U.S. application Ser. No. 17/224,685, filed Apr. 7, 2021, which is a divisional of U.S. application Ser. No. 16/233,895, filed Dec. 27, 2018, which claims priority to U.S. Provisional Application No. 62/611,778, filed Dec. 29, 2017, and U.S. Provisional Application No. 62/630,704, filed Feb. 14, 2018, all of which are incorporated herein by reference in their entireties.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,660,155 Byte XML file named “ABBTL-36107.305-ST26,” created on Apr. 18, 2024.

### FIELD

The present disclosure relates to methods for diagnosing and evaluating a subject that has sustained or may have sustained an injury to the head, such as a traumatic brain injury (TBI). In particular, the present disclosure identifies various biomarkers, the detection and/or differential expression of which can be used to assess the presence or absence of a TBI in a subject, and can be used as a basis for diagnosing a subject as having a specific type of TBI (e.g., severe TBI or subclasses of mild TBI). The various TBI biomarkers can be detected individually or in combination and can be used as an important diagnostic, prognostic, and/or TBI risk stratification tool as part of assessing a subject's TBI status.

### BACKGROUND

More than 5 million mild traumatic brain injuries (mTBIs) occur each year in the United States alone. Currently, there is no simple, objective, accurate measurement available to help in patient assessment. In fact, much of TBI evaluation and diagnosis is based on subjective data. Unfortunately, objective measurements such as head CT and Glasgow Coma Score (GCS) are not very comprehensive or sensitive in evaluating mild TBI. Moreover, head CT is unrevealing for the vast majority of the time for mTBI, is expensive, and exposes the patient to unnecessary radiation. Additionally, a negative head CT does not mean the patient has been cleared from having a concussion; rather, it may simply indicate that certain interventions, such as surgery, are not warranted. Clinicians and patients need objective, reliable information to accurately evaluate this condition to promote appropriate triage and recovery. To date, limited data have been available for the use of biomarkers to aid in patient diagnosis, evaluation, and management.

The majority of TBI's are mild; however, the ability to diagnose and treat mTBI is insufficient, in large part because the ability to characterize accurately the different TBI disease states or “disease signatures” is lacking. This insufficiency generally stems from challenges associated with interpreting ambiguous clinically presented symptoms and ineffective imaging techniques. Thus, researchers have begun to explore cellular and molecular based approaches to improve both diagnosis and prognosis. This has been met with a variety of challenges, including difficulty in relating biological markers to current clinical symptoms, and overcoming our lack of fundamental understanding of the pathophysiology of mTBI. However, recent adoption of techniques such as high-throughput technologies and computational biology have provided the means for more accurately determining and characterizing TBI disease states.

Because the underlying pathophysiology of TBI remains undetermined, effective and efficient diagnostic, prognostic, risk stratification, and/or therapeutic tools are not yet currently available, especially in a clinical setting. Researchers have begun to investigate TBI at the cellular and molecular level, as shortcomings in current brain imaging techniques and flawed clinical diagnostic approaches have increased the appeal of utilizing the peripheral blood to identify immune and damage related signaling between the brain and the periphery. The ultimate goal of this approach is to uncover single TBI biomarkers or panels of biomarkers to aid in early detection and diagnosis, to effectively distinguish between various TBI disease states (e.g., mTBI vs. severe TBI or sTBI), and to help predict patient outcomes. Furthermore, these approaches may help elucidate underlying biological mechanisms and provide greater insight into therapeutic strategies.

### SUMMARY

Embodiments of the present disclosure include a method of measuring or detecting at least one biomarker. In accordance with these embodiments, the method includes obtaining a sample from a subject after an actual or suspected head injury; and measuring or detecting at least one biomarker or fragment thereof selected from the group consisting of AL9A1, ATPG, C1RL, CAND1, EPIPL, GLO2, IGHA2, PZP, SYTC, SYYC, or any combinations thereof in the sample; and/or measuring or detecting at least one biomarker of fragment thereof selected from the group consisting of ABHEB, AL9A1, DNM1L, FCN2, INF2, K22E, M3K5, NCOR1, SBSN, SYEP, TPP2, or any combinations thereof in the sample. In some embodiments, the measurement or detection of the at least one biomarker indicates that the subject has sustained or may have sustained a traumatic brain injury (TBI).

Embodiments of the present disclosure also include a method of measuring or detecting at least one biomarker that includes obtaining a sample from a subject after an actual or suspected head injury; and measuring or detecting at least one biomarker or fragment thereof selected from the group consisting of 1433G, ACK1, ACY1, AKA12, ARGI1, CADH5, CLH1, COPG2, DPOD2, DSG2, HV307, IQGA2, K1C14, K1C19, KV105, LAMC1, MDHM, NQO2, PERM, PLST, PNCB, PTPRC, SEPT7, SYRC, TRXR2, TXNL1, UGGG1, WDR1 or any combinations thereof in the sample. In some embodiments, the measurement or detection of the at least one biomarker indicates that the subject has sustained or may have sustained a mild traumatic brain injury (mTBI).

Embodiments of the present disclosure also include a biomarker panel for determining traumatic brain injury (TBI) status of a subject. In accordance with these embodiments, the panel includes at least one of the following biomarkers: TPP2, CAND1, NCOR1, K22E, AL9A1, ABHEB, DNM1L, INF2, or any combinations thereof; wherein measurement or detection of the at least one biomarker indicates that the subject has sustained or may have sustained a mild TBI of subclass 4.

Embodiments of the present disclosure also include a biomarker panel for determining traumatic brain injury (TBI) status of a subject that includes at least one of the following biomarkers: TPP2, NCOR1, HV103, INF2, IGHD, CK054, M3K5, ABHEB, AL9A1, DNM1L, or any combinations thereof; wherein measurement or detection of the at least one biomarker indicates that the subject has sustained or may have sustained a mild TBI of subclass 3.

Embodiments of the present disclosure also include a biomarker panel for determining traumatic brain injury (TBI) status of a subject that includes at least one of the following biomarkers: NCOR1, TPP2, K22E, ABHEB, INF2, SBSN, AL9A1, MA2B2, or any combinations thereof; wherein measurement or detection of the at least one biomarker indicates that the subject has sustained or may have sustained a mild TBI of subclass 2.

Embodiments of the present disclosure also include a biomarker panel for determining traumatic brain injury (TBI) status of a subject that includes at least one of the following biomarkers: K22E, DNM1L, DIAP1, ABHEB, PLOD1, SYEP, KV133, AL9A1, EPHB4, or any combinations thereof; wherein measurement or detection of the at least one biomarker indicates that the subject has sustained or may have sustained a mild TBI of subclass 1.

Embodiments of the present disclosure also include a biomarker panel for determining traumatic brain injury (TBI) status of a subject that includes at least one of the following biomarkers: CAND1, NCOR1, K22E, ABHEB, DNM1L, SBSN, GLO2, SYEP, or any combinations thereof; wherein measurement or detection of higher levels of the at least one biomarker in the subject as compared to levels of the at least one biomarkers in a healthy subject indicates that the subject has sustained or may have sustained a mild TBI of subclass 4.

Embodiments of the present disclosure also include a biomarker panel for determining traumatic brain injury (TBI) status of a subject that includes at least one of the following biomarkers: NCOR1, HV103, IGHD, ABHEB, DNM1L, ALBU, THIM, IGHA2, KV139, and, or any combinations thereof; wherein measurement or detection of higher levels of the at least one biomarker in the subject as compared to levels of the at least one biomarkers in a healthy subject indicates that the subject has sustained or may have sustained a mild TBI of subclass 3.

Embodiments of the present disclosure also include a biomarker panel for determining traumatic brain injury (TBI) status of a subject that includes at least one of the following biomarkers: NCOR1, K22E, ABHEB, SBSN, DNM1L, DIAP1, DYL1, PSA, EPHB4, or any combinations thereof; wherein measurement or detection of higher levels of the at least one biomarker in the subject as compared to levels of the at least one biomarkers in a healthy subject indicates that the subject has sustained or may have sustained a mild TBI of subclass 2.

Embodiments of the present disclosure also include a biomarker panel for determining traumatic brain injury (TBI) status of a subject that includes at least one of the following biomarkers: K22E, DNM1L, DIAP1, ABHEB, PLOD1, SYEP, EPHB4, FBLN3, or any combinations thereof; wherein measurement or detection of higher levels of the at least one biomarker in the subject as compared to levels of the at least one biomarkers in a healthy subject indicates that the subject has sustained or may have sustained a mild TBI of subclass 1.

Embodiments of the present disclosure also include a biomarker panel for determining that a subject has not sustained a traumatic brain injury (TBI) that includes at least one of the following biomarkers: ACTBL, ALDH2, ANXA5, CAMP, CPNE3, CRAC1, CYTC, DNPEP, EIF3I, GSHB, ICAM1, HV323, HNRPD, KVD33, FA9, FHR4, FRPD1, HS90B, MA2A1, PCYOX, PNPH, PROC, RL3, SH3L3, SRRM2, TBB1, TENA, TRAP1 or any combinations thereof; wherein measurement or detection of the at least one biomarker in the subject indicates that the subject has not sustained a TBI.

### BRIEF DESCRIPTION OF THE DRAWINGS

FIGS. 1A-IC include representative graphs obtained from unsupervised clustering analysis of quantitative mass spectrometry discovery proteomic data from plasma samples obtained from subjects classified as healthy or as having mild TBI based on clinical physiological information, as well as pooled plasma from severe (i.e. CT positive) subjects. All data points are labeled according to their cluster locations in FIG. 1A. FIG. 1A is a representative phylogenic plot resulting from unsupervised clustering analysis of whole proteome profile data showing two major clusters (control cluster) and TBI clusters (TBI subclass 1=TBI-1; TBI subclass 2=TBI-2; TBI subclass 3=TBI-3; and TBI subclass 4=Complex TBI), along with associated patient and sample characteristics that were extracted post-hoc including Age; Gender; Normalized GFAP MS peak area (using targeted mass spectrometry-based GFAP assay based on quantification of individual prototypic peptides); CT scan (positive/negative; gray indicates no data); and ELISA-based GFAP biomarker scores (positive/negative; gray indicates no data). Predominant pathological clusters of mild TBI subclasses include subclass 1 (TBI-1), subclass 2 (TBI-2), subclass 3 (TBI-3), and subclass 4 (Complex TBI), which is also referred to herein as “complicated mild TBI.” FIG. 1B includes results from principal component analysis of the whole proteome profile. FIG. 1C includes representative phylogenic clustering depicted as a constellation plot.

FIGS. 2A-2C include representative clustering of proteins that are shared and/or unique between sample clusters based on unsupervised analysis. Predominant pathological clusters of mild TBI subclasses include subclass 1 (TBI-1), subclass 2 (TBI-2), subclass 3 (TBI-3), and subclass 4, or complicated mild TBI (Complex TBI); a healthy control cluster is also shown (Control). Each of five main sample sub-clusters derived from unsupervised clustering analysis has a minimum of five uniquely expressed proteins. FIG. 2A is a representative Venn diagram that includes shared and/or unique proteins between the five identified subgroup clusters. FIG. 3B includes a numeric count of total proteins and/or unique proteins quantified in each subgroup cluster. FIG. 3C includes Uniprot ID and short name designations for the unique proteins quantified in each subgroup cluster.

FIGS. 3A-3B include a representative Venn plot of proteins that are shared or unique between healthy (left circle) and TBI patient samples (including the pooled severe sample; right circle). Only proteins detected in all the samples in each group were included in this analysis (FIG. 3A). FIG. 3B includes a list of 8 proteins uniquely expressed in all TBI samples, including their Uniprot ID and protein name. AL9A1 and SYTC are expressed in brain while FA5 is related to coagulation (both designations are based on annotated information from UniProt (uniprot.org)).

FIG. 4A-4E include representative schematics of Bootstrap Forest Analysis as an alternative approach to traditional supervised analysis of quantitative independent acquisition-mass spectrometry (DIA-MS) data. Contributions of individual proteins were analyzed and summarized in the five separate decision trees that met the criteria of the prediction model (FIGS. 4A-4E). This analysis ascribed a quantitative cutoff for the ability of a given protein to serve as a biomarker capable of distinguishing patients with TBI from their healthy counterparts, along with an associated diagnostic probability. In the case where a single protein was insufficiently capable of making this distinction, a combination of multiple proteins is identified and defined.

FIG. 5 is a table of a Bootstrap Forest Analysis that was performed as an alternative approach to traditional supervised analysis of quantitative DIA-MS data. This analysis ranks proteins according to their contributing portion of a whole.

### DETAILED DESCRIPTION

The present disclosure relates to methods for diagnosing and evaluating a subject that has sustained or may have sustained an injury to the head, such as a traumatic brain injury (TBI). In particular, the present disclosure identifies various biomarkers, the detection and/or differential expression of which can be used to assess the presence or absence of a TBI in a subject, and can be used as a basis for diagnosing a subject as having a specific type of TBI (e.g., severe TBI or subclasses of mTBI). The various TBI biomarkers can be detected individually or in combination and can be used as an important diagnostic and therapeutic tool for assessing a subject's TBI status.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, i.e. regardless of whether it is positive or negative.

This disclosure provides exemplary reference levels and absolute amounts (e.g., calculated by comparing reference levels at different time points). However, it is well-known that reference levels and absolute amounts may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels and absolute amounts for those other immunoassays based on the description provided by this disclosure. Whereas the precise value of the reference level and absolute amount may vary between assays, the findings as described herein should be generally applicable and capable of being extrapolated to other assays.

“Affinity matured antibody” is used herein to refer to an antibody with one or more alterations in one or more CDRs, which result in an improvement in the affinity (i.e. K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2).

“Antibody fragment” as used herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Bispecific antibody” is used herein to refer to a full-length antibody that is generated by quadroma technology (see Milstein et al., Nature, 305 (5934): 537-540 (1983)), by chemical conjugation of two different monoclonal antibodies (see, Staerz et al., Nature, 314 (6012): 628-631 (1985)), or by knob-into-hole or similar approaches, which introduce mutations in the Fc region (see Holliger et al., Proc. Natl. Acad. Sci. USA, 90 (14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds to.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196:901-917 (1987); and Chothia et al., Nature, 342:877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9:133-139 (1995), and MacCallum, J. Mol. Biol., 262 (5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Coefficient of variation” (CV), also known as “relative variability,” is equal to the standard deviation of a distribution divided by its mean.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, serum or plasma sample, in accordance with the methods described herein and other methods known in the art. Some components can be in solution or lyophilized for reconstitution for use in an assay.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like). As used herein, a “control subject” relates to a subject or subjects that have not sustained a traumatic brain injury (TBI). An “ortho control” as used herein relates to (e.g., is based on) samples or information from a subject or subjects that have sustained an orthopedic injury but have not sustained an apparent TBI. As used herein, an “ortho control subject” relates to a subject or subjects that have sustained an orthopedic injury but have not sustained an apparent TBI. In some cases, “ortho control subjects” are adult orthopedic patients who have an Abbreviated Injury Score of ≤4 (not life threatening) for their extremity and/or pelvis injury and/or rib fracture. A “healthy control” as used herein relates to (e.g., is based on) samples or information from a subject or subjects that are considered healthy and have sustained no apparent TBI or orthopedic injury. As used herein, a “healthy control subject” relates to a subject or subjects that are considered to be healthy and have sustained no apparent TBI or orthopedic injury. As used herein, “TBI control” as used herein relates to (e.g., is based on) samples or information from a subject or subjects that have sustained a head injury but have not sustained an apparent TBI. As used herein, a “TBI control subject” relates to a subject or subjects that have sustained a head injury but have not sustained an apparent TBI.

“Correlated to” as used herein refers to compared to.

“CT scan” as used herein refers to a computerized tomography (CT) scan. A CT scan combines a series of X-ray images taken from different angles and uses computer processing to create cross-sectional images, or slices, of the bones, blood vessels and soft tissues inside your body. The CT scan may use X-ray CT, positron emission tomography (PET), single-photon emission computed tomography (SPECT), computed axial tomography (CAT scan), or computer aided tomography. The CT scan may be a conventional CT scan or a spiral/helical CT scan. In a conventional CT scan, the scan is taken slice by slice and after each slice the scan stops and moves down to the next slice, e.g., from the top of the abdomen down to the pelvis. The conventional CT scan requires patients to hold their breath to avoid movement artefact. The spiral/helical CT scan is a continuous scan which is taken in a spiral fashion and is a much quicker process where the scanned images are contiguous.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“DIA-MS” or “data-independent acquisition mass spectrometry” is used herein to refer to a method of molecular structure determination in which all ions within a selected m/z range are fragmented and analyzed in a second stage of tandem mass spectrometry. Typically, a complex protein mixture (e.g., plasma samples) is digested into peptides and the peptides are analyzed in a mass spectrometer. Tandem mass spectra are generally acquired on each peptide either by fragmenting all ions that enter the mass spectrometer at a given time (i.e. DIA-MS) or by sequentially isolating and fragmenting ranges of m/z (i.e. data-dependent acquisition-MS or DDA-MS). DIA-MS is an alternative to DDA-MS where a fixed number of precursor ions are selected and analyzed by tandem mass spectrometry, but most often can provide the same protein information. Proteotypic peptides (a peptide that is unique to a specific protein identification) are used for quantification of each protein. (See: Holewinski, R. J., et al. Methods Mol Biol. 2016; 1410:165-279; Kirk, J. A., et al. Sci Transl Med. 2015 Dec. 23; 7 (319): 319ra; and Parker, S. J., et al. Proteomics. 2016 August; 16 (15-16): 2221-2237.)

“Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e. capable of binding one antigen (or one specific epitope), or multispecific, i.e. capable of binding two or more antigens (i.e. two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule, but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e. two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25:1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

“Dynamic range” as used herein refers to range over which an assay readout is proportional to the amount of target molecule or analyte in the sample being analyzed. The dynamic range can be the range of linearity of the standard curve.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment,” “biomarker fragment,” or “biomarker peptide” as used herein includes any identifying fragment of any of the TBI biomarkers identified and described herein. “Fragment(s)” include peptides, prototypic peptides, proteolytic peptides, isoforms, including SNPs or post-translationally modified forms, and any endogenously or exogenously induced forms, of any TBI biomarker identified and described herein. Biomarker peptides can be used to represent the quantity of their representative protein in DIA-MS, DDA-MS, multiple reaction monitoring (MRM, also known as selective reaction monitoring or SRM) mass spectrometry assays, or parrell reaction monitoring (PRM) mass spectrometry assays. Proteolytic peptide(s) can be targeted for MS quantification individual or along with internal standards. (Sec: Fu, Q., et al. J Protcome Res. 2017 November (doi: 10.1021/acs.jproteome.7b00623); Fu, Q., et al. Methods Mol Biol. 2016; 1410:249-264; and Liu, X., et al. Methods. 2013 Jun. 15; 61 (3): 304-312.)

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international ImMunoGeneTics® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

“GFAP” is used herein to describe glial fibrillary acidic protein. GFAP is a protein that is encoded by the GFAP gene in humans, and which can be produced (e.g., by recombinant means, in other species). “GFAP status” can mean either the level or amount of GFAP that is circulating at a point in time (such as with a single measure of GFAP), the level or amount of GFAP associated with monitoring (such as with a repeat test on a subject to identify an increase or decrease in GFAP amount), the level or amount of GFAP associated with treatment for traumatic brain injury (whether a primary brain injury and/or a secondary brain injury) or combinations thereof. GFAP was measured by ELISA and by targeted mass spectrometry to specific peptides that were unique to GFAP.

“Glasgow Coma Scale” or “GCS” as used herein refers to a 15 point scale for estimating and categorizing the outcomes of brain injury on the basis of overall social capability or dependence on others. The test measures the motor response, verbal response and eye opening response with these values: I. Motor Response (6—Obeys commands fully; 5—Localizes to noxious stimuli; 4—Withdraws from noxious stimuli; 3—Abnormal flexion, i.e. decorticate posturing; 2—Extensor response, i.e. decerebrate posturing; and 1—No response); II. Verbal Response (5—Alert and Oriented; 4—Confused, yet coherent, speech; 3—Inappropriate words and jumbled phrases consisting of words; 2—Incomprehensible sounds; and 1—No sounds); and III. Eye Opening (4—Spontaneous eye opening; 3—Eyes open to speech; 2—Eyes open to pain; and 1—No eye opening). The final score is determined by adding the values of I+II+III. The final score can be categorized into four possible levels for survival, with a lower number indicating a more severe injury and a poorer prognosis: Mild (13-15); Moderate Disability (9-12) (Loss of consciousness greater than 30 minutes; Physical or cognitive impairments which may or may resolve; and Benefit from Rehabilitation); Severe Disability (3-8) (Coma: unconscious state. No meaningful response, no voluntary activities); and Vegetative State (Less Than 3) (Sleep wake cycles; Arousal, but no interaction with environment; No localized response to pain). Moderate brain injury is defined as a brain injury resulting in a loss of consciousness from 20 minutes to 6 hours and a Glasgow Coma Scale of 9 to 12. Severe brain injury is defined as a brain injury resulting in a loss of consciousness of greater than 6 hours and a Glasgow Coma Scale of 3 to 8.

“Glasgow Outcome Scale” as used herein refers to a global scale for functional outcome that rates patient status into one of five categories: Dead, Vegetative State, Severe Disability, Moderate Disability or Good Recovery.

“Extended Glasgow Outcome Scale” or “GOSE” as used interchangeably herein provides more detailed categorization into eight categories by subdividing the categories of severe disability, moderate disability and good recovery into a lower and upper category as shown in Table 1.

TABLE 1

| | | | |
|---|---|---|---|
| 1 | Death | D | |
| 2 | Vegetative state | VX | |
| 3 | Lower severe | SD− | Condition of unawareness with only |
| | disability | | reflex responses but with periods of |
| 4 | Upper severe | SD+ | spontaneous eye opening |
| | disability | | |
| 5 | Lower | MD− | Patient who is dependent for daily support |
| | moderate | | for mental or physical disability, |
| | disability | | usually a combination of both. If the |
| 6 | Upper | MD+ | patient can be left alone for more than |
| | moderate | | 8 hours at home it is upper level of SD, |
| | disability | | if not then it is low level of SD. |
| 7 | Lower good | GR− | Patients have some disability such as |
| | recovery | | aphasia, hemiparesis or epilepsy and/or |
| 8 | Upper good | GR+ | deficits of memory or personality but |
| | recovery | | are able to look after themselves. They are |
| | | | independent at home but dependent outside. |
| | | | If they are able to return to work even with |
| | | | special arrangement it is upper level of MD, |
| | | | if not then it is low level of MD. |

“Humanized antibody” is used herein to describe an antibody that comprises heavy and light chain variable region sequences from a non-human species (e.g., a mouse) but in which at least a portion of the VH and/or VL sequence has been altered to be more “human-like,” i.e. more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e. donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s).” In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the amino acid or nucleotide sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Imaging procedure” as used herein refers to a medical test that allows the inside of a body to be seen in order to diagnose, treat, and monitor health conditions. An imaging procedure can be a non-invasive procedure that allows diagnosis of diseases and injuries without being intrusive. Examples of imaging procedures include MRI, CT scan, X-rays, positron emission tomography (PET) scan, single-photon emission computed tomography (SPECT), and diffusion tensor imaging (DTI) scan.

“Injury to the head” or “head injury” as used interchangeably herein, refers to any trauma to the scalp, skull, or brain. Such injuries may include only a minor bump on the skull or may be a serious brain injury. Such injuries include primary injuries to the brain and/or secondary injuries to the brain. Primary brain injuries occur during the initial insult and result from displacement of the physical structures of the brain. More specifically, a primary brain injury is the physical damage to parenchyma (tissue, vessels) that occurs during the traumatic event, resulting in shearing and compression of the surrounding brain tissue. Secondary brain injuries occur subsequent to the primary injury and may involve an array of cellular processes. More specifically, a secondary brain injury refers to the changes that evolve over a period of time (from hours to days) after the primary brain injury. It includes an entire cascade of cellular, chemical, tissue, or blood vessel changes in the brain that contribute to further destruction of brain tissue.

An injury to the head can be either closed or open (penetrating). A closed head injury refers to a trauma to the scalp, skull or brain where there is no penetration of the skull by a striking object. An open head injury refers a trauma to the scalp, skull or brain where there is penetration of the skull by a striking object. An injury to the head may be caused by physical shaking of a person, by blunt impact by an external mechanical or other force that results in a closed or open head trauma (e.g., vehicle accident such as with an automobile, plane, train, etc.; blow to the head such as with a baseball bat, or from a firearm), a cerebral vascular accident (e.g., stroke), one or more falls (e.g., as in sports or other activities), explosions or blasts (collectively, “blast injuries”) and by other types of blunt force trauma. Alternatively, an injury to the head may be caused by the ingestion and/or exposure to a chemical, toxin or a combination of a chemical and toxin. Examples of such chemicals and/or toxins include fires, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. Alternatively, an injury to the head may be caused as a result of a subject suffering from an autoimmune disease, a metabolic disorder, a brain tumor, one or more viruses, meningitis, hydrocephalus, hypoxia or any combinations thereof. In some cases, it is not possible to be certain whether any such event or injury has occurred or taken place. For example, there may be no history on a patient or subject, the subject may be unable to speak, the subject may not be aware of or have full information on what events they were exposed to, etc. Such circumstances are described herein as the subject “may have sustained an injury to the head.” In certain embodiments herein, the closed head injury does not include and specifically excludes a cerebral vascular accident, such as stroke.

“Intracranial lesion” as used herein refers to an area of injury within the brain. An intracranial lesion can be an abnormality seen on a imaging procedure or brain-imaging test, such as MRI or CT scan. On CT or MRI scans, brain lesions can appear as dark or light spots that do not look like normal brain tissue.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5 (6)-carboxyfluorescein, 6-hexachloro-fluorescein, 6-tetrachlorofluorescein, fluorescein isothiocyanate, and the like)), rhodamine, phycobiliproteins, R-phycocrythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oregon. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16:1324-1328 (2006); Adamczyk et al., Bioorg. Med. Chem. Lett. 4:2313-2317 (2004); Adamczyk et al., Biorg. Med. Chem. Lett. 14:3917-3921 (2004); and Adamczyk et al., Org. Lett. 5:3779-3782 (2003)).

In one aspect, the acridinium compound is an acridinium-9-carboxamide. Methods for preparing acridinium 9-carboxamides are described in Mattingly, J. Biolumin. Chemilumin. 6:107-114 (1991); Adamczyk et al., J. Org. Chem. 63:5636-5639 (1998); Adamczyk et al., Tetrahedron 55:10899-10914 (1999); Adamczyk et al., Org. Lett. 1:779-781 (1999); Adamczyk et al., Bioconjugate Chem. 11:714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5:3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl) acridinium fluorosulfonate (available from Cayman Chemical, Ann Arbor, MI). Methods for preparing acridinium 9-carboxylate aryl esters are described in McCapra et al., Photochem. Photobiol. 4:1111-21 (1965); Razavi et al., Luminescence 15:245-249 (2000); Razavi et al., Luminescence 15:239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e. in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Limit of Blank (LoB)” as used herein refers to the highest apparent analyte concentration expected to be found when replicates of a blank sample containing no analyte are tested.

“Limit of Detection (LoD)” as used herein refers to the lowest concentration of the measurand (i.e. a quantity intended to be measured) that can be detected at a specified level of confidence. The level of confidence is typically 95%, with a 5% likelihood of a false negative measurement. LoD is the lowest analyte concentration likely to be reliably distinguished from the LoB and at which detection is feasible. LoD can be determined by utilizing both the measured LoB and test replicates of a sample known to contain a low concentration of analyte. The LoD term used herein is based on the definition from Clinical and Laboratory Standards Institute (CLSI) protocol EP17-A2 (“Protocols for Determination of Limits of Detection and Limits of Quantitation; Approved Guideline—Second Edition,” EP17A2E, by James F. Pierson-Perry et al., Clinical and Laboratory Standards Institute, Jun. 1, 2012).

“Limit of Quantitation (LoQ)” as used herein refers to the lowest concentration at which the analyte can not only be reliably detected but at which some predefined goals for bias and imprecision are met. The LoQ may be equivalent to the LoD or it could be at a much higher concentration.

“Linearity” refers to how well the method or assay's actual performance across a specified operating range approximates a straight line. Linearity can be measured in terms of a deviation, or non-linearity, from an ideal straight line. “Deviations from linearity” can be expressed in terms of percent of full scale. In some of the methods disclosed herein, less than 10% deviation from linearity (DL) is achieved over the dynamic range of the assay. “Linear” means that there is less than or equal to about 20%, about 19%, about 18%, about 17%, about 16%, about 15%, about 14%, about 13%, about 12%, about 11%, about 10%, about 9%, or about 8% variation for or over an exemplary range or value recited.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Linking sequences can be used for many purposes, including in recombinant Fabs. Exemplary linking sequences include, but are not limited to: (i) Histidine (His) tags, such as a 6× His tag, which has an amino acid sequence of HHHHHH (SEQ ID NO: 1), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO: 2) and derivatives thereof (e.g., ADDDDK (SEQ ID NO: 3), etc.); (iii) Miscellaneous sequences can be used to link or connect the light and/or heavy chain variable regions of single chain variable region fragments. Examples of other linking sequences can be found in Bird et al., Science 242:423-426 (1988); Huston et al., PNAS USA 85:5879-5883 (1988); and McCafferty et al., Nature 348:552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Magnetic resonance imaging” or “MRI” as used interchangeably herein refers to a medical imaging technique used in radiology to form pictures of the anatomy and the physiological processes of the body in both health and disease. MRI is a form of medical imaging that measures the response of the atomic nuclei of body tissues to high-frequency radio waves when placed in a strong magnetic field, and that produces images of the internal organs. MRI scanners, which is based on the science of nuclear magnetic resonance (NMR), use strong magnetic fields, radio waves, and field gradients to generate images of the inside of the body.

“Monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Multivalent binding protein” is used herein to refer to a binding protein comprising two or more antigen binding sites (also referred to herein as “antigen binding domains”). A multivalent binding protein is preferably engineered to have three or more antigen binding sites, and is generally not a naturally occurring antibody. The term “multispecific binding protein” refers to a binding protein that can bind two or more related or unrelated targets, including a binding protein capable of binding two or more different epitopes of the same target molecule.

“Point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long term care and/or hospice facility). Examples of point-of-care devices include those produced by Abbott Laboratories (Abbott Park, IL) (e.g., i-STAT and i-STAT Alinity, Universal Biosensors (Rowville, Australia) (see US 2006/0134713), Axis-Shield PoC AS (Oslo, Norway) and Clinical Lab Products (Los Angeles, USA).

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low”, “medium”, or “high” levels), can be used. Multiple calibrators (i.e. more than one calibrator or a varying amount of calibrator(s)) can be used in conjunction to comprise a “sensitivity panel.”

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e. the bifunctional antibodies have a dual specificity.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.) This disclosure provides exemplary reference levels. However, it is well-known that reference levels may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels for those other immunoassays based on the description provided by this disclosure. Whereas the precise value of the reference level may vary between assays, the findings as described herein should be generally applicable and capable of being extrapolated to other assays.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” and “patient sample” as used herein may be used interchangeably and may be a sample of blood, such as whole blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

A variety of cell types, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, red blood cells, platelets, interstitial fluid, cerebral spinal fluid, etc. Cell types and tissues may also include lymph fluid, cerebrospinal fluid, a fluid collected by A tissue or cell type may be provided by removing a sample of cells from a human and a non-human animal, but can also be accomplished by using previously isolated cells (e.g., isolated by another person, at another time, and/or for another purpose). As well as IPSC or IPSC-derived cell types (e.g., motor neurons) from individuals. Archival tissues, such as those having treatment or outcome history, may also be used. Protein or nucleotide isolation and/or purification may not be necessary.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, aptamers (e.g., RNA and DNA aptamers), and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Subject” and “patient” as used herein interchangeably refers to any vertebrate, including, but not limited to, a mammal and a human. In some embodiments, the subject may be a human or a non-human. The subject or patient may be undergoing other forms of treatment. In some embodiments, when the subject is a human, the subject does not include any humans who have suffered a cerebrovascular accident (e.g., a stroke). In some embodiments, the subject is suspected to have sustained an injury to the head. In some embodiments, the subject is known to have sustained an injury to the head. In some embodiments, the subject is suspected to be suffering from mild, moderate or severe TBI. In some embodiments, the subject is suspected to be suffering from mild TBI. In some embodiments, the subject is suspected to be suffering from moderate TBI. In some embodiments, the subject is suspected to be suffering from severe TBI.

“Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

As used herein the term “single molecule detection” refers to the detection and/or measurement of a single molecule of an analyte in a test sample at very low levels of concentration (such as pg/mL or femtogram/mL levels). A number of different single molecule analyzers or devices are known in the art and include nanopore and nanowell devices. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety.

“Traumatic Brain Injury” or “TBI” as used interchangeably herein refers to a complex injury with a broad spectrum of symptoms and disabilities. TBI is most often an acute event similar to other injuries. TBI can be classified as “mild,” “moderate,” or “severe.” When referred to herein merely as “TBI,” generally this means any category of TBI (e.g., mild, moderate, or severe). The causes of TBI are diverse and include, for example, physical shaking by a person, a car accident, injuries from firearms, cerebral vascular accidents (e.g., strokes), falls, explosions or blasts and other types of blunt force trauma. Other causes of TBI include the ingestion and/or exposure to one or more chemicals or toxins (such as fires, molds, asbestos, pesticides and insecticides, organic solvents, paints, glues, gases (such as carbon monoxide, hydrogen sulfide, and cyanide), organic metals (such as methyl mercury, tetraethyl lead and organic tin), one or more drugs of abuse or combinations thereof). Alternatively, TBI can occur in subjects suffering from an autoimmune disease, a metabolic disorder, a brain tumor, hypoxia, one or more viruses, meningitis, hydrocephalus or combinations thereof. Young adults and the elderly are the age groups at highest risk for TBI. In certain embodiments herein, traumatic brain injury or TBI does not include and specifically excludes cerebral vascular accidents such as strokes.

“Mild TBI” as used herein refers to a brain injury where loss of consciousness is brief and usually a few seconds or minutes and/or confusion and disorientation is shorter than 1 hour. Mild TBI is also referred to as a concussion, minor head trauma, minor TBI, minor brain injury, and minor head injury. While MRI and CT scans are often normal, the individual with mild TBI may have cognitive problems such as headache, difficulty thinking, memory problems, attention deficits, mood swings and frustration.

Mild TBI is the most prevalent TBI and is often missed at time of initial injury. Typically, a subject has a Glasgow Coma scale number of between 13-15 (such as 13-15 or 14-15). Fifteen percent (15%) of people with mild TBI have symptoms that last 3 months or more. Mild TBI is defined as the result of the forceful motion of the head or impact causing a brief change in mental status (confusion, disorientation or loss of memory) or loss of consciousness for less than 30 minutes. Common symptoms of mild TBI include fatigue, headaches, visual disturbances, memory loss, poor attention/concentration, sleep disturbances, dizziness/loss of balance, irritability-emotional disturbances, feelings of depression, and seizures. Other symptoms associated with mild TBI include nausea, loss of smell, sensitivity to light and sounds, mood changes, getting lost or confused, and/or slowness in thinking.

“Mild TBI subclass 1” (TBI-1) refers to subjects who are classified as having mild TBI and also exhibit a plasma proteome signature that is different from controls (e.g., healthy controls, or controls that have not sustained a TBI), different from subjects with moderate to severe TBI, and different from subjects with a mild TBI of each of subclasses 2, 3, or 4.

“Mild TBI subclass 2” (TBI-2) refers to subjects who are classified to having mild TBI and also exhibit a plasma proteome signature that is different from controls (e.g., healthy controls, or controls that have not sustained a TBI), different from subjects with moderate to severe TBI, and different from subjects with a mild TBI of each of subclasses 1, 3, or 4.

“Mild TBI subclass 3” (TBI-3) refers to subjects who are classified to having mild TBI and also exhibit a plasma proteome signature that is different from controls (e.g., healthy controls, or controls that have not sustained a TBI), different from subjects with moderate to severe TBI, and different from subjects with a mild TBI of each of subclasses 1, 2, or 4.

“Mild TBI subclass 4” (Complex TBI), also referred to herein as “complicated mild TBI,” refers to subjects who are classified to having mild TBI, and also exhibit plasma proteome signature that is different from controls (e.g., healthy controls, or controls that have not sustained a TBI), different from subjects with moderate to severe TBI, and different from subjects with a mild TBI of each of subclasses 1, 2, or 3. Additionally, subjects with mild TBI subclass 4 exhibit a proteome signature that resembles the proteome signature obtained from pooled samples of subjects having severe TBI. In data disclosed herein, the mild TBI subclass 4 cluster contained the highest number of subjects having elevated GFAP levels (based on ELISA-based GFAP assay and GFAP mass spectrometry), and the only subject having a positive CT scan.

“Severe TBI” as used herein refers to a brain injury where loss of consciousness and/or confusion and disorientation is between 1 and 24 hours and the subject has a Glasgow Coma scale number of between 9-12. The individual with moderate TBI have abnormal brain imaging results. “Severe TBI” as used herein refers to a brain injury where loss of consciousness is more than 24 hours and memory loss after the injury or penetrating skull injury longer than 24 hours and the subject has a Glasgow Coma scale number between 3-8. The deficits range from impairment of higher level cognitive functions to comatose states. Survivors may have limited function of arms or legs, abnormal speech or language, loss of thinking ability or emotional problems. Individuals with severe injuries can be left in long-term unresponsive states. For many people with severe TBI, long-term rehabilitation is often necessary to maximize function and independence.

Common symptoms of moderate to severe TBI include cognitive deficits including difficulties with attention, concentration, distractibility, memory, speed of processing, confusion, perseveration, impulsiveness, language processing, and/or “executive functions”, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), hearing, such as decrease or loss of hearing, ringing in the cars (tinnitus), increased sensitivity to sounds, loss or diminished sense of smell (anosmia), loss or diminished sense of taste, the convulsions associated with epilepsy that can be several types and can involve disruption in consciousness, sensory perception, or motor movements, control of bowel and bladder, sleep disorders, loss of stamina, appetite changes, regulation of body temperature, menstrual difficulties, dependent behaviors, emotional ability, lack of motivation, irritability, aggression, depression, disinhibition, or denial/lack of awareness.

“Variant” is used herein to describe a peptide or polypeptide that differs in amino acid sequence by the insertion, deletion, or conservative substitution of amino acids, but retain at least one biological activity. “SNP” refers to a variant that is a single nucleotide polymorphism. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e. replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. DIAGNOSING AND EVALUATING WHETHER A SUBJECT HAS SUSTAINED A TRAUMATIC BRAIN INJURY

The present disclosure relates to methods of aiding in the diagnosis, prognosis, risk stratification, and evaluation of whether a subject has sustained or may have sustained an injury to the head, including whether the subject sustained a TBI or types of mild TBI. These methods can aid in determining the extent and/or severity of TBI in a human subject with a suspected injury to the head, including for example, determining whether the subject has sustained a mild TBI, and if so, the subclass of mild TBI sustained, whether the subject has sustained a moderate to severe TBI, or whether the subject has not sustained a TBI. More specifically, the biomarkers of the present disclosure can be used in diagnostic tests to determine, qualify, and/or assess brain injury status, for example, to diagnose TBI, in an individual, subject or patient. In some embodiments, TBI status can include determining a patient's subclinical brain injury status or SCI status, for example, to diagnose SCI, in an individual, subject or patient. Detection or measuring the biomarkers of the present disclosure can aid in diagnosing TBI, and can aid in generating a TBI signature, for example, for subclasses of mild TBI.

Determining whether a subject has mild TBI (or a subclass of mTBI) or moderate to severe TBI can include measuring or detecting one or more TBI biomarkers and integrating that information with other information (e.g., clinical assessment data), to determine that the subject is more likely than not to have sustained a TBI, and if so, what type of TBI was sustained. The method can include performing an assay on a sample obtained from the human subject within about 24 hours, such as within about 2 hours, after a suspected injury to the head to measure or detect a level of one or more TBI biomarkers in the sample and determining whether the subject has sustained a mild or a moderate to severe traumatic brain injury (sTBI). In some embodiments, the subject is determined as having a TBI and/or a subclass of mild TBI when the level of one or more TBI biomarkers in a sample is altered (e.g., higher or lower expression level), as compared to a reference level of one or more TBI biomarkers (e.g., level of the TBI biomarker in a control sample). In other embodiments, the subject is determined as having a TBI and/or a subclass of mild TBI when the level of one or more TBI biomarkers in a sample is detected, without the need to for ascertaining biomarker levels or comparing to a reference or control sample.

The sample can be a biological sample. “Sample,” as used herein may be used interchangeably (e.g., sample, test sample, or biological sample) and may be a sample of blood, such as whole blood, tissue, urine, serum, plasma, amniotic fluid, cerebrospinal fluid, placental cells or tissue, endothelial cells, leukocytes, or monocytes. In some embodiments, the method can include obtaining a sample from a subject within about 48 hours of a suspected injury to the subject and contacting the sample with an antibody for a TBI biomarker to allow formation of a complex of the antibody and the TBI biomarker. The method also includes detecting the resulting antibody-TBI biomarker complex.

In some embodiments, the subject may have received a Glasgow Coma Scale score before or after the level of a TBI biomarker is determined at one or more time points. In certain embodiments, the subject may be suspected of having a mild TBI based on the Glasgow Coma Scale score. In certain embodiments, the subject may be suspected of having a mild TBI based on an abnormal head CT. In some embodiments, the subject has received a CT scan before or after the assay is performed. In some embodiments, the subject has a normal head CT. In some embodiments, the reference level of a TBI biomarker is correlated with subjects having a TBI. In some embodiments, the reference level of a TBI biomarker is correlated with a Glasgow Coma Scale score.

Generally, a reference level of a TBI biomarker can also be employed as a benchmark against which to assess results obtained upon assaying a test sample for a TBI biomarker. Generally, in making such a comparison, the reference level of a TBI biomarker is obtained by running a particular assay a sufficient number of times and under appropriate conditions such that a linkage or association of analyte presence, amount or concentration with a particular stage or endpoint of TBI or with particular indicia can be made. Typically, the reference level of a TBI biomarker is obtained with assays of reference subjects (or populations of subjects). The TBI biomarker measured can include fragments thereof, degradation products thereof, and/or enzymatic cleavage products thereof. In certain embodiments, the reference level may be correlated with control subjects that have not sustained a head injury.

The nature of the assay employed in the methods described herein is not critical and the test can be any assay known in the art such as, for example, immunoassays, protein immunoprecipitation, immunoelectrophoresis, Western blot, or protein immunostaining, or spectrometry methods, such as high-performance liquid chromatography (HPLC), liquid chromatography-mass spectrometry (LC/MS), DIA-MS, DDA-MS, PRM-MS or SRM/MRM-MS mass spectrometry assays directly or with enrichment (e.g., enrichment can be via an antibody to the target protein(s)). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g., diabodies etc.) imprinted polymers, avimers, peptidomimetics, peptoids, peptide nucleic acids, threose nucleic acid, a hormone receptor, a cytokine receptor, and synthetic receptors, and modifications and fragments of these. With enrichment, matrix assisted laser desorption/ionization time-of-flight (MALDI-TOF MS or MALDI-TOF) can also be used. Also, the assay can be employed in clinical chemistry format such as would be known by one skilled in the art.

### 3. TBI BIOMARKER PANELS

Biomarkers of the present disclosure can be used in diagnostic tests to assess, determine, and/or qualify (used interchangeably herein) brain injury status in a patient, such as TBI status. The phrase “brain injury status” includes any distinguishable manifestation of the condition, including not having brain injury. For example, brain injury status includes, without limitation, the presence or absence of brain injury in a patient, the risk of developing brain injury, the stage or severity of brain injury, the progress of brain injury (e.g., progress of brain injury over time), the effectiveness or response to treatment of brain injury (e.g., clinical follow up and surveillance of brain injury after treatment), and type of brain injury, such as TBI or a subclass of TBI. Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. A ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

Analysis of the data described in the present disclosure, as well as clinical data from a cohort of TBI and control patients, resulted in the generation of various TBI biomarkers that can be used individually, or in various combinations with each other and with other biomarkers in the form of a panel, to diagnose and/or evaluate a brain injury in a subject. TBI biomarker panels may include any one of the TBI biomarkers disclosed herein, and may include more than one and up to 20 different biomarkers corresponding to distinct proteins. TBI biomarker panels can also include non-TBI biomarkers (e.g., assay control biomarkers), and biomarkers previously identified to be associated with TBI (e.g., GFAP and/or UCH-L1 and/or NSE). In some embodiments, the biomarker panels of the present disclosure may show a statistical difference in different TBI statuses. Diagnostic tests that use these biomarkers may show an ROC of at least 0.6, at least about 0.7, at least about 0.8, or at least about 0.9.

TBI biomarkers can be differentially present/expressed depending on the type or subclass of TBI (e.g., a TBI signature) and, therefore, panels of more than one TBI biomarker can be useful in aiding in the determination of brain injury status. In some embodiments, biomarkers are measured in a patient sample using the methods described herein and compared, for example, to predefined biomarker levels and correlated to TBI status. In some embodiments, the measurement(s) may then be compared with a relevant diagnostic amount(s), cut-off(s), or multivariate model scores that distinguish a positive TBI status from a negative TBI status. The diagnostic amount(s) represents a measured amount of a biomarker(s) above which or below which a patient is classified as having a particular TBI status. For example, if the biomarker(s) is/are up-regulated compared to a control subject (e.g., a subject that has not sustained a TBI) during brain injury, then a measured amount(s) above the diagnostic cutoff(s) can provide a diagnosis of TBI. Additionally, if the biomarker(s) is/are present during brain injury and not detectable in controls, then any detectably measured amount(s) can provide a diagnosis of brain injury. Alternatively, if the biomarker(s) is/are down-regulated during brain injury, then a measured amount(s) at or below the diagnostic cutoff(s) can provide a diagnosis of non-brain injury. Additionally, if the biomarker(s) is/are not present during brain injury and are detectable in controls, then any detectably measured amount(s) can provide a diagnosis of non-brain injury. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarkers in a statistically significant number of samples from patients with the different brain injury statuses, and drawing the cut-off to suit the desired levels of specificity and sensitivity.

Indeed, as the skilled artisan will appreciate there are many ways to use the measurements of two or more biomarkers in order to improve the diagnostic question under investigation. In a quite simple, but nonetheless often effective approach, a positive result is assumed if a sample is positive for at least one of the markers investigated.

Furthermore, in certain embodiments, the values measured for markers of a biomarker panel are mathematically combined and the combined value is correlated to the underlying diagnostic question. Biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating a biomarker combination of the present invention, e.g. to diagnose brain injury, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

### 4. ASSESSMENT OF TBI CHARACTERISTICS USING BIOMARKERS

In some embodiments, the present disclosure provides methods for characterizing and/or categorizing TBI based on the detection, non-detection, and/or detection levels of one or more TBI biomarkers, such as characterizing the severity of TBI different types of TBI. Each class or subclass of TBI likely has a characteristic level of a biomarker or relative levels of a set of biomarkers (a signature). In one embodiment, the present disclosure provides methods for evaluating the progress of TBI status in a patient over time, including progression (worsening) and regression (improvement). Over time, the amount or relative amount (e.g., the pattern or signature) of the TBI biomarkers may change. For example, biomarker “X” may be increased with brain injury, while biomarker “Y” may be decreased with brain injury. Therefore, the trend of these biomarkers, either increased or decreased over time toward brain injury or non-brain injury indicates the course of the condition. Accordingly, this method involves measuring the level of one or more biomarkers in a patient at least two different time points (e.g., a first time and a second time, and comparing the change, if any).

In some embodiments, a class or subclass of TBI can be characterized by measuring the relevant biomarkers and then either submitting them to a classification algorithm or comparing them with a reference amount (e.g., a predefined level or pattern of biomarkers that is associated with the particular class or subclass).

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows® or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

The learning algorithms described above are useful both for developing classification algorithms for the biomarkers already discovered, and for finding new biomarker biomarkers. The classification algorithms, in turn, form the base for diagnostic tests by providing diagnostic values (e.g., cut-off points) for biomarkers used singly or in combination.

### 5. TREATMENT AND MONITORING OF SUBJECTS SUFFERING FROM TBI

The subject identified or assessed as having TBI may be treated or monitored based on the assessment, which can include detecting or measuring various TBI biomarkers. In some embodiments, the method further includes treating the human subject assessed as having TBI with a treatment, which can take a variety of forms depending on the severity of the injury to the head. For example, for subjects suffering from mild TBI, the treatment may include one or more of rest, abstaining from physical activities, such as sports, avoiding light or wearing sunglasses when out in the light, medication for relief of a headache or migraine, anti-nausea medication, etc. Treatment for patients suffering from severe TBI might include administration of one or more appropriate medications (such as, for example, diuretics, anti-convulsant medications, medications to sedate and put an individual in a drug-induced coma, or other pharmaceutical or biopharmaceutical medications (either known or developed in the future for treatment of TBI), one or more surgical procedures (such as, for example, removal of a hematoma, repairing a skull fracture, decompressive craniectomy, etc.) and one or more therapies (such as, for example one or more rehabilitation, cognitive behavioral therapy, anger management, counseling psychology, etc.). In some embodiments, the method further includes monitoring the human subject assessed as having traumatic brain injury (e.g., mild or moderate to severe traumatic). In some embodiments, a subject identified as having traumatic brain injury, such as mild traumatic brain injury or severe traumatic brain injury, may be monitored with CT scan or MRI.

In one embodiment, the present disclosure provides methods for determining the risk of developing TBI in a patient. TBI biomarker percentages, amounts or patterns are characteristic of various risk states (e.g., high, medium or low). The risk of developing a TBI can be determined by measuring the relevant biomarkers and then either submitting them to a classification algorithm or comparing them with a reference amount (e.g., a predefined level or signature of biomarkers that is associated with the particular risk level).

In some embodiments, treating a subject that has sustained a TBI can include managing patient treatment based on TBI status as established using one or more TBI biomarkers. Such management can include the actions of the physician or clinician subsequent to determining TBI status. For example, if a physician makes a diagnosis of mild TBI, then a certain regime of monitoring would follow. An assessment of the course of TBI using the methods of the present disclosure may then require a certain TBI therapy regimen. Alternatively, a diagnosis of non-TBI might be followed with further testing to determine a specific disease that the patient might be suffering from. Also, further tests may be called for if the diagnostic test gives an inconclusive result on TBI status.

In another embodiment, the present disclosure provides methods for determining the therapeutic efficacy of a pharmaceutical drug in the context of TBI treatment. These methods can be useful in performing clinical trials of a drug, as well as monitoring the progress of a patient on a drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or signature) of one or more of the TBI biomarkers of the present invention may change toward a non-brain injury profile. Therefore, one can follow the course of one or more TBI biomarkers in the patient during the course of treatment.

Accordingly, this method may involve measuring one or more TBI biomarkers in a patient receiving drug therapy, and correlating the biomarker levels with the TBI status of the patient (e.g., by comparison to predefined levels of the biomarkers that correspond to different brain injury statuses). One embodiment of this method can involve determining the levels of one or more TBI biomarkers at least two different time points during a course of drug therapy (e.g., a first time and a second time, and comparing the change in levels of the biomarkers, if any). For example, the levels of one or more TBI biomarkers can be measured before and after drug administration or at two different time points during drug administration. The effect of therapy is determined based on these comparisons. If a treatment is effective, then the one or more TBI biomarkers will trend toward normal, while if treatment is ineffective, the one or more TBI biomarkers will trend toward brain injury indications.

### 6. BIOMARKERS OF TBI

The methods described herein may be used to identify one or more TBI biomarkers or candidate TBI biomarkers that can aid in the diagnosis and evaluation of in a subject that may have sustained a TBI. Exemplary TBI biomarkers are described below.

a. Rule-In TBI Biomarkers

As described below, the following TBI biomarkers were identified as being capable of distinguishing a healthy subject from a subject that has sustained a TBI when detected alone or in combination:

### Ezrin (EZR).

EZR is also known as cytovillin or villin-2, and encodes a protein that in humans is encoded by the EZR gene. The N-terminus of Ezrin contains a FERM domain which is further subdivided into three subdomains. The C-terminus contains a ERM domain. Ezrin is thought to be involved in connections of major cytoskeletal structures to the plasma membrane. In epithelial cells, required for the formation of microvilli and membrane ruffles on the apical pole. Along with PLEKHG6, required for normal micropinocytosis. (UniProt Primary accession number: P15311.)

### 4-Trimethylaminobutyraldehyde Dehydrogenase (ALDH9A1 or AL9A1).

AL9A1 is also known as 4-trimethylaminobutyraldehyde dehydrogenase, TMABADH, Aldehyde dehydrogenase E3 isozyme, Aldehyde dehydrogenase family 9 member A1, Gamma-aminobutyraldehyde dehydrogenase, R-aminobutyraldehyde dehydrogenase. The protein converts gamma-trimethylaminobutyraldehyde into gamma-butyrobetaine, and catalyzes the irreversible oxidation of a broad range of aldehydes to the corresponding acids in an NAD-dependent reaction. The protein is highly expressed in adult liver, skeletal muscle, and kidney, expressed in low levels in heart, pancreas, lung and brain, and is expressed in all regions of the brain. Expression levels are variable in the different brain areas, with the highest levels in the spinal cord and the lowest in the occipital pole. (UniProt Primary accession number: P49189.) ATP synthase subunit gamma, mitochondrial (ATPG).

Mitochondrial membrane ATP synthase (F₁F₀ ATP synthase or Complex V) produces ATP from ADP in the presence of a proton gradient across the membrane which is generated by electron transport complexes of the respiratory chain. F-type ATPases consist of two structural domains, F₁—containing the extramembrancous catalytic core, and F₀—containing the membrane proton channel, linked together by a central stalk and a peripheral stalk. During catalysis, ATP synthesis in the catalytic domain of F₁ is coupled via a rotary mechanism of the central stalk subunits to proton translocation. Part of the complex F₁ domain and the central stalk which is part of the complex rotary element. The gamma subunit protrudes into the catalytic domain formed of alpha3beta3. Rotation of the central stalk against the surrounding alpha3beta3 subunits leads to hydrolysis of ATP in three separate catalytic sites on the beta subunits. (UniProt Primary accession number: P36542.)

### Complement C1r Subcomponent-Like Protein (C1RL).

C1RL mediates the proteolytic cleavage of HP/haptoglobin in the endoplasmic reticulum. Diseases associated with C1RL include Ovary Adenocarcinoma. (UniProt Primary accession number: Q9NZP8.)

### Cullin-Associated NEDD8-Dissociated Protein 1 (CAND1).

CAND1 is a key assembly factor of SCF (SKP1-CUL1-F-box protein) E3 ubiquitin ligase complexes that promotes the exchange of the substrate-recognition F-box subunit in SCF complexes, thereby playing a key role in the cellular repertoire of SCF complexes. Acts as a F-box protein exchange factor. The exchange activity of CAND1 is coupled with cycles of neddylation conjugation: in the deneddylated state, cullin-binding CAND1 binds CUL1-RBX1, increasing dissociation of the SCF complex and promoting exchange of the F-box protein. Probably plays a similar role in other cullin-RING E3 ubiquitin ligase complexes. (UniProt Primary accession number: Q86VP6.)

### Epiplakin (EPIPL or EPPK1).

EPIPL is a cytoskeletal linker protein that connects to intermediate filaments and controls their reorganization in response to stress. In response to mechanical stress like wound healing, is associated with the machinery for cellular motility by slowing down keratinocyte migration and proliferation and accelerating keratin bundling in proliferating keratinocytes thus contributing to tissue architecture. However in wound healing in corneal epithelium it also positively regulates cell differentiation and proliferation and negatively regulates migration thereby controlling corneal epithelium morphogenesis and integrity. In response to cellular stress, plays a role in keratin filament reorganization, probably by protecting keratin filaments against disruption. During liver and pancreas injuries, the protein plays a protective role by chaperoning disease-induced intermediate filament reorganization (By similarity). (UniProt Primary accession number: P58107.)

### Hydroxyacylglutathione Hydrolase, Mitochondrial (GLO2 or HAGH).

GLO2 is a thiolesterase that catalyzes the hydrolysis of S-D-lactoyl-glutathione to form glutathione and D-lactic acid. (UniProt Primary accession number: Q16775.)

### Immunoglobulin Heavy Constant Alpha 2 (IGHA2).

IGHA2 is a constant region of immunoglobulin heavy chains. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. Ig alpha is the major immunoglobulin class in body secretions. (UniProt Primary accession number: P01877.)

### Pregnancy Zone Protein (PZP).

PZP is able to inhibit all four classes of proteinases by a unique “trapping” mechanism. This protein has a peptide stretch, called the ‘bait region’ which contains specific cleavage sites for different proteinases. When a proteinase cleaves the bait region, a conformational change is induced in the protein which traps the proteinase. The entrapped enzyme remains active against low molecular weight substrates (activity against high molecular weight substrates is greatly reduced). Following cleavage in the bait region a thioester bond is hydrolyzed and mediates the covalent binding of the protein to the proteinase. (UniProt Primary accession number: P20742.)

### Threonine-tRNA Ligase, Cytoplasmic (SYTC or TARS).

SYTC catalyzes the reaction: ATP+L-threonine+tRNA (Thr)=AMP+diphosphate+L-threonyl-tRNA (Thr). It is inhibited by borrelidin (BN, IC 50 is 7 nM), which binds to 4 distinct subsites in the protein, preventing binding of all 3 substrates. (UniProt Primary accession number: P26639.)

### Tyrosine-tRNA Ligase, Cytoplasmic (SYYC or YARS).

SYYC catalyzes the attachment of tyrosine to tRNA (Tyr) in a two-step reaction: tyrosine is first activated by ATP to form Tyr-AMP and then transferred to the acceptor end of tRNA (Tyr). (UniProt Primary accession number: P54577.)

### Protein ABHD14B (ABHEB of ABHD14B).

ABHEB exhibits hydrolase activity towards p-nitrophenyl butyrate (in vitro) and may activate transcription. (UniProt Primary accession number: Q96IU4.)

### Dynamin-1-Like Protein (DNM1L).

DNM1L functions in mitochondrial and peroxisomal division. The protein mediates membrane fission through oligomerization into membrane-associated tubular structures that wrap around the scission site to constrict and sever the mitochondrial membrane through a GTP hydrolysis-dependent mechanism. Through its function in mitochondrial division, it ensures the survival of at least some types of postmitotic neurons, including Purkinje cells, by suppressing oxidative damage. It is required for normal brain development, including that of cerebellum. It facilitates developmentally regulated apoptosis during neural tube formation. It is required for a normal rate of cytochrome c release and caspase activation during apoptosis; this requirement may depend upon the cell type and the physiological apoptotic cues. It plays an important role in mitochondrial fission during mitosis. It is required for formation of endocytic vesicles. It is proposed to regulate synaptic vesicle membrane dynamics through association with BCL2L1 isoform Bcl-X (L) which stimulates its GTPase activity in synaptic vesicles; the function may require its recruitment by MFF to clathrin-containing vesicles. And it is required for programmed necrosis execution. (UniProt Primary accession number: 000429.)

### Ficolin-2 (FCN2).

FCN2 may function in innate immunity through activation of the lectin complement pathway. It exhibits calcium-dependent and GlcNAc-binding to lectin. It enhances phagocytosis of S. typhimurium by neutrophils, suggesting an opsonic effect via the collagen region. (UniProt Primary accession number: Q15485.)

### Inverted Formin-2 (IFN2).

IFN2 is involved in the severing of actin filaments and accelerates their polymerization and depolymerization. (UniProt Primary accession number: Q27J81.)

### Keratin, Type II Cytoskeletal 2 Epidermal (K22E or KRT2).

K22E is an intermediate protein and is thought to contribute to terminal cornification. The protein is associated with keratinocyte activation, proliferation and keratinization. (UniProt Primary accession number: P35908.)

### Mitogen-Activated Protein Kinase Kinase Kinase 5 (M3K5 or MAP3K5).

M3K5 is a serine/threonine kinase which acts as an essential component of the MAP kinase signal transduction pathway. It plays an important role in the cascades of cellular responses evoked by changes in the environment. It mediates signaling for determination of cell fate such as differentiation and survival. It plays a crucial role in the apoptosis signal transduction pathway through mitochondria-dependent caspase activation. MAP3K5/ASK1 is required for the innate immune response, which is essential for host defense against a wide range of pathogens. It mediates signal transduction of various stressors like oxidative stress as well as by receptor-mediated inflammatory signals, such as the tumor necrosis factor (TNF) or lipopolysaccharide (LPS). Once activated, it acts as an upstream activator of the MKK/JNK signal transduction cascade and the p38 MAPK signal transduction cascade through the phosphorylation and activation of several MAP kinase kinases like MAP2K4/SEK1, MAP2K3/MKK3, MAP2K6/MKK6 and MAP2K7/MKK7. These MAP2Ks in turn activate p38 MAPKs and c-jun N-terminal kinases (JNKs). Both p38 MAPK and JNKs control the transcription factors activator protein-1 (AP-1). (UniProt Primary accession number: Q99683.)

### Nuclear Receptor Corepressor 1 (NCOR1).

NCOR1 mediates transcriptional repression by certain nuclear receptors. It is part of a complex which promotes histone deacetylation and the formation of repressive chromatin structures which may impede the access of basal transcription factors. It participates in the transcriptional repressor activity produced by BCL6. (UniProt Primary accession number: 075376.)

### Suprabasin (SBSN).

SBSN is a novel gene expressed in mouse and human differentiating keratinocytes. It is thought to be secreted from the spinous layer of the stratified epithelia and may form a novel gene complex on chromosome 2 with dermokine-alpha/-beta and Kdap. (UniProt Primary accession number: Q6UWP8.)

### Bifunctional Glutamate/Proline-tRNA Ligase (SYEP or EPRS).

SYEP catalyzes the attachment of the cognate amino acid to the corresponding tRNA in a two-step reaction: the amino acid is first activated by ATP to form a covalent intermediate with AMP and is then transferred to the acceptor end of the cognate tRNA. It is a component of the GAIT (gamma interferon-activated inhibitor of translation) complex which mediates interferon-gamma-induced transcript-selective translation inhibition in inflammation processes. Upon interferon-gamma activation and subsequent phosphorylation, it dissociates from the multisynthetase complex and assembles into the GAIT complex which binds to stem loop-containing GAIT elements in the 3′-UTR of diverse inflammatory mRNAs (such as ceruplasmin) and suppresses their translation. (UniProt Primary accession number: P07814.)

### Tripeptidyl-Peptidase 2 (TPP2).

TPP2 is a component of the proteolytic cascade acting downstream of the 26S proteasome in the ubiquitin-proteasome pathway. It may be able to complement the 26S proteasome function to some extent under conditions in which the latter is inhibited. It stimulates adipogenesis (By similarity). (UniProt Primary accession number: P29144.)

### Annexin A6 (ANXA6).

ANXA6 may associate with CD21, and may regulate the release of Ca²⁺ from intracellular stores. It can be secreted. (UniProt Primary accession number: P08133.)

### Endoplasmic Reticulum Aminopeptidase 1 (ERAP1).

ERAP1 is an aminopeptidase that plays a central role in peptide trimming, a step required for the generation of most HLA class I-binding peptides. Peptide trimming is essential to customize longer precursor peptides to fit them to the correct length required for presentation on MHC class I molecules. Strongly prefers substrates 9-16 residues long. Rapidly degrades 13-mer to a 9-mer and then stops. It preferentially hydrolyzes the residue Leu and peptides with a hydrophobic C-terminus, while it has weak activity toward peptides with charged C-terminus. May play a role in the inactivation of peptide hormones. It may be involved in the regulation of blood pressure through the inactivation of angiotensin II and/or the generation of bradykinin in the kidney. (UniProt Primary accession number: Q9NZ08.)

### Coagulation Factor V (FA5 or F5).

FA5 is a central regulator of hemostasis. It serves as a critical cofactor for the prothrombinase activity of factor Xa that results in the activation of prothrombin to thrombin. (UniProt Primary accession number: P12259.)

### Glucose-6-Phosphate Isomerase (G6PI or GPI).

G6PI is a glycolytic enzyme, and mammalian G6PI can function as a tumor-secreted cytokine and an angiogenic factor (AMF) that stimulates endothelial cell motility. In the cytoplasm, the gene product functions as a glycolytic enzyme (glucose-6-phosphate isomerase) that interconverts glucose-6-phosphate (G6P) and fructose-6-phosphate (F6P). Extracellularly, the encoded protein (also referred to as neuroleukin) functions as a neurotrophic factor that promotes survival of skeletal motor neurons and sensory neurons, and as a lymphokine that induces immunoglobulin secretion. The encoded protein is also referred to as autocrine motility factor (AMF) based on an additional function as a tumor-secreted cytokine and angiogenic factor. Defects in this gene are the cause of nonspherocytic hemolytic anemia, and a severe enzyme deficiency can be associated with hydrops fetalis, immediate neonatal death and neurological impairment. Alternative splicing results in multiple transcript variants. (UniProt Primary accession number: P06744.)

### Myosin Light Chain Kinase, Smooth Muscle (MYLK).

MYLK is a calcium/calmodulin-dependent myosin light chain kinase implicated in smooth muscle contraction via phosphorylation of myosin light chains (MLC). It also regulates actin-myosin interaction through a non-kinase activity. It phosphorylates PTK2B/PYK2 and myosin light-chains. Involved in the inflammatory response (e.g. apoptosis, vascular permeability, leukocyte diapedesis), cell motility and morphology, airway hyperreactivity and other activities relevant to asthma. It is required for tonic airway smooth muscle contraction that is necessary for physiological and asthmatic airway resistance. It is necessary for gastrointestinal motility. It is implicated in the regulation of endothelial as well as vascular permeability, probably via the regulation of cytoskeletal rearrangements. In the nervous system it has been shown to control the growth initiation of astrocytic processes in culture and to participate in transmitter release at synapses formed between cultured sympathetic ganglion cells. It is critical participant in signaling sequences that result in fibroblast apoptosis. It plays a role in the regulation of epithelial cell survival. Required for epithelial wound healing, especially during actomyosin ring contraction during purse-string wound closure. It mediates RhoA-dependent membrane blebbing. It triggers TRPC5 channel activity in a calcium-dependent signaling, by inducing its subcellular localization at the plasma membrane. It promotes cell migration (including tumor cells) and tumor metastasis. PTK2B/PYK2 activation by phosphorylation mediates ITGB2 activation and is thus essential to trigger neutrophil transmigration during acute lung injury (ALI). It may regulate optic nerve head astrocyte migration. It is probably involved in mitotic cytoskeletal regulation. Regulates tight junction probably by modulating ZO-1 exchange in the perijunctional actomyosin ring. It mediates burn-induced microvascular barrier injury; triggers endothelial contraction in the development of microvascular hyperpermeability by phosphorylating MLC. It is essential for intestinal barrier dysfunction. It me=ediates Giardia spp.-mediated reduced epithelial barrier function during giardiasis intestinal infection via reorganization of cytoskeletal F-actin and tight junctional ZO-1. It is necessary for hypotonicity-induced Ca²⁺ entry and subsequent activation of volume-sensitive organic osmolyte/anion channels (VSOAC) in cervical cancer cells. It is responsible for high proliferative ability of breast cancer cells through anti-apoptosis. (UniProt Primary accession number: Q15746.)

### Serum Amyloid P-Component (SAMP or APCS).

SAMP can interact with DNA and histones and may scavenge nuclear material released from damaged circulating cells. It may also function as a calcium-dependent lectin. (UniProt Primary accession number: P02743.)

In some embodiments, one or more of the TBI biomarkers listed above can be used to determine that a subject has sustained a TBI based on detection of one or more of these TBI biomarkers in a sample from the subject, and a lack of detection of the one or more TBI biomarkers in a control subject (e.g., a subject that has not sustained a TBI). These TBI biomarkers can include one or more of AL9A1, ATPG, C1RL, EPIPL, IGHA2, PZP, SYTC, SYYC, ABHEB, DNM1L, FCN2, INF2, K22E, M3K5, NCOR1, SBSN, SYEP, TPP2, ANXA6, ERAP1, EZRI, FA5, G6PI, MYLK, SAMP, or any combinations thereof. The measurement or detection of one or more of these TBI biomarkers in a subject can be sufficient to indicate that the subject has sustained a TBI, independent of the need to detect, measure, compare, and/or quantify the amount, concentration, and/or expression level of the one or more TBI biomarkers in a control subject. Although one or more of these TBI biomarkers may be present in a control subject, or in a subject that has not sustained a TBI, it is generally present in an amount that is not able to be detected though conventional means, as described herein. Thus, in some cases, detection of one or more of these TBI biomarkers in a subject indicates that a subject has sustained a TBI.

In some embodiments, one or more of the TBI biomarkers listed above can be used to determine that a subject has sustained a severe TBI based on detection of one or more of these TBI biomarkers in a sample from the subject, and a lack of detection of the one or more TBI biomarkers in a control subject (e.g., a subject that has not sustained a TBI) or a lack of detection in a subject that has sustained a mild TBI. These TBI biomarkers can include one or more of ATPG, C1RL, SYYC, or any combinations thereof. The measurement or detection of one or more of these TBI biomarkers in a subject can be sufficient to indicate that the subject has sustained a severe TBI, independent of the need to detect, measure, compare, or quantify the amount, concentration, or expression level of the one or more TBI biomarkers in a control subject or a subject that has sustained a mild TBI.

In some embodiments, one or more of the TBI biomarkers listed above can be used to determine that a subject has sustained a TBI based on detection of one or more of these TBI biomarkers in a sample from the subject at an amount, concentration, and/or expression level that is higher than an amount, concentration and/or expression level of the corresponding TBI biomarker in a control subject. These TBI biomarkers can include one or more of CAND1 and GLO2, or any combinations thereof. The measurement or detection of an increased level of one or more of these TBI biomarkers in a subject as compared to a control subject (e.g., a subject that has not sustained a TBI) can be sufficient to indicate that the subject has sustained a TBI, and in some cases, can be sufficient to indicate that the subject has sustained a severe TBI.

In some embodiments, one or more of the TBI biomarkers listed above can be used to determine that a subject has sustained a TBI based on detection of one or more of these TBI biomarkers in a sample from the subject at an amount, concentration, and/or expression level that is higher or lower than an amount, concentration and/or expression level of the corresponding TBI biomarker in a control subject. These TBI biomarkers can include one or more of ABHEB, AL9A1, DNM1L, or any combinations thereof. The measurement or detection of an increased or decreased level of one or more of these TBI biomarkers in a subject as compared to a control subject (e.g., a subject that has not sustained a TBI) can be sufficient to indicate that the subject has sustained a TBI, and in some cases, can be sufficient to indicate that the subject has sustained a severe TBI. For example, detection or measurement of a higher level of ABHED, and/or a lower level of AL9A1 and/or DNM1L in a subject that may have sustained a TBI as compared to levels from a control subject can indicate that the subject has indeed sustained a TBI. Levels of these TBI biomarkers can be detected or measured alone or in combination as part of a TBI panel or signature.

In some embodiments, one or more of the TBI biomarkers listed above can be used to determine that a subject has sustained a mild TBI based on detection of one or more of these TBI biomarkers in a sample from the subject at an amount, concentration, and/or expression level that is higher or lower than an amount, concentration and/or expression level of the corresponding TBI biomarker in a control subject. These TBI biomarkers can include one or more of M3K5, SBSN, SYEP, or any combinations thereof. The measurement or detection of an increased or decreased level of one or more of these TBI biomarkers in a subject as compared to a control subject (e.g., a subject that has not sustained a TBI) can be sufficient to indicate that the subject has sustained a TBI, and in some cases, can be sufficient to indicate that the subject has sustained a mild TBI. For example, detection or measurement of a higher level of SBSN and/or SYEP, and/or a lower level of M3K5 in a subject that may have sustained a TBI as compared to levels from a control subject can indicate that the subject has indeed sustained a mild TBI. Levels of these TBI biomarkers can be detected or measured alone or in combination as part of a TBI panel or signature.

In some embodiments, one or more of the TBI biomarkers listed above can be included with other biomarkers that may or may not have been identified as TBI biomarkers. For example, one or more of the TBI biomarkers listed above can be included in a panel of biomarkers that may include one or more of ANXA6, ERAP1, EZRI, FA5, G6PI, MYLK, SAMP, or combinations thereof. In some cases, a panel of TBI biomarkers, in conjunction with other TBI biomarkers and non-TBI biomarkers can aid in the diagnosis of TBI to a greater extent than individual biomarkers alone.

b. Rule-In Mild TBI Biomarkers

As described below, the following TBI biomarkers were identified as being capable of distinguishing a healthy subject from a subject that has sustained a mild TBI when detected alone or in combination:

### Poly(rC)-Binding Protein 2 (Alpha-CP2) (PCBP2).

Poly(rC)-binding protein 2 is a protein that in humans is encoded by the PCBP2 gene. The protein encoded by this gene appears to be multifunctional. It along with PCBP-1 and hnRNPK corresponds to the major cellular poly(rC)-binding proteins. It contains three K-homologous (KH) domains which may be involved in RNA binding. This encoded protein together with PCBP-1 also functions as translational coactivators of poliovirus RNA via a sequence-specific interaction with stem-loop IV of the IRES and promote poliovirus RNA replication by binding to its 5′-terminal cloverleaf structure. It has also been implicated in translational control of the 15-lipoxygenase mRNA, human Papillomavirus type 16 L2 mRNA, and hepatitis A virus RNA. The encoded protein is also suggested to play a part in formation of a sequence-specific alpha-globin mRNP complex which is associated with alpha-globin mRNA stability. This multiexon structural mRNA is thought to be retrotransposed to generate PCBP-1 intronless gene which has similar functions. This gene and PCBP-1 has paralogues PCBP3 and PCBP4 which is thought to arose as a result of duplication events of entire genes. It also has two processed pseudogenes PCBP2P1 and PCBP2P2. There are presently two alternatively spliced transcript variants described for this gene. In humans, the PCBP2 gene overlaps with TUC338, a transcribed ultra-conserved element implicated in Hepatocellular carcinoma. (UniProt Primary accession number: Q15366.)

### Thioredoxin Reductase 2, Mitochondrial (TRXR2).

Thioredoxin reductases (TR, TrxR) (EC 1.8.1.9) are the only known enzymes to reduce thioredoxin (Trx). Two classes of thioredoxin reductase have been identified: one class in bacteria and some eukaryotes and one in animals. Both classes are flavoproteins which function as homodimers. Each monomer contains a FAD prosthetic group, a NADPH binding domain, and an active site containing a redox-active disulfide bond. Thioredoxin reductase is the only enzyme known to catalyze the reduction of thioredoxin and hence is a central component in the thioredoxin system. Together with thioredoxin (Trx) and NADPH this system's most general description is as a method of forming reduced disulfide bonds in cells. Electrons are taken from NADPH via TrxR and are transferred to the active site of Trx, which goes on to reduce protein disulfides or other substrates. The Trx system exists in all living cells and has an evolutionary history tied to DNA as a genetic material, defense against oxidative damage due to oxygen metabolism, and redox signaling using molecules like hydrogen peroxide and nitric oxide. (UniProt Primary accession number: Q9NNW7.)

### 14-3-3 Protein Gamma (1433G or YWHAG).

1433G is an adapter protein implicated in the regulation of a large spectrum of both general and specialized signaling pathways. Binds to a large number of partners, usually by recognition of a phosphoserine or phosphothreonine motif. Binding generally results in the modulation of the activity of the binding partner. (UniProt Primary accession number: P61981.)

### Activated CDC42 Kinase 1 (ACK1 or TKN2).

ACK1 is a non-receptor tyrosine-protein and serine/threonine-protein kinase that is implicated in cell spreading and migration, cell survival, cell growth and proliferation. Transduces extracellular signals to cytosolic and nuclear effectors. It phosphorylates AKT1, AR, MCF2, WASL and WWOX. Implicated in trafficking and clathrin-mediated endocytosis through binding to epidermal growth factor receptor (EGFR) and clathrin. It binds to both poly- and mono-ubiquitin and regulates ligand-induced degradation of EGFR, thereby contributing to the accumulation of EGFR at the limiting membrane of early endosomes. It is a downstream effector of CDC42 which mediates CDC42-dependent cell migration via phosphorylation of BCAR1. It may be involved both in adult synaptic function and plasticity and in brain development. Activates AKT1 by phosphorylating it on “Tyr-176.” Phosphorylates AR on “Tyr-267” and “Tyr-363” thereby promoting its recruitment to androgen-responsive enhancers (AREs). It phosphorylates WWOX on “Tyr-287.” It phosphorylates MCF2, thereby enhancing its activity as a guanine nucleotide exchange factor (GEF) toward Rho family proteins. It contributes to the control of AXL receptor levels. It confers metastatic properties on cancer cells and promotes tumor growth by negatively regulating tumor suppressor such as WWOX and positively regulating pro-survival factors such as AKT1 and AR. It phosphorylates WASP. (UniProt Primary accession number: Q07912.)

### Aminoacylase-1 (ACY1).

ACY1 is involved in the hydrolysis of N-acylated or N-acetylated amino acids (except L-aspartate). (UniProt Primary accession number: Q03154.)

### A-Kinase Anchor Protein 12 (AKA12 or AKAP12).

AKA12 is an anchoring protein that mediates the subcellular compartmentation of protein kinase A (PKA) and protein kinase C (PKC). (UniProt Primary accession number: Q02952.)

### Arginase-1 (ARGI1 or ARG1).

ARGI1 is key element of the urea cycle converting L-arginine to urea and L-ornithine, which is further metabolized into metabolites proline and polyamides that drive collagen synthesis and bioenergetic pathways critical for cell proliferation, respectively; the urea cycle takes place primarily in the liver and, to a lesser extent, in the kidneys. It functions in L-arginine homeostasis in nonhepatic tissues characterized by the competition between nitric oxide synthase (NOS) and arginase for the available intracellular substrate arginine. Arginine metabolism is a critical regulator of innate and adaptive immune responses. It is involved in an antimicrobial effector pathway in polymorphonuclear granulocytes (PMN). Upon PMN cell death, it is liberated from the phagolysosome and depletes arginine in the microenvironment leading to suppressed T cell and natural killer (NK) cell proliferation and cytokine secretion. In group 2 innate lymphoid cells (ILC2s), it promotes acute type 2 inflammation in the lung and is involved in optimal ILC2 proliferation but not survival (By similarity). (UniProt Primary accession number: P05089.)

### Cadherin-5 (CADH5 or CDH5).

CADH5 is a cadherin; cadherins are calcium-dependent cell adhesion proteins. They preferentially interact with themselves in a homophilic manner in connecting cells; cadherins may thus contribute to the sorting of heterogeneous cell types. This cadherin may play a important role in endothelial cell biology through control of the cohesion and organization of the intercellular junctions. It associates with alpha-catenin forming a link to the cytoskeleton. It acts in concert with KRIT1 to establish and maintain correct endothelial cell polarity and vascular lumen. These effects are mediated by recruitment and activation of the Par polarity complex and RAP1B. It is required for activation of PRKCZ and for the localization of phosphorylated PRKCZ, PARD3, TIAM1 and RAP1B to the cell junction. (UniProt Primary accession number: P33151.)

### Clathrin Heavy Chain 1 (CLH1 of CLTC).

CLH1 is the major protein of the polyhedral coat of coated pits and vesicles. Two different adapter protein complexes link the clathrin lattice either to the plasma membrane or to the trans-Golgi network. It acts as component of the TACC3/ch-TOG/clathrin complex proposed to contribute to stabilization of kinetochore fibers of the mitotic spindle by acting as inter-microtubule bridge. The TACC3/ch-TOG/clathrin complex is required for the maintenance of kinetochore fiber tension. It plays a role in early autophagosome formation. (UniProt Primary accession number: Q00610.)

### Coatomer Subunit Gamma-2 (COPG2).

COPG2 is a cytosolic protein complex that binds to dilysine motifs and reversibly associates with Golgi non-clathrin-coated vesicles, which further mediate biosynthetic protein transport from the ER, via the Golgi up to the trans Golgi network. A coatomer complex is required for budding from Golgi membranes, and is essential for the retrograde Golgi-to-ER transport of dilysine-tagged proteins. In mammals, the coatomer can only be recruited by membranes associated to ADP-ribosylation factors (ARFs), which are small GTP-binding proteins; the complex also influences the Golgi structural integrity, as well as the processing, activity, and endocytic recycling of LDL receptors (By similarity). (UniProt Primary accession number: Q9UBF2.)

### DNA Polymerase Delta Subunit 2 (DPOD2 or POLD2).

As a component of the trimeric and tetrameric DNA polymerase delta complexes (Pol-delta3 and Pol-delta4, respectively), DPOD2 plays a role in high fidelity genome replication, including in lagging strand synthesis, and repair. Pol-delta3 and Pol-delta4 are characterized by the absence or the presence of POLD4. They exhibit differences in catalytic activity. Most notably, Pol-delta3 shows higher proofreading activity than Pol-delta4. Although both Pol-delta3 and Pol-delta4 process Okazaki fragments in vitro, Pol-delta3 may also be better suited to fulfill this task, exhibiting near-absence of strand displacement activity compared to Pol-delta4 and stalling on encounter with the 5′-blocking oligonucleotides. Pol-delta3 idling process may avoid the formation of a gap, while maintaining a nick that can be readily ligated. Along with DNA polymerase kappa, DNA polymerase delta carries out approximately half of nucleotide excision repair (NER) synthesis following UV irradiation. Under conditions of DNA replication stress, required for the repair of broken replication forks through break-induced replication (BIR). Involved in the translesion synthesis (TLS) of templates carrying 06-methylguanine or abasic sites performed by Pol-delta4, independently of DNA polymerase zeta (REV3L) or eta (POLH). Facilitates abasic site bypass by DNA polymerase delta by promoting extension from the nucleotide inserted opposite the lesion. Also involved in TLS as a component of the POLZ complex. Along with POLD3, dramatically increases the efficiency and processivity of DNA synthesis of the minimal DNA polymerase zeta complex, consisting of only REV3L and REV7. (UniProt Primary accession number: P49005.)

### Desmoglein-2 (DSG2).

DSG2 is a component of intercellular desmosome junctions. It is involved in the interaction of plaque proteins and intermediate filaments mediating cell-cell adhesion. (UniProt Primary accession number: Q14126.)

### Immunoglobulin Heavy Variable 3-7 (HV307 or IGHV3-7).

HV307 is the V region of the variable domain of immunoglobulin heavy chains that participates in the antigen recognition. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. (UniProt Primary accession number: P01780.)

### Ras GTPase-Activating-Like Protein IQGAP2 (IQGA2 or IQGAP2).

IQGA2 binds to activated CDC42 and RACI but does not seem to stimulate their GTPase activity. It is associates with calmodulin. (UniProt Primary accession number: Q13576.)

### Keratin, Type I Cytoskeletal 14 (K1C14 or KRT14).

K1C14 is an intermediate filament protein and the nonhelical tail domain is involved in promoting KRT5-KRT14 filaments to self-organize into large bundles and enhances the mechanical properties involved in resilience of keratin intermediate filaments in vitro. (UniProt Primary accession number: P02533.)

### Keratin, Type I Cytoskeletal 19 (K1C19 or KRT19).

K1C19 is involved in the organization of myofibers. Together with KRT8, it helps to link the contractile apparatus to dystrophin at the costameres of striated muscle. (UniProt Primary accession number: P08727.)

### Immunoglobulin Kappa Variable 1-5 (KV105 of IGKV1-5).

KV105 is the V region of the variable domain of immunoglobulin light chains that participates in the antigen recognition. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. (UniProt Primary accession number: P01602.)

### Laminin Subunit Gamma-1 (LAMC1).

Binding to cells via a high affinity receptor, LAMC1 is thought to mediate the attachment, migration and organization of cells into tissues during embryonic development by interacting with other extracellular matrix components. (UniProt Primary accession number: P11047.)

### Malate Dehydrogenase, Mitochondrial (MDHM or MDH2).

MDHM catalyzes the reversible oxidation of malate to oxaloacetate, utilizing the NAD/NADH cofactor system in the citric acid cycle. The protein encoded by this gene is localized to the mitochondria and may play pivotal roles in the malate-aspartate shuttle that operates in the metabolic coordination between cytosol and mitochondria. Several transcript variants encoding different isoforms have been found for this gene. (UniProt Primary accession number: P40926.)

### Ribosyldihydronicotinamide Dehydrogenase [Quinone] (NQO2).

NQO2 serves as a quinone reductase in connection with conjugation reactions of hydroquinones involved in detoxification pathways as well as in biosynthetic processes such as the vitamin K-dependent gamma-carboxylation of glutamate residues in prothrombin synthesis. (UniProt Primary accession number: P16083.)

### Myeloperoxidase (PERM or MPO).

PERM is part of the host defense system of polymorphonuclear leukocytes. It is responsible for microbicidal activity against a wide range of organisms. In the stimulated PMN, MPO catalyzes the production of hypohalous acids, primarily hypochlorous acid in physiologic situations, and other toxic intermediates that greatly enhance PMN microbicidal activity. (UniProt Primary accession number: P05164.)

### Plastin-3 (PLST or PLS3).

PLST is an actin-bundling protein found in intestinal microvilli, hair cell stereocilia, and fibroblast filopodia. It may play a role in the regulation of bone development. (UniProt Primary accession number: P13797.)

### Nicotinate Phosphoribosyltransferase (PNCB or NAPRT).

PNCB catalyzes the conversion of nicotinic acid (NA) to NA mononucleotide (NaMN). It is essential for NA to increase cellular NAD levels and prevent oxidative stress of the cells. It catalyzes the synthesis of beta-nicotinate D-ribonucleotide from nicotinate and 5-phospho-D-ribose 1-phosphate at the expense of ATP. (UniProt Primary accession number: Q6XQN6.)

### Receptor-Type Tyrosine-Protein Phosphatase C (PTPRC).

PTPRC is a protein tyrosine-protein phosphatase required for T-cell activation through the antigen receptor. It acts as a positive regulator of T-cell coactivation upon binding to DPP4. The first PTPase domain has enzymatic activity, while the second one seems to affect the substrate specificity of the first one. Upon T-cell activation, it recruits and dephosphorylates SKAP1 and FYN. It dephosphorylates LYN, and thereby modulates LYN activity (By similarity). (UniProt Primary accession number: P08575.)

### Septin-7 (SEPT7).

SEPT7 is a filament-forming cytoskeletal GTPase. It is required for normal organization of the actin cytoskeleton. It is required for normal progress through mitosis. It is involved in cytokinesis. It is required for normal association of CENPE with the kinetochore. It plays a role in ciliogenesis and collective cell movements. It forms a filamentous structure with SEPT12, SEPT6, SEPT2 and probably SEPT4 at the sperm annulus which is required for the structural integrity and motility of the sperm tail during postmeiotic differentiation. (UniProt Primary accession number: Q16181.)

### Arginine-tRNA Ligase, Cytoplasmic (STRC or RARS).

SYRC forms part of a macromolecular complex that catalyzes the attachment of specific amino acids to cognate tRNAs during protein synthesis. It modulates the secretion of AIMP1 and may be involved in generation of the inflammatory cytokine EMAP2 from AIMP1. (UniProt Primary accession number: P54136.)

### Thioredoxin-Like Protein 1 (TXNL1).

TXNL1 is an active thioredoxin with a redox potential of about-250 mV. (UniProt Primary accession number: 043396.)

### UDP-Glucose: Glycoprotein Glucosyltransferase 1 (UGGG1 or UGGT1).

UGGG1 recognizes glycoproteins with minor folding defects. It reglucosylates single N-glycans near the misfolded part of the protein, thus providing quality control for protein folding in the endoplasmic reticulum. Reglucosylated proteins are recognized by calreticulin for recycling to the endoplasmic reticulum and refolding or degradation. (UniProt Primary accession number: Q9NYU2.)

### WD Repeat-Containing Protein 1 (WDR1).

WDR1 induces disassembly of actin filaments in conjunction with ADF/cofilin family proteins. It enhances cofilin-mediated actin severing (By similarity). It is involved in cytokinesis. It is involved in chemotactic cell migration by restricting lamellipodial membrane protrusions. It is involved in myocardium sarcomere organization. It is required for cardiomyocyte growth and maintenance (By similarity). It is involved in megakaryocyte maturation and platelet shedding. It is required for the establishment of planar cell polarity (PCP) during follicular epithelium development and for cell shape changes during PCP; the function seems to implicate cooperation with CFL1 and/or DSTN/ADF. It is involved in the generation/maintenance of cortical tension (By similarity). It is involved in assembly and maintenance of epithelial apical cell junctions and plays a role in the organization of the perijunctional actomyosin belt. (UniProt Primary accession number: 075083.)

### Neuroblast Differentiation-Associated Protein AHNAK (AHNK of AHNAK).

AHNK is thought to be required for neuronal cell differentiation. (UniProt Primary accession number: Q09666.)

### Retinal Dehydrogenase 1 (ALIA1 of ALDHIA1).

ALIA1 converts/oxidizes retinaldehyde to retinoic acid. It binds free retinal and cellular retinol-binding protein-bound retinal (By similarity). It may have a broader specificity and oxidize other aldehydes in vivo. (UniProt Primary accession number: P00352.)

### Aminopeptidase N (AMPN or ANPEP).

AMPN is a broad specificity aminopeptidase which plays a role in the final digestion of peptides generated from hydrolysis of proteins by gastric and pancreatic proteases. It is also involved in the processing of various peptides including peptide hormones, such as angiotensin III and IV, neuropeptides, and chemokines. It may also be involved the cleavage of peptides bound to major histocompatibility complex class II molecules of antigen presenting cells. It may have a role in angiogenesis and promote cholesterol crystallization. IT acts as a receptor for human coronavirus 229E/HCoV-229E. In case of human coronavirus 229E (HCoV-229E) infection, serves as receptor for HCoV-229E spike glycoprotein. It mediates as well human cytomegalovirus (HCMV) infection. (UniProt Primary accession number: P15144.)

### F-Actin-Capping Protein Subunit Beta (CAPZB).

CAPZB is an F-actin-capping protein that bind in a Ca²⁺-independent manner to the fast growing ends of actin filaments (barbed end) thereby blocking the exchange of subunits at these ends. Unlike other capping proteins (such as gelsolin and severin), these proteins do not sever actin filaments. It plays a role in the regulation of cell morphology and cytoskeletal organization. (UniProt Primary accession number: P47756.)

### Cathepsin D (CATD of CTSD).

CATD is an acid protease active in intracellular protein breakdown. It plays a role in APP processing following cleavage and activation by ADAM30 which leads to APP degradation. It is involved in the pathogenesis of several diseases such as breast cancer and possibly Alzheimer disease. (UniProt Primary accession number: P07339.)

### CAP-Gly Domain-Containing Linker Protein 2 (CLIP2).

CLIP2 links microtubules to dendritic lamellar body (DLB), a membranous organelle predominantly present in bulbous dendritic appendages of neurons linked by dendrodendritic gap junctions. It may operate in the control of brain-specific organelle translocations (By similarity). (UniProt Primary accession number: Q9UDT6.)

### Chromogranin-A (CMGA or CHGA).

Pancreastatin: Strongly inhibits glucose induced insulin release from the pancreas. Catestatin: Inhibits catecholamine release from chromaffin cells and noradrenergic neurons by acting as a non-competitive nicotinic cholinergic antagonist. Displays antibacterial activity against Gram-positive bacteria S. aureus and M. luteus, and Gram-negative bacteria E. coli and P. aeruginosa. Can induce mast cell migration, degranulation and production of cytokines and chemokines. Acts as a potent scavenger of free radicals in vitro. May play a role in the regulation of cardiac function and blood pressure. Serpinin: Regulates granule biogenesis in endocrine cells by up-regulating the transcription of protease nexin 1 (SERPINE2) via a cAMP-PKA-SP1 pathway. This leads to inhibition of granule protein degradation in the Golgi complex which in turn promotes granule formation. (UniProt Primary accession number: P10645.)

### Fascin (FSCN1).

FSCN1 organizes filamentous actin into bundles with a minimum of 4.1:1 actin/fascin ratio. It plays a role in the organization of actin filament bundles and the formation of microspikes, membrane ruffles, and stress fibers. It is important for the formation of a diverse set of cell protrusions, such as filopodia, and for cell motility and migration. (UniProt Primary accession number: Q16658.)

### GMP Reductase 2 (GMPR2).

GMPR2 catalyzes the irreversible NADPH-dependent deamination of GMP to IMP. It functions in the conversion of nucleobase, nucleoside and nucleotide derivatives of G to A nucleotides, and in maintaining the intracellular balance of A and G nucleotides. It plays a role in modulating cellular differentiation. (UniProt Primary accession number: Q9P2T1.)

### 78 kDa Glucose-Regulated Protein (GRP78 or HSPA5).

GRP78 plays a role in facilitating the assembly of multimeric protein complexes inside the endoplasmic reticulum. It is involved in the correct folding of proteins and degradation of misfolded proteins via its interaction with DNAJC10, probably to facilitate the release of DNAJC10 from its substrate (By similarity). It is thought to be a secreted protein. (UniProt Primary accession number: P11021.)

### Glutamate-Cysteine Ligase Catalytic Subunit (GSH1 or GCLC).

GSH1, also known as gamma-glutamylcysteine synthetase, is the first rate-limiting enzyme of glutathione synthesis. The enzyme consists of two subunits: a heavy catalytic subunit and a light regulatory subunit. This locus encodes the catalytic subunit, while the regulatory subunit is derived from a different gene located on chromosome 1p22-p21. Mutations at this locus have been associated with hemolytic anemia due to deficiency of gamma-glutamylcysteine synthetase and susceptibility to myocardial infarction. (UniProt Primary accession number: P48506.)

### Isocitrate Dehydrogenase [NADP] Cytoplasmic (IDHC of IDH1).

IDHC is an enzyme that in humans is encoded by the IDH1 gene on chromosome 2. Isocitrate dehydrogenases catalyze the oxidative decarboxylation of isocitrate to 2-oxoglutarate. These enzymes belong to two distinct subclasses, one of which uses NAD⁺ as the electron acceptor and the other NADP⁺. Five isocitrate dehydrogenases have been reported: three NAD⁺-dependent isocitrate dehydrogenases, which localize to the mitochondrial matrix, and two NADP⁺-dependent isocitrate dehydrogenases, one of which is mitochondrial and the other predominantly cytosolic. Each NADP⁺-dependent isozyme is a homodimer. The protein encoded by this gene is the NADP⁺-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2,4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production. Alternatively spliced transcript variants encoding the same protein have been found for this gene. (UniProt Primary accession number: 075874.)

### Keratin, Type I Cytoskeletal 20 (K1C20 or KRT20).

K1C20 plays a significant role in maintaining keratin filament organization in intestinal epithelia. When phosphorylated, it plays a role in the secretion of mucin in the small intestine (By similarity). (UniProt Primary accession number: P35900.)

### KRR1 Small Subunit Processome Component Homolog (KRR1).

KRR1 is required for 40S ribosome biogenesis. It is involved in nucleolar processing of pre-18S ribosomal RNA and ribosome assembly (By similarity). (UniProt Primary accession number: Q13601.)

### Mannose-Binding Protein C (MBL2).

MBL2 is a calcium-dependent lectin involved in innate immune defense. It binds mannose, fucose and N-acetylglucosamine on different microorganisms and activates the lectin complement pathway. It binds to late apoptotic cells, as well as to apoptotic blebs and to necrotic cells, but not to early apoptotic cells, facilitating their uptake by macrophages. It may bind DNA. (UniProt Primary accession number: P11226.)

### Nuclear Transport Factor 2 (NTF2 or NUTF2).

NTF2 mediates the import of GDP-bound RAN from the cytoplasm into the nucleus which is essential for the function of RAN in cargo receptor-mediated nucleocytoplasmic transport. Thereby, it plays indirectly a more general role in cargo receptor-mediated nucleocytoplasmic transport. It interacts with GDP-bound RAN in the cytosol, recruits it to the nuclear pore complex via its interaction with nucleoporins and promotes its nuclear import. (UniProt Primary accession number: P61970.)

### Phosphoglycerate Kinase 1 (PGK1).

In addition to its role as a glycolytic enzyme, PGK-1 acts as a polymerase alpha cofactor protein (primer recognition protein). It may play a role in sperm motility. (UniProt Primary accession number: P00558.)

### Serum Amyloid A-1 Protein (SAA1).

SAA1 is a protein that in humans is encoded by the SAA1 gene. SAA1 is a major acute-phase protein mainly produced by hepatocytes in response to infection, tissue injury and malignancy. When released into blood circulation, SAA1 is present as an apolipoprotein associated with high-density lipoprotein (HDL). SAA1 is a major precursor of amyloid A (AA), the deposit of which leads to inflammatory amyloidosis. (UniProt Primary accession number: PODJI8.)

### Transferrin Receptor Protein 1 (TFR1 or TFRC).

Cellular uptake of iron occurs via receptor-mediated endocytosis of ligand-occupied transferrin receptor into specialized endosomes. Endosomal acidification leads to iron release. The apotransferrin-receptor complex is then recycled to the cell surface with a return to neutral pH and the concomitant loss of affinity of apotransferrin for its receptor. Transferrin receptor is necessary for development of erythrocytes and the nervous system (By similarity). A second ligand, the hereditary hemochromatosis protein HFE, competes for binding with transferrin for an overlapping C-terminal binding site. Positively regulates T and B cell proliferation through iron uptake. (UniProt Primary accession number: P02786.)

In some embodiments, one or more of the TBI biomarkers listed above can be used to determine that a subject has sustained a TBI based on detection of one or more of these TBI biomarkers in a sample from the subject, and a lack of detection of the one or more TBI biomarkers in a control subject (e.g., a subject that has not sustained a TBI), or in some cases, lack of detection in a subject that has sustained a severe TBI. These TBI biomarkers can include one or more of 1433G, ACK1, ACY1, AKA12, ARGI1, CADH5, CLH1, COPG2, DPOD2, DSG2, HV307, IQGA2, K1C14, K1C19, KV105, LAMC1, MDHM, NQO2, PERM, PLST, PNCB, PTPRC, SEPT7, SYRC, TRXR2, TXNL1, UGGG1, WDR1, AHNK, AL1A1, AMPN, CAPZB, CATD, CLIP2, CMGA, FSCN1, GMPR2, GRP78, GSH1, IDHC, K1C20, KRR1, MBL2, NTF2, PCBP2, PGK1, SAA1, TFR1, or any combinations thereof. The measurement or detection of one or more of these TBI biomarkers in a subject can be sufficient to indicate that the subject has sustained a mild TBI, independent of the need to detect, measure, compare, and/or quantify the amount, concentration, and/or expression level of the one or more TBI biomarkers in a control subject, or a subject that has sustained a severe TBI. Although one or more of these TBI biomarkers may be present in a control subject, or in a subject that has not sustained a TBI, it is generally present in an amount that is not able to be detected though conventional means, as described herein. Thus, in some cases, detection of one or more of these TBI biomarkers in a subject indicates that a subject has sustained a mild TBI.

In some embodiments, one or more of the TBI biomarkers listed above can be used to determine that a subject has sustained a mild TBI of a particular subclassification. Without being bound to a particular theory, underlying etiology, or disease mechanism, one or more of these TBI biomarkers can be used to classify a subject that has a sustained a mild TBI into one of four subclasses (e.g., subclass 1, subclass 2, subclass 3, or subclass 4). For example, detection or measurement of one or more of ACK1, ACY1, PLST, PNCB, PTPRC, UGGG1, or any combinations thereof in a subject that may have sustained a TBI can indicate that the subject has indeed sustained a mild TBI of subclass 1. Detection or measurement of one or more of AKA12, HV307, PERM, KV105, NQO2, SEPT7, SYRC, TRXR2, or any combinations thereof in a subject that may have sustained a TBI can indicate that the subject has indeed sustained a mild TBI of subclass 2. And detection or measurement of one or more of 1433B, ARGI1, CADH5, CLH1, COPG2, DPOD2, DSG2, IQGA2, K1C14, LAMC1, MDHM, TXNL1, or any combinations thereof in a subject that may have sustained a TBI can indicate that the subject has indeed sustained a mild TBI of subclass 3. Levels of these TBI biomarkers can be detected or measured alone or in combination as part of a mild TBI panel or signature.

In some embodiments, one or more of the TBI biomarkers listed above can be included with other biomarkers that may or may not have been identified as TBI biomarkers. For example, one or more of the TBI biomarkers listed above can be included in a panel of biomarkers that may include one or more of AHNK, AL1A1, AMPN, CAPZB, CATD, CLIP2, CMGA, FSCN1, GMPR2, GRP78, GSH1, IDHC, K1C20, KRR1, MBL2, NTF2, PCBP2, PGK1, SAA1, TFR1, or combinations thereof. In some cases, a panel of TBI biomarkers, in conjunction with other TBI biomarkers and non-TBI biomarkers can aid in the diagnosis of TBI to a greater extent than individual biomarkers alone.

c. Mild TBI Signatures

As described below, the following mild TBI biomarkers were identified as being capable of distinguishing a healthy subject from a subject that has sustained a mild TBI and/or a mild TBI of a specific subclass when detected alone or in combination:

### Ephrin Type-B Receptor 4 (EPHB4).

Ephrin type-B receptor 4 is a protein that in humans is encoded by the EPHB4 gene. Ephrin receptors and their ligands, the ephrins, mediate numerous developmental processes, particularly in the nervous system. Based on their structures and sequence relationships, ephrins are divided into the ephrin-A (EFNA) class, which are anchored to the membrane by a glycosylphosphatidylinositol linkage, and the ephrin-B (EFNB) class, which are transmembrane proteins. The Eph family of receptors are divided into 2 groups based on the similarity of their extracellular domain sequences and their affinities for binding ephrin-A and ephrin-B ligands. Ephrin receptors make up the largest subgroup of the receptor tyrosine kinase (RTK) family. The protein encoded by this gene binds to ephrin-B2 and plays an essential role in vascular development. (UniProt Primary accession number: P54760.)

### Immunoglobulin Heavy Variable 1-3 (HV103 or IGHV1-3).

HV103 is the V region of the variable domain of immunoglobulin heavy chains that participates in the antigen recognition. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. (UniProt Primary accession number: A0A0C4DH29.)

### Immunoglobulin Heavy Constant Delta (IGHD).

IGHD is a constant region of immunoglobulin heavy chains. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. IgD is the major antigen receptor isotype on the surface of most peripheral B-cells, where it is coexpressed with IgM. The membrane-bound IgD (mIgD) induces the phosphorylation of CD79A and CD79B by the Src family of protein tyrosine kinases. Soluble IgD (sIgD) concentration in serum below those of IgG, IgA, and IgM but much higher than that of IgE. IgM and IgD molecules present on B cells have identical V regions and antigen-binding sites. After the antigen binds to the B-cell receptor, the secreted form sIgD is shut off. IgD is a potent inducer of TNF, IL1B, and IL1RN. IgD also induces release of IL6, IL10, and LIF from peripheral blood mononuclear cells. Monocytes seem to be the main producers of cytokines in vitro in the presence of IgD. (UniProt Primary accession number: P01880.)

### Ester Hydrolase C11orf54 (CK054 of C11orf54).

CK054 is a protein that in humans is encoded by the C11orf54 gene. The human gene, C11orf54, is also known as PTD012 and PTOD12. C11orf54 exhibits hydrolase activity on p-nitrophenyl acetate and acts on ester bonds, though the overall function is still not fully understood by the scientific community. The protein is highly conserved with the most distant homolog found is in bacteria (UniProt Primary accession number: Q9H0W9.)

### Epididymis-Specific Alpha-Mannosidase (MA2B2 or MAN2B2).

MA2B2 catalyzes the hydrolysis of terminal, non-reducing alpha-D-mannose residues in alpha-D-mannosides. (UniProt Primary accession number: Q9Y2E5.)

### Protein Diaphanous Homolog 1 (DIAP1 or DIAPH1).

DIAP1 acts in a Rho-dependent manner to recruit PFY 1 to the membrane. It is required for the assembly of F-actin structures, such as actin cables and stress fibers. Nucleates actin filaments. It binds to the barbed end of the actin filament and slows down actin polymerization and depolymerization. It is required for cytokinesis, and transcriptional activation of the serum response factor. DFR proteins couple Rho and Src tyrosine kinase during signaling and the regulation of actin dynamics. It functions as a scaffold protein for MAPRE1 and APC to stabilize microtubules and promote cell migration (By similarity). It has neurite outgrowth promoting activity (By similarity). In hear cells, it may play a role in the regulation of actin polymerization in hair cells. The MEMO1-RHOA-DIAPH1 signaling pathway plays an important role in ERBB2-dependent stabilization of microtubules at the cell cortex. It controls the localization of APC and CLASP2 to the cell membrane, via the regulation of GSK3B activity. In turn, membrane-bound APC allows the localization of the MACF1 to the cell membrane, which is required for microtubule capture and stabilization. It plays a role in the regulation of cell morphology and cytoskeletal organization. It is required in the control of cell shape. It plays a role in brain development. (UniProt Primary accession number: 060610.)

### Procollagen-Lysine, 2-Oxoglutarate 5-Dioxygenase 1 (PLOD1).

PLOD1 is part of a complex composed of PLOD1, P3H3 and P3H4 that catalyzes hydroxylation of lysine residues in collagen alpha chains and is required for normal assembly and cross-linking of collagen fibrils (By similarity). It forms hydroxylysine residues in-Xaa-Lys-Gly-sequences in collagens. These hydroxylysines serve as sites of attachment for carbohydrate units and are essential for the stability of the intermolecular collagen cross-links (Probable). (UniProt Primary accession number: Q02809.)

### Immunoglobulin Kappa Variable 1-33 (KV133 or IGKV1-33).

KV133 is the V region of the variable domain of immunoglobulin light chains that participates in the antigen recognition. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. (UniProt Primary accession number: P01594.)

As disclosed herein, the following biomarkers were also identified as being capable of distinguishing a healthy subject from a subject that has sustained a mild TBI and/or a mild TBI of a specific subclass when detected alone or in combination with each other and with the other biomarkers in this section: TPP2, CAND1, NCOR1, K22E, AL9A1, ABHEB, DNM1L, INF2, M3K5, SBSN, SYEP, MYLK, and SAMP. Descriptions of these biomarkers were provided above and are not replicated here.

In some embodiments, one or more of the TBI biomarkers listed above can be used to determine that a subject has sustained a mild TBI of a particular subclassification. Without being bound to a particular theory, underlying etiology, or disease mechanism, one or more of these TBI biomarkers can be used to classify a subject that has a sustained a mild TBI into one of four subclasses (e.g., subclass 1, subclass 2, subclass 3, or subclass 4). In some embodiments, one or more of the TBI biomarkers listed above can be used to determine that a subject has sustained a mild TBI based on detection of one or more of these TBI biomarkers in a sample from the subject at an amount, concentration, and/or expression level that is higher or lower than an amount, concentration and/or expression level of the corresponding TBI biomarker in a control subject. The measurement or detection of an increased or decreased level of one or more of these TBI biomarkers in a subject as compared to a control subject (e.g., a subject that has not sustained a TBI) can be sufficient to indicate that the subject has sustained a mild TBI, and in some cases, can be sufficient to indicate that the subject has sustained a mild TBI of a particular subclass.

For example, detection or measurement of one or more of TPP2, CAND1, NCOR1, K22E, AL9A1, ABHEB, DNM1L, INF2, or any combinations thereof in a subject that may have sustained a TBI can indicate that the subject has indeed sustained a mild TBI of subclass 4. In some cases, detection or measurement of a higher level of CAND1, NCOR1, K22E, ABHEB, DNM1L, or any combination thereof and/or a lower level of TPP2, AL9A1, INF2, or any combination thereof in a subject that may have sustained a TBI as compared to levels from a control subject can indicate that the subject has indeed sustained a mild TBI of subclass 4.

In some embodiments, detection or measurement of one or more of TPP2, NCOR1, HV103, INF2, IGHD, CK054, M3K5, ABHEB, AL9A1, DNM1L, or any combinations thereof in a subject that may have sustained a TBI can indicate that the subject has indeed sustained a mild TBI of subclass 3. In some cases, detection or measurement of a higher level of NCOR1, HV103, IGHD, ABHEB, DNM1L, or any combination thereof and/or a lower level of TPP2, IGHD, CK054, M3K5, AL9A1, or any combination thereof in a subject that may have sustained a TBI as compared to levels from a control subject can indicate that the subject has indeed sustained a mild TBI of subclass 3.

In some embodiments, detection or measurement of one or more of NCOR1, TPP2, K22E, ABHEB, INF2, SBSN, AL9A1, MA2B2, or any combinations thereof in a subject that may have sustained a TBI can indicate that the subject has indeed sustained a mild TBI of subclass 2. In some cases, detection or measurement of a higher level of NCOR1, K22E, ABHEB, SBSN, or any combination thereof and/or a lower level of TPP2, INF2, AL9A1, MA2B2, or any combination thereof in a subject that may have sustained a TBI as compared to levels from a control subject can indicate that the subject has indeed sustained a mild TBI of subclass 2.

In other embodiments, detection or measurement of one or more of K22E, DNM1L, DIAP1, ABHEB, PLOD1, SYEP, KV133, AL9A1, EPHB4, or any combinations thereof in a subject that may have sustained a TBI can indicate that the subject has indeed sustained a mild TBI of subclass 1. In some cases, detection or measurement of a higher level of K22E, DNM1L, DIAP1, ABHEB, PLOD1, SYEP, EPHB4, or any combination thereof and/or a lower level of KV133 and AL9A1, or any combination thereof in a subject that may have sustained a TBI as compared to levels from a control subject can indicate that the subject has indeed sustained a mild TBI of subclass 1.

Levels of these TBI biomarkers can be detected or measured alone or in combination as part of a mild TBI panel or signature.

In some embodiments, one or more of the TBI biomarkers listed above can be included with other biomarkers that may or may not have been identified as TBI biomarkers. For example, one or more of the TBI biomarkers listed above can be included in a panel of biomarkers that may include one or more of MYLK, SAMP or combinations thereof. In some cases, a panel of TBI biomarkers, in conjunction with other TBI biomarkers and non-TBI biomarkers can aid in the diagnosis of TBI to a greater extent than individual biomarkers alone.

As described below, the following mild TBI biomarkers were identified as being capable of distinguishing a healthy subject from a subject that has sustained a mild TBI and/or a mild TBI of a specific subclass when detected alone or in combination:

### Dynein Light Chain 1, Cytoplasmic (DYL1 or DYNLL1).

DYL1 acts as one of several non-catalytic accessory components of the cytoplasmic dynein 1 complex that are thought to be involved in linking dynein to cargos and to adapter proteins that regulate dynein function. Cytoplasmic dynein 1 acts as a motor for the intracellular retrograde motility of vesicles and organelles along microtubules. It may play a role in changing or maintaining the spatial distribution of cytoskeletal structures. It binds and inhibits the catalytic activity of neuronal nitric oxide synthase. It promotes transactivation functions of ESR1 and plays a role in the nuclear localization of ESR1. It regulates apoptotic activities of BCL2L11 by sequestering it to microtubules. Upon apoptotic stimuli the BCL2L11-DYNLL1 complex dissociates from cytoplasmic dynein and translocates to mitochondria and sequesters BCL2 thus neutralizing its antiapoptotic activity. (UniProt Primary accession number: P63167.)

### Puromycin-Sensitive Aminopeptidase (PSA or NPEPPS).

PSA is an aminopeptidase with broad substrate specificity for several peptides. It is involved in proteolytic events essential for cell growth and viability. It may act as regulator of neuropeptide activity. It plays a role in the antigen-processing pathway for MHC class I molecules. It is involved in the N-terminal trimming of cytotoxic T-cell epitope precursors. It digests the poly-Q peptides found in many cellular proteins. It digests tau from normal brain more efficiently than tau from Alzheimer disease brain. (UniProt Primary accession number: P55786.)

### EGF-Containing Fibulin-Like Extracellular Matrix Protein 1 (FBLN3 or EFEMP1).

FBLN3 binds EGFR, the EGF receptor, inducing EGFR autophosphorylation and the activation of downstream signaling pathways. It may play a role in cell adhesion and migration. It may function as a negative regulator of chondrocyte differentiation. In the olfactory epithelium, it may regulate glial cell migration, differentiation and the ability of glial cells to support neuronal neurite outgrowth. (UniProt Primary accession number: Q12805.)

### Serum Albumin (ALBU or ALB).

ALBU is the main protein of plasma, has a good binding capacity for water, Ca²⁺, Na⁺, K⁺, fatty acids, hormones, bilirubin and drugs. Its main function is the regulation of the colloidal osmotic pressure of blood. It is a major zinc transporter in plasma, typically binds about 80% of all plasma zinc. (UniProt Primary accession number: P02768.)

### 3-Ketoacyl-CoA Thiolase, Mitochondrial (THIM or ACAA2).

THIM is involved in abolishing BNIP3-mediated apoptosis and mitochondrial damage. (UniProt Primary accession number: P42765.)

### Immunoglobulin Kappa Variable 1-39 (KV139 or IGKV1-39).

KV139 is the V region of the variable domain of immunoglobulin light chains that participates in the antigen recognition. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. (UniProt Primary accession number: P01597.)

### Mannan-Binding Lectin Serine Protease 2 (MASP2).

MASP2 is a serum protease that plays an important role in the activation of the complement system via mannose-binding lectin. After activation by auto-catalytic cleavage it cleaves C2 and C4, leading to their activation and to the formation of C3 convertase. (UniProt Primary accession number: 000187.)

As disclosed herein, the following mild TBI biomarkers were identified as being capable of distinguishing a healthy subject from a subject that has sustained a mild TBI and/or a mild TBI of a specific subclass when detected alone or in combination with each other and with the other biomarkers in this section: ANXA6, CAND1, NCOR1, K22E, ABHEB, DIAP1, DNM1L, EPHB4, GLO2, HV103, IGHA2, IGHD, PLOD1, SBSN, SYEP, MYLK, and SAMP. Descriptions of these biomarkers were provided above and are not replicated here.

In some embodiments, one or more of the TBI biomarkers listed above can be used to determine that a subject has sustained a mild TBI of a particular subclassification. Without being bound to a particular theory, underlying etiology, or disease mechanism, one or more of these TBI biomarkers can be used to classify a subject that has a sustained a mild TBI into one of four subclasses (e.g., subclass 1, subclass 2, subclass 3, or subclass 4). In some embodiments, one or more of the TBI biomarkers listed above can be used to determine that a subject has sustained a mild TBI based on detection of one or more of these TBI biomarkers in a sample from the subject at an amount, concentration, and/or expression level that is higher or lower than an amount, concentration and/or expression level of the corresponding TBI biomarker in a control subject. The measurement or detection of an increased or decreased level of one or more of these TBI biomarkers in a subject as compared to a control subject (e.g., a subject that has not sustained a TBI) can be sufficient to indicate that the subject has sustained a mild TBI, and in some cases, can be sufficient to indicate that the subject has sustained a mild TBI of a particular subclass.

For example, detection or measurement of one or more of CAND1, NCOR1, K22E, ABHEB, DNM1L, SBSN, GLO2, SYEP, or any combinations thereof in a subject that may have sustained a TBI can indicate that the subject has indeed sustained a mild TBI of subclass 4. In some cases, detection or measurement of a higher level of CAND1, NCOR1, K22E, ABHEB, DNM1L, SBSN, GLO2, SYEP, or any combinations thereof in a subject that may have sustained a TBI as compared to levels from a control subject can indicate that the subject has indeed sustained a mild TBI of subclass 4.

In some embodiments, detection or measurement of one or more of NCOR1, HV103, IGHD, ABHEB, DNM1L, ALBU, THIM, IGHA2, KV139, or any combinations thereof in a subject that may have sustained a TBI can indicate that the subject has indeed sustained a mild TBI of subclass 3. In some cases, detection or measurement of a higher level of NCOR1, HV103, IGHD, ABHEB, DNM1L, ALBU, THIM, IGHA2, KV139, or any combinations thereof in a subject that may have sustained a TBI as compared to levels from a control subject can indicate that the subject has indeed sustained a mild TBI of subclass 3.

In some embodiments, detection or measurement of one or more of NCOR1, K22E, ABHEB, SBSN, DNM1L, DIAP1, DYL1, PSA, EPHB4, or any combinations thereof in a subject that may have sustained a TBI can indicate that the subject has indeed sustained a mild TBI of subclass 2. In some cases, detection or measurement of a higher level of NCOR1, K22E, ABHEB, SBSN, DNM1L, DIAP1, DYL1, PSA, EPHB4, or any combinations thereof in a subject that may have sustained a TBI as compared to levels from a control subject can indicate that the subject has indeed sustained a mild TBI of subclass 2.

In other embodiments, detection or measurement of one or more of K22E, DNM1L, DIAP1, ABHEB, PLOD1, SYEP, EPHB4, FBLN3, or any combinations thereof in a subject that may have sustained a TBI can indicate that the subject has indeed sustained a mild TBI of subclass 1. In some cases, detection or measurement of a higher level of K22E, DNM1L, DIAP1, ABHEB, PLOD1, SYEP, EPHB4, FBLN3, or any combinations thereof in a subject that may have sustained a TBI as compared to levels from a control subject can indicate that the subject has indeed sustained a mild TBI of subclass 1.

Levels of these TBI biomarkers can be detected or measured alone or in combination as part of a mild TBI panel or signature.

In some embodiments, one or more of the TBI biomarkers listed above can be included with other biomarkers that may or may not have been identified as TBI biomarkers. For example, one or more of the TBI biomarkers listed above can be included in a panel of biomarkers that may include one or more of ANXA6, MASP2, MYLK, SAMP, or combinations thereof. In some cases, a panel of TBI biomarkers, in conjunction with other TBI biomarkers and non-TBI biomarkers can aid in the diagnosis of TBI to a greater extent than individual biomarkers alone.

d. Rule-Out TBI Biomarkers

As described below, the following TBI biomarkers were identified as being capable of distinguishing a healthy subject from a subject that has sustained a TBI when detected alone or in combination:

SH3 domain-binding glutamic acid-rich-like protein (SH3BGRL).

SH3 domain-binding glutamic acid-rich-like protein 3 is a protein that in humans is encoded by the SH3BGRL3 gene. The 10.5 kDa protein SH3 binding glutamic acid-rich protein-like 3 has an isoelectric point of 5.0. SH3 binding glutamic acid-rich (SH3BGR) gene is located to human chromosome 21. Two homologous genes, SH3BGRL and SH3BGRL3 are located to chromosome Xq13.3 and 1p34.3-35, respectively and code for small proteins similar to the N-terminal region of the SH3BGR protein. SH3BGRL3 protein shows a significant similarity to glutaredoxin 1 of E. coli, and all the three proteins are predicted to belong to thioredoxin-like protein family. Glutaredoxins (GRXs) are ubiquitous oxidoreductases, which catalyze the reduction of many intra-cellular protein disulfides and play an important role in many redox pathways. However, the SH3BGRL3 protein lacks the enzymatic function of glutaredoxins and may have a role as a regulator of redox activity. (UniProt Primary accession number: 075368.)

### Beta-Actin-Like Protein 2 (ACTBL or ACTBL2).

ACTBL is a member of the actin family. Actins are highly conserved proteins that are involved in various types of cell motility and are ubiquitously expressed in all eukaryotic cells. (UniProt Primary accession number: Q562R1.)

### Aldehyde Dehydrogenase, Mitochondrial (ALDH2).

ALDH2 catalyzes the oxidation of aldehydes. Despite the name “dehydrogenase”, their mode of oxidation is by addition of oxygen rather than by removal of hydrogen—that is, they convert aldehydes (R—C(═O)—H) to carboxylic acids (R—C(═O)—O—H). To date, nineteen ALDH genes have been identified within the human genome. These genes participate in a wide variety of biological processes including the detoxification of exogenously and endogenously generated aldehydes. (UniProt Primary accession number: P05091.)

### Annexin A5 (ANXA5).

ANXA5 is an anticoagulant protein that acts as an indirect inhibitor of the thromboplastin-specific complex, which is involved in the blood coagulation cascade. (UniProt Primary accession number: P08758.)

### Cathelicidin Antimicrobial Peptide (CAMP).

CAMP binds to bacterial lipopolysaccharides (LPS), and exhibits antibacterial activity. (UniProt Primary accession number: P49913.)

### Copine-3 (CPNE3).

CPNE3 is a calcium-dependent phospholipid-binding protein that plays a role in ERBB2-mediated tumor cell migration in response to growth factor heregulin stimulation. (UniProt Primary accession number: 075131.)

### Cartilage Acidic Protein 1 (CRAC1 or CRTAC1).

CRAC1 encodes a glycosylated extracellular matrix protein that is found in the interterritorial matrix of articular deep zone cartilage. This protein is used as a marker to distinguish chondrocytes from osteoblasts and mesenchymal stem cells in culture. The presence of FG-GAP motifs and an RGD integrin-binding motif suggests that this protein may be involved in cell-cell or cell-matrix interactions. Copy number alterations in this gene have been observed in neurofibromatosis type 1-associated glomus tumors. Alternative splicing results in multiple transcript variants. (UniProt Primary accession number: Q9NQ79.)

### Cystatin-C (CYTC or CST3).

As an inhibitor of cysteine proteinases, CYTC is thought to serve an important physiological role as a local regulator of this enzyme activity. (UniProt Primary accession number: P01034.)

### Aspartyl Aminopeptidase (DNPEP).

DNPEP is an aminopeptidase with specificity towards an acidic amino acid at the N-terminus. It is likely to play an important role in intracellular protein and peptide metabolism. (UniProt Primary accession number: Q9ULA0.)

### Eukaryotic Translation Initiation Factor 3 Subunit I (EIF3I).

EIF3I is a component of the eukaryotic translation initiation factor 3 (eIF-3) complex, which is required for several steps in the initiation of protein synthesis. The eIF-3 complex associates with the 40S ribosome and facilitates the recruitment of eIF-1, eIF-1A, eIF-2: GTP: methionyl-tRNAi and eIF-5 to form the 43S pre-initiation complex (43S PIC). The eIF-3 complex stimulates mRNA recruitment to the 43S PIC and scanning of the mRNA for AUG recognition. The eIF-3 complex is also required for disassembly and recycling of post-termination ribosomal complexes and subsequently prevents premature joining of the 40S and 60S ribosomal subunits prior to initiation. The eIF-3 complex specifically targets and initiates translation of a subset of mRNAs involved in cell proliferation, including cell cycling, differentiation and apoptosis, and uses different modes of RNA stem-loop binding to exert either translational activation or repression. (UniProt Primary accession number: Q13347.)

### Glutathione Synthetase (GSHB of GSS).

GSHB is the second enzyme in the glutathione (GSH) biosynthesis pathway. It catalyses the condensation of gamma-glutamylcysteine and glycine, to form glutathione. Glutathione synthetase is also a potent antioxidant. It is found in a large number of species including bacteria, yeast, mammals, and plants. In humans, defects in GSS are inherited in an autosomal recessive way and are the cause of severe metabolic acidosis, 5-oxoprolinuria, increased rate of haemolysis, and defective function of the central nervous system. Deficiencies in GSS can cause a spectrum of deleterious symptoms in plants and human beings alike. In eukaryotes, this is a homodimeric enzyme. The substrate-binding domain has a 3-layer alpha/beta/alpha structure. This enzyme utilizes and stabilizes an acylphosphate intermediate to later perform a favorable nucleophilic attack of glycine. (UniProt Primary accession number: P48637.)

### Intercellular Adhesion Molecule 1 (ICAM1).

ICAM1 proteins are ligands for the leukocyte adhesion protein LFA-1 (integrin alpha-L/beta-2). During leukocyte trans-endothelial migration, ICAM1 engagement promotes the assembly of endothelial apical cups through ARHGEF26/SGEF and RHOG activation. It acts as a receptor for major receptor group rhinovirus A-B capsid proteins. Acts as a receptor for Coxsackievirus A21 capsid proteins. Upon Kaposi's sarcoma-associated herpesvirus/HHV-8 infection, it is degraded by viral E3 ubiquitin ligase MIR2, presumably to prevent lysis of infected cells by cytotoxic T-lymphocytes and NK cell. (UniProt Primary accession number: P05362.)

### (HV323 or IGHV3-23).

HV323 is V region of the variable domain of immunoglobulin heavy chains that participates in the antigen recognition. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. (UniProt Primary accession number: P01764.)

### Heterogeneous Nuclear Ribonucleoprotein DO (HNRPD or HNRNPD).

HNRPD binds with high affinity to RNA molecules that contain AU-rich elements (AREs) found within the 3′-UTR of many proto-oncogenes and cytokine mRNAs. It also binds to double- and single-stranded DNA sequences in a specific manner and functions a transcription factor. Each of the RNA-binding domains specifically can bind solely to a single-stranded non-monotonous 5′-UUAG-3′ sequence and also weaker to the single-stranded 5′-TTAGGG-3′ telomeric DNA repeat. It binds RNA oligonucleotides with 5′-UUAGGG-3′ repeats more tightly than the telomeric single-stranded DNA 5′-TTAGGG-3′ repeats. Binding of RRM1 to DNA inhibits the formation of DNA quadruplex structure which may play a role in telomere elongation. It may be involved in translationally coupled mRNA turnover. It is implicated with other RNA-binding proteins in the cytoplasmic deadenylation/translational and decay interplay of the FOS mRNA mediated by the major coding-region determinant of instability (mCRD) domain. It may play a role in the regulation of the rhythmic expression of circadian clock core genes. It directly binds to the 3′UTR of CRY1 mRNA and induces CRY1 rhythmic translation. It may also be involved in the regulation of PER2 translation. (UniProt Primary accession number: Q14103.)

### Immunoglobulin Kappa Variable 1D-33 (KVD33 or IGKV1D-33).

KVD33 is the V region of the variable domain of immunoglobulin light chains that participates in the antigen recognition. Immunoglobulins, also known as antibodies, are membrane-bound or secreted glycoproteins produced by B lymphocytes. In the recognition phase of humoral immunity, the membrane-bound immunoglobulins serve as receptors which, upon binding of a specific antigen, trigger the clonal expansion and differentiation of B lymphocytes into immunoglobulins-secreting plasma cells. Secreted immunoglobulins mediate the effector phase of humoral immunity, which results in the elimination of bound antigens. The antigen binding site is formed by the variable domain of one heavy chain, together with that of its associated light chain. Thus, each immunoglobulin has two antigen binding sites with remarkable affinity for a particular antigen. The variable domains are assembled by a process called V-(D)-J rearrangement and can then be subjected to somatic hypermutations which, after exposure to antigen and selection, allow affinity maturation for a particular antigen. (UniProt Primary accession number: P01593.)

### Coagulation Factor IX (FA9 or F9).

FA9 is a factor IX is a vitamin K-dependent plasma protein that participates in the intrinsic pathway of blood coagulation by converting factor X to its active form in the presence of Ca²⁺ ions, phospholipids, and factor VIIIa. (UniProt Primary accession number: P00740.)

### Complement Factor H-Related Protein 4 (FHR4 or CFHR4).

FHR4 is involved in complement regulation. It can associate with lipoproteins and may play a role in lipid metabolism. (UniProt Primary accession number: Q92496.)

### FERM and PDZ Domain-Containing Protein 1 (FRPD1 or FRMPD1).

FRPD1 acts to stabilize membrane-bound GPSM1, and thereby promotes its interaction with GNAI1. (UniProt Primary accession number: Q5SYB0.)

### Heat Shock Protein HSP 90-Beta (HS90B or HSP90AB1).

HS90B is a molecular chaperone that promotes the maturation, structural maintenance and proper regulation of specific target proteins involved for instance in cell cycle control and signal transduction. It undergoes a functional cycle that is linked to its ATPase activity. This cycle probably induces conformational changes in the client proteins, thereby causing their activation. It interacts dynamically with various co-chaperones that modulate its substrate recognition, ATPase cycle and chaperone function. It engages with a range of client protein classes via its interaction with various co-chaperone proteins or complexes, which act as adapters, simultaneously able to interact with the specific client and the central chaperone itself. Recruitment of ATP and co-chaperone followed by client protein forms a functional chaperone. After the completion of the chaperoning process, properly folded client protein and co-chaperone leave HSP90 in an ADP-bound partially open conformation and finally, ADP is released from HSP90 which acquires an open conformation for the next cycle. Apart from its chaperone activity, it also plays a role in the regulation of the transcription machinery. HSP90 and its co-chaperones modulate transcription at least at three different levels. In the first place, they alter the steady-state levels of certain transcription factors in response to various physiological cues. Second, they modulate the activity of certain epigenetic modifiers, such as histone deacetylases or DNA methyl transferases, and thereby respond to the change in the environment. Third, they participate in the eviction of histones from the promoter region of certain genes and thereby turn on gene expression. It antagonizes STUB1-mediated inhibition of TGF-beta signaling via inhibition of STUB1-mediated SMAD3 ubiquitination and degradation. It promotes cell differentiation by chaperoning BIRC2 and thereby protecting from auto-ubiquitination and degradation by the proteasomal machinery. It is the main chaperone that is involved in the phosphorylation/activation of the STAT1 by chaperoning both JAK2 and PRKCE under heat shock and in turn, activates its own transcription. (UniProt Primary accession number: P08238.)

### Alpha-Mannosidase 2 (MA2A1 or MAN2A1).

MA2A1 catalyzes the first committed step in the biosynthesis of complex N-glycans. It controls conversion of high mannose to complex N-glycans; the final hydrolytic step in the N-glycan maturation pathway. (UniProt Primary accession number: Q16706.)

### Prenylcysteine Oxidase 1 (PCYOX or PCYOX1).

PCYOX is involved in the degradation of prenylated proteins. It cleaves the thioether bond of prenyl-L-cysteines, such as farnesylcysteine and geranylgeranylcysteine. (UniProt Primary accession number: Q9UHG3.)

### Purine Nucleoside Phosphorylase (PNPH of PNP).

PNPH is a purine nucleoside phosphorylase that catalyzes the phosphorolytic breakdown of the N-glycosidic bond in the beta-(deoxy) ribonucleoside molecules, with the formation of the corresponding free purine bases and pentose-1-phosphate. (UniProt Primary accession number: P00491.)

### Vitamin K-Dependent Protein C (PROC).

PROC is a vitamin K-dependent serine protease that regulates blood coagulation by inactivating factors Va and VIIIa in the presence of calcium ions and phospholipids. It exerts a protective effect on the endothelial cell barrier function. (UniProt Primary accession number: P04070.)

### 60S Ribosomal Protein L3 (RL3 or RPL3).

RL3 is a component of the large subunit of cytoplasmic ribosomes. (UniProt Primary accession number: P39023.)

### Serine/Arginine Repetitive Matrix Protein 2 (SRRM2).

SRRM2 is involved in pre-mRNA splicing. It may function at or prior to the first catalytic step of splicing at the catalytic center of the spliceosome. It may do so by stabilizing the catalytic center or the position of the RNA substrate (By similarity). It binds to RNA. (UniProt Primary accession number: Q9UQ35.)

### Tubulin Beta-1 Chain (TBB1 or TUBB1).

TBB1 is a subunit of tubulin, which is the major constituent of microtubules. It binds two moles of GTP, one at an exchangeable site on the beta chain and one at a non-exchangeable site on the alpha chain (By similarity). (UniProt Primary accession number: Q9H4B7.)

### Tenascin (TENA or TNC).

TENA is an extracellular matrix protein implicated in guidance of migrating neurons as well as axons during development, synaptic plasticity as well as neuronal regeneration. It promotes neurite outgrowth from cortical neurons grown on a monolayer of astrocytes. It is a ligand for integrins alpha-8/beta-1, alpha-9/beta-1, alpha-V/beta-3 and alpha-V/beta-6. In tumors, it stimulates angiogenesis by elongation, migration and sprouting of endothelial cells. (UniProt Primary accession number: P24821.)

Heat Shock Protein 75 kDa, Mitochondrial (TRAP1).

TRAP1 is a chaperone that expresses an ATPase activity. It is involved in maintaining mitochondrial function and polarization, downstream of PINK1 and mitochondrial complex I. It is a negative regulator of mitochondrial respiration able to modulate the balance between oxidative phosphorylation and aerobic glycolysis. The impact of TRAP1 on mitochondrial respiration is probably mediated by modulation of mitochondrial SRC and inhibition of SDHA. (UniProt Primary accession number: Q12931.)

In some embodiments, one or more of the TBI biomarkers listed above can be used to determine that a subject has not sustained a TBI based on detection of one or more of these TBI biomarkers in a sample from the subject (rule-out TBI). These TBI biomarkers can include one or more of ACTBL, ALDH2, ANXA5, CAMP, CPNE3, CRAC1, CYTC, DNPEP, EIF3I, GSHB, ICAM1, HV323, HNRPD, KVD33, FA9, FHR4, FRPD1, HS90B, MA2A1, PCYOX, PNPH, PROC, RL3, SH3L3, SRRM2, TBB1, TENA, TRAP1, or any combinations thereof. The measurement or detection of one or more of these TBI biomarkers in a subject can be sufficient to indicate that the subject has not sustained a TBI, independent of the need to detect, measure, compare, and/or quantify the amount, concentration, and/or expression level of the one or more TBI biomarkers in a control subject. Although one or more of these TBI biomarkers may be present in a subject that has sustained a TBI, it is generally present in an amount that is not able to be detected though conventional means, as described herein. Thus, in some cases, detection of one or more of these TBI biomarkers in a subject indicates that a subject has not sustained a TBI. Levels of these TBI biomarkers can be detected or measured alone or in combination as part of a mild TBI panel or signature.

### 7. METHODS FOR MEASURING THE LEVEL OF A TBI BIOMARKER

In the methods described above, TBI biomarker levels can be measured by any means, such as antibody dependent methods, such as immunoassays, protein immunoprecipitation, immunoelectrophoresis, chemical analysis, SDS-PAGE and Western blot analysis, protein immunostaining, electrophoresis analysis, a protein assay, a competitive binding assay, a functional protein assay, or chromatography or spectrometry methods, such as high-performance liquid chromatography (HPLC), mass spectrometry, or liquid chromatography-mass spectrometry (LC/MS) or capillary electrophoresis (CE)-MS, or direct infusion, or any separating front end coupled with MS. Also, the assay can be employed in clinical chemistry format such as would be known by one skilled in the art.

In some embodiments, measuring the level of a TBI biomarker includes contacting the sample with a first specific binding member and second specific binding member. In some embodiments the first specific binding member is a capture antibody and the second specific binding member is a detection antibody. In some embodiments, measuring the level of a TBI biomarker includes contacting the sample, either simultaneously or sequentially, in any order: (1) a capture antibody (e.g., a TBI biomarker-capture antibody), which binds to an epitope on a TBI biomarker or a TBI biomarker fragment to form a capture antibody-TBI biomarker antigen complex (e.g., TBI biomarker-capture antibody-TBI biomarker antigen complex), and (2) a detection antibody (e.g., TBI biomarker-detection antibody), which includes a detectable label and binds to an epitope on a TBI biomarker that is not bound by the capture antibody, to form a TBI biomarker antigen-detection antibody complex (e.g., TBI biomarker antigen-TBI biomarker-detection antibody complex), such that a capture antibody-TBI biomarker antigen-detection antibody complex (e.g., TBI biomarker-capture antibody-TBI biomarker antigen-TBI biomarker-detection antibody complex) is formed, and measuring the amount or concentration of a TBI biomarker in the sample based on the signal generated by the detectable label in the capture antibody-TBI biomarker antigen-detection antibody complex.

In some embodiments, the first specific binding member is immobilized on a solid support. In some embodiments, the second specific binding member is immobilized on a solid support. In some embodiments, the first specific binding member is a TBI biomarker antibody as described below.

In some embodiments, the sample is diluted or undiluted. The sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less.

Some instruments (such as, for example the Abbott Laboratories instruments ARCHITECT®, Abbott Alinity instruments, and other core laboratory instruments) other than a point-of-care device may be capable of measuring levels of a TBI biomarker in a sample at about 0.032 μg/L at 10% CV or lower. Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety.

a. Detection by Mass Spectrometry

As used herein, “MS data” generally refers to raw MS data obtained from a mass spectrometer and/or processed MS data in which peptides and their fragments (e.g., transitions and MS peaks) are already identified, analyzed and/or quantified. In some embodiments of the present disclosure, methods based on MRM-MS or SRM-MS and/or PRM-MS allow for the detection and accurate quantification of specific peptides in complex mixtures. SRM/MRM-MS is a technology with the potential for reliable and comprehensive quantification of substances of low abundance in complex samples. SRM/MRM-MS is performed on triple quadrupole-like instruments, in which increased selectivity is obtained through collision-induced dissociation. It is a non-scanning mass spectrometry technique, where two mass analyzers (Q1 and Q3) are used as static mass filters, to monitor a particular fragment of a selected precursor. On triple quadrapole instruments, various ionization methods can be used, including without limitation, electrospray ionization, chemical ionization, electron ionization, atmospheric pressure chemical ionization, and matrix-assisted laser desorption ionization. Both the first mass analyzer and the collision cell are continuously exposed to ions from the source in a time dependent manner. Once the ions move into the third mass analyzer time dependence becomes a factor. On triple quadrupole instruments, the first quadrapole mass filter, Q1, is the primary m/z selector after the sample leaves the ionization source. Any ions with mass-to-charge ratios other than the one selected for will not be allowed to infiltrate Q1. The collision cell, denoted as “q2”, located between the first quadrapole mass filter Q1 and second quadrapole mass filter Q3, is where fragmentation of the sample occurs in the presence of an inert gas like argon, helium, or nitrogen. Upon exiting the collision cell, the fragmented ions then travel onto the second quadrapole mass filter Q3, where m/z selection can occur again.

The specific pair of mass-over-charge (m/z) values associated to the precursor and fragment ions selected is referred to as a “transition.” The detector acts as a counting device for the ions matching the selected transition thereby returning an intensity distribution over time. MRM-MS is when multiple SRM-MS transitions are measured within the same experiment on the chromatographic time scale by rapidly switching between the different precursor/fragment pairs. Typically, the triple quadrupole instrument cycles through a series of transitions and records the signal of each transition as a function of the elution time. The method allows for additional selectivity by monitoring the chromatographic co-elution of multiple transitions for a given analyte.

For general references on mass spectrometry and proteomics, see e.g., Salvatore Sechi, Quantitative Proteomics by Mass Spectrometry (Methods in Molecular Biology) 2nd ed. 2016 Edition, Humana Press (New York, NY, 2009); Daniel Martins-de-Souza, Shotgun Proteomics: Methods and Protocols 2014 edition, Humana Press (New York, NY, 2014); Jörg Reinders and Albert Sickmann, Proteomics: Methods and Protocols (Methods in Molecular Biology) 2009 edition, Humana Press (New York, NY, 2009); and Jörg Reinders, Proteomics in Systems Biology: Methods and Protocols (Methods in Molecular Biology) 1^{st} ed. 2016 edition, Humana Press (New York, NY, 2009).

In addition to PRM-MS is also an application of SRM with parallel detection of all transitions in a single analysis using a high resolution mass spectrometer. PRM-MS provides high selectivity, high sensitivity and high-throughput to quantify selected peptide (Q1), hence quantify proteins (MS1). Again, multiple peptides can be specifically selected for each protein. PRM-MS methodology uses the quadrupole of a mass spectrometer to isolate a target precursor ion, fragments the targeted precursor ion in the collision cell, and then detects the resulting product ions in the Orbitrap mass analyzer. Quantification is carried out after data acquisition by extracting one or more fragment ions with 5-10 ppm mass windows. PRM-MS uses a quadrupole time-of-flight (QTOF) or hybrid quadrupole-orbitrap (QOrbitrap) mass spectrometer to carry out the peptides/proteins quantitation. Examples of QTOF include but are not limited to: TripleTOF 6600 or 5600 System (Sciex); X500R QTOF System (Sciex); 6500 Series Accurate-Mass Quadrupole Time-of-Flight (Q-TOF) (Agilent); or Xevo G2-XS QTof Quadrupole Time-of-Flight Mass Spectrometry (Waters). Examples of QObitrap include but are not limited to: Q Exactive Hybrid Quadrupole-Orbitrap Mass Spectrometer (Thermo Scientific); or Orbitrap Fusion Tribrid (Thermo Scientific).

In some embodiments, the developed methods herein can be applied to the quantification of polypeptides(s) or protein(s) in biological sample(s). Any kind of biological samples comprising polypeptides or proteins can be the starting point and be analyzed by the methods disclosed herein. Indeed, any protein/peptide containing sample can be used for and analyzed by the methods produced here (e.g., tissues, cells). The methods herein can also be used with peptide mixtures obtained by digestion. Digestion of a polypeptide or protein includes any kind of cleavage strategies, such as, enzymatic, chemical, physical or combinations thereof. According to some embodiments, the following parameters of the methods provided herein are determined: trypsin (or other protease) digestion and peptide clean up, best responding polypeptides, best responding proteins, best responding peptides, best responding fragments, fragment intensity ratios (increased high and reproducible peak intensities), optimal collision energies, and all the optimal parameters to maximize sensitivity and/or specificity of the methods.

In other embodiments, quantification of the polypeptides and/or of the corresponding proteins or activity/regulation of the corresponding proteins is desired. A selected peptide is labeled with a stable-isotope and used as an internal standard (SIL) to achieve absolute quantification of a protein of interest. The addition of a quantified stable-labeled peptide analogue of the tag to the peptide sample in known amount; and subsequently the tag and the peptide of interest is quantified by mass spectrometry and absolute quantification of the endogenous levels of the proteins is obtained.

In some embodiments, biomarkers of the present disclosure can be detected by mass spectrometry, a method that employs a mass spectrometer to detect gas phase ions, as described above. Examples of mass spectrometers are time-of-flight, magnetic sector, quadrupole filter, ion trap, ion cyclotron resonance, electrostatic sector analyzer, hybrids or combinations of the foregoing, and the like. In one embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein or with enrichment. In another embodiment, the mass spectrometric technique is multiple reaction monitoring (MRM) or quantitative MRM.

In some embodiments, the mass spectrometry method involves first enrichment by capturing one or more biomarkers on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In one embodiment, one could fractionate on an anion exchange resin and detect by MALDI directly. In another embodiment, one could capture the biomarkers on an immuno-chromatographic resin that comprises antibodies that bind the biomarkers, wash the resin to remove unbound material, elute the biomarkers from the resin and detect the eluted biomarkers by MALDI or on to another MS instrument (using any method for quantification).

The biomarkers of the present disclosure can also be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry) or by mass spectrometry using any MS instrument and any MS method.

### 8. TBI BIOMARKER-RECOGNIZING ANTIBODIES

The methods described herein may use an isolated antibody that specifically binds to a TBI biomarker or fragments thereof, referred to as “TBI biomarker antibody.” TBI biomarker antibodies can be used to assess the status of a TBI biomarker as a measure of traumatic brain injury, detect the presence of a TBI biomarker in a biological sample, quantify the amount of a TBI biomarker present in a biological sample, or detect the presence of and quantify the amount of a TBI biomarker in a biological sample.

TBI biomarker antibodies include any antibody that binds to a TBI biomarker, a fragment thereof, an epitope of a TBI biomarker, or a variant thereof. The antibody may be a fragment of the anti-TBI biomarker antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-TBI biomarker antibodies may be a chimeric anti-TBI biomarker antibody or a humanized anti-TBI biomarker antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008 (8): 85. Therefore, the antibody may be a product of the human and not animal repertoire. Because it is of human origin, the risks of reactivity against self-antigens may be minimized. Alternatively, standard yeast display libraries and display technologies may be used to select and isolate human anti-TBI biomarker antibodies. For example, libraries of naïve human single chain variable fragments (scFv) may be used to select human anti-TBI biomarker I antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

The antibody may immunospecifically bind to the peptide of a TBI biomarker, a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region.

### 9. ANTIBODY PREPARATION/PRODUCTION

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, in order to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77:4216-4220 (1980)), used with a DHFR selectable marker, e.g., as described in Kaufman and Sharp, J. Mol. Biol., 159:601-621 (1982), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e. binds human troponin I) and the other heavy and light chain are specific for an antigen other than a human TBI biomarker by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with a TBI biomarker or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F (ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); Hawkins et al, J. Mol. Biol., 226:889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. Sec, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFv's), including tobacco seeds and potato tubers. Sec, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al., (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (131I), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F (ab).

Antibody production via the use of hybridoma technology, the selected lymphocyte antibody method (SLAM), transgenic animals, and recombinant antibody libraries is described in more detail below.

### 10. TBI BIOMARKER-RECOGNIZING APTAMERS

The methods of the present disclosure include the use of aptamers to detect or identify one or more TBI biomarkers. Aptamers are suitable for use in developing probes having high affinity and selectivity for target molecules, such as TBI peptide biomarkers. Aptamers include single-stranded DNA (ssDNA), RNA, or modified nucleic acids, which have the ability to bind specifically to their targets, which range from small organic molecules to proteins and peptides. The basis for target recognition is the tertiary structures formed by the single-stranded oligonucleotides, as known in the art. In some embodiments, aptamers used to detect or identify one or more TBI biomarkers can be obtained through an in vitro selection process known as SELEX, in which aptamers are selected from a library of random sequences of synthetic DNA or RNA by repetitive binding of the oligonucleotides to target molecules.

In some embodiments, nucleic acids that constitute an aptamer library mixture used for screening for candidate TBI biomarker capture agents can be single-stranded DNA or RNA with or without chemical modifications. The introduction of additional chemical entities into DNA during the selection process can include, for example, the use of a 5-alkyne modified nucleobase, (e.g., thymine). Additionally, 5-C8-alkyne modified nucleotide-triphosphates, for example deoxythymidines, are commercially available or can be synthesized. Such 5-C8-alkyne modified nucleobases can be introduced into DNA by PCR. Such modifications can be further derivatized with so called bio-orthogonal chemistry, for example, using the Cu(I) catalyzed 1,3-dipolar cycloaddition of respective azides with the alkyne. Beside the Cu(I) catalysed azide-alkyne cycloaddition (CuAAC), copper-free strain-promoted azide-alkyne cycloaddition (SPAAC) reactions also are useful. In some embodiments involving cellular or living systems, the strain-promoted azide-alkyne cycloaddition can overcome toxicity issues associated with the use of Cu(I). Any number of desirable chemical modifications can be added to the oligonucleotide library used for screening purposes. Examples of such modifications include without limitation aliphatic-aromatic-, charged-, basic-, acidic, heteroaromatic-, sugar-kind of-, metal-containing- or peptide-residues.

In some embodiments, a nucleobase that is to be modified to contain an azide-alkyne chemical group can include an ethynyl-, propynyl- or butynyl-dU, dA, dC or dG nucleotide. In other embodiments, a nucleobase that is to be modified to contain an azide-alkyne chemical group may be an ethynyl-dU nucleotide, or an ethynyl-dA nucleotide, an ethynyl-dC nucleotide or an ethynyl-dG nucleotide. Nucleotide aptamer libraries with these example modifications can be used in various SELEX-based selection methods, in order to enhance the chemical diversity of DNA aptamer libraries. The starting, or candidate, mixture of nucleic acids can be modified such that at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, at least 99%, or 100% of the members of the mixture are modified to comprise the functionalization introduced by click chemistry, for example. Less than 100% modification may allow for enhanced diversity by allowing certain positions in an oligonucleotide to be modified but not others, whereas 100% modification ensures consistency during the selection process. In some embodiments, different modifications are made at different positions in the oligonucleotide to further enhance diversity.

TBI biomarker-recognizing aptamers can be used in various methods to detect a presence or level of one or more TBI biomarker in a biological sample (e.g., biological entities of interest such as proteins, nucleic acids, or microvesicles). The aptamer can function as a binding agent or capture agent to assess presence or level of the cognate TBI biomarker. In various embodiments of the present disclosure directed to diagnostics and/or prognostics, one or more aptamers can be configured in a ligand-target based assay, where one or more aptamer can be contacted with a selected biological sample to allow the or more aptamer to associate with or binds to its target TBI biomarker molecule. Aptamers can also be used to identify a profile of multiple TBI biomarkers (a “biomarker” profile or signature) based on the biological samples assessed and biomarkers detected. A biomarker profile of a biological sample may comprise a presence, level or other characteristic of one or more biomarker of interest that can be assessed, including without limitation a presence, level, sequence, mutation, rearrangement, translocation, deletion, epigenetic modification, methylation, post-translational modification, allele, activity, complex partners, stability, half-life, and the like.

Biomarker profiles or signatures can be used to evaluate diagnostic and/or prognostic criteria such as presence of disease, disease staging, disease monitoring, disease stratification, or surveillance for detection, metastasis or recurrence or progression of disease. For example, methods of the present disclosure can include methods for correlating a TBI biomarker profile to a selected condition or disease, such as severe TBI, mild TBI, or a subclass of mild TBI. A biomarker profile can also be used clinically in making decisions concerning treatment modalities including therapeutic intervention. A biomarker profile based on aptamer detection, identification, and/or quantification can further be used clinically to make treatment decisions, including whether to perform an imaging procedure (e.g., MRI).

### 11. VARIATIONS ON METHODS

The disclosed methods of determining the presence or amount of analyte of interest (e.g., TBI biomarker) present in a sample may be as described herein. The methods may also be adapted in view of other methods for analyzing analytes. Examples of well-known variations include, but are not limited to, immunoassay, such as sandwich immunoassay (e.g., monoclonal-monoclonal sandwich immunoassays, monoclonal-polyclonal sandwich immunoassays, including enzyme detection (enzyme immunoassay (EIA) or enzyme-linked immunosorbent assay (ELISA), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, etc.

a. Immunoassay

The analyte of interest, and/or peptides of fragments thereof (e.g., TBI biomarker and/or peptides or fragments thereof), may be analyzed using TBI biomarker antibodies in an immunoassay. The presence or amount of analyte (e.g., TBI biomarker) can be determined using antibodies and detecting specific binding to the analyte. For example, the antibody, or antibody fragment thereof, may specifically bind to the analyte. If desired, one or more of the antibodies can be used in combination with one or more commercially available monoclonal/polyclonal antibodies. Such antibodies are available from companies such as R&D Systems, Inc. (Minneapolis, MN) and Enzo Life Sciences International, Inc. (Plymouth Meeting, PA).

The presence or amount of analyte (e.g., TBI biomarker) present in a body sample may be readily determined using an immunoassay, such as sandwich immunoassay (e.g., monoclonal-monoclonal sandwich immunoassays, monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, MN)). An example of a point-of-care device that can be used is i-STAT® (Abbott, Laboratories, Abbott Park, IL). Other methods that can be used include a chemiluminescent microparticle immunoassay, in particular one employing the ARCHITECT® automated analyzer (Abbott Laboratories, Abbott Park, IL), as an example. Other methods include, for example, mass spectrometry, and immunohistochemistry (e.g., with sections from tissue biopsies), using anti-analyte (e.g., anti-TBI biomarker) antibodies (monoclonal, polyclonal, chimeric, humanized, human, etc.) or antibody fragments thereof against analyte (e.g., TBI biomarker). Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of the antibody to the analyte can be detected via direct labels, such as fluorescent or luminescent tags, metals and radionuclides attached to the antibody or via indirect labels, such as alkaline phosphatase or horseradish peroxidase.

The use of immobilized antibodies or antibody fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

A homogeneous format may be used. For example, after the test sample is obtained from a subject, a mixture is prepared. The mixture contains the test sample being assessed for analyte (e.g., TBI biomarker) and a specific binding partner. The order in which the test sample and the specific binding partner are added to form the mixture is not critical. The test sample is simultaneously contacted with the specific binding partner. In some embodiments, the specific binding partner and any TBI biomarker contained in the test sample may form a specific binding partner-analyte (e.g., TBI biomarker)-antigen complex. The specific binding partner may be an anti-analyte antibody (e.g., anti-TBI biomarker antibody that binds to an epitope having an amino acid sequence comprising at least three contiguous (3) amino acids of the TBI biomarker. Moreover, the specific binding partner may be labeled with or contains a detectable label as described above.

A heterogeneous format may be used. For example, after the test sample is obtained from a subject, a first mixture is prepared. The mixture contains the test sample being assessed for analyte (e.g., TBI biomarker) and a first specific binding partner, wherein the first specific binding partner and any TBI biomarker contained in the test sample form a first specific binding partner-analyte (e.g., TBI biomarker)-antigen complex. The first specific binding partner may be an anti-analyte antibody (e.g., anti-TBI biomarker antibody that binds to an epitope having an amino acid sequence comprising at least three contiguous (3) amino acids of the TBI biomarker. The order in which the test sample and the first specific binding partner are added to form the mixture is not critical.

The first specific binding partner may be immobilized on a solid phase. The solid phase used in the immunoassay (for the specific binding partner) can be any solid phase known in the art, such as, but not limited to, a magnetic particle, a bead, a test tube, a microtiter plate, a cuvette, a membrane, a scaffolding molecule, a film, a filter paper, a disc, and a chip. In those embodiments where the solid phase is a bead, the bead may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFc₂O₄, CoFC₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The solid support on which the first specific binding member is immobilized may be stored in dry form or in a liquid. The magnetic beads may be subjected to a magnetic field prior to or after contacting with the sample with a magnetic bead on which the first specific binding member is immobilized.

After the mixture containing the first specific binding partner-analyte (e.g., TBI biomarker) antigen complex is formed, any unbound analyte (e.g., TBI biomarker) is removed from the complex using any technique known in the art. For example, the unbound analyte can be removed by washing. Desirably, however, the first specific binding partner is present in excess of any analyte present in the test sample, such that all analyte that is present in the test sample is bound by the first specific binding partner.

After any unbound analyte (e.g., TBI biomarker) is removed, a second specific binding partner is added to the mixture to form a first specific binding partner-analyte of interest (e.g., TBI biomarker)-second specific binding partner complex. The second specific binding partner may be an anti-analyte antibody (e.g., TBI biomarker antibody that binds to an epitope having an amino acid sequence comprising at least three contiguous (3) amino acids of the TBI biomarker. Moreover, the second specific binding partner is labeled with or contains a detectable label as described above.

The use of immobilized antibodies or antibody fragments thereof may be incorporated into the immunoassay. The antibodies may be immobilized onto a variety of supports, such as magnetic or chromatographic matrix particles (such as a magnetic bead), latex particles or modified surface latex particles, polymer or polymer film, plastic or plastic film, planar substrate, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

### (1) Sandwich Immunoassay

A sandwich immunoassay measures the amount of antigen between two layers of antibodies (i.e. at least one capture antibody) and a detection antibody (i.e. at least one detection antibody). The capture antibody and the detection antibody bind to different epitopes on the antigen, e.g., analyte of interest such as a TBI biomarker). Desirably, binding of the capture antibody to an epitope does not interfere with binding of the detection antibody to an epitope. Either monoclonal or polyclonal antibodies may be used as the capture and detection antibodies in the sandwich immunoassay.

Generally, at least two antibodies are employed to separate and quantify analyte (e.g., TBI biomarker) in a test sample. More specifically, the at least two antibodies bind to certain epitopes of analyte forming an immune complex which is referred to as a “sandwich.” One or more antibodies can be used to capture the analyte in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. Antibodies are selected so that the one or more first antibodies brought into contact with a test sample suspected of containing analyte do not bind to all or part of an epitope recognized by the second or subsequent antibodies, thereby interfering with the ability of the one or more second detection antibodies to bind to the analyte.

The antibodies may be used as a first antibody in said immunoassay. The antibody immunospecifically binds to epitopes on analyte (e.g., TBI biomarker). In addition to the antibodies of the present disclosure, said immunoassay may comprise a second antibody that immunospecifically binds to epitopes that are not recognized or bound by the first antibody.

A test sample suspected of containing analyte (e.g., TBI biomarker) can be contacted with at least one first capture antibody (or antibodies) and at least one second detection antibodies either simultaneously or sequentially. In the sandwich assay format, a test sample suspected of containing analyte is first brought into contact with the at least one first capture antibody that specifically binds to a particular epitope under conditions which allow the formation of a first antibody-analyte antigen complex. If more than one capture antibody is used, a first multiple capture antibody-TBI biomarker antigen complex is formed. In a sandwich assay, the antibodies, preferably, the at least one capture antibody, are used in molar excess amounts of the maximum amount of analyte expected in the test sample. For example, from about 5 μg/mL to about 1 mg/mL of antibody per ml of microparticle coating buffer may be used.

i. Anti-TBI Biomarker Capture Antibody

Optionally, prior to contacting the test sample with the at least one first capture antibody, the at least one first capture antibody can be bound to a solid support which facilitates the separation the first antibody-analyte (e.g., TBI biomarker) complex from the test sample. Any solid support known in the art can be used, including but not limited to, solid supports made out of polymeric materials in the forms of wells, tubes, or beads (such as a microparticle). The antibody (or antibodies) can be bound to the solid support by adsorption, by covalent bonding using a chemical coupling agent or by other means known in the art, provided that such binding does not interfere with the ability of the antibody to bind analyte. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl) carbodiimide.

After the test sample suspected of containing analyte (e.g., TBI biomarker) is incubated in order to allow for the formation of a first capture antibody (or multiple antibody)-analyte complex. The incubation can be carried out at a pH of from about 4.5 to about 10.0, at a temperature of from about 2° C. to about 45° C., and for a period from at least about one (1) minute to about eighteen (18) hours, from about 2-6 minutes, from about 7-12 minutes, from about 5-15 minutes, or from about 3-4 minutes.

ii. Detection Antibody

After formation of the first/multiple capture antibody-analyte (e.g., TBI biomarker) complex, the complex is then contacted with at least one second detection antibody (under conditions that allow for the formation of a first/multiple antibody-analyte antigen-second antibody complex). In some embodiments, the test sample is contacted with the detection antibody simultaneously with the capture antibody. If the first antibody-analyte complex is contacted with more than one detection antibody, then a first/multiple capture antibody-analyte-multiple antibody detection complex is formed. As with first antibody, when the at least second (and subsequent) antibody is brought into contact with the first antibody-analyte complex, a period of incubation under conditions similar to those described above is required for the formation of the first/multiple antibody-analyte-second/multiple antibody complex. Preferably, at least one second antibody contains a detectable label. The detectable label can be bound to the at least one second antibody prior to, simultaneously with or after the formation of the first/multiple antibody-analyte-second/multiple antibody complex. Any detectable label known in the art can be used.

Chemiluminescent assays can be performed in accordance with the methods described in Adamczyk et al., Anal. Chim. Acta 579 (1): 61-67 (2006). While any suitable assay format can be used, a microplate chemiluminometer (Mithras LB-940, Berthold Technologies U.S.A., LLC, Oak Ridge, TN) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the test sample and the specific binding partner(s) are added to form the mixture for chemiluminescent assay is not critical. If the first specific binding partner is detectably labeled with an acridinium compound, detectably labeled first specific binding partner-antigen (e.g., TBI biomarker) complexes form. Alternatively, if a second specific binding partner is used and the second specific binding partner is detectably labeled with an acridinium compound, detectably labeled first specific binding partner-analyte-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of analyte (e.g., TBI biomarker) is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample. Other labels other than chemiluminescent labels can be employed. For instance, enzymatic labels (including but not limited to alkaline phosphatase) can be employed.

The chemiluminescent signal, or other signal, that is generated can be detected using routine techniques known to those skilled in the art. Based on the intensity of the signal generated, the amount of analyte of interest (e.g., TBI biomarker) in the sample can be quantified. Specifically, the amount of analyte in the sample is proportional to the intensity of the signal generated. The amount of analyte present can be quantified by comparing the amount of light generated to a standard curve for analyte or by comparison to a reference standard. The standard curve can be generated using serial dilutions or solutions of known concentrations of analyte by mass spectroscopy, gravimetric methods, and other techniques known in the art.

### (2) Forward Competitive Inhibition Assay

In a forward competitive format, an aliquot of labeled analyte of interest (e.g., TBI biomarker) having a fluorescent label, a tag attached with a cleavable linker, etc.) of a known concentration is used to compete with analyte of interest in a test sample for binding to analyte of interest antibody (e.g., a TBI biomarker antibody).

In a forward competition assay, an immobilized specific binding partner (such as an antibody) can either be sequentially or simultaneously contacted with the test sample and a labeled analyte of interest, analyte of interest fragment or analyte of interest variant thereof. The analyte of interest peptide, analyte of interest fragment or analyte of interest variant can be labeled with any detectable label, including a detectable label comprised of tag attached with a cleavable linker. In this assay, the antibody can be immobilized on to a solid support. Alternatively, the antibody can be coupled to an antibody, such as an antispecies antibody, that has been immobilized on a solid support, such as a microparticle or planar substrate.

The labeled analyte of interest, the test sample and the antibody are incubated under conditions similar to those described above in connection with the sandwich assay format. Two different species of antibody-analyte of interest complexes may then be generated. Specifically, one of the antibody-analyte of interest complexes generated contains a detectable label (e.g., a fluorescent label, etc.) while the other antibody-analyte of interest complex does not contain a detectable label. The antibody-analyte of interest complex can be, but does not have to be, separated from the remainder of the test sample prior to quantification of the detectable label. Regardless of whether the antibody-analyte of interest complex is separated from the remainder of the test sample, the amount of detectable label in the antibody-analyte of interest complex is then quantified. The concentration of analyte of interest (such as membrane-associated analyte of interest, soluble analyte of interest, fragments of soluble analyte of interest, variants of analyte of interest (membrane-associated or soluble analyte of interest) or any combinations thereof) in the test sample can then be determined, e.g., as described above.

### (3) Reverse Competitive Inhibition Assay

In a reverse competition assay, an immobilized analyte of interest (e.g., TBI biomarker) can either be sequentially or simultaneously contacted with a test sample and at least one labeled antibody.

The analyte of interest can be bound to a solid support, such as the solid supports discussed above in connection with the sandwich assay format.

The immobilized analyte of interest, test sample and at least one labeled antibody are incubated under conditions similar to those described above in connection with the sandwich assay format. Two different species analyte of interest-antibody complexes are then generated. Specifically, one of the analyte of interest-antibody complexes generated is immobilized and contains a detectable label (e.g., a fluorescent label, etc.) while the other analyte of interest-antibody complex is not immobilized and contains a detectable label. The non-immobilized analyte of interest-antibody complex and the remainder of the test sample are removed from the presence of the immobilized analyte of interest-antibody complex through techniques known in the art, such as washing. Once the non-immobilized analyte of interest antibody complex is removed, the amount of detectable label in the immobilized analyte of interest-antibody complex is then quantified following cleavage of the tag. The concentration of analyte of interest in the test sample can then be determined by comparing the quantity of detectable label as described above.

### (4) One-Step Immunoassay or “Capture on the Fly” Assay

In a capture on the fly immunoassay, a solid substrate is pre-coated with an immobilization agent. The capture agent, the analyte (e.g., TBI biomarker) and the detection agent are added to the solid substrate together, followed by a wash step prior to detection. The capture agent can bind the analyte and comprises a ligand for an immobilization agent. The capture agent and the detection agents may be antibodies or any other moiety capable of capture or detection as described herein or known in the art. The ligand may comprise a peptide tag and an immobilization agent may comprise an anti-peptide tag antibody. Alternately, the ligand and the immobilization agent may be any pair of agents capable of binding together so as to be employed for a capture on the fly assay (e.g., specific binding pair, and others such as are known in the art). More than one analyte may be measured. In some embodiments, the solid substrate may be coated with an antigen and the analyte to be analyzed is an antibody. This method can also be coupled with MS detection and quantification.

In certain other embodiments, in a one-step immunoassay or “capture on the fly”, a solid support (such as a microparticle) pre-coated with an immobilization agent (such as biotin, streptavidin, etc.) and at least a first specific binding member and a second specific binding member (which function as capture and detection reagents, respectively) are used. The first specific binding member comprises a ligand for the immobilization agent (for example, if the immobilization agent on the solid support is streptavidin, the ligand on the first specific binding member may be biotin) and also binds to the analyte of interest (e.g., TBI biomarker). The second specific binding member comprises a detectable label and binds to an analyte of interest. The solid support and the first and second specific binding members may be added to a test sample (either sequentially or simultaneously). The ligand on the first specific binding member binds to the immobilization agent on the solid support to form a solid support/first specific binding member complex. Any analyte of interest present in the sample binds to the solid support/first specific binding member complex to form a solid support/first specific binding member/analyte complex. The second specific binding member binds to the solid support/first specific binding member/analyte complex and the detectable label is detected. An optional wash step may be employed before the detection. In certain embodiments, in a one-step assay more than one analyte may be measured. In certain other embodiments, more than two specific binding members can be employed. In certain other embodiments, multiple detectable labels can be added. In certain other embodiments, multiple analytes of interest can be detected, or their amounts, levels or concentrations, measured, determined or assessed, including using mass spectrometry.

The use of a capture on the fly assay can be done in a variety of formats as described herein, and known in the art. For example the format can be a sandwich assay such as described above, but alternately can be a competition assay, can employ a single specific binding member, or use other variations such as are known. This method can also be coupled with MS detection and quantification.

### 12. OTHER FACTORS

The methods of diagnosing, prognosticating, risk stratifying, and/or assessing, as described above, can further include using other factors for the diagnosis, prognostication, and assessment. In some embodiments, traumatic brain injury may be diagnosed using the Glasgow Coma Scale or the Extended Glasgow Outcome Scale (GOSE). Other tests, scales or indices can also be used either alone or in combination with the Glasgow Coma Scale. An example is the Ranchos Los Amigos Scale. The Ranchos Los Amigos Scale measures the levels of awareness, cognition, behavior and interaction with the environment. The Ranchos Los Amigos Scale includes: Level I: No Response; Level II: Generalized Response; Level III: Localized Response; Level IV: Confused-agitated; Level V: Confused-inappropriate; Level VI: Confused-appropriate; Level VII: Automatic-appropriate; and Level VIII: Purposeful-appropriate.

### 13. SAMPLES

In some embodiments, the sample is obtained after the human subject sustained an injury to the head caused by physical shaking, blunt impact by an external mechanical or other force that results in a closed or open head trauma, one or more falls, explosions or blasts or other types of blunt force trauma. In some embodiments, the sample is obtained after the human subject has ingested or been exposed to a chemical, toxin or combination of a chemical and toxin. Examples of such chemicals and/or toxins include, fires, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. In some embodiments, the sample is obtained from a human subject that suffers from an autoimmune disease, a metabolic disorder, a brain tumor, hypoxia, one or more viruses, meningitis, hydrocephalus or combinations thereof.

In yet another embodiment, the methods described herein use samples that also can be used to determine whether or not a subject has or is at risk of developing mild traumatic brain injury by determining the levels of a TBI biomarker in a subject using the anti-TBI biomarker antibodies described below, or antibody fragments thereof. Thus, in particular embodiments, the disclosure also provides a method for determining whether a subject having, or at risk for, traumatic brain injuries, discussed herein and known in the art, is a candidate for therapy or treatment. Generally, the subject is at least one who: (i) has experienced an injury to the head; (ii) ingested and/or been exposed to one or more chemicals and/or toxins; (iii) suffers from an autoimmune disease, a metabolic disorder, a brain tumor, hypoxia, one or more viruses, meningitis, hydrocephalus or suffers from any combinations thereof; or (iv) any combinations of (i)-(iii); or, who has actually been diagnosed as having, or being at risk for TBI (such as, for example, subjects suffering from an autoimmune disease, a metabolic disorder, a brain tumor, hypoxia, one or more viruses, meningitis, hydrocephalus or combinations thereof), and/or who demonstrates an unfavorable (i.e. clinically undesirable) concentration or amount of a TBI biomarker or a TBI biomarker fragment, as described herein.

a. Test or Biological Sample

As used herein, “sample”, “test sample”, “biological sample” refer to fluid sample containing or suspected of containing a TBI biomarker. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing a TBI biomarker may be assayed directly. In a particular example, the source containing a TBI biomarker is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

In some cases, the fluid sample may be diluted prior to use in an assay. For example, in embodiments where the source containing a TBI biomarker is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source containing a TBI biomarker is a human body fluid (e.g., blood, serum), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

b. Controls

It may be desirable to include a control. The control may be analyzed concurrently with the sample from the subject as described above. The results obtained from the subject sample can be compared to the results obtained from the control sample. Standard curves may be provided, with which assay results for the sample may be compared. Such standard curves present levels of marker as a function of assay units, i.e. fluorescent signal intensity, if a fluorescent label is used. Using samples taken from multiple donors, standard curves can be provided for reference levels of a TBI biomarker in normal healthy tissue, as well as for “at-risk” levels of the TBI biomarker in tissue taken from donors, who may have one or more of the characteristics set forth above.

Thus, in view of the above, a method for determining the presence, amount, or concentration of a TBI biomarker in a test sample is provided. The method comprises assaying the test sample for a TBI biomarker by an immunoassay, for example, employing at least one capture antibody that binds to an epitope on a TBI biomarker and at least one detection antibody that binds to an epitope on a TBI biomarker which is different from the epitope for the capture antibody and optionally includes a detectable label, and comprising comparing a signal generated by the detectable label as a direct or indirect indication of the presence, amount or concentration of a TBI biomarker in the test sample to a signal generated as a direct or indirect indication of the presence, amount or concentration of a TBI biomarker in a calibrator. The calibrator is optionally, and is preferably, part of a series of calibrators in which each of the calibrators differs from the other calibrators in the series by the concentration of the TBI biomarker.

### 14. KIT

Provided herein is a kit, which may be used for assaying or assessing a test sample for one or more TBI biomarkers and/or fragments thereof. The kit comprises at least one component for assaying the test sample for a TBI biomarker and instructions for assaying the test sample for a TBI biomarker. For example, the kit can comprise instructions for assaying the test sample for a TBI biomarker by immunoassay (e.g., chemiluminescent microparticle immunoassay) or by mass spectrometry assay (e.g., PRM-MS or MRM/SRM-MS). Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The at least one component may include at least one composition comprising one or more isolated antibodies or antibody fragments thereof that specifically bind to a TBI biomarker. The antibody may be a TBI biomarker detection antibody and/or capture antibody.

Alternatively or additionally, the kit can comprise a calibrator or control (e.g., purified, and optionally lyophilized, TBI biomarker) and/or at least one container (e.g., tube, microtiter plates or strips, which can be already coated with an anti-TBI biomarker antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e. reagents, standards, buffers, diluents, etc., which are necessary to perform the assay. The instructions also can include instructions for generating a standard curve.

The kit may further comprise reference standards for quantifying a TBI biomarker. The reference standards may be employed to establish standard curves for interpolation and/or extrapolation of TBI biomarker concentrations. Standards cans include proteins or peptide fragments composed of amino acids residues or N15 stable isotopic labeled proteins or peptide fragments for various analytes, as well as standards for sample processing, including standards involving spikes in proteins and quantitative peptides. In some embodiments, the reference standards for a TBI biomarker can correspond to the 99th percentile derived from a healthy reference population. Such reference standards can be determined using routine techniques known in the art.

Any antibodies, which are provided in the kit, such as recombinant antibodies specific for a TBI biomarker, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes (e.g., TBI biomarker) or reagents for detecting the analyte (e.g., TBI biomarker). The antibodies, standard peptides or peptide fragments, calibrators, and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc, or chip.

If desired, the kit can further comprise one or more components, alone or in further combination with instructions, for assaying the test sample for another analyte, which can be a biomarker, such as a biomarker of traumatic brain injury or disorder.

a. Adaptation of Kit and Method

The kit (or components thereof), as well as the method for assessing or determining the concentration of a TBI biomarker in a test sample by an immunoassay as described herein, can be adapted for use in a variety of automated and semi-automated systems (including those wherein the solid phase comprises a microparticle), as described, e.g., U.S. Pat. No. 5,063,081, U.S. Patent Application Publication Nos. 2003/0170881, 2004/0018577, 2005/0054078, and 2006/0160164 and as commercially marketed e.g., by Abbott Laboratories (Abbott Park, IL) as Abbott Point of Care (i-STAT® or i-STAT Alinity, Abbott Laboratories) as well as those described in U.S. Pat. Nos. 5,089,424 and 5,006,309, and as commercially marketed, e.g., by Abbott Laboratories (Abbott Park, IL) as ARCHITECT® or the series of Abbott Alinity devices.

Some of the differences between an automated or semi-automated system as compared to a non-automated system (e.g., ELISA) include the substrate to which the first specific binding partner (e.g., analyte antibody or capture antibody) is attached (which can affect sandwich formation and analyte reactivity), and the length and timing of the capture, detection, and/or any optional wash steps. Whereas a non-automated format such as an ELISA may require a relatively longer incubation time with sample and capture reagent (e.g., about 2 hours), an automated or semi-automated format (e.g., ARCHITECT®, Alinity, and any successor platform, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT®, Alinity, and any successor platform).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits, and kit components can be employed in other formats, for example, on electrochemical or other hand-held or point-of-care assay systems. As mentioned previously, the present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Patent App. Publication Nos. 2003/0170881, 2004/0018577, 2005/0054078, and 2006/0160164, which are incorporated in their entireties by reference for their teachings regarding same.

In particular, with regard to the adaptation of an assay to the i-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an i-STAT® cartridge with a fluidics format suitable for immunoassay. On a portion of the silicon chip, there is a specific binding partner for a TBI biomarker, such as one or more TBI biomarker antibodies one or more monoclonal/polyclonal antibody or a fragment thereof, a variant thereof, or a fragment of a variant thereof that can bind a TBI biomarker) or one or more anti-TBI biomarker DVD-Igs (or a fragment thereof, a variant thereof, or a fragment of a variant thereof that can bind a TBI biomarker), any of which can be detectably labeled. Within the fluid pouch of the cartridge is an aqueous reagent that includes p-aminophenol phosphate.

In operation, a sample from a subject suspected of suffering from TBI is added to the holding chamber of the test cartridge, and the cartridge is inserted into the i-STAT® reader. A pump element within the cartridge pushes the sample into a conduit containing the chip. The sample is brought into contact with the sensors allowing the enzyme conjugate to dissolve into the sample. The sample is oscillated across the sensors to promote formation of the sandwich of approximately 2-12 minutes. In the penultimate step of the assay, the sample is pushed into a waste chamber and wash fluid, containing a substrate for the alkaline phosphatase enzyme, is used to wash excess enzyme conjugate and sample off the sensor chip. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of a TBI biomarker in the sample by means of an embedded algorithm and factory-determined calibration curve.

The methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, IL) and containing 2-(N-morpholino) ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, IL), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an i-STAT® cartridge format, using a nucleic acid sequence linked to the signal antibody as a signal amplifier. Adaptation of a cartridge for multiplex use, such as used for i-Stat, has been described in the patent literature, such as for example, U.S. Pat. No. 6,438,498, the contents of which are herein incorporated by reference.

The methods and kits as described herein may also involve single molecule counting. In certain embodiments, a method for analyte analysis may involve assessing an analyte present in a sample. In certain embodiments, the assessing may be used for determining presence of and/or concentration of an analyte in a sample. In certain embodiments, the method may also be used for determining presence of and/or concentration of a plurality of different analytes present in a sample.

Any device known in the art that allows for the detection of a single molecule of one or more analytes of interest can be used in the systems described herein. For example, the device can be a microfluidics device, digital microfluidics device (DMF), a surface acoustic wave based microfluidic device (SAW), an integrated DMF and analyte detection device, an integrated SAW and analyte detection device, or robotics based assay processing unit. Examples of other devices that can be used include the Quanterix SIMOA™ (Lexington, MA), Singulex's single molecule counting (SMC™) technology (Alameda, CA, see for example, U.S. Pat. No. 9,239,284, the contents of which are herein incorporated by reference), etc.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety.

The methods and kits as described herein can involve mass spectrometry using DIA-MS, DDA-MS or SRM/MRM-MS or PRM-MS. In certain embodiments, methods for analyte analysis can involve assessing a sample for the presence of an analyte. In certain embodiments, assessing a sample for the presence of an analyte can be used for determining presence of and/or concentration of an analyte or a fragment in a sample. In certain embodiments, a method can also be used for determining presence of and/or concentration of a plurality of different analytes or analyte fragments present in a sample. Quantification can be performed using internal control proteins or peptide fragments.

While certain embodiments herein are advantageous when employed to assess disease, such as traumatic brain injury, the assays and kits also optionally can be employed to assess various biomarkers in other diseases, disorders, and conditions as appropriate.

The method of assay also can be used to identify a compound that ameliorates diseases, such as traumatic brain injury. For example, a cell that expresses any of the various biomarkers described herein can be contacted with a candidate compound. The level of expression of one or more of these biomarkers in the cell contacted with the compound can be compared to that in a control cell using the methods and assays described herein.

The present disclosure has multiple aspects, illustrated by the following non-limiting examples.

### 15. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Development of a Multi-Modality Classification Scheme for Traumatic Brain Injury

This study was directed at developing a classification scheme for brain injury that indicates the nature (type) and severity of injury. Trauma patients were divided into three groups for analysis: only brain injured, only non-brain injured, and combined injury. Brain injured and non-brain injured trauma groups were compared to each other and to the combination of brain/nonbrain trauma. These trauma groups were compared to non-trauma controls. CSF from trauma patients was compared to CSF from non-trauma patients. A secondary goal was to determine whether any of the measures, alone or in combination, had utility as a predictor of clinical outcome after TBI.

An objective multi-modality classification scheme and outcome measure for traumatic brain injury was developed based on several measures: 1) blood-based biomarkers; 2) physiologic measures and evaluation; and 3) radiographic measures (CT and 3Tesla (also known as 3T) MRI). Blood-based biomarkers can indicate which cell types are damaged (e.g., glial vs. neuronal) and radiography can detect structural changes.

Study Site: Trauma patient were recruited at Hennepin County Medical Center (HCMC) in the state of Minnesota. Participants included trauma patients of all ages presenting to the HCMC Emergency Department (ED), trauma bay, or as direct transfer to neurosurgery. Trauma patients were excluded if they had a major psychiatric or neurologic disorder, were developmentally abnormal, or were prisoners. Subjects were identified by searching medical records for all trauma admissions and cross-checking with the American College of Surgeons trauma registry utilized in the hospital.

All trauma patients were recruited for screening at the time of presentation and underwent: 1) a standardized (templated) history and physical examination; 2) analysis of serum biomarkers if blood was drawn for other indications; 3) radiographic study as clinically indicated; 4) follow-up as clinically indicated with 1)-3); 5) pathologic specimen analysis in patients going to the operating room only; 6) CSF analysis in patients receiving ventriculostomy catheters only; 7) brain tissue oxygenation analysis in patients receiving Licox only; and 8) outcome assessment in the TBI center as clinically indicated. At the time of admission, the potential participants underwent a 24 hour screening process (Table 2) before providing informed consent.

TABLE 2
Screening Assessments

| | Adult | Pediatric |
|---|---|---|
| All | Surgery-Trauma History and Physical | |
| | Selections from Neurosurgery-Trauma History and Physical | |
| Awake | SCAT3: SAC, SSS-C | Child SCAT3: SAC-C, SSS-C |

| | |
|---|---|
| OR | Pathogenic Specimen |
| VC | CSF Analysis |
| Licox | Brain Tissue Oxygenation |

OR: Patients going to the operating room;
VC: Patients receiving ventriculostomy catheters;
Licox: Patients receiving Licox

Patients who consented and controls (age and gender matched) who had undergone the above were subject to the following additional studies: 1) genomic, serum, and CSF screening; and 2) 3T MRI in select circumstances (serum markers in the test group; normal markers in the control group). Trauma patients included the full spectrum ranging from non-brain injured, CT-negative to structurally brain injured, requiring surgery. Patients and controls were recruited over approximately 15 months. Surviving trauma subjects were followed until they were discharged from HCMC services. Subjects evaluated in the ER and released were invited for research follow-up.

The screening process included a standardized and templated medical history and physical examination. The template was the current “Surgery-Trauma History and Physical” template in EPIC with one additional question that asks if the patient suffered a head trauma. If the patient did, three sections automatically dropped down for additional information. The first was information from the Neurosurgery trauma history and physical template, including subarachnoid grade, hemorrhage grade, intracerebral hemorrhage, and social history (level of education, employment, living arrangements, and ethnicity). The final two were standardized brain injury assessment tools: the Standardized Assessment of Concussion (SAC) and the Symptom Severity Score (SSS). Pediatric versions of these assessments were available and used when indicated. Additionally, the loss of consciousness question already included in the “Surgery Trauma History and Physical” template was copied into this drop down section with a subset of questions that provided a more clear understanding of the loss of consciousness event and the patient's current orientation. The most clinically accurate assessment taken during the first 24 hours of admission was used for future data analysis.

Non-TBI subjects were also included based on the following criteria: be between 15 and 50 years of age at the time of enrollment; have similar characteristics as the TBI population in terms of gender, age, handedness, educational level, and scanner criteria; and be capable of sufficiently clear communication and language fluency to allow the subject to provide written informed consent, or assent with parental or guardian consent for minors, and to complete study assessments for participation in all parts of the study. Non-TBI subjects were excluded if they have: been diagnosed of mild TBI within the past 6 months; a prior moderate to severe TBI (GCS<13) within past 10 years; epilepsy with recurring seizures in the past 10 years; drug abuse (except marijuana) in the past 10 years based on DAST-10 screening; alcohol abuse based on AUDIT-C screening; current primary Axis I or II psychiatric disorders, except for disorders classified as minor and not expected to impact study conduct or integrity; a history of brain mass, neurosurgery, stroke, white matter disease, and/or dementia; a known cognitive dysfunction or structural brain disease/malformation; a structural brain injury on prior neuroimaging findings; been prescribed antipsychotic/antiepileptic medications; been unable (such as due to urgent medical care needs) or unwilling to complete study procedures accurately or have any conflict of interest that could affect study results, in the opinion of the investigator; or contraindications to MRI scanning, including: a. current or suspected pregnancy per site practice; b. other conditions that may constitute a hazard to the subject during study participation, per investigator; and c. inability to comply with any part of the site's MR safety policy.

Specimen Collection and Handling. Up to 40 mL (approximately 3 tablespoons) of blood were obtained at each specimen collection. 2 tubes of serum and 2 tubes of plasma were drawn at Encounters 1, 2 4, and 5. At Encounter 3, 2 tubes of serum, 1 tube of plasma and 1 or 2 tubes of whole blood were drawn. These study specimens were processed, aliquoted, frozen, and shipped to Abbott Laboratories for biomarker testing and storage. Sample aliquots were sent to testing sites for additional TBI biomarker testing. A specimen was considered unevaluable for the study if: it contained insufficient volume to perform necessary measurements, it was grossly hemolyzed, lipemic, or icteric; it was not collected in the proper type of collection tube; it was not properly labeled; or it was not properly stored by the collection site or at Abbott Laboratories.

Serum/plasma specimens were obtained via blood draw. If a blood draw was obtained at the time of admission for clinical purposes, additional specimen was obtained and retained for research purposes. If blood was not drawn for clinical purposes, trained research personnel drawn the blood required for research. The blood was drawn through venipuncture unless a central venous access was required by the standard of care, in which case the blood was withdrawn via that access. The first blood draw was taken upon admission, the second 3-6 hours after the first, and the third was taken at 24 hours after the trauma. Discovery efforts were also ongoing to find genetic markers of susceptibility to TBI or predictive markers of TBI. At each time point, 40 mL (less than 3 tablespoons) of blood was collected: 20 mL of serum (2 tubes) and 20 mL of plasma (2 tubes). During Encounter 1, only 2 tubes of serum and 1 tube of plasma were collected for blood biomarker analysis. This whole blood collection was 6.0 mL in a whole blood tube. If a patient was enrolled at Encounter 2 instead of Encounter 1, they had 2 tubes of serum and 1 tube of plasma collected for blood biomarker analysis. This whole blood collection was 6.0 mL in a whole blood tube.

The amount of blood drawn was limited according to the NINOS standardized table (Table 3). The number of attempts to draw blood was limited for children under the age of seven to two attempts. In the case that the NINOS standardized table did not allow enough blood to be drawn for the study or there were two failed attempts to draw blood in a child patient, access to leftover blood samples drawn under the clinical standard of care was requested for complete biomarker analysis in this study. This blood draw allowed for the analysis of up to 390 blood-based biomarkers related to traumatic brain injuries.

TABLE 3
Maximum Allowable Total Blood Draw Volumes

| | | | Maximum | Maximum | | Minimum Hgb |
|---|---|---|---|---|---|---|
| | | | allowable | volume | | required at time |
| | | Total | volume (mL) in | (clinical + | | of blood draw |
| Body | Body | blood | one blood draw | research) | Minimum Hgb | if subject has |
| Wt | Wt | volume | (=2.5% of total | (mL) in | required at time | respiratory/CV |
| (Kg) | (lbs) | (mL) | blood volume) | a 30-day period | of blood draw | compromise |
| 1 | 2.2 | 100 | 2.5 | 5 | 7.0 | 9.0-10.0 |
| 2 | 4.4 | 200 | 5 | 10 | 7.0 | 9.0-10.0 |
| 3 | 6.3 | 240 | 6 | 12 | 7.0 | 9.0-10.0 |
| 4 | 8.8 | 320 | 8 | 16 | 7.0 | 9.0-10.0 |
| 5 | 11 | 400 | 10 | 20 | 7.0 | 9.0-10.0 |
| 6 | 13.2 | 480 | 12 | 24 | 7.0 | 9.0-10.0 |
| 7 | 15.4 | 560 | 14 | 28 | 7.0 | 9.0-10.0 |
| 8 | 17.6 | 640 | 16 | 32 | 7.0 | 9.0-10.0 |
| 9 | 19.8 | 720 | 18 | 36 | 7.0 | 9.0-10.0 |
| 10 | 22 | 800 | 20 | 40 | 7.0 | 9.0-10.0 |
| 11-15 | 24-33 | 880-1200 | 22-30 | 44-60 | 7.0 | 9.0-10.0 |
| 16-20 | 35-44 | 1280-1600 | 32-40 | 64-80 | 7.0 | 9.0-10.0 |
| 21-25 | 46-55 | 1680-2000 | 42-50 | 64-100 | 7.0 | 9.0-10.0 |
| 26-30 | 57-66 | 2080-2400 | 52-60 | 104-120 | 7.0 | 9.0-10.0 |
| 31-35 | 68-77 | 2480-2800 | 62-70 | 124-140 | 7.0 | 9.0-10.0 |
| 36-40 | 79-88 | 2880-3200 | 72-80 | 144-160 | 7.0 | 9.0-10.0 |
| 41-45 | 90-99 | 3280-3600 | 82-90 | 164-180 | 7.0 | 9.0-10.0 |
| 46-50 | 101-110 | 3680-4000 | 92-100 | 184-200 | 7.0 | 9.0-10.0 |
| 51-55 | 112-121 | 4080-4400 | 102-110 | 204-220 | 7.0 | 9.0-10.0 |
| 56-60 | 123-132 | 4480-4800 | 112-120 | 224-240 | 7.0 | 9.0-10.0 |
| 61-65 | 134-143 | 4880-5200 | 122-130 | 244-260 | 7.0 | 9.0-10.0 |
| 66-70 | 145-154 | 5280-5600 | 132-140 | 264-280 | 7.0 | 9.0-10.0 |
| 71-75 | 156-185 | 5680-6000 | 142-150 | 284-300 | 7.0 | 9.0-10.0 |
| 76-80 | 167-176 | 6080-6400 | 152-160 | 304-360 | 7.0 | 9.0-10.0 |
| 81-85 | 178-187 | 6480-6800 | 162-170 | 324-340 | 7.0 | 9.0-10.0 |
| 86-90 | 189-198 | 6880-7200 | 172-180 | 344-360 | 7.0 | 9.0-10.0 |
| 91-95 | 200-209 | 7280-7600 | 182-190 | 364-380 | 7.0 | 9.0-10.0 |
| 96-100 | 211-220 | 7680-8000 | 192-200 | 384-400 | 7.0 | 9.0-10.0 |

In addition to the initial physical exam, those patients that were sent to the operating room undergone pathologic specimen analysis, those patients that received Licox had brain tissue oxygenation information recorded, and those patients that received ventriculostomy catheters had CSF collected for analysis. In order to analyze the CSF, 5.0 mL was collected at the same intervals that blood was drawn. Radiographic studies were performed in accordance with the standard of care. None of the assessments performed during the screening processes were analyzed with the rest of the data until informed consent was obtained. If the patient did not ultimately consent to research, the specimens, and initial assessments were discarded.

After the participants were discharged, the patients' medical records were accessed for information about the clinical course, including time spent in the ED, any surgeries or other neuromonitoring methods used, and the acute care outcome evaluation. If the patient spent time in the ICU, information was extracted from that time period as well, including data from Moberg monitors and daily therapeutic intensity level.

Trauma patients were divided into three groups for analysis: only brain injured, only non-brain injured, and combined injury. Two age- and gender-matched control groups were included in this study and recruited from the ED: non-trauma and CSF controls. Non-trauma controls were those who did not experience any trauma, and this group composed largely of family and friends of the patients admitted for brain injury. Both control groups were consented to undergo a single intensive assessment that included a blood draw and cognitive, neurological, and quality of life assessments (SAC, NOS-TBI, QoLABI). Patients receiving elective ventriculostomy or lumbar drain catheters were (pre-operatively) consented to be a part of the CSF control group. 5 mL of CSF was collected from the ventriculostomy catheter of patients in this control group for comparison with the CSF collected from the portion of the study group that received a ventriculostomy catheter as a part of their standard of care. The CSF control group was also offered the chance to participate in the same intensive assessment as the other two controls groups that included a blood draw and a 3T MRI scan.

Follow-Up: All patients that consented to participate in the follow-up portion of the study were asked to return to the hospital. Patients who returned were seen in the TBI Outpatient Clinic at 2 weeks, 4 weeks, months, 6 months, and 1 year. If they did not have a scheduled appointment at the TBI Outpatient Clinic, they were scheduled a time to come into the Brain Injury Research Lab (PL.610) at those time points. Table 4 provides a timeline for each of the assessments. Blood draws for biomarker analysis were done at each of the five follow-up time points in the same method, as described above. The outcome assessment battery listed in Tables 10 and 11 were completed at 3 months, 6 months, and one year. Radiographic scans that were a part of the standard of care were accessed through the participant's medical records, but select consented participants and controls also underwent 3T MRI scans at 2 weeks and 6 months after their brain injury. Each MRI examination took approximately one hour and included the following pulse sequences: (1) Sagittal short TR localizer, (2) Axial Fse, (3) Axial FLAIR, (4) Axial SWI, (5) Axial T2* imaging. In the case that patients were not able to come into the hospital for follow-up, they were contacted via phone at three months and one year after their injury to complete the BT ACT, which was a 15-20 minute cognitive assessment designed to be administered over the phone.

TABLE 4
Outcome Timeline

| | Blood | 3 T | CSF | CT | | Total |
|---|---|---|---|---|---|---|
| | Draw | MRI | Collection | Scan | Assessments | Time (minutes) |
| ADM | X | | X (if indicated) | | | 10 |
| 2 weeks | X | X | | | | 70 |
| 4 weeks | X | | | | | 10 |
| 3 months | X | | | | X | 70 |
| 6 months | X | X | | | | 70 |
| 1 year | X | X | | X* | X | 160 |
| | | | | | | (130 without CT) |

TABLE 5
Outcome Assessments - Not Finalized

| | | Adult | Pediatric |
|---|---|---|---|
| Outcome | All | GOS and GOSE | Pediatric GOSE |
| | Awake | SCAT3: SSS and SAC | Child SCAT3: Child & Parent Report; SAC-C |
| | | GOAT | COAT |
| | | Duration of Amnesia | Duration of Amnesia |
| | | NOS-TBI | NOS-TBI |
| | | Quality of Life: | Quality of Life: |
| | | MPAI-4 | Neuropsychiatric Rating Schedule |
| | | | Pediatric Quality of Life Inventory |
| | | | (For Child and for Proxy) |

| | |
|---|---|
| Not | CRS-R (Just Brain Stem Reflex Grid′) |
| Awake | |

TABLE 6
Possible Infant Assessments

| Name | Age | Time | Description |
|---|---|---|---|
| Bayley III, BSID* | 0- | 30- | Cognitive, language (receptive and expressive) and |
| | 3.5 | 90 | motor development |
| | | | Most commonly used in test for this age range |
| BITSEA* | 1-3 | 7-12 | Parent perception of Social and Emotional behavior |
| | | | 17 items of 42 are for autism, so may be able to be |
| | | | made shorter |
| CBCL | 1.5- | 25- | Parent perception of performance on Activities, Social, |
| | 5 | 30 | and School performance |
| MSEL* | 1 | 15 | Cognitive and Motor Ability (Gross Motor, Visual |
| | 3 | 25- | Reception, Fine Motor, Language |
| | 5 | 35 | Mostly for readiness for school |
| | | 40- | |
| | | 60 | |
| Shape School* | 3-6 | 45- | Inhibition and Switching Processes: Emerging |
| | | 75 | Executive Functions |
| Trails-Preschool* | 2-6 | 5-10 | Neuropsychological Function: Psychomotor speed, |
| | | | Complex Attention, Executive Function |
| | | | Advanced Trail Making Test |

*Requested Access

Statistical Analysis Plan. Biomarker data was analyzed by examining maximum concentration draw for each biomarker per patient, or by time from incident buckets, or both. To address the primary objective of determining associations between biomarker concentrations in blood and clinical neurological and magnetic resonance imaging data, multiple analyses was employed. Principal components analysis was used to examine which biomarkers may be explaining the same variance, or if a biomarker was contributing very little variance. The biomarkers were used in a logistic regression analysis, and some biomarkers were excluded based on the results from the principal components analysis, and clinical input. A significance level of 0.05 was used for the logistic regression analysis. ROC analysis was also used to examine the predictive ability of each biomarker in determining MRI status or neurological testing outcomes, for this set of data.

### Example 2

### DIA-MS Analysis

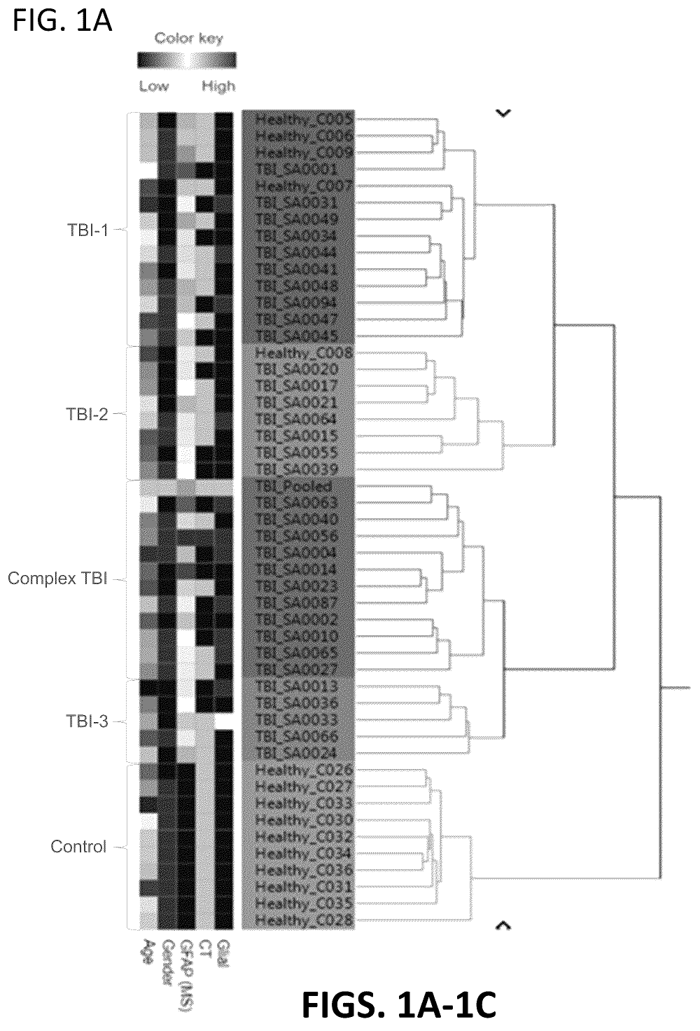

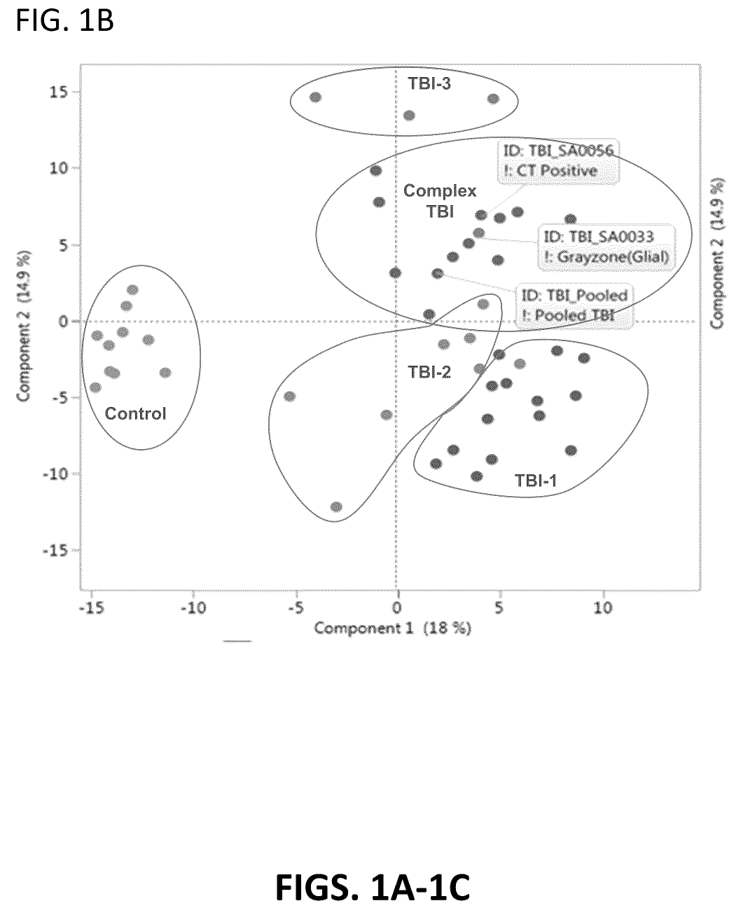

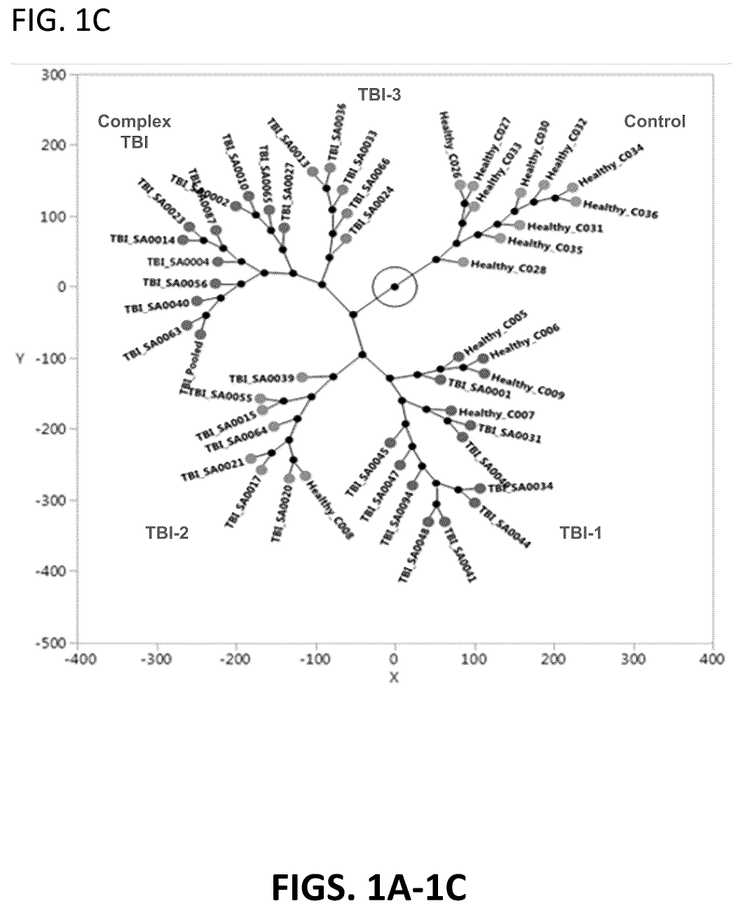

Discovery based mass spectrometry using data independent acquisition (DIA-MS) was carried out on 49 plasmas from individuals with mild TBI and controls. Fifteen (15) of these samples were derived from healthy patients who had no known traumatic brain injury. Of the remaining 34 samples, 33 samples were derived from patients who were physician-diagnosed as having endured mild traumatic brain injury (mTBI), with the remaining sample being a pool of plasma from patients who were physician-diagnosed as having endured a severe traumatic brain injury (sTBI). GCS scores for each of the patients in the cohort were also obtained, and all where within normal range (with the exception of the pooled severe TBI samples). CT scans were performed on a subset of subjects, and samples from a CT-positive subject was included (see FIG. 1A). Plasma samples were digested with trypsin to generate peptides which were analyzed using DIA-MS (peptide identities are included below). All quantifiable peptides and proteins were included. GFAP detection based on 2 unique peptides was also selectively pulled out of the data and included in FIG. 1, and GFAP levels assessed independently using an immunoassay (ELISA-based GFAP assay) were also included in FIG. 1.

The methods described above were used to identify TBI biomarkers for the various classes and subclasses of TBI. Table 7 shows candidate TBI biomarkers associated with severe TBI (clustered with the pooled sTBI sample with detectable levels of GFAP). SAMP was downregulated in the sTBI group (and upregulated in the healthy control group), and CAND1 and GLO2 were upregulated in the sTBI group (downregulated in the healthy control group). ERAP1, SYYC, C1RL, and ATPG were only detected in the sTBI group and not the healthy controls (i.e. unique expression).

TABLE 7
Candidate Biomarkers for Severe TBI

| UniProtID\|Protein | log2FC (severe/healthy) |
|---|---|
| Q86VP6\|CAND1 | 2.67 |
| P02743\|SAMP | −1.6 |
| Q16775\|GLO2 | 1.38 |
| Q9NZ08 ERAP1 | Only in severe |
| P54577 SYYC | Only in severe |
| Q9NZP8 C1RL | Only in severe |
| P36542 ATPG | Only in severe |

Unsupervised Analysis. Unsupervised clustering analysis was applied to the proteomic data obtained by DIA-MS to determine how the patient cohort samples clustered according to their molecular proteomic profiles, unbiased by any clinical grouping. The null hypothesis in this analysis is that the proteomic data is randomly scattered across all samples, without any clustering pattern that relates to the presence or severity of a TBI. As shown in FIG. 1A, the Control branch of the clustering analysis (bottom most grouping of the five groupings) successfully discerned 10 of 15 healthy samples from TBI samples. Ultimately, four additional sub-clusters emerged, and correspond to subclasses of mild TBI. These data are summarized and outlined below.

Unsupervised cluster analysis yielded 5 clusters (FIGS. 1A-1C). This includes a cluster of “healthy” subjects (i.e. Control grouping) that consists of the original ten samples designated as healthy individuals. Additionally, these data and the accompanying analysis demonstrated the presence of four distinct subclasses of mild TBI. For the purposes of clarity and differentiation, each of these clusters was assigned a number: the TBI-1 cluster=mild TBI subclass 1; the TBI-2 cluster=mild TBI subclass 2; the TBI-3 cluster=mild TBI subclass 3; and the Complex TBI cluster=mild TBI subclass 4 (also referred to as “complicated mild” TBI).

“Mild TBI subclass 1” (TBI-1) refers to subjects who are classified as having mild TBI and also exhibit a plasma proteome signature that is different from controls (e.g., healthy controls, or controls that have not sustained a TBI), different from subjects with moderate to severe TBI, and different from subjects with a mild TBI of each of subclasses 2, 3, or 4. “Mild TBI subclass 2” (TBI-2) refers to subjects who are classified to having mild TBI and also exhibit a plasma proteome signature that is different from controls (e.g., healthy controls, or controls that have not sustained a TBI), different from subjects with moderate to severe TBI, and different from subjects with a mild TBI of each of subclasses 1, 3, or 4. “Mild TBI subclass 3” (TBI-3) refers to subjects who are classified to having mild TBI and also exhibit a plasma proteome signature that is different from controls (e.g., healthy controls, or controls that have not sustained a TBI), different from subjects with moderate to severe TBI, and different from subjects with a mild TBI of each of subclasses 1, 2, or 4. “Mild TBI subclass 4” (Complex TBI) refers to subjects who are classified as having mild TBI, and also exhibit plasma proteome signature that is different from controls (e.g., healthy controls, or controls that have not sustained a TBI), different from subjects with moderate to severe TBI, and different from subjects with a mild TBI of each of subclasses 1, 2, or 3. Additionally, subjects with mild TBI subclass 4 exhibit a proteome signature that resembles the proteome signature obtained from pooled samples of subjects having severe TBI; thus, this subclass is referred to herein as “complicated mild TBI.” In data disclosed herein, the mild TBI subclass 4 cluster (“Complex TBI”) contained the highest number of subjects having elevated GFAP levels (based on ELISA-based GFAP assay and GFAP mass spectrometry), and the only subject having a positive CT scan.

Sample origins were de-identified upon completion of unsupervised clustering, and four key parameters were separately included and shown in FIG. 1A. These parameters included a Glial biomarker score (positive (gray) (ELISA-based GFAP assay), negative (lack), border zone (white), inconclusive, or lacking information (light grey)); a CT scan score (positive (gray), negative (black), or not performed (light grey)); a GFAP protein expression level obtained using MS (numerical continuum between lowest (black) to highest (gray)); Gender (male (gray) or female (black)); and Age (numeric continuum between 20 (black) and 59 (gray) years).

Based on this analysis, it was concluded that TBI as a disease indication includes a spectrum of injury states that can be described and categorized based on TBI biomarker signatures. Four different clusters were identified as corresponding to four different subclasses of mild TBI (FIGS. 1B-1C). The Complex TBI cluster represents the most severe of the mild TBI subclasses, as this cluster contains the severe TBI pooled sample, the only individual with a positive CT scan, the most number of GFAP biomarker positive samples (detected by immunoassay), and the most number of GFAP positive samples (based on MS data).

The control cluster represents healthy individuals, as it is negative for all clinical and biochemical markers and exclusively contains samples from patients without known TBI. This group was used as the healthy control set for all subsequent analysis.

The TBI-1 cluster represents the least severe subclass of mild TBI. This cluster contains some designated control samples and samples with low but detectable levels of GFAP by MS. Additionally, as shown in FIGS. 1B-1C, unsupervised clustering analysis led to the identification of two additional mild TBI subclasses, the TBI-2 cluster and the TBI-3 cluster. Both the TBI-2 and TBI-3 groupings represent discrete clusters that were shown to be distinct from both the TBI-1 cluster (least severe) and the Complex TBI cluster (most severe).

Thus, these data and the accompanying analysis demonstrate the presence of four distinct subclasses of mild TBI.

Next, these four “non-healthy” sub-clusters were compared against the “healthy” cluster (controls), and this comparison was expressed as log₂[fold change] values. Tables 8A and 8B include the identities of the ten most differentially regulated proteins and the ten most upregulated proteins, respectively, according to their log₂[fold change] values.

TABLE 8A
Differentially Regulated TBI Biomarkers Among Control and mTBI Subclasses
Table 8A. Top 10 proteins in each subclass with the largest changes

| Subclass 4/Healthy | | Subclass 2/Healthy | | Subclass 1/Healthy | | Subclass 3/Healthy | |
|---|---|---|---|---|---|---|---|
| UniProtID \| | | UniProtID \| | | UniProtID \| | | UniProtID \| | |
| Protein | log2FC | Protein | | Protein | | Protein | |
| name | NML1 | name | log2FC | name | log2FC | name | log2FC |
| P29144 \| TPP2 | −3.07 | O75376 \| NCOR1 | 2.93 | Q15746 \| MYLK | 2.26 | P29144 \| TPP2 | −3.42 |
| Q86VP6 \| CAND1 | 2.67 | P29144 \| TPP2 | −2.35 | P35908 \| K22E | 2.09 | O75376 \| NCOR1 | 2.72 |
| O75376 \| NCOR1 | 2.64 | P35908 \| K22E | 2.03 | P00429 \| DNM1L | 1.67 | P23083 \| HV103 | 2.07 |
| P35908 \| K22E | 2.04 | Q15746 \| MYLK | 1.99 | O60610 \| DIAP1 | 1.66 | Q27J81 \| INF2 | −2.01 |
| Q15746 \| MYLK | 1.84 | Q96IU4 \| ABHEB | 1.98 | Q96IU4 \| ABHEB | 1.59 | P01880 \| 1GHD | 1.98 |
| P49189 \| AL9A1 | −1.79 | P02743 \| SAMP | −1.82 | Q02809 \| PLOD1 | 1.49 | Q9H0W9 \| CK054 | −1.94 |
| Q96IU4 \| ABHEB | 1.72 | Q27J81 \| INF2 | −1.75 | P07814 \| SYEP | 1.42 | A99683 \| M3K5 | −1.75 |
| O00429 \| DNM1L | 1.68 | Q6UWP8 \| SBSN | 1.71 | P01594 \| KV102 | −1.41 | Q96IU4 \| ABHEB | 1.75 |
| Q27J81 \| INF2 | −1.67 | P49189 \| AL9A1 | −1.69 | P49189 \| AL9A1 | −1.40 | P49189 \| AL9A1 | −1.65 |
| P02743 \| SAMP | −1.60 | Q9Y2E5 \| MA2B2 | −1.53 | P54760 \| EPHB4 | 1.38 | O00429 \| DNM1L | 1.48 |

TABLE 8B
Upregulated TBI Biomarkers Among Control and mTBI Subclasses

| Subclass | | Subclass | | Subclass | | Subclass | |
|---|---|---|---|---|---|---|---|
| 4/Healthy | | 2/Healthy | | 1/Healthy | | 3/Healthy | |
| UniProtID \| | | UniProtID \| | | UniProtID \| | | UniProtID \| | |
| Protein | | Protein | | Protein | | Protein | |
| name | log2FC | name | log2FC | name | log2FC | name | log2FC |
| Q86VP6 \| CAND1 | 2.67 | O75376 \| NCOR1 | 2.93 | Q15746 \| MYLK | 2.26 | O75376 \| NCOR1 | 2.72 |
| O75376 \| NCOR1 | 2.64 | P35908 \| K22E | 2.03 | P35908 \| K22E | 2.09 | P23083 \| HV103 | 2.07 |
| P35908 \| K22E | 2.04 | Q15746 \| MYLK | 1.99 | O00429 \| DNM1L | 1.67 | P01880 \| IGHD | 1.98 |
| Q15746 \| MYLK | 1.84 | Q96IU4 \| ABHEB | 1.98 | O60610 \| DIAP1 | 1.66 | Q96IU4 \| ABHEB | 1.75 |
| P96IU4 \| ABHEB | 1.72 | Q6UWP8 \| SBSN | 1.71 | Q96IU4 \| ABHEB | 1.59 | O00429 \| DNM1L | 1.48 |
| O00429 \| DNM1L | 1.68 | O00429 \| DNM1L | 1.40 | Q02809 \| PLOD1 | 1.49 | P02768 \| ALBU | 1.21 |
| Q6UWP8 \| SBSN | 1.54 | O60610 \| DIAP1 | 1.33 | P07814 \| SYEP | 1.42 | P42765 \| THIM | 1.19 |
| P08133 \| ANXA6 | 1.49 | P63167 \| DYL1 | 1.30 | P54760 \| EPHB4 | 1.38 | P01877 \| IGHA2 | 1.13 |
| Q16775 \| GLO2 | 1.38 | P55786 \| PSA | 1.28 | P08133 \| ANXA6 | 1.32 | P01597 \| HV105 | 1.11 |
| P07814 \| SYEP | 1.30 | P54760 \| EPHB4 | 1.26 | Q12805 \| FBLN3 | 1.31 | O00187 \| MASP2 | 1.09 |

Additionally, to differentiate between each of the disease clusters, preliminary stratification analysis was carried out based on putative classes or severities of the mild TBI subclasses. Relative pairwise comparisons were performed between each combination of “unhealthy” clusters in the absence of the “healthy” control cluster. These data are summarized below in Table 9 as ten proteins in each group with the largest magnitude log₂[fold change] values, along with their UniProt DD's.

| Subclass 4/Subclass 2 | | Subclass 4/Subclass 1 | | Subclass 3/Subclass 4 | | Subclass 3/Subclass 1 | |
|---|---|---|---|---|---|---|---|
| UniProtID\| | | UniProtID\| | | UniProtID\| | | UniProtID\| | |
| Protein | | Protein | | Protein | | Protein | |
| name | log2FC | name | log2FC | name | log2FC | name | log2FC |
| P08519 \| | 1.31 | P80748 \| | −1.54 | P23083 \| | 1.53 | P23083 \| | 2.31 |
| APOA | | ILV302 | | HV103 | | HV103 | |
| B9A064 \| | −1.17 | P01594 \| | 1.35 | P01880 \| | 1.13 | P01594 \| | 1.73 |
| IGLL5 | | KV102 | | IGHD | | KV102 | |
| Q9Y2H5 \| | 1.01 | P01860 \| | 0.92 | P01871 \| | −0.98 | P48681 \| | −1.54 |
| PKHA6 | | IGHG3 | | IGHM | | NEST | |
| P04220 \| | 0.98 | P23083 \| | 0.78 | P04220 \| | −0.90 | P12931 \| | −1.35 |
| MUCB | | HV103 | | MUCB | | SRC | |
| Q13790 \| | 0.97 | P21291 \| | −0.76 | P12931 \| | −0.84 | O43583 \| | −1.29 |
| APOF | | CSRP1 | | SRC | | DENR | |
| P13727 \| | 0.89 | P08582 \| | 0.68 | P02751 \| | −0.78 | P01880 \| | 1.28 |
| PRG2 | | TRFM | | FINC | | IGHD | |
| P02745 \| | 0.82 | P07195 \| | 0.65 | O43583 \| | −0.76 | P23526 \| | −1.27 |
| C1QA | | LDHB | | DENR | | SAHH | |
| P04207 \| | 0.80 | P15311 \| | 0.63 | P23526 \| | −0.75 | P01860 \| | 1.10 |
| KV308 | | EZRI | | SAHH | | IGHG3 | |
| Q15848 \| | 0.76 | O43396 \| | −0.62 | O43866 \| | −0.71 | 075368 \| | −1.06 |
| ADIPO | | TXNL1 | | CD5L | | SH3L1 | |
| P29144 \| | −0.72 | P18065 \| | −0.61 | Q15848 \| | −0.68 | P19652 \| | 0.97 |
| TPP2 | | IBP2 | | ADIPO | | A1AG2 | |

| Subclass 3/Subclass 2 | | Subclass 2/Subclass 1 | |
|---|---|---|---|
| UniProtID\| | | UniProtID\| | |
| Protein | | Protein | |
| name | log2FC | name | log2FC |
| P01880 \| GHD | 1.71 | P01594 \| KV102 | 1.58 |
| P12931 \| SRC | −1.49 | Q9Y2H5 \| PKHA6 | −1.26 |
| P08519 \| APOA | 1.42 | P01860 \| IGHG3 | 1.16 |
| P23083 \| HV103 | 1.21 | P23083 \| HV103 | 1.10 |
| O43583 \| DENR | −1.18 | P13727 \| PRG2 | −1.08 |
| O75368 \| SH3L1 | −1.09 | P21291 \| CSRP1 | −0.92 |
| P29144 \| TPP2 | −1.07 | P02775 \| CXCL7 | −0.86 |
| P01019 \| ANGT | 1.04 | B9A064 \| IGLL5 | 0.86 |
| B9A064 \| IGLL5 | −1.02 | P02745 \| C1QA | −0.80 |
| Q13790 \| APOF | 1.01 | P00338 \| LDHA | −0.80 |

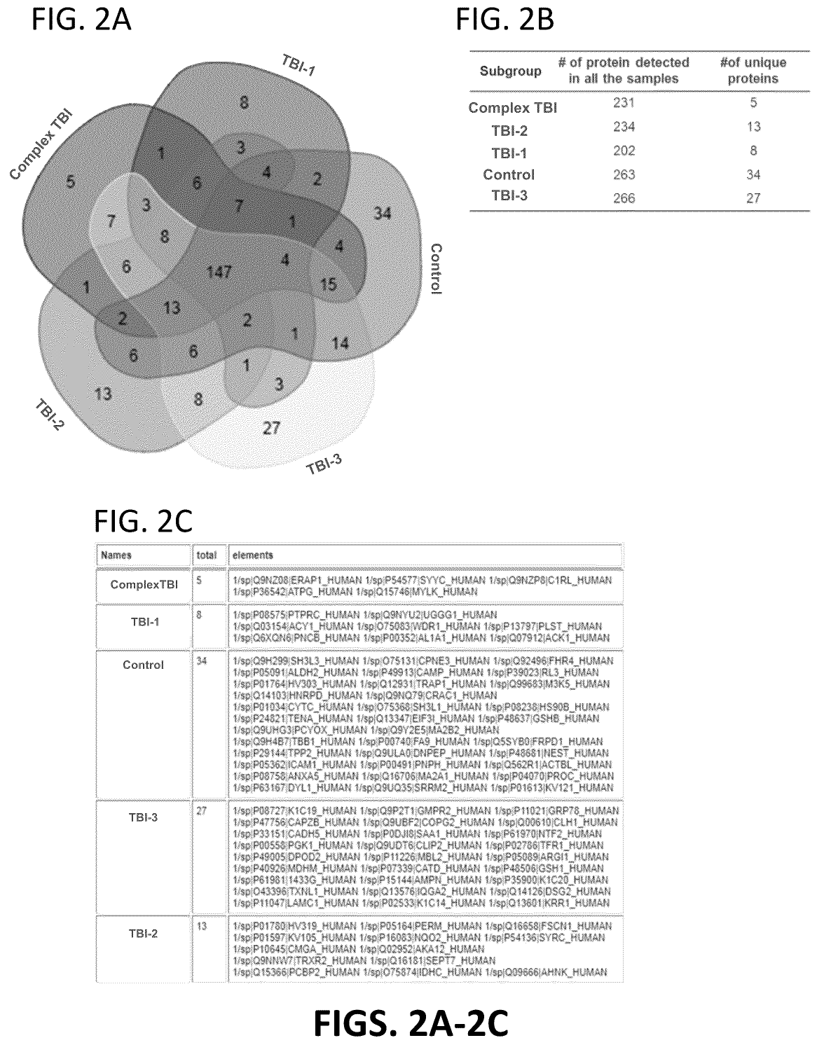

The data was then assessed in an effort to identify proteins that were unique to each cluster, as these could potentially be included as exclusive candidate TBI biomarkers for a given subclass of TBI (FIGS. 2A-2C). Each of five main sample sub-clusters derived from unsupervised clustering analysis contained a minimum of five uniquely expressed proteins. These proteins can be included with the proteins which are uniquely increased in each subclass (Tables 8 and 9). FIGS. 2A-2C include proteins that are shared and unique between sample clusters based on unsupervised analysis. Each of five main sample sub-clusters derived from unsupervised clustering analysis has a minimum of five uniquely expressed proteins. FIG. 2A includes a Venn diagram with shared and unique proteins between the five identified subgroup clusters; and FIG. 2B includes a numeric count of total proteins and unique proteins quantified in each subgroup cluster. FIG. 2C includes the UniProt ID and short name for the unique proteins quantified in each subgroup cluster.

Supervised Analysis. Once the blinded unsupervised analyses were complete, the samples were de-identified in terms of the TBI status of the patients from which they were derived. This additional information enabled several iterations of supervised analyses to be performed.

In the first iteration of this supervised analysis, all proteins quantities obtained from the TBI-derived samples were compared with those form the non-TBI samples (based on patient characterizations), and the proteins were ranked according to the magnitude of their differential expression. This was done according to two separate criteria: 1) ranking the proteins according to the magnitude of their differential expression irrespective of the directionality of the change (upregulated and downregulated); and 2) because upregulated proteins lend themselves more favorably to detection as a biomarker, ranking the proteins according to the magnitude of their differentially upregulated expression. These are high probability TBI biomarker candidates useful for distinguishing mild TBI from a control. These data are summarized in Table 10 and FIGS. 2A-2C.

TABLE 10
Supervised Analysis Between TBI Samples and Healthy Controls
Table 10: Top TBI changes compared to all
healthy individuals based on supervised analysis

| Top differential | | Top upregulated | |
|---|---|---|---|
| Protein | | protein | |
| (UniProtID \| | | (UniProtID \| | |
| Protein Name) | log₂FC | Protein Name) | log₂FC |
| O75376 \| NCOR1 | 3.42 | O75376 \| NCOR1 | 3.42 |
| P29144 \| TPP2 | −2.78 | Q96IU4 \| ABHEB | 1.13 |
| P02743 \| SAMP | −1.25 | P08133 \| ANXA6 | 0.98 |
| Q27J81 \| INF2 | −1.15 | O00429 \| DNM1L | 0.98 |
| Q99683 \| MEK5 | −1.13 | Q15485 \| FCN2 | 0.98 |
| Q96IU4 \| ABHEB | 1.13 | P69905 \| HBA | 0.97 |
| P49189 \| AL9A1 | −1.01 | Q15746 \| MYLK | 0.95 |
| P08133 \| ANXA6 | 0.98 | Q6UWP8 \| SBSN | 0.94 |
| O00429 \| DNM1L | 0.98 | Q09666 \| AHNK | 0.93 |
| Q15485 \| FCN2 | 0.98 | P35908 \| K22E | 0.91 |

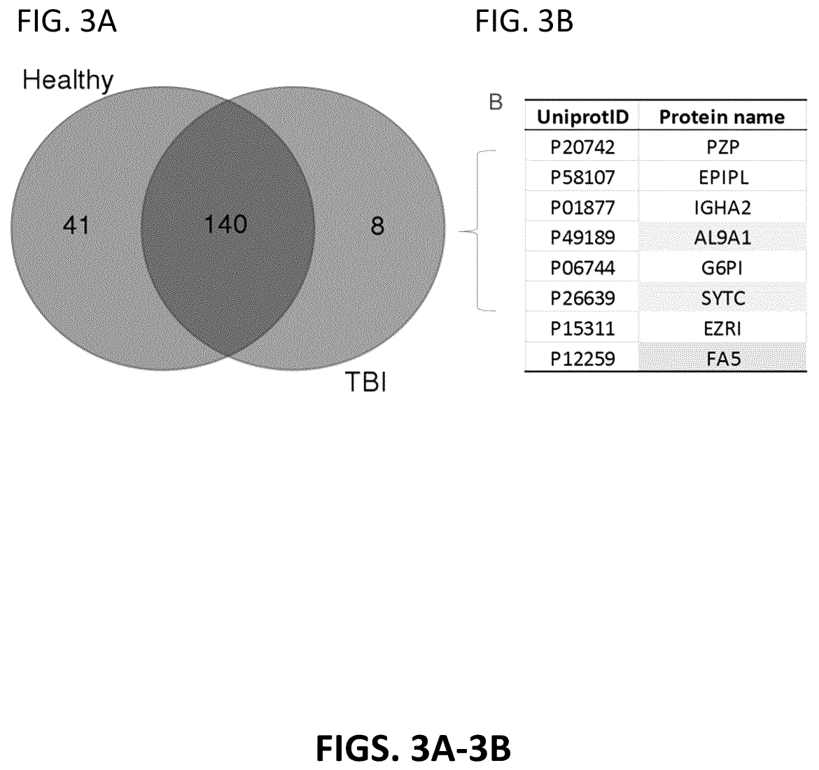

FIGS. 3A-3B include a representative Venn plot of proteins that are shared or unique between healthy and TBI patient samples. Only proteins detected in all the samples in each group were included in this analysis (FIG. 3A). FIG. 3B includes a list of 8 proteins uniquely expressed in all TBI samples, including their UniProt ID and protein name. AL9A1 and SYTC are expressed in brain while FA5 is related to coagulation (both designations are based on annotated information from UniProt (uniprot.org)). These proteins show increased or unique expression in all of the 34 TBI samples, encompassing all subclasses 1-3 (TBI-1, TBI-2, TBI-3) and the more severe mild TBI subclass 4 (Complex TBI), compared to the 15-healthy control sample cohort (see FIGS. 1 and 2). FIGS. 3A-3B represent a high probability grouping, and some of these proteins were found as part of the protein panels from the supervised and unsupervised models.

Additionally, Table 11 below provides more of a holistic approach for determining a subject's TBI, such as severe TBI, mild TBI, or absence of TBI. Table 11 includes proteins found across all classes of TBI using difference models.

TABLE 11
Commonly Found TBI Biomarkers
Table 11. Proteins found in TBI (all classes)
and commonly found by difference models
P29144 | TPP2
Q99683 | M3K5
O75376 | NCOR1
P35908 | K22E
Q15746 | MYLK
P49189 | AL9A1
Q96IU4 | ABHEB
O00429 | DNM1L
Q27J81 | INF2
P02743 | SAMP
Q6UWP8 | SBSN
P08133 | ANXA6
Q15485 | FCN2
P07814 | SYEP

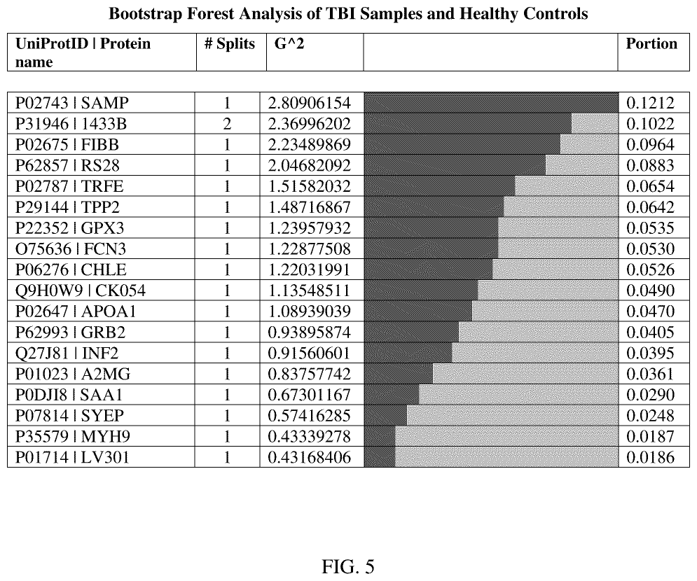

Bootstrap Forest Analysis. In another iteration of supervised analysis, Bootstrap Forest Analysis was performed as an alternative approach to traditional supervised analysis of quantitative DIA-MS data. This analysis ranks proteins according to their contributing portion of a whole, and is summarized in FIG. 5.

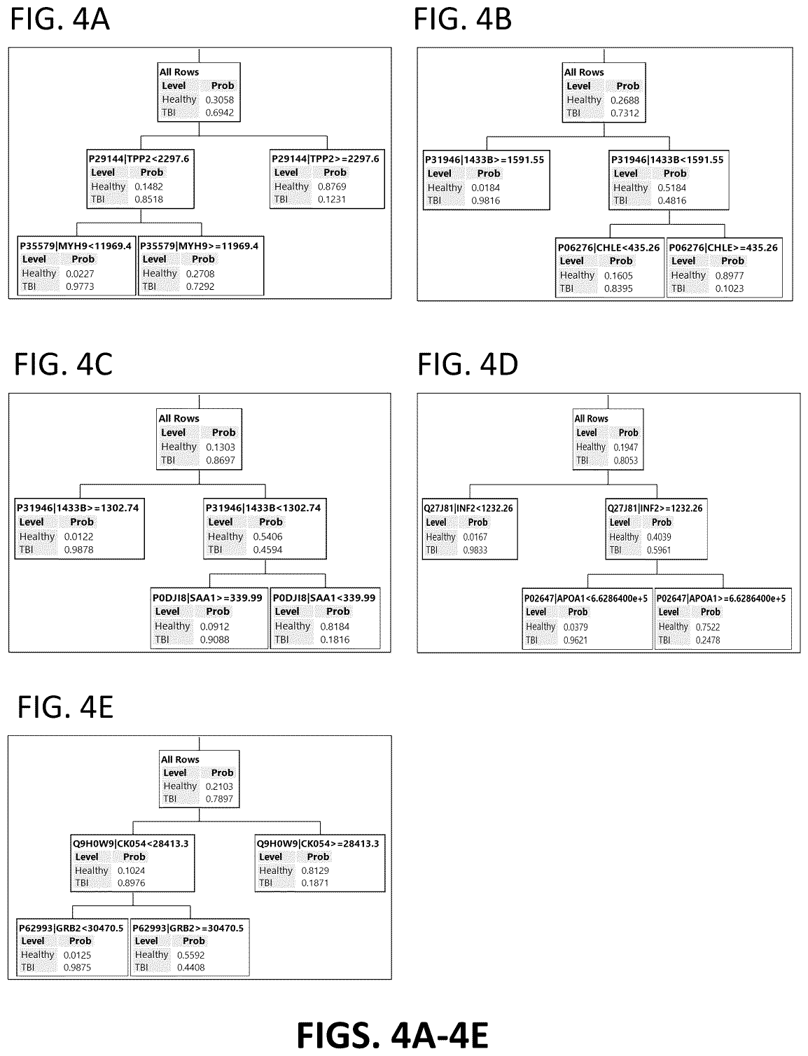

In addition to summarizing the overall dataset through Bootstrap Forest Analysis, the contribution of individual proteins was assessed. This approach is summarized as a total of five separate decision trees that met the criteria of the prediction model, as shown in FIGS. 4A-4E. This analysis ascribed a quantitative cutoff for the ability of a given protein to serve as a biomarker capable of distinguishing patients with TBI from their healthy counterparts, along with an associated diagnostic probability. In the case where a single protein was insufficiently capable of making this distinction, a combination of multiple proteins is identified and defined.

### Example 3

### TBI Biomarker Proteins and Peptide Fragments

Mass spectrometry techniques typically involve the detection of ions that have undergone physical change(s) in a mass spectrometer. Frequently, the physical change involves fragmenting a selected precursor ion and recording the mass spectrum of the resultant fragment ions. The information in the fragment ion mass spectrum is often a useful aid in elucidating the structure of the precursor ion. The general approach used to obtain a mass spectrometry/mass spectrometry (MS/MS or MS2) spectrum is to isolate a selected precursor ion with a suitable m/z analyzer, to subject the precursor ion to energetic collisions with a neutral gas in order to induce dissociation, and finally to mass analyze the fragment ions in order to generate a mass spectrum.

As would be recognized by one of ordinary skill in the art based on the present disclosure, such peptide fragments used in DIA-MS (or DDA-MS) can be used to identify proteins/biomarkers. More specifically, the following tables include the UniProt ID Nos. and short names of the various TBI biomarkers identified and described herein, listed alongside their corresponding peptide fragment sequences.

In addition to DIA-MS, DDA-MS and SRM/MRM-MS can be used for reliable and comprehensive quantification of various peptide analytes. SRM/MRM-MS is generally performed on triple quadrupole-like instruments, in which increased selectivity is obtained through collision-induced dissociation. It is a non-scanning mass spectrometry technique, where two mass analyzers (Q1 and Q3) are used as static mass filters, to monitor a particular fragment of a selected precursor. On triple quadrapole instruments, various ionization methods can be used including without limitation electrospray ionization, chemical ionization, electron ionization, atmospheric pressure chemical ionization, and matrix-assisted laser desorption ionization. The specific pair of mass-over-charge (m/z) values associated to the precursor and fragment ions selected is referred to as a “transition”. The detector acts as a counting device for the ions matching the selected transition thereby returning an intensity distribution over time. MRM-MS is when multiple SRM-MS transitions are measured within the same experiment on the chromatographic time scale by rapidly switching between the different precursor/fragment pairs. Typically, the triple quadrupole instrument cycles through a series of transitions and records the signal of each transition as a function of the elution time. The method allows for additional selectivity by monitoring the chromatographic co-elution of multiple transitions for a given analyte. Potential peptides are listed in Tables 13-21 for the different classifications outlined in the present disclosure.

In addition, PRM-MS can be used in which there is parallel detection of all transitions in a single analysis using a high resolution mass spectrometer. PRM-MS provides high selectivity, high sensitivity and high-throughput to quantify selected peptide (Q1), hence quantify proteins. Again, multiple peptides can be specifically selected for each protein. PRM-MS methodology uses the quadrupole of a mass spectrometer to isolate a target precursor ion, fragments the targeted precursor ion in the collision cell, and then detects the resulting product ions in the Orbitrap mass analyzer. PRM-MS uses a quadrupole time-of-flight (QTOF) or hybrid quadrupole-orbitrap (QOrbitrap) mass spectrometer to carry out the peptides/proteins quantitation. Potential peptides are listed in Tables 13-21 for the different classifications outlined in the present disclosure.

In some embodiments, the methods described herein can be applied to the quantification of polypeptides(s) or protein(s) in biological sample(s). Any kind of biological samples comprising polypeptides or proteins can be the starting point and be analyzed by the methods herein. Indeed, any protein/peptide containing sample can be used for and analyzed by the methods produced here (e.g., tissues, cells). The methods herein can also be used with peptide mixtures obtained by digestion. Digestion of a polypeptide or protein includes any kind of cleavage strategies, such as, enzymatic, chemical, physical or combinations thereof. According to some embodiments, the following parameters of the methods provided herein are determined: trypsin (or other protease) digestion and peptide clean up, best responding polypeptides, best responding proteins, best responding peptides, best responding fragments, fragment intensity ratios (increased high and reproducible peak intensities), optimal collision energies, and all the optimal parameters to maximize sensitivity and/or specificity of the methods.

In other embodiments, quantification of the polypeptides and/or of the corresponding proteins or activity/regulation of the corresponding proteins is desired. A selected peptide is labeled with a stable-isotope and used as an internal standard (SIL) to achieve absolute quantification of a protein of interest. The addition of a quantified stable-labeled peptide analogue of the tag to the peptide sample in known amount; and subsequently the tag and the peptide of interest is quantified by mass spectrometry and absolute quantification of the endogenous levels of the proteins is obtained.

TABLE 13
Rule-In TBI Biomarkers-Unique

| | | | UniProt | |
|---|---|---|---|---|
| | | | Primary | |
| Unique | | | Accession | SEQ ID |
| Protein | Abbreviated Name | Descriptor | Number | NO: |
| | PZP | PZP_HUMAN | P20742 | |
| | EPIPL | EPIPL_HUMAN | P58107 | |
| | IGHA2 | IGHA2_HUMAN | P01877 | |
| | AL9A1 | AL9A1_HUMAN | P49189 | |
| | G6PI | G6PI_HUMAN | P06744 | |
| | SYTC | SYTC_HUMAN | P26639 | |
| | EZRI | EZRI_HUMAN | P15311 | |
| | FA5 | FA5_HUMAN | P12259 | |
| Protein | Peptide Sequence | | | |
| P06744 | DPQFQK | | | 4 |
| P06744 | LQQWYR | | | 5 |
| P06744 | SELNLR | | | 6 |
| P06744 | FNHFSLTLNTNHGHILVDYSK | | | 7 |
| P06744 | INYTEGR | | | 8 |
| P06744 | AVLHVALR | | | 9 |
| P06744 | SNTPILVDGK | | | 10 |
| P06744 | VWYVSNIDGTHIAK | | | 11 |
| P06744 | TLAQLNPESSLFIIASK | | | 12 |
| P06744 | TFTTQETITNAETAK | | | 13 |
| P06744 | EWFLQAAK | | | 14 |
| P06744 | DPSAVAK | | | 15 |
| P06744 | HFVALSTNTTK | | | 16 |
| P06744 | ELQAAGK | | | 17 |
| P06744 | VFEGNRPTNSIVFTK | | | 18 |
| P06744 | IFVQGIIWDINSFDQWGVELGK | | | 19 |
| P12259 | LWVLVVLGTSWVGWGSQGTEAAQLR | | | 20 |
| P12259 | EYEPYFK | | | 21 |
| P12259 | EKPQSTISGLLGPTLYAEVGDIIK | | | 22 |
| P12259 | ADKPLSIHPQGIR | | | 23 |
| P12259 | LSEGASYLDHTFPAEK | | | 24 |
| P12259 | GTLTEGGTQK | | | 25 |
| P12259 | QIVLLFAVFDESK | | | 26 |
| P12259 | WIISSLTPK | | | 27 |
| P12259 | SQHLDNFSNQIGK | | | 28 |
| P12259 | EDGILGPIIR | | | 29 |
| P12259 | AVQPGETYTYK | | | 30 |
| P12259 | AADIEQQAVFAVFDENK | | | 31 |
| P12259 | SWYLEDNINK | | | 32 |
| P12259 | LEPEDEESDADYDYQNR | | | 33 |
| P12259 | LAAALGIR | | | 34 |
| P12259 | FTVNNLAEPQK | | | 35 |
| P12259 | APSHQQATTAGSPLR | | | 36 |
| P12259 | LLSLGAGEFK | | | 37 |
| P12259 | SQEHAK | | | 38 |
| P12259 | DPPSDLLLLK | | | 39 |
| P12259 | QSNSSK | | | 40 |
| P12259 | WHLASEK | | | 41 |
| P12259 | AWGESTPLANKPGK | | | 42 |
| P12259 | QSGHPK | | | 43 |
| P12259 | SQFLIK | | | 44 |
| P12259 | HTHHAPLSPR | | | 45 |
| P12259 | TFHPLR | | | 46 |
| P12259 | SEAYNTFSER | | | 47 |
| P12259 | HSLVLHK | | | 48 |
| P12259 | EFNPLVIVGLSK | | | 49 |
| P12259 | DGTDYIEIIPK | | | 50 |
| P12259 | EEVQSSEDDYAEIDYVPYDDPYK | | | 51 |
| P12259 | TNINSSR | | | 52 |
| P12259 | DPDNIAAWYLR | | | 53 |
| P12259 | SNNGNR | | | 54 |
| P12259 | NYYIAAEEISWDYSEFVQR | | | 55 |
| P12259 | ETDIEDSDDIPEDTTYK | | | 56 |
| P12259 | YLDSTFTK | | | 57 |
| P12259 | GEYEEHLGILGPIIR | | | 58 |
| P12259 | AEVDDVIQVR | | | 59 |
| P12259 | NLASRPYSLHAHGLSYEK | | | 60 |
| P12259 | TYEDDSPEWFK | | | 61 |
| P12259 | EDNAVQPNSSYTYVWHATER | | | 62 |
| P12259 | AWAYYSAVNPEK | | | 63 |
| P12259 | SWYYEK | | | 64 |
| P12259 | QHQLGVWPLLPGSFK | | | 65 |
| P12259 | ASKPGWWLLNTEVGENQR | | | 66 |
| P12259 | ASEFLGYWEPR | | | 67 |
| P12259 | LNNGGSYNAWSVEK | | | 68 |
| P12259 | EVIITGIQTQGAK | | | 69 |
| P12259 | ENQFDPPIVAR | | | 70 |
| P12259 | AYNRPTLR | | | 71 |
| P12259 | QITASSFK | | | 72 |
| P12259 | SWWGDYWEPFR | | | 73 |
| P12259 | VNAWQAK | | | 74 |
| P12259 | QWLEIDLLK | | | 75 |
| P12259 | SYTIHYSEQGVEWKPYR | | | 76 |
| P12259 | IFEGNTNTK | | | 77 |
| P12259 | NFFNPPIISR | | | 78 |
| P12259 | TWNQSIALR | | | 79 |
| P15311 | EVWYFGLHYVDNK | | | 80 |
| P15311 | GFPTWLK | | | 81 |
| P15311 | VSAQEVR | | | 82 |
| P15311 | FYPEDVAEELIQDITQK | | | 83 |
| P15311 | FGDYNK | | | 84 |
| P15311 | SGYLSSER | | | 85 |
| P15311 | DQWEDR | | | 86 |
| P15311 | IQVWHAEHR | | | 87 |
| P15311 | GTDLWLGVDALGLNIYEK | | | 88 |
| P15311 | QQLETEK | | | 89 |
| P15311 | LQDYEEK | | | 90 |
| P15311 | ELSEQIQR | | | 91 |
| P15311 | ALQLEEER | | | 92 |
| P15311 | QAVDQIK | | | 93 |
| P15311 | SQEQLAAELAEYTAK | | | 94 |
| P15311 | IALLEEAR | | | 95 |
| P15311 | EDEVEEWQHR | | | 96 |
| P15311 | EAQDDLVK | | | 97 |
| P15311 | QLLTLSSELSQAR | | | 98 |
| P15311 | IDEFEAL | | | 99 |
| P20742 | VVSVDENFRPR | | | 100 |
| P20742 | NELIPLIYLENPR | | | 101 |
| P20742 | IAQWQSLK | | | 102 |
| P20742 | LEAGINQLSFPLSSEPIQGSYR | | | 103 |
| P20742 | VVVQTESGGR | | | 104 |
| P20742 | IQHPFTVEEFVLPK | | | 105 |
| P20742 | EEGTDLEVTANR | | | 106 |
| P20742 | ISEITNIVSK | | | 107 |
| P20742 | QGIPFFAQVLLVDGK | | | 108 |
| P20742 | GSFALSFPVESDVAPIAR | | | 109 |
| P20742 | VFTNSK | | | 110 |
| P20742 | GEVFTLK | | | 111 |
| P20742 | LPSNVVK | | | 112 |
| P20742 | AVGYLITGYQR | | | 113 |
| P20742 | HQDGSYSTFGER | | | 114 |
| P20742 | EGTHGSHVYTK | | | 115 |
| P20742 | ALLAYAFSLLGK | | | 116 |
| P20742 | EILNSLDK | | | 117 |
| P20742 | EDNLVHWERPQRPK | | | 118 |
| P20742 | QQNAQGGFSSTQDTVVALHALSR | | | 119 |
| P20742 | EDSPFALK | | | 120 |
| P20742 | SLFTDLVAEK | | | 121 |
| P20742 | ISASSEVAFLSIQIK | | | 122 |
| P20742 | GPTQDFR | | | 123 |
| P20742 | VSVQLK | | | 124 |
| P20742 | ASPAFLASQNTK | | | 125 |
| P20742 | QTLSWTVTPK | | | 126 |
| P20742 | TLLVEAEGIEQEK | | | 127 |
| P26639 | ASSPSGK | | | 128 |
| P26639 | EGSGDGGR | | | 129 |
| P26639 | AELNPWPEYIYTR | | | 130 |
| P26639 | AEHDSILAEK | | | 131 |
| P26639 | DSKPIK | | | 132 |
| P26639 | VTLPDGK | | | 133 |
| P26639 | QVDAESWK | | | 134 |
| P26639 | VNTPTTTVYR | | | 135 |
| P26639 | NSSTYWEGK | | | 136 |
| P26639 | IYGISFPDPK | | | 137 |
| P26639 | GAYIYNALIEFIR | | | 138 |
| P26639 | GFQEVVTPNIFNSR | | | 139 |
| P26639 | LADFGVLHR | | | 140 |
| P26639 | NELSGALTGLTR | | | 141 |
| P26639 | TVYSVFGFSFK | | | 142 |
| P26639 | LNLSTRPEK | | | 143 |
| P26639 | FLGDIEVWDQAEK | | | 144 |
| P26639 | QLENSLNEFGEK | | | 145 |
| P26639 | WELNSGDGAFYGPK | | | 146 |
| P26639 | IDIQIK | | | 147 |
| P26639 | FNLTYVSHDGDDK | | | 148 |
| P26639 | RPVIVHR | | | 149 |
| P26639 | QQFHDAK | | | 150 |
| P26639 | ISGTVNIR | | | 151 |
| P26639 | TISETIER | | | 152 |
| P49189 | ILLEAAR | | | 153 |
| P49189 | SIFEAR | | | 154 |
| P49189 | GIKPVTLELGGK | | | 155 |
| P49189 | FTEEVVK | | | 156 |
| P49189 | IGDPLLEDTR | | | 157 |
| P49189 | VLGFVK | | | 158 |
| P49189 | ANDTTFGLAAGVFTR | | | 159 |
| P49189 | VTIEYYSQLK | | | 160 |
| P49189 | VEPADASGTEK | | | 161 |
| P49189 | AFEPATGR | | | 162 |
| P49189 | EVNLAVQNAK | | | 163 |
| P58107 | GQLLPVSK | | | 164 |
| P58107 | ALQQGLVGLELK | | | 165 |
| P58107 | ATTGYPDPYGGEK | | | 166 |
| P58107 | LALFQAIGK | | | 167 |
| P58107 | LSELEPGTGDLR | | | 168 |
| P58107 | FLNPNTLER | | | 169 |
| P58107 | LTYHQLLER | | | 170 |
| P58107 | APGSGLALLPLK | | | 171 |
| P58107 | LAAVDVSAR | | | 172 |
| P58107 | YLEGTGSVAGVVLLPEGHK | | | 173 |
| P58107 | LWVDEAVR | | | 174 |
| P58107 | GLVDRPLALR | | | 175 |
| P58107 | LPLEAALR | | | 176 |
| P58107 | QLSQAGSFSDGTHGGLR | | | 177 |
| P58107 | GGEPQGPPFIK | | | 178 |
| P58107 | QALSTATATVSVGK | | | 179 |
| P58107 | GRPVSLWELLFSEAISSEQR | | | 180 |
| P58107 | LAAELSATLEQAAATAR | | | 181 |
| P58107 | VTFSGLR | | | 182 |
| P58107 | DTVTPGELLK | | | 183 |
| P58107 | AEIIDQDLYER | | | 184 |
| P58107 | LEHGQATAK | | | 185 |
| P58107 | DVGSLASAQR | | | 186 |
| P58107 | LSIYEAR | | | 187 |
| P58107 | GLLRPGTALILLEAQAATGFIIDPK | | | 188 |
| P58107 | GHSVEEALR | | | 189 |
| P58107 | AAVIGPDVFAK | | | 190 |
| P58107 | VSAWELINSEYFSEGR | | | 191 |
| P58107 | EVTLGQVAK | | | 192 |
| P58107 | LLEAETQR | | | 193 |
| P58107 | GVVGPELYGR | | | 194 |
| P58107 | AEGAIAGFR | | | 195 |
| P58107 | GLIPWEQAAR | | | 196 |
| P58107 | SLQAVPGAK | | | 197 |
| P58107 | GLLEDVQEGR | | | 198 |
| P58107 | TTVPQLLASVQR | | | 199 |
| P58107 | ASIAQAVR | | | 200 |
| P58107 | DGLLPTGLGQR | | | 201 |
| P58107 | LLEAQVASGFLVDPLNNQR | | | 202 |
| P58107 | LSVEDAVK | | | 203 |
| P58107 | VGLVGR | | | 204 |
| P58107 | ELSEQLGQAER | | | 205 |
| P58107 | AAAGYPDPYSR | | | 206 |
| P58107 | LGLLDTQTSQVLTAVDK | | | 207 |
| P58107 | FFFDPSAR | | | 208 |
| P58107 | DQVTYQQLR | | | 209 |
| P58107 | VPVSTGR | | | 210 |
| P58107 | QVVSAVTALVEAAER | | | 211 |
| P58107 | QPLQATFR | | | 212 |
| P58107 | TLDELSQGTTTVK | | | 213 |
| P58107 | SLEGGNFIAGVLIQGTQER | | | 214 |
| P58107 | LTVEEAFK | | | 215 |
| P58107 | ILADPSDDTK | | | 216 |
| P58107 | GENYVYINEATR | | | 217 |
| P58107 | FADQVVSFWDLLSSPYFTEDR | | | 218 |
| P58107 | ELIQEYGAQSGGLEK | | | 219 |
| P58107 | LLEIITTTIEETETQNQGIK | | | 220 |
| P58107 | GEVTAADLFNSR | | | 221 |
| P58107 | TLHTLR | | | 222 |
| P58107 | LSVDEAVDVGLVNEELR | | | 223 |
| P58107 | AATGYR | | | 224 |
| P58107 | LLEVQVATGGVIDPQHHHR | | | 225 |
| P58107 | LPLETAYR | | | 226 |
| P58107 | DIYALISDQK | | | 227 |
| P58107 | FVDPNTQEK | | | 228 |
| P58107 | DSEHIDDETR | | | 229 |
| P58107 | ALEAEQVEITVGR | | | 230 |
| P58107 | GQKPTLWALLNSEYVTEEK | | | 231 |
| P58107 | ALQTVAQLILELIEK | | | 232 |
| P58107 | QETSNK | | | 233 |
| P58107 | HLWFQGIR | | | 234 |
| P58107 | LSVEEPVPAGVVGSEIQEK | | | 235 |
| P58107 | GFFDPNTHENLTYVQLLR | | | 236 |
| P58107 | GSAVHQLSEELR | | | 237 |
| P58107 | VTPGSGALQGQSVSVWELLFYR | | | 238 |
| P58107 | EVSEDR | | | 239 |
| P58107 | AGTLTVEELGATLTSLLAQAQAQAR | | | 240 |
| P58107 | AEAEAGSPRPDPR | | | 241 |
| P58107 | AVPVWDVLASGYVSR | | | 242 |
| P58107 | EELLAEFGSGTLDLPALTR | | | 243 |
| P58107 | LTAIIEEAEEAPGARPQLQDAR | | | 244 |
| P58107 | EPGPAGR | | | 245 |
| P58107 | GDGDSGR | | | 246 |
| P58107 | EGQGEGETQEAAAAAAAAR | | | 247 |
| P58107 | QEQTLR | | | 248 |
| P58107 | GQFQGRPVSVWDVLFSSYLSEAR | | | 249 |
| P58107 | DELLAQHAAGALGLPDLVAVLTR | | | 250 |
| P58107 | VIEETEER | | | 251 |
| P58107 | QVSASELHTSGILGPETLR | | | 252 |
| P58107 | DLAQGTK | | | 253 |
| P58107 | LSVEEAVAAGVVGGEIQEK | | | 254 |
| P58107 | AVPVWDVLASGYVSGAAR | | | 255 |
| P58107 | LTAIIEEAEEAPGARPQLQDAWR | | | 256 |
| P58107 | ASTLTVEELGATLTSLLAQAQAQAR | | | 257 |
| P58107 | EGQGEGETQEAAAATAAAR | | | 258 |
| P58107 | GFFDPNTHENLTYLQLLQR | | | 259 |
| P58107 | ATLDPETGLLFLSLSLQ | | | 260 |

TABLE 14
Rule-In TBI Biomarkers-Supervised Analysis

| | | | UniProt | |
|---|---|---|---|---|
| | | | Primary | |
| Unique | | | Accession | SEQ ID |
| Protein | Abbreviated Name | Descriptor | Number | NO: |
| | NCOR1 | NCOR1_HUMAN | O75376 | |
| | ABHEB | ABHEB_HUMAN | Q96IU4 | |
| | ANXA6 | ANXA6_HUMAN | P08133 | |
| | DNM1L | DNM1L_HUMAN | O00429 | |
| | FCN2 | FCN2_HUMAN | Q15485 | |
| | HBA | HBA_HUMAN | P69905 | |
| | MYLK | MYLK_HUMAN | Q15746 | |
| | SBSN | SBSN_HUMAN | Q6UWP8 | |
| | AHNK | AHNK_HUMAN | Q09666 | |
| | K22E | K22E_HUMAN | P35908 | |
| Protein | Peptide Sequence | | | |
| O00429 | SSVLESLVGR | | | 261 |
| O00429 | GTGIVTR | | | 262 |
| O00429 | RPLILQLVHVSQEDK | | | 263 |
| O00429 | TTGEENGVEAEEWGK | | | 264 |
| O00429 | LYTDFDEIR | | | 265 |
| O00429 | QEIENETER | | | 266 |
| O00429 | ISGNNK | | | 267 |
| O00429 | GVSPEPIHLK | | | 268 |
| O00429 | VPVGDQPK | | | 269 |
| O00429 | DIELQIR | | | 270 |
| O00429 | EVDPDGR | | | 271 |
| O00429 | TLAVITK | | | 272 |
| O00429 | LGIIGVVNR | | | 273 |
| O00429 | SQLDINNK | | | 274 |
| O00429 | SVTDSIR | | | 275 |
| O00429 | DEYAFLQK | | | 276 |
| O00429 | YPSLANR | | | 277 |
| O00429 | INVLAAQYQSLLNSYGEPVDDK | | | 278 |
| O00429 | SATLLQLITK | | | 279 |
| O00429 | TLESVDPLGGLNTIDILTAIR | | | 280 |
| O00429 | NATGPRPALFVPEVSFELLVK | | | 281 |
| O00429 | LEEPSLR | | | 282 |
| O00429 | ELPSAVSR | | | 283 |
| O00429 | VPSALAPASQEPSPAASAEADGK | | | 284 |
| O00429 | LIQDSR | | | 285 |
| O00429 | NVASGGGGVGDGVQEPTTGNWR | | | 286 |
| O00429 | AEELLAEEK | | | 287 |
| O00429 | GHAVNLLDVPVPVAR | | | 288 |
| O00429 | SYFLIVR | | | 289 |
| O00429 | NIQDSVPK | | | 290 |
| O00429 | DTLQSELVGQLYK | | | 291 |
| O00429 | ALQGASQIIAEIR | | | 292 |
| O75376 | YPPHSVQYTFPNTR | | | 293 |
| O75376 | HQQEFAVPDYR | | | 294 |
| O75376 | SSHLEVSQASQLLQQQQQQQLR | | | 295 |
| O75376 | RPSLLSEFHPGSDRPQER | | | 296 |
| O75376 | TSYEPFHPGPSPVDHDSLESK | | | 297 |
| O75376 | LEQVSDSHFQR | | | 298 |
| O75376 | VSAAVLPLVHPLPEGLR | | | 299 |
| O75376 | ASADAK | | | 300 |
| O75376 | SIVQIIYDENR | | | 301 |
| O75376 | IFEGLGPK | | | 302 |
| O75376 | VELPLYNQPSDTK | | | 303 |
| O75376 | VYHENIK | | | 304 |
| O75376 | LILEEK | | | 305 |
| O75376 | GAGLSATIAR | | | 306 |
| O75376 | SEHEISEIIDGLSEQENNEK | | | 307 |
| O75376 | FIQHPK | | | 308 |
| O75376 | NFGLIASYLER | | | 309 |
| O75376 | NQQIARPSQEEK | | | 310 |
| O75376 | IDGTAEETEER | | | 311 |
| O75376 | EQATPR | | | 312 |
| O75376 | GLVEHGR | | | 313 |
| O75376 | NWAAIAK | | | 314 |
| O75376 | HNLDNLLQQHK | | | 315 |
| O75376 | ADSVDVEVR | | | 316 |
| O75376 | VPENHASK | | | 317 |
| O75376 | VEGDNTK | | | 318 |
| O75376 | EWEVLQPAPHQVITNLPEGVR | | | 319 |
| O75376 | LPTTRPTRPPPPLIPSSK | | | 320 |
| O75376 | QQESAK | | | 321 |
| O75376 | SATLPYIK | | | 322 |
| O75376 | QEEFSPR | | | 323 |
| O75376 | SQNSQPEGLLVR | | | 324 |
| O75376 | AQHEGVVR | | | 325 |
| O75376 | GTAGAIQEGSITR | | | 326 |
| O75376 | ISVESIPSLR | | | 327 |
| O75376 | GSITQGTPALPQTGIPTEALVK | | | 328 |
| O75376 | SGHILSYDNIK | | | 329 |
| O75376 | TAHEISLK | | | 330 |
| O75376 | SYESVEGNIK | | | 331 |
| O75376 | GSPHSDLK | | | 332 |
| O75376 | ATTESFEDGLK | | | 333 |
| O75376 | ESPPIR | | | 334 |
| O75376 | AFEGAITK | | | 335 |
| O75376 | GKPYDGITTIK | | | 336 |
| O75376 | QDILTQESR | | | 337 |
| O75376 | TPEVVQSTRPIIEGSISQGTPIK | | | 338 |
| O75376 | FDNNSGQSAIK | | | 339 |
| O75376 | SLITGPSK | | | 340 |
| O75376 | AGETVR | | | 341 |
| O75376 | HTSVVSSGPSVLR | | | 342 |
| O75376 | STLHEAPK | | | 343 |
| O75376 | AQLSPGIYDDTSAR | | | 344 |
| O75376 | TSDVTISSNK | | | 345 |
| O75376 | STNHER | | | 346 |
| O75376 | STLTPTQR | | | 347 |
| O75376 | ESIPAK | | | 348 |
| O75376 | SPVPGVDPVVSHSPFDPHHR | | | 349 |
| O75376 | GSTAGEVYR | | | 350 |
| O75376 | ALDPAAAAYLFQR | | | 351 |
| O75376 | EQPLGLPYPATR | | | 352 |
| O75376 | YETPSDAIEVISPASSPAPPQEK | | | 353 |
| O75376 | LQTYQPEVVK | | | 354 |
| O75376 | ANQAENDPTR | | | 355 |
| O75376 | YSPESQAQSVHHQRPGSR | | | 356 |
| O75376 | VSPENLVDK | | | 357 |
| O75376 | SHVSSEPYEPISPPQVPVVHEK | | | 358 |
| O75376 | QDSLLLLSQR | | | 359 |
| O75376 | GAEPAEQR | | | 360 |
| O75376 | SPGSISYLPSFFTK | | | 361 |
| O75376 | GHSFADPASNLGLEDIIR | | | 362 |
| O75376 | EPAPLLSAQYETLSDSDD | | | 363 |
| O75376 | SAAVSEQQQLEQK | | | 364 |
| O75376 | TLEVEK | | | 365 |
| O75376 | TTITAANFIDVIITR | | | 366 |
| O75376 | QIASDK | | | 367 |
| O75376 | GSQSSDSSSSLSSHR | | | 368 |
| P08133 | GFGSDK | | | 369 |
| P08133 | EAILDIITSR | | | 370 |
| P08133 | DLIADLK | | | 371 |
| P08133 | YELTGK | | | 372 |
| P08133 | DAISGIGTDEK | | | 373 |
| P08133 | DLEADIIGDTSGHFQK | | | 374 |
| P08133 | EEDDVVSEDLVQQDVQDLYEAGELK | | | 375 |
| P08133 | WGTDEAQFIYILGNR | | | 376 |
| P08133 | LVFDEYLK | | | 377 |
| P08133 | TTGKPIEASIR | | | 378 |
| P08133 | GELSGDFEK | | | 379 |
| P08133 | STPEYFAER | | | 380 |
| P08133 | NDTSGEYK | | | 381 |
| P08133 | GTVRPANDFNPDADAK | | | 382 |
| P08133 | GLGTDEDTIIDIITHR | | | 383 |
| P08133 | SEISGDLAR | | | 384 |
| P08133 | ALIEILATR | | | 385 |
| P08133 | TNAEIR | | | 386 |
| P08133 | AINEAYK | | | 387 |
| P08133 | SLEDALSSDTSGHFR | | | 388 |
| P08133 | ILISLATGHR | | | 389 |
| P08133 | EEGGENLDQAR | | | 390 |
| P08133 | EDAQVAAEILEIADTPSGDK | | | 391 |
| P08133 | SYPHLR | | | 392 |
| P08133 | VFQEFIK | | | 393 |
| P08133 | DAFVAIVQSVK | | | 394 |
| P08133 | NKPLFFADK | | | 395 |
| P08133 | GAGTDEK | | | 396 |
| P08133 | SEIDLLNIR | | | 397 |
| P08133 | SLHQAIEGDTSGDFLK | | | 398 |
| P35908 | GFSSGSAVVSGGSR | | | 399 |
| P35908 | HGGGGGGFGGGGFGSR | | | 400 |
| P35908 | SLVGLGGTK | | | 401 |
| P35908 | SISISVAGGGGGFGAAGGFGGR | | | 402 |
| P35908 | VDPEIQNVK | | | 403 |
| P35908 | YLDGLTAER | | | 404 |
| P35908 | TAAENDFVTLK | | | 405 |
| P35908 | VELQSK | | | 406 |
| P35908 | VDLLNQEIEFLK | | | 407 |
| P35908 | EEAEALYHSK | | | 408 |
| P35908 | YEELQVTVGR | | | 409 |
| P35908 | LQGEIAHVK | | | 410 |
| P35908 | NVQDAIADAEQR | | | 411 |
| P35908 | GEHALK | | | 412 |
| P35908 | LNDLEEALQQAK | | | 413 |
| P35908 | AAFGGSGGR | | | 414 |
| P35908 | GSSSGGGYSSGSSSYGSGGR | | | 415 |
| P35908 | GGSGGGGSISGGGYGSGGGSGGR | | | 416 |
| P35908 | YGSGGGSK | | | 417 |
| P35908 | GGSISGGGYGSGGGK | | | 418 |
| P35908 | HSSGGGSR | | | 419 |
| P35908 | GGSSSGGGYGSGGGGSSSVK | | | 420 |
| P35908 | GSSGEAFGSSVTFSFR | | | 421 |
| P69905 | VGAHAGEYGAEALER | | | 422 |
| P69905 | TYFPHFDLSHGSAQVK | | | 423 |
| P69905 | FLASVSTVLTSK | | | 424 |
| Q09666 | ELLLPNWQGSGSHGLTIAQR | | | 425 |
| Q09666 | DDGVFVQEVTQNSPAAR | | | 426 |
| Q09666 | SPEPGQTWTR | | | 427 |
| Q09666 | SEDGVEGDLGETQSR | | | 428 |
| Q09666 | TITVTR | | | 429 |
| Q09666 | VTAYTVDVTGR | | | 430 |
| Q09666 | DIDISSPEFK | | | 431 |
| Q09666 | HELTEISNVDVETQSGK | | | 432 |
| Q09666 | LPSGSGAASPTGSAVDIR | | | 433 |
| Q09666 | AGAISASGPELQGAGHSK | | | 434 |
| Q09666 | VGGSGVNVNAK | | | 435 |
| Q09666 | GLDLGGR | | | 436 |
| Q09666 | GGVQVPAVDISSSLGGR | | | 437 |
| Q09666 | AVEVQGPSLESGDHGK | | | 438 |
| Q09666 | FGVSTGR | | | 439 |
| Q09666 | EGQTPK | | | 440 |
| Q09666 | VSAPEVSVGHK | | | 441 |
| Q09666 | GPQITGPSLEGDLGLK | | | 442 |
| Q09666 | FSVSGAK | | | 443 |
| Q09666 | VSAPGVQGDVK | | | 444 |
| Q09666 | GPQVALK | | | 445 |
| Q09666 | VDIETPNLEGTLTGPR | | | 446 |
| Q09666 | LGSPSGK | | | 447 |
| Q09666 | GGVDVTLPR | | | 448 |
| Q09666 | VPEVDVR | | | 449 |
| Q09666 | VDVSAPDVEAHGPEWNLK | | | 450 |
| Q09666 | GDISISGPK | | | 451 |
| Q09666 | VNVEAPDVNLEGLGGK | | | 452 |
| Q09666 | GEYDVTVPK | | | 453 |
| Q09666 | VDIDAPDVDVHGPDWHLK | | | 454 |
| Q09666 | FSVPGFK | | | 455 |
| Q09666 | AEGPEVDVNLPK | | | 456 |
| Q09666 | ADVDISGPK | | | 457 |
| Q09666 | IDVTAPDVSIEEPEGK | | | 458 |
| Q09666 | VPDVELK | | | 459 |
| Q09666 | VGVEVPDVNIEGPEGK | | | 460 |
| Q09666 | GEGPEFDVNLSK | | | 461 |
| Q09666 | ANVDISAPK | | | 462 |
| Q09666 | VDTNAPDLSLEGPEGK | | | 463 |
| Q09666 | GNVDISAPK | | | 464 |
| Q09666 | APDVEGQGLDWSLK | | | 465 |
| Q09666 | GEGPEVDVNLPK | | | 466 |
| Q09666 | ADVVVSGPK | | | 467 |
| Q09666 | VDIEAPDVSLEGPEGK | | | 468 |
| Q09666 | GDVDVSVPK | | | 469 |
| Q09666 | VPDVEIK | | | 470 |
| Q09666 | EVDVNLPK | | | 471 |
| Q09666 | ADIDVSGPK | | | 472 |
| Q09666 | VDVEVPDVSLEGPEGK | | | 473 |
| Q09666 | VDISAPDVDVHGPDWHLK | | | 474 |
| Q09666 | GEGPEVDVK | | | 475 |
| Q09666 | ADVDVSGPK | | | 476 |
| Q09666 | ISIPDVGLHLK | | | 477 |
| Q09666 | GDYDVTVPK | | | 478 |
| Q09666 | VEGEIK | | | 479 |
| Q09666 | APDVDIK | | | 480 |
| Q09666 | VDINAPDVEVHGPDWHLK | | | 481 |
| Q09666 | ADLGVSGPK | | | 482 |
| Q09666 | VDIDVPDVNLEAPEGK | | | 483 |
| Q09666 | TDVDVSLPK | | | 484 |
| Q09666 | VEGDLK | | | 485 |
| Q09666 | GPEIDVK | | | 486 |
| Q09666 | VDIDAPDVEVHDPDWHLK | | | 487 |
| Q09666 | ADIDVSGPSVDTDAPDLDIEGPEGK | | | 488 |
| Q09666 | GEIDASVPELEGDLR | | | 489 |
| Q09666 | GDADVSVPK | | | 490 |
| Q09666 | GDVDVSLPK | | | 491 |
| Q09666 | VPDVDIR | | | 492 |
| Q09666 | GDVDVSAPK | | | 493 |
| Q09666 | GPELDVK | | | 494 |
| Q09666 | GEVDVDVPK | | | 495 |
| Q09666 | GPHVDVSGPDIDIEGPEGK | | | 496 |
| Q09666 | GDVDVSVPEVEGK | | | 497 |
| Q09666 | VDVNAPDVQAPDWHLK | | | 498 |
| Q09666 | VDIEGPDVNIEGPEGK | | | 499 |
| Q09666 | GPEVDIK | | | 500 |
| Q09666 | VDINAPDVGVQGPDWHLK | | | 501 |
| Q09666 | GEGPDGDVK | | | 502 |
| Q09666 | GEGPDVDVNLPK | | | 503 |
| Q09666 | GDVDVTGPK | | | 504 |
| Q09666 | GPEVDLK | | | 505 |
| Q09666 | VDIDVPDVNVQGPDWHLK | | | 506 |
| Q09666 | VDVEGPDVNIEGPEGK | | | 507 |
| Q09666 | GDVDISLPK | | | 508 |
| Q09666 | GPEVDIR | | | 509 |
| Q09666 | GPQVDIDVPDVGVQGPDWHLK | | | 510 |
| Q09666 | ADLDVSGPK | | | 511 |
| Q09666 | VDIDVPDVNIEGPEGK | | | 512 |
| Q09666 | VDINAPDVDVQGPDWHLK | | | 513 |
| Q09666 | VDVDVPDVNIEGPDAK | | | 514 |
| Q09666 | GEVDVSLANVEGDLK | | | 515 |
| Q09666 | GPALDIK | | | 516 |
| Q09666 | IDVDAPDIDIHGPDAK | | | 517 |
| Q09666 | GDVDVSGPK | | | 518 |
| Q09666 | APSLDIK | | | 519 |
| Q09666 | GPEVDVSGPK | | | 520 |
| Q09666 | LNIEGK | | | 521 |
| Q09666 | FNFSGSK | | | 522 |
| Q09666 | VQTPEVDVK | | | 523 |
| Q09666 | KPDIDITGPK | | | 524 |
| Q09666 | VDINAPDVEVQGK | | | 525 |
| Q09666 | GDLDIAGPNLEGDFK | | | 526 |
| Q09666 | APEVNLNAPDVDVHGPDWNLK | | | 527 |
| Q09666 | AEGPDVAVDLPK | | | 528 |
| Q09666 | VDINAPDVDVHGPDWHLK | | | 529 |
| Q09666 | GEGPEVDVTLPK | | | 530 |
| Q09666 | ADIDISGPNVDVDVPDVNIEGPDAK | | | 531 |
| Q09666 | GDVVVSLPK | | | 532 |
| Q09666 | VDIDTPDINIEGSEGK | | | 533 |
| Q09666 | VDINAPDVDVR | | | 534 |
| Q09666 | GPDWHLK | | | 535 |
| Q09666 | APEVDIK | | | 536 |
| Q09666 | VDIDTPDIDIHGPEGK | | | 537 |
| Q09666 | VDIDVPDVDVQGPDWHLK | | | 538 |
| Q09666 | VDIDVPDVNIEGPDAK | | | 539 |
| Q09666 | GEGPDVDVTLPK | | | 540 |
| Q09666 | ADIEISGPK | | | 541 |
| Q09666 | VDIDAPDVSIEGPDAK | | | 542 |
| Q09666 | GPSLDIDTPDVNIEGPEGK | | | 543 |
| Q09666 | GPEVDIEGPEGK | | | 544 |
| Q09666 | VGIDTPDIDIHGPEGK | | | 545 |
| Q09666 | GDVDVTLPK | | | 546 |
| Q09666 | GPEADIK | | | 547 |
| Q09666 | VDINTPDVDVHGPDWHLK | | | 548 |
| Q09666 | GEGPDVDVSLPK | | | 549 |
| Q09666 | VDVDIPDVNIEGPDAK | | | 550 |
| Q09666 | ISIPDVDLDLK | | | 551 |
| Q09666 | GDFDVSVPK | | | 552 |
| Q09666 | VEGTLK | | | 553 |
| Q09666 | LDFEGPDAK | | | 554 |
| Q09666 | LSGPSLK | | | 555 |
| Q09666 | VTAPDVDLHLK | | | 556 |
| Q09666 | IGFSGPK | | | 557 |
| Q09666 | LEGGEVDLK | | | 558 |
| Q09666 | FGFGAK | | | 559 |
| Q09666 | SPSLDVTVPEAELNLETPEISVGGK | | | 560 |
| Q09666 | QGFDLNVPGGEIDASLK | | | 561 |
| Q09666 | APDVDVNIAGPDAALK | | | 562 |
| Q09666 | AEAPLPSPK | | | 563 |
| Q09666 | LEGELQAPDLELSLPAIHVEGLDIK | | | 564 |
| Q09666 | IEGDLK | | | 565 |
| Q09666 | VQANLGAPDINIEGLDAK | | | 566 |
| Q09666 | TPSFGISAPQVSIPDVNVNLK | | | 567 |
| Q09666 | GDVPSVGLEGPDVDLQGPEAK | | | 568 |
| Q09666 | IGIPGVK | | | 569 |
| Q09666 | LEGPDVSLK | | | 570 |
| Q09666 | VSGPDLDLNLK | | | 571 |
| Q09666 | VHAPGLNLSGVGGK | | | 572 |
| Q09666 | VPGIDATTK | | | 573 |
| Q09666 | LNVGAPDVTLR | | | 574 |
| Q09666 | GPSLQGDLAVSGDIK | | | 575 |
| Q09666 | VSVGAPDLSLEASEGSIK | | | 576 |
| Q09666 | LPQFGISTPGSDLHVNAK | | | 577 |
| Q09666 | GPQVSGELK | | | 578 |
| Q09666 | GPGVDVNLK | | | 579 |
| Q09666 | ISAPNVDFNLEGPK | | | 580 |
| Q09666 | GSLGATGEIK | | | 581 |
| Q09666 | LPTGQISGPEIK | | | 582 |
| Q09666 | GSEVGFHGAAPDISVK | | | 583 |
| Q09666 | GGADVSGGVSAPDISLGEGHLSVK | | | 584 |
| Q09666 | GSGGEWK | | | 585 |
| Q09666 | GPQVSSALNLDTSK | | | 586 |
| Q09666 | FAGGLHFSGPK | | | 587 |
| Q09666 | VEGGVK | | | 588 |
| Q09666 | FTFSGR | | | 589 |
| Q09666 | AEASIQAGAGDGEWEESEVK | | | 590 |
| Q09666 | FNFSKPK | | | 591 |
| Q09666 | GGVTGSPEASISGSK | | | 592 |
| Q09666 | ASLGSLEGEAEAEASSPK | | | 593 |
| Q09666 | SNSFSDER | | | 594 |
| Q09666 | EFSGPSTPTGTLEFEGGEVSLEGGK | | | 595 |
| Q09666 | FGTFGGLGSK | | | 596 |
| Q09666 | GHYEVTGSDDETGK | | | 597 |
| Q09666 | LQGSGVSLASK | | | 598 |
| Q09666 | LSSSSSNDSGNK | | | 599 |
| Q09666 | VGIQLPEVELSVSTK | | | 600 |
| Q15485 | VDGSVDFYR | | | 601 |
| Q15485 | DWATYK | | | 602 |
| Q15485 | LGEFWLGNDNIHALTAQGTSELR | | | 603 |
| Q15485 | VDLVDFEDNYQFAK | | | 604 |
| Q15485 | GTHGSFANGINWK | | | 605 |
| Q15485 | GYNYSYK | | | 606 |
| Q15485 | GEAGTNGK | | | 607 |
| Q15746 | LVASSHISK | | | 608 |
| Q15746 | TSLSVDPSR | | | 609 |
| Q15746 | GYPEPQVTWHR | | | 610 |
| Q15746 | NGQPITSGGR | | | 611 |
| Q15746 | GTFSLVIHAVHEEDR | | | 612 |
| Q15746 | QVTVELTVEGSFAK | | | 613 |
| Q15746 | QLGQPVVSK | | | 614 |
| Q15746 | ITGRPQPQVTWLK | | | 615 |
| Q15746 | GNVPLQPSAR | | | 616 |
| Q15746 | VSVSEK | | | 617 |
| Q15746 | ATNSDVR | | | 618 |
| Q15746 | EVTNVISK | | | 619 |
| Q15746 | LDSLEAAAK | | | 620 |
| Q15746 | GGSPPWAANSQPQPPR | | | 621 |
| Q15746 | TAPQTPVLQK | | | 622 |
| Q15746 | TSSSITLQAAR | | | 623 |
| Q15746 | VQPEPR | | | 624 |
| Q15746 | APGLGVLSPSGEER | | | 625 |
| Q15746 | RPAPPRPATFPTR | | | 626 |
| Q15746 | QPGLGSQDVVSK | | | 627 |
| Q15746 | DSAFPK | | | 628 |
| Q15746 | FESKPQSQEVK | | | 629 |
| Q15746 | ENQTVK | | | 630 |
| Q15746 | ITWLLNGQPIQYAR | | | 631 |
| Q15746 | SEYLLPVAPSKPTAPIFLQGLSDLK | | | 632 |
| Q15746 | TQAVLTVQEPHDGTQPWFISKPR | | | 633 |
| Q15746 | DTGHFEVLQNEDVFTLVLK | | | 634 |
| Q15746 | VQPWHAGQYEILLK | | | 635 |
| Q15746 | YGSLRPGWPAR | | | 636 |
| Q15746 | GQGWLEEEDGEDVR | | | 637 |
| Q15746 | QHTEEAIR | | | 638 |
| Q15746 | QQEVEQLDFR | | | 639 |
| Q15746 | TLSEDDLK | | | 640 |
| Q15746 | TVSEEER | | | 641 |
| Q15746 | VHSPQQVDFR | | | 642 |
| Q15746 | VPPPKPATPDFR | | | 643 |
| Q15746 | LPAENGSSSAETLNAK | | | 644 |
| Q15746 | GHAGTTDNEK | | | 645 |
| Q15746 | SESQGTAPAFK | | | 646 |
| Q15746 | LQDVHVAEGK | | | 647 |
| Q15746 | ALPEDR | | | 648 |
| Q15746 | SSLPPVLGTESDATVK | | | 649 |
| Q15746 | AGESVELFGK | | | 650 |
| Q15746 | VENSENGSK | | | 651 |
| Q15746 | LTILAAR | | | 652 |
| Q15746 | STSFNVQDLLPDHEYK | | | 653 |
| Q15746 | DEVEVSDDDEK | | | 654 |
| Q15746 | EPEVDYR | | | 655 |
| Q15746 | TVTINTEQK | | | 656 |
| Q15746 | VSDFYDIEER | | | 657 |
| Q15746 | IIDEDFELTER | | | 658 |
| Q15746 | QISEGVEYIHK | | | 659 |
| Q15746 | LIDFGLAR | | | 660 |
| Q15746 | LENAGSLK | | | 661 |
| Q15746 | DFISNLLK | | | 662 |
| Q15746 | TGNAVR | | | 663 |
| Q15746 | SSTGSPTSPLNAEK | | | 664 |
| Q15746 | DLEVVEGSAAR | | | 665 |
| Q15746 | IEGYPDPEVVWFK | | | 666 |
| Q15746 | DDQSIR | | | 667 |
| Q6UWP8 | VIEGINR | | | 668 |
| Q6UWP8 | GLSNAER | | | 669 |
| Q6UWP8 | ALDGINSGITHAGR | | | 670 |
| Q6UWP8 | VAHEINHGIGQAGK | | | 671 |
| Q6UWP8 | AVQGFHTGVHQAGK | | | 672 |
| Q6UWP8 | LGQGVNHAADQAGK | | | 673 |
| Q6UWP8 | LGQGAHHAAGQAGK | | | 674 |
| Q6UWP8 | ELQNAHNGVNQASK | | | 675 |
| Q6UWP8 | LGHGVNNAAGQVGK | | | 676 |
| Q6UWP8 | LIHHGVHHGANQAGSEAGK | | | 677 |
| Q6UWP8 | FGQGVDNAAGQAGNEAGR | | | 678 |
| Q6UWP8 | FGQGVHHAAGQAGNEAGR | | | 679 |
| Q6UWP8 | FGQGAHHGLSEGWK | | | 680 |
| Q6UWP8 | FGQGIHHAAGQVGK | | | 681 |
| Q6UWP8 | FGQGAHHAAGQAGNEAGR | | | 682 |
| Q6UWP8 | FGQGVHHGLSEGWK | | | 683 |
| Q6UWP8 | FGQGVHHTAGQVGK | | | 684 |
| Q6UWP8 | FGQGVHHAASQFGK | | | 685 |
| Q6UWP8 | LGHGVHHGVNEAWK | | | 686 |
| Q6UWP8 | FGQGVHHAASQVGK | | | 687 |
| Q6UWP8 | VVQGLHHGVSQAGR | | | 688 |
| Q6UWP8 | EAGQFGHDIHHTAGQAGK | | | 689 |
| Q6UWP8 | EGDIAVHGVQPGVHEAGK | | | 690 |
| Q6UWP8 | EAGQFGQGVHHTLEQAGK | | | 691 |
| Q96IU4 | INAANYASVK | | | 692 |
| Q96IU4 | QLPNHR | | | 693 |
| Q96IU4 | EGTIQVQGQALFFR | | | 694 |
| Q96IU4 | EALPGSGQAR | | | 695 |
| Q96IU4 | FSVLLLHGIR | | | 696 |
| Q96IU4 | FSSETWQNLGTLHR | | | 697 |
| Q96IU4 | AVAIDLPGLGHSK | | | 698 |

TABLE 15
Rule-In TBI Biomarkers-Unsupervised Analysis

| | | | UniProt | |
|---|---|---|---|---|
| | | | Primary | |
| Unique | | | Accession | SEQ ID |
| Protein | Abbreviated Name | Descriptor | Number | NO: |
| | CAND1 | CAND1_HUMAN | Q86VP6 | |
| | SYEP | SYEP_HUMAN | P07814 | |
| | GLO2 | GLO2_HUMAN | Q16775 | |
| | DIAP1 | DIAP1_HUMAN | O60610 | |
| | DYL1 | DYL1_HUMAN | P63167 | |
| | PSA | PSA_HUMAN | P55786 | |
| | EPHB4 | EPHB4_HUMAN | P54760 | |
| | PLOD1 | PLOD1_HUMAN | Q02809 | |
| | FBLN3 | FBLN3_HUMAN | Q12805 | |
| | HV103 | HV103_HUMAN | A0A0C4DH29 | |
| | IGHD | IGHD_HUMAN | P01880 | |
| | THIM | THIM_HUMAN | P42765 | |
| | IGHA2 | IGHA2_HUMAN | P01877 | |
| | KV105 | KV105_HUMAN | P01602 | |
| | MASP2 | MASP2_HUMAN | O00187 | |
| Protein | Peptide Sequence | | | |
| A0A0C4DH29 | QAPGQR | | | 699 |
| O00187 | WPEPVFGR | | | 700 |
| O00187 | LASPGFPGEYANDQER | | | 701 |
| O00187 | WTLTAPPGYR | | | 702 |
| O00187 | DTFYSLGSSLDITFR | | | 703 |
| O00187 | AGYVLHR | | | 704 |
| O00187 | SGELSSPEYPRPYPK | | | 705 |
| O00187 | SNTVTITFVTDESGDHTGWK | | | 706 |
| O00187 | VEYITGPGVTTYK | | | 707 |
| O00187 | IYGGQK | | | 708 |
| O00187 | HDASALDIR | | | 709 |
| O00187 | TDDIGTASGWGLTQR | | | 710 |
| O00187 | GDSGGALVFLDSETER | | | 711 |
| O00187 | VINYIPWIENIISDF | | | 712 |
| O60610 | SPDELPSAGGDGGK | | | 713 |
| O60610 | EKPNSAHR | | | 714 |
| O60610 | EETAGSYDSR | | | 715 |
| O60610 | FQPLLDGLK | | | 716 |
| O60610 | SGTTIALK | | | 717 |
| O60610 | LGLHQVLQDLR | | | 718 |
| O60610 | VQLNVFDEQGEEDSYDLK | | | 719 |
| O60610 | AEPHFLSILQHLLLVR | | | 720 |
| O60610 | NDYEARPQYYK | | | 721 |
| O60610 | NGADPDFK | | | 722 |
| O60610 | LQDLQGEK | | | 723 |
| O60610 | DALHSEK | | | 724 |
| O60610 | QQIATEK | | | 725 |
| O60610 | QDLEAEVSQLTGEVAK | | | 726 |
| O60610 | LYKPEVQLR | | | 727 |
| O60610 | RPNWSK | | | 728 |
| O60610 | FENNELFAK | | | 729 |
| O60610 | LTLTFSAQTK | | | 730 |
| O60610 | DQEGGEEK | | | 731 |
| O60610 | TAQNLSIFLGSFR | | | 732 |
| O60610 | LNAILFK | | | 733 |
| O60610 | FPDELAHVEK | | | 734 |
| O60610 | VSAENLQK | | | 735 |
| O60610 | QISDVER | | | 736 |
| O60610 | DVQNFPAATDEK | | | 737 |
| O60610 | DAQEQYNK | | | 738 |
| O60610 | ELGEYFLFDPK | | | 739 |
| O60610 | NSETFPTILEEAK | | | 740 |
| P01602 | VPAQLLGLLLLWLPGAK | | | 741 |
| P01602 | ASQSISSWLAWYQQKPGK | | | 742 |
| P01880 | EVNTSGFAPARPPPQPR | | | 743 |
| P07814 | DDVSISVEEGK | | | 744 |
| P07814 | ENILHVSENVIFTDVNSILR | | | 745 |
| P07814 | GNAAWQEQLK | | | 746 |
| P07814 | APVHVK | | | 747 |
| P07814 | WFGFLEAQQAFQSVGTK | | | 748 |
| P07814 | WDVSTTK | | | 749 |
| P07814 | FPPEASGYLHIGHAK | | | 750 |
| P07814 | AALLNQHYQVNFK | | | 751 |
| P07814 | IQPHPR | | | 752 |
| P07814 | TTEYHDR | | | 753 |
| P07814 | DEQFYWIIEALGIR | | | 754 |
| P07814 | KPYIWEYSR | | | 755 |
| P07814 | LNLNNTVLSK | | | 756 |
| P07814 | LTWFVNEGLVDGWDDPR | | | 757 |
| P07814 | QFIAAQGSSR | | | 758 |
| P07814 | IWAFNK | | | 759 |
| P07814 | VIDPVAPR | | | 760 |
| P07814 | YVALLK | | | 761 |
| P07814 | NPEVGLKPVWYSPK | | | 762 |
| P07814 | IISLDAK | | | 763 |
| P07814 | LNLENK | | | 764 |
| P07814 | GDIIQLQR | | | 765 |
| P07814 | NETSAPFK | | | 766 |
| P07814 | VAVQGDVVR | | | 767 |
| P07814 | EDVDAAVK | | | 768 |
| P07814 | QLLSLK | | | 769 |
| P07814 | SLYDEVAAQGEVVR | | | 770 |
| P07814 | EYIPGQPPLSQSSDSSPTR | | | 771 |
| P07814 | NSEPAGLETPEAK | | | 772 |
| P07814 | VASQGEVVR | | | 773 |
| P07814 | DQVDIAVQELLQLK | | | 774 |
| P07814 | SLIGVEYKPVSATGAEDK | | | 775 |
| P07814 | QNKPQK | | | 776 |
| P07814 | QNDGQR | | | 777 |
| P07814 | NQGGGLSSSGAGEGQGPK | | | 778 |
| P07814 | EENLADWYSQVITK | | | 779 |
| P07814 | DFFDAEIK | | | 780 |
| P07814 | THVADFAPEVAWVTR | | | 781 |
| P07814 | WVQSHR | | | 782 |
| P07814 | HPQPFLR | | | 783 |
| P07814 | FAGGDYTTTIEAFISASGR | | | 784 |
| P07814 | AIQGGTSHHLGQNFSK | | | 785 |
| P07814 | QFAYQNSWGLTTR | | | 786 |
| P07814 | EALIAK | | | 787 |
| P07814 | LLSVNIR | | | 788 |
| P07814 | DNYSPGWK | | | 789 |
| P07814 | FNHWELK | | | 790 |
| P07814 | LTVAENEAETK | | | 791 |
| P07814 | LQAILEDIQVTLFTR | | | 792 |
| P07814 | YYTLFGR | | | 793 |
| P42765 | GVFVVAAK | | | 794 |
| P42765 | TPFGAYGGLLK | | | 795 |
| P42765 | DFTATDLSEFAAK | | | 796 |
| P42765 | ETPALTINR | | | 797 |
| P42765 | YALQSQQR | | | 798 |
| P42765 | HNFTPLAR | | | 799 |
| P42765 | SLDLDISK | | | 800 |
| P42765 | TNVNGGAIALGHPLGGSGSR | | | 801 |
| P42765 | ITAHLVHELR | | | 802 |
| P54760 | LETADLK | | | 803 |
| P54760 | WVTFPQVDGQWEELSGLDEEQHSVR | | | 804 |
| P54760 | APGQAHWLR | | | 805 |
| P54760 | TGWVPR | | | 806 |
| P54760 | GAVHVYATLR | | | 807 |
| P54760 | VDTVAAEHLTR | | | 808 |
| P54760 | RPGAEATGK | | | 809 |
| P54760 | LGPLSK | | | 810 |
| P54760 | FPETVPR | | | 811 |
| P54760 | LNGSSLHLEWSAPLESGGR | | | 812 |
| P54760 | EDLTYALR | | | 813 |
| P54760 | DLVEPWVVVR | | | 814 |
| P54760 | EVPPAVSDIR | | | 815 |
| P54760 | SSPSSLSLAWAVPR | | | 816 |
| P54760 | APSGAVLDYEVK | | | 817 |
| P54760 | GAEGPSSVR | | | 818 |
| P54760 | GASYLVQVR | | | 819 |
| P54760 | SEAGYGPFGQEHHSQTQLDESEGWR | | | 820 |
| P54760 | EAEYSDK | | | 821 |
| P54760 | HGQYLIGHGTK | | | 822 |
| P54760 | EIDVSYVK | | | 823 |
| P54760 | GGYTER | | | 824 |
| P54760 | FLEENSSDPTYTSSLGGK | | | 825 |
| P54760 | FPQVVSALDK | | | 826 |
| P54760 | ENGGASHPLLDQR | | | 827 |
| P54760 | QPHYSAFGSVGEWLR | | | 828 |
| P54760 | SQAKPGTPGGTGGPAPQY | | | 829 |
| P55786 | NVKPDQWVK | | | 830 |
| P55786 | LNLGTVGFYR | | | 831 |
| P55786 | DLSLPPVDR | | | 832 |
| P55786 | LGLQNDLFSLAR | | | 833 |
| P55786 | AGIISTVEVLK | | | 834 |
| P55786 | DVFSPIGER | | | 835 |
| P55786 | LGWDPKPGEGHLDALLR | | | 836 |
| P55786 | GLVLGK | | | 837 |
| P55786 | ATLEEAR | | | 838 |
| P55786 | DHVEGK | | | 839 |
| P55786 | QILSADLR | | | 840 |
| P55786 | SPVYLTVLK | | | 841 |
| P55786 | VLGATLLPDLIQK | | | 842 |
| P55786 | DNWEELYNR | | | 843 |
| P55786 | YQGGFLISR | | | 844 |
| P55786 | LSVEGFAVDK | | | 845 |
| P55786 | AFFESHPAPSAER | | | 846 |
| P55786 | DAESIHQYLLQR | | | 847 |
| P63167 | DIAAHIK | | | 848 |
| Q02809 | ETEGFR | | | 849 |
| Q02809 | SAQFFNYK | | | 850 |
| Q02809 | IQALGLGEDWNVEK | | | 851 |
| Q02809 | GTSAGGGQK | | | 852 |
| Q02809 | EDLVILFADSYDVLFASGPR | | | 853 |
| Q02809 | SQVVFSAEELIYPDR | | | 854 |
| Q02809 | YPVVSDGK | | | 855 |
| Q02809 | FLGSGGFIGYAPNLSK | | | 856 |
| Q02809 | LVAEWEGQDSDSDQLFYTK | | | 857 |
| Q02809 | IFLDPEK | | | 858 |
| Q02809 | EQINITLDHR | | | 859 |
| Q02809 | IFQNLDGALDEVVLK | | | 860 |
| Q02809 | NLAYDTLPVLIHGNGPTK | | | 861 |
| Q02809 | LQLNYLGNYIPR | | | 862 |
| Q02809 | LHYPQK | | | 863 |
| Q02809 | LFIHNHEQHHK | | | 864 |
| Q02809 | AQVEEFLAQHGSEYQSVK | | | 865 |
| Q02809 | LLIQQNK | | | 866 |
| Q02809 | LWSNFWGALSADGYYAR | | | 867 |
| Q02809 | SEDYVDIVQGR | | | 868 |
| Q02809 | VGVWNVPYISNIYLIK | | | 869 |
| Q02809 | GELQSSDLFHHSK | | | 870 |
| Q02809 | HTLGHLLSLDSYR | | | 871 |
| Q02809 | TTHLHNDLWEVFSNPEDWK | | | 872 |
| Q02809 | YIHQNYTK | | | 873 |
| Q02809 | LYPGYYTR | | | 874 |
| Q02809 | AQFDLAFVVR | | | 875 |
| Q02809 | LTHYHEGLPTTR | | | 876 |
| Q02809 | YIAVSFVDP | | | 877 |
| Q02809 | GDAKPEDNLLVLTVATK | | | 878 |
| Q12805 | NNFVIR | | | 879 |
| Q12805 | NPADPQR | | | 880 |
| Q12805 | IPSNPSHR | | | 881 |
| Q12805 | ELPQSIVYK | | | 882 |
| Q12805 | SVPSDIFQIQATTIYANTINTFR | | | 883 |
| Q12805 | SGNENGEFYLR | | | 884 |
| Q12805 | LTIIVGPFSF | | | 885 |
| Q16775 | EAAIVDPVQPQK | | | 886 |
| Q16775 | LTTVLTTHHHWDHAGGNEK | | | 887 |
| Q16775 | LESGLK | | | 888 |
| Q16775 | VYGGDDR | | | 889 |
| Q16775 | IGALTHK | | | 890 |
| Q16775 | ITHLSTLQVGSLNVK | | | 891 |
| Q16775 | ALLEVLGR | | | 892 |
| Q16775 | HVEPGNAAIR | | | 893 |
| Q86VP6 | LDDDSER | | | 894 |
| Q86VP6 | DISSIGLK | | | 895 |
| Q86VP6 | LTSAIAK | | | 896 |
| Q86VP6 | IGEYLEK | | | 897 |
| Q86VP6 | TVSPALISR | | | 898 |
| Q86VP6 | ADVFHAYLSLLK | | | 899 |
| Q86VP6 | ITSEALLVTQQLVK | | | 900 |
| Q86VP6 | ALTLIAGSPLK | | | 901 |
| Q86VP6 | IDLRPVLGEGVPILASFLR | | | 902 |
| Q86VP6 | LGTLSALDILIK | | | 903 |
| Q86VP6 | VYPSSLSK | | | 904 |
| Q86VP6 | ISGSILNELIGLVR | | | 905 |
| Q86VP6 | QSYYSIAK | | | 906 |
| Q86VP6 | EGPAVVGQFIQDVK | | | 907 |
| Q86VP6 | STDSIR | | | 908 |
| Q86VP6 | LLALLSLGEVGHHIDLSGQLELK | | | 909 |
| Q86VP6 | SVILEAFSSPSEEVK | | | 910 |
| Q86VP6 | QYLLLHSLK | | | 911 |
| Q86VP6 | EIISSASVVGLKPYVENIWALLLK | | | 912 |
| Q86VP6 | LTLIDPETLLPR | | | 913 |
| Q86VP6 | GYLISGSSYAR | | | 914 |
| Q86VP6 | SSVVTAVK | | | 915 |
| Q86VP6 | FTISDHPQPIDPLLK | | | 916 |
| Q86VP6 | TLEDPDLNVR | | | 917 |
| Q86VP6 | VALVTFNSAAHNKPSLIR | | | 918 |
| Q86VP6 | DLLDTVLPHLYNETK | | | 919 |
| Q86VP6 | HTVDDGLDIR | | | 920 |
| Q86VP6 | LDIFEFLNHVEDGLK | | | 921 |
| Q86VP6 | DHYDIK | | | 922 |
| Q86VP6 | LVEPLR | | | 923 |
| Q86VP6 | AVAALLTIPEAEK | | | 924 |
| Q86VP6 | VIRPLDQPSSFDATPYIK | | | 925 |
| Q86VP6 | AADIDQEVK | | | 926 |

TABLE 16
Rule-In TBI Biomarkers-Bootstrapping Analysis

| | | | UniProt | |
|---|---|---|---|---|
| | | | Primary | |
| Unique | | | Accession | SEQ ID |
| Protein | Abbreviated Name | Descriptor | Number | NO: |
| | SAMP | SAMP_HUMAN | P02743 | |
| | 1433B | 1433B_HUMAN | P31946 | |
| | FIBB | FIBB_HUMAN | P02675 | |
| | RS28 | RS28_HUMAN | P62857 | |
| | TRFE | TRFE_HUMAN | P02787 | |
| Protein | Peptide Sequence | | | |
| P02675 | SQGVNDNEEGFFSAR | | | 927 |
| P02675 | GHRPLDK | | | 928 |
| P02675 | EEAPSLRPAPPPISGGGYR | | | 929 |
| P02675 | DNENVVNEYSSELEK | | | 930 |
| P02675 | HQLYIDETVNSNIPTNLR | | | 931 |
| P02675 | SILENLR | | | 932 |
| P02675 | QDGSVDFGR | | | 933 |
| P02675 | QGFGNVATNTDGK | | | 934 |
| P02675 | AHYGGFTVQNEANK | | | 935 |
| P02675 | YQISVNK | | | 936 |
| P02675 | DNDGWLTSDPR | | | 937 |
| P02675 | EDGGGWWYNR | | | 938 |
| P02675 | IRPFFPQQ | | | 939 |
| P02743 | VFVFPR | | | 940 |
| P02743 | AYSDLSR | | | 941 |
| P02743 | AYSLFSYNTQGR | | | 942 |
| P02743 | DNELLVYK | | | 943 |
| P02743 | VGEYSLYIGR | | | 944 |
| P02743 | QGYFVEAQPK | | | 945 |
| P02743 | IVLGQEQDSYGGK | | | 946 |
| P02743 | GYVIIKPLVWV | | | 947 |
| P02787 | EDPQTFYYAVAVVK | | | 948 |
| P02787 | DGAGDVAFVK | | | 949 |
| P02787 | HSTIFENLANK | | | 950 |
| P02787 | KPVDEYK | | | 951 |
| P02787 | EDLIWELLNQAQEHFGK | | | 952 |
| P02787 | EFQLFSSPHGK | | | 953 |
| P02787 | DSAHGFLK | | | 954 |
| P02787 | SASDLTWDNLK | | | 955 |
| P02787 | EGYYGYTGAFR | | | 956 |
| P02787 | HQTVPQNTGGK | | | 957 |
| P02787 | NPDPWAK | | | 958 |
| P02787 | APNHAVVTR | | | 959 |
| P02787 | YLGEEYVK | | | 960 |
| P02787 | AVGNLR | | | 961 |
| P31946 | AVTEQGHELSNEER | | | 962 |
| P31946 | YLIPNATQPESK | | | 963 |
| P31946 | YLSEVASGDNK | | | 964 |
| P31946 | QTTVSNSQQAYQEAFEISK | | | 965 |
| P31946 | TAFDEAIAELDTLNEESYK | | | 966 |
| P31946 | DNLTLWTSENQGDEGDAGEGEN | | | 967 |
| P62857 | EGDVLTLLESER | | | 968 |

TABLE 17
Rule-In Mild TBI Biomarkers-Unique

| | | | UniProt | |
|---|---|---|---|---|
| | | | Primary | |
| Unique | | | Accession | SEQ ID |
| Protein | Abbreviated Name | Descriptor | Number | NO: |
| | DYL1 | DYL1_HUMAN | P63137 | |
| | FBLN3 | FBLN3_HUMAN | Q12805 | |
| | PSA | PSA_HUMAN | P55786 | |
| | HV103 | HV103_HUMAN | A0A0C4DH29 | |
| | IGHD | IGHD_HUMAN | P01880 | |
| Protein | Peptide Sequence | | | |
| A0A0C4DH29 | QAPGQR | | | 969 |
| P01880 | EVNTSGFAPARPPPQPR | | | 970 |
| P55786 | NVKPDQWVK | | | 971 |
| P55786 | LNLGTVGFYR | | | 972 |
| P55786 | DLSLPPVDR | | | 973 |
| P55786 | LGLQNDLFSLAR | | | 974 |
| P55786 | AGIISTVEVLK | | | 975 |
| P55786 | DVFSPIGER | | | 976 |
| P55786 | LGWDPKPGEGHLDALLR | | | 977 |
| P55786 | GLVLGK | | | 978 |
| P55786 | ATLEEAR | | | 979 |
| P55786 | DHVEGK | | | 980 |
| P55786 | QILSADLR | | | 981 |
| P55786 | SPVYLTVLK | | | 982 |
| P55786 | VLGATLLPDLIQK | | | 983 |
| P55786 | DNWEELYNR | | | 984 |
| P55786 | YQGGFLISR | | | 985 |
| P55786 | LSVEGFAVDK | | | 986 |
| P55786 | AFFESHPAPSAER | | | 987 |
| P55786 | DAESIHQYLLQR | | | 988 |
| P63167 | DIAAHIK | | | 989 |
| Q12805 | NNFVIR | | | 990 |
| Q12805 | NPADPQR | | | 991 |
| Q12805 | IPSNPSHR | | | 992 |
| Q12805 | ELPQSIVYK | | | 993 |
| Q12805 | SVPSDIFQIQATTIYANTINTFR | | | 994 |
| Q12805 | SGNENGEFYLR | | | 995 |
| Q12805 | LTIIVGPFSF | | | 996 |

TABLE 18
Rule-In Mild TBI Biomarkers-Supervised Analysis

| | | | UniProt |
|---|---|---|---|
| | | | Primary |
| Unique | | | Accession |
| Protein | Abbreviated Name | Descriptor | Number |
| | FCN2 | FCN2_HUMAN | Q15485 |
| | HBA | HBA_HUMAN | P69905 |
| | AHNK | AHNK_HUMAN | Q09666 |
| Protein | Peptide Sequence | | |
| P69905 | VGAHAGEYGAEALER | | 997 |
| P69905 | TYFPHFDLSHGSAQVK | | 998 |
| P69905 | FLASVSTVLTSK | | 999 |
| Q09666 | ELLLPNWQGSGSHGLTIAQR | | 1000 |
| Q09666 | DDGVFVQEVTQNSPAAR | | 1001 |
| Q09666 | SPEPGQTWTR | | 1002 |
| Q09666 | SEDGVEGDLGETQSR | | 1003 |
| Q09666 | TITVTR | | 1004 |
| Q09666 | VTAYTVDVTGR | | 1005 |
| Q09666 | DIDISSPEFK | | 1006 |
| Q09666 | HELTEISNVDVETQSGK | | 1007 |
| Q09666 | LPSGSGAASPTGSAVDIR | | 1008 |
| Q09666 | AGAISASGPELQGAGHSK | | 1009 |
| Q09666 | VGGSGVNVNAK | | 1010 |
| Q09666 | GLDLGGR | | 1011 |
| Q09666 | GGVQVPAVDISSSLGGR | | 1012 |
| Q09666 | AVEVQGPSLESGDHGK | | 1013 |
| Q09666 | FGVSTGR | | 1014 |
| Q09666 | EGQTPK | | 1015 |
| Q09666 | VSAPEVSVGHK | | 1016 |
| Q09666 | GPQITGPSLEGDLGLK | | 1017 |
| Q09666 | FSVSGAK | | 1018 |
| Q09666 | VSAPGVQGDVK | | 1019 |
| Q09666 | GPQVALK | | 1020 |
| Q09666 | VDIETPNLEGTLTGPR | | 1021 |
| Q09666 | LGSPSGK | | 1022 |
| Q09666 | GGVDVTLPR | | 1023 |
| Q09666 | VPEVDVR | | 1024 |
| Q09666 | VDVSAPDVEAHGPEWNLK | | 1025 |
| Q09666 | GDISISGPK | | 1026 |
| Q09666 | VNVEAPDVNLEGLGGK | | 1027 |
| Q09666 | GEYDVTVPK | | 1028 |
| Q09666 | VDIDAPDVDVHGPDWHLK | | 1029 |
| Q09666 | FSVPGFK | | 1030 |
| Q09666 | AEGPEVDVNLPK | | 1031 |
| Q09666 | ADVDISGPK | | 1032 |
| Q09666 | IDVTAPDVSIEEPEGK | | 1033 |
| Q09666 | VPDVELK | | 1034 |
| Q09666 | VGVEVPDVNIEGPEGK | | 1035 |
| Q09666 | GEGPEFDVNLSK | | 1036 |
| Q09666 | ANVDISAPK | | 1037 |
| Q09666 | VDTNAPDLSLEGPEGK | | 1038 |
| Q09666 | GNVDISAPK | | 1039 |
| Q09666 | APDVEGQGLDWSLK | | 1040 |
| Q09666 | GEGPEVDVNLPK | | 1041 |
| Q09666 | ADVVVSGPK | | 1042 |
| Q09666 | VDIEAPDVSLEGPEGK | | 1043 |
| Q09666 | GDVDVSVPK | | 1044 |
| Q09666 | VPDVEIK | | 1045 |
| Q09666 | EVDVNLPK | | 1046 |
| Q09666 | ADIDVSGPK | | 1047 |
| Q09666 | VDVEVPDVSLEGPEGK | | 1048 |
| Q09666 | VDISAPDVDVHGPDWHLK | | 1049 |
| Q09666 | GEGPEVDVK | | 1050 |
| Q09666 | ADVDVSGPK | | 1051 |
| Q09666 | ISIPDVGLHLK | | 1052 |
| Q09666 | GDYDVTVPK | | 1053 |
| Q09666 | VEGEIK | | 1054 |
| Q09666 | APDVDIK | | 1055 |
| Q09666 | VDINAPDVEVHGPDWHLK | | 1056 |
| Q09666 | ADLGVSGPK | | 1057 |
| Q09666 | VDIDVPDVNLEAPEGK | | 1058 |
| Q09666 | TDVDVSLPK | | 1059 |
| Q09666 | VEGDLK | | 1060 |
| Q09666 | GPEIDVK | | 1061 |
| Q09666 | VDIDAPDVEVHDPDWHLK | | 1062 |
| Q09666 | ADIDVSGPSVDTDAPDLDIEGPEGK | | 1063 |
| Q09666 | GEIDASVPELEGDLR | | 1064 |
| Q09666 | GDADVSVPK | | 1065 |
| Q09666 | GDVDVSLPK | | 1066 |
| Q09666 | VPDVDIR | | 1067 |
| Q09666 | GDVDVSAPK | | 1068 |
| Q09666 | GPELDVK | | 1069 |
| Q09666 | GEVDVDVPK | | 1070 |
| Q09666 | GPHVDVSGPDIDIEGPEGK | | 1071 |
| Q09666 | GDVDVSVPEVEGK | | 1072 |
| Q09666 | VDVNAPDVQAPDWHLK | | 1073 |
| Q09666 | VDIEGPDVNIEGPEGK | | 1074 |
| Q09666 | GPEVDIK | | 1075 |
| Q09666 | VDINAPDVGVQGPDWHLK | | 1076 |
| Q09666 | GEGPDGDVK | | 1077 |
| Q09666 | GEGPDVDVNLPK | | 1078 |
| Q09666 | GDVDVTGPK | | 1079 |
| Q09666 | GPEVDLK | | 1080 |
| Q09666 | VDIDVPDVNVQGPDWHLK | | 1081 |
| Q09666 | VDVEGPDVNIEGPEGK | | 1082 |
| Q09666 | GDVDISLPK | | 1083 |
| Q09666 | GPEVDIR | | 1084 |
| Q09666 | GPQVDIDVPDVGVQGPDWHLK | | 1085 |
| Q09666 | ADLDVSGPK | | 1086 |
| Q09666 | VDIDVPDVNIEGPEGK | | 1087 |
| Q09666 | VDINAPDVDVQGPDWHLK | | 1088 |
| Q09666 | VDVDVPDVNIEGPDAK | | 1089 |
| Q09666 | GEVDVSLANVEGDLK | | 1090 |
| Q09666 | GPALDIK | | 1091 |
| Q09666 | IDVDAPDIDIHGPDAK | | 1092 |
| Q09666 | GDVDVSGPK | | 1093 |
| Q09666 | APSLDIK | | 1094 |
| Q09666 | GPEVDVSGPK | | 1095 |
| Q09666 | LNIEGK | | 1096 |
| Q09666 | FNFSGSK | | 1097 |
| Q09666 | VQTPEVDVK | | 1098 |
| Q09666 | KPDIDITGPK | | 1099 |
| Q09666 | VDINAPDVEVQGK | | 1100 |
| Q09666 | GDLDIAGPNLEGDFK | | 1101 |
| Q09666 | APEVNLNAPDVDVHGPDWNLK | | 1102 |
| Q09666 | AEGPDVAVDLPK | | 1103 |
| Q09666 | VDINAPDVDVHGPDWHLK | | 1104 |
| Q09666 | GEGPEVDVTLPK | | 1105 |
| Q09666 | ADIDISGPNVDVDVPDVNIEGPDAK | | 1106 |
| Q09666 | GDVVVSLPK | | 1107 |
| Q09666 | VDIDTPDINIEGSEGK | | 1108 |
| Q09666 | VDINAPDVDVR | | 1109 |
| Q09666 | GPDWHLK | | 1110 |
| Q09666 | APEVDIK | | 1111 |
| Q09666 | VDIDTPDIDIHGPEGK | | 1112 |
| Q09666 | VDIDVPDVDVQGPDWHLK | | 1113 |
| Q09666 | VDIDVPDVNIEGPDAK | | 1114 |
| Q09666 | GEGPDVDVTLPK | | 1115 |
| Q09666 | ADIEISGPK | | 1116 |
| Q09666 | VDIDAPDVSIEGPDAK | | 1117 |
| Q09666 | GPSLDIDTPDVNIEGPEGK | | 1118 |
| Q09666 | GPEVDIEGPEGK | | 1119 |
| Q09666 | VGIDTPDIDIHGPEGK | | 1120 |
| Q09666 | GDVDVTLPK | | 1121 |
| Q09666 | GPEADIK | | 1122 |
| Q09666 | VDINTPDVDVHGPDWHLK | | 1123 |
| Q09666 | GEGPDVDVSLPK | | 1124 |
| Q09666 | VDVDIPDVNIEGPDAK | | 1125 |
| Q09666 | ISIPDVDLDLK | | 1126 |
| Q09666 | GDFDVSVPK | | 1127 |
| Q09666 | VEGTLK | | 1128 |
| Q09666 | LDFEGPDAK | | 1129 |
| Q09666 | LSGPSLK | | 1130 |
| Q09666 | VTAPDVDLHLK | | 1131 |
| Q09666 | IGFSGPK | | 1132 |
| Q09666 | LEGGEVDLK | | 1133 |
| Q09666 | FGFGAK | | 1134 |
| Q09666 | SPSLDVTVPEAELNLETPEISVGGK | | 1135 |
| Q09666 | QGFDLNVPGGEIDASLK | | 1136 |
| Q09666 | APDVDVNIAGPDAALK | | 1137 |
| Q09666 | AEAPLPSPK | | 1138 |
| Q09666 | LEGELQAPDLELSLPAIHVEGLDIK | | 1139 |
| Q09666 | IEGDLK | | 1140 |
| Q09666 | VQANLGAPDINIEGLDAK | | 1141 |
| Q09666 | TPSFGISAPQVSIPDVNVNLK | | 1142 |
| Q09666 | GDVPSVGLEGPDVDLQGPEAK | | 1143 |
| Q09666 | IGIPGVK | | 1144 |
| Q09666 | LEGPDVSLK | | 1145 |
| Q09666 | VSGPDLDLNLK | | 1146 |
| Q09666 | VHAPGLNLSGVGGK | | 1147 |
| Q09666 | VPGIDATTK | | 1148 |
| Q09666 | LNVGAPDVTLR | | 1149 |
| Q09666 | GPSLQGDLAVSGDIK | | 1150 |
| Q09666 | VSVGAPDLSLEASEGSIK | | 1151 |
| Q09666 | LPQFGISTPGSDLHVNAK | | 1152 |
| Q09666 | GPQVSGELK | | 1153 |
| Q09666 | GPGVDVNLK | | 1154 |
| Q09666 | ISAPNVDFNLEGPK | | 1155 |
| Q09666 | GSLGATGEIK | | 1156 |
| Q09666 | LPTGQISGPEIK | | 1157 |
| Q09666 | GSEVGFHGAAPDISVK | | 1158 |
| Q09666 | GGADVSGGVSAPDISLGEGHLSVK | | 1159 |
| Q09666 | GSGGEWK | | 1160 |
| Q09666 | GPQVSSALNLDTSK | | 1161 |
| Q09666 | FAGGLHFSGPK | | 1162 |
| Q09666 | VEGGVK | | 1163 |
| Q09666 | FTFSGR | | 1164 |
| Q09666 | AEASIQAGAGDGEWEESEVK | | 1165 |
| Q09666 | FNFSKPK | | 1166 |
| Q09666 | GGVTGSPEASISGSK | | 1167 |
| Q09666 | ASLGSLEGEAEAEASSPK | | 1168 |
| Q09666 | SNSFSDER | | 1169 |
| Q09666 | EFSGPSTPTGTLEFEGGEVSLEGGK | | 1170 |
| Q09666 | FGTFGGLGSK | | 1171 |
| Q09666 | GHYEVTGSDDETGK | | 1172 |
| Q09666 | LQGSGVSLASK | | 1173 |
| Q09666 | LSSSSSNDSGNK | | 1174 |
| Q09666 | VGIQLPEVELSVSTK | | 1175 |
| Q15485 | VDGSVDFYR | | 1176 |
| Q15485 | DWATYK | | 1177 |
| Q15485 | LGEFWLGNDNIHALTAQGTSELR | | 1178 |
| Q15485 | VDLVDFEDNYQFAK | | 1179 |
| Q15485 | GTHGSFANGINWK | | 1180 |
| Q15485 | GYNYSYK | | 1181 |
| Q15485 | GEAGTNGK | | 1182 |

TABLE 19
Rule-In Mild TBI Biomarkers-Unsupervised Analysis

| | | | UniProt | |
|---|---|---|---|---|
| | | | Primary | |
| Unique | | | Accession | SEQ ID |
| Protein | Abbreviated Name | Descriptor | Number | NO: |
| | HV103 | HV103_HUMAN | A0A0C4DH29 | |
| | IGHD | IGHD_HUMAN | P01880 | |
| | THIM | THIM_HUMAN | P42765 | |
| | IGHA2 | IGHA2_HUMAN | P01877 | |
| | KV105 | KV105_HUMAN | P01602 | |
| | MASP2 | MASP2_HUMAN | O00187 | |
| | DIAP1 | DIAP1_HUMAN | O60610 | |
| | PLOD1 | PLOD1_HUMAN | Q02809 | |
| | EPHB4 | EPHB4_HUMAN | P54760 | |
| | FBLN3 | FBLN3_HUMAN | Q12805 | |
| | DYL1 | DYL1_HUMAN | P63167 | |
| | PSA | PSA_HUMAN | P55786 | |
| Protein | Peptide Sequence | | | |
| A0A0C4DH29 | QAPGQR | | | 1183 |
| O00187 | WPEPVFGR | | | 1184 |
| O00187 | LASPGFPGEYANDQER | | | 1185 |
| O00187 | WTLTAPPGYR | | | 1186 |
| O00187 | DTFYSLGSSLDITFR | | | 1187 |
| O00187 | AGYVLHR | | | 1188 |
| O00187 | SGELSSPEYPRPYPK | | | 1189 |
| O00187 | SNTVTITFVTDESGDHTGWK | | | 1190 |
| O00187 | VEYITGPGVTTYK | | | 1191 |
| O00187 | IYGGQK | | | 1192 |
| O00187 | HDASALDIR | | | 1193 |
| O00187 | TDDIGTASGWGLTQR | | | 1194 |
| O00187 | GDSGGALVFLDSETER | | | 1195 |
| O00187 | VINYIPWIENIISDF | | | 1196 |
| O60610 | SPDELPSAGGDGGK | | | 1197 |
| O60610 | EKPNSAHR | | | 1198 |
| O60610 | EETAGSYDSR | | | 1199 |
| O60610 | FQPLLDGLK | | | 1200 |
| O60610 | SGTTIALK | | | 1201 |
| O60610 | LGLHQVLQDLR | | | 1202 |
| O60610 | VQLNVFDEQGEEDSYDLK | | | 1203 |
| O60610 | AEPHFLSILQHLLLVR | | | 1204 |
| O60610 | NDYEARPQYYK | | | 1205 |
| O60610 | NGADPDFK | | | 1206 |
| O60610 | LQDLQGEK | | | 1207 |
| O60610 | DALHSEK | | | 1208 |
| O60610 | QQIATEK | | | 1209 |
| O60610 | QDLEAEVSQLTGEVAK | | | 1210 |
| O60610 | LYKPEVQLR | | | 1211 |
| O60610 | RPNWSK | | | 1212 |
| O60610 | FENNELFAK | | | 1213 |
| O60610 | LTLTFSAQTK | | | 1214 |
| O60610 | DQEGGEEK | | | 1215 |
| O60610 | TAQNLSIFLGSFR | | | 1216 |
| O60610 | LNAILFK | | | 1217 |
| O60610 | FPDELAHVEK | | | 1218 |
| O60610 | VSAENLQK | | | 1219 |
| O60610 | QISDVER | | | 1220 |
| O60610 | DVQNFPAATDEK | | | 1221 |
| O60610 | DAQEQYNK | | | 1222 |
| O60610 | ELGEYFLFDPK | | | 1223 |
| O60610 | NSETFPTILEEAK | | | 1224 |
| P01602 | VPAQLLGLLLLWLPGAK | | | 1225 |
| P01602 | ASQSISSWLAWYQQKPGK | | | 1226 |
| P01880 | EVNTSGFAPARPPPQPR | | | 1227 |
| P42765 | GVFVVAAK | | | 1228 |
| P42765 | TPFGAYGGLLK | | | 1229 |
| P42765 | DFTATDLSEFAAK | | | 1230 |
| P42765 | ETPALTINR | | | 1231 |
| P42765 | YALQSQQR | | | 1232 |
| P42765 | HNFTPLAR | | | 1233 |
| P42765 | SLDLDISK | | | 1234 |
| P42765 | TNVNGGAIALGHPLGGSGSR | | | 1235 |
| P42765 | ITAHLVHELR | | | 1236 |
| P54760 | LETADLK | | | 1237 |
| P54760 | WVTFPQVDGQWEELSGLDEEQH | | | 1238 |
| | SVR | | | |
| P54760 | APGQAHWLR | | | 1239 |
| P54760 | TGWVPR | | | 1240 |
| P54760 | GAVHVYATLR | | | 1241 |
| P54760 | VDTVAAEHLTR | | | 1242 |
| P54760 | RPGAEATGK | | | 1243 |
| P54760 | LGPLSK | | | 1244 |
| P54760 | FPETVPR | | | 1245 |
| P54760 | LNGSSLHLEWSAPLESGGR | | | 1246 |
| P54760 | EDLTYALR | | | 1247 |
| P54760 | DLVEPWVVVR | | | 1248 |
| P54760 | EVPPAVSDIR | | | 1249 |
| P54760 | SSPSSLSLAWAVPR | | | 1250 |
| P54760 | APSGAVLDYEVK | | | 1251 |
| P54760 | GAEGPSSVR | | | 1252 |
| P54760 | GASYLVQVR | | | 1253 |
| P54760 | SEAGYGPFGQEHHSQTQLDESEG | | | 1254 |
| | WR | | | |
| P54760 | EAEYSDK | | | 1255 |
| P54760 | HGQYLIGHGTK | | | 1256 |
| P54760 | EIDVSYVK | | | 1257 |
| P54760 | GGYTER | | | 1258 |
| P54760 | FLEENSSDPTYTSSLGGK | | | 1259 |
| P54760 | FPQVVSALDK | | | 1260 |
| P54760 | ENGGASHPLLDQR | | | 1261 |
| P54760 | QPHYSAFGSVGEWLR | | | 1262 |
| P54760 | SQAKPGTPGGTGGPAPQY | | | 1263 |
| P55786 | NVKPDQWVK | | | 1264 |
| P55786 | LNLGTVGFYR | | | 1265 |
| P55786 | DLSLPPVDR | | | 1266 |
| P55786 | LGLQNDLFSLAR | | | 1267 |
| P55786 | AGIISTVEVLK | | | 1268 |
| P55786 | DVFSPIGER | | | 1269 |
| P55786 | LGWDPKPGEGHLDALLR | | | 1270 |
| P55786 | GLVLGK | | | 1271 |
| P55786 | ATLEEAR | | | 1272 |
| P55786 | DHVEGK | | | 1273 |
| P55786 | QILSADLR | | | 1274 |
| P55786 | SPVYLTVLK | | | 1275 |
| P55786 | VLGATLLPDLIQK | | | 1276 |
| P55786 | DNWEELYNR | | | 1277 |
| P55786 | YQGGFLISR | | | 1278 |
| P55786 | LSVEGFAVDK | | | 1279 |
| P55786 | AFFESHPAPSAER | | | 1280 |
| P55786 | DAESIHQYLLQR | | | 1281 |
| P63167 | DIAAHIK | | | 1282 |
| Q02809 | ETEGFR | | | 1283 |
| Q02809 | SAQFFNYK | | | 1284 |
| Q02809 | IQALGLGEDWNVEK | | | 1285 |
| Q02809 | GTSAGGGQK | | | 1286 |
| Q02809 | EDLVILFADSYDVLFASGPR | | | 1287 |
| Q02809 | SQVVFSAEELIYPDR | | | 1288 |
| Q02809 | YPVVSDGK | | | 1289 |
| Q02809 | FLGSGGFIGYAPNLSK | | | 1290 |
| Q02809 | LVAEWEGQDSDSDQLFYTK | | | 1291 |
| Q02809 | IFLDPEK | | | 1292 |
| Q02809 | EQINITLDHR | | | 1293 |
| Q02809 | IFQNLDGALDEVVLK | | | 1294 |
| Q02809 | NLAYDTLPVLIHGNGPTK | | | 1295 |
| Q02809 | LQLNYLGNYIPR | | | 1296 |
| Q02809 | LHYPQK | | | 1297 |
| Q02809 | LFIHNHEQHHK | | | 1298 |
| Q02809 | AQVEEFLAQHGSEYQSVK | | | 1299 |
| Q02809 | LLIQQNK | | | 1300 |
| Q02809 | LWSNFWGALSADGYYAR | | | 1301 |
| Q02809 | SEDYVDIVQGR | | | 1302 |
| Q02809 | VGVWNVPYISNIYLIK | | | 1303 |
| Q02809 | GELQSSDLFHHSK | | | 1304 |
| Q02809 | HTLGHLLSLDSYR | | | 1305 |
| Q02809 | TTHLHNDLWEVFSNPEDWK | | | 1306 |
| Q02809 | YIHQNYTK | | | 1307 |
| Q02809 | LYPGYYTR | | | 1308 |
| Q02809 | AQFDLAFVVR | | | 1309 |
| Q02809 | LTHYHEGLPTTR | | | 1310 |
| Q02809 | YIAVSFVDP | | | 1311 |
| Q02809 | GDAKPEDNLLVLTVATK | | | 1312 |
| Q12805 | NNFVIR | | | 1313 |
| Q12805 | NPADPQR | | | 1314 |
| Q12805 | IPSNPSHR | | | 1315 |
| Q12805 | ELPQSIVYK | | | 1316 |
| Q12805 | SVPSDIFQIQATTIYANTINTFR | | | 1317 |
| Q12805 | SGNENGEFYLR | | | 1318 |
| Q12805 | LTIIVGPFSF | | | 1319 |

TABLE 20
Rule-In Severe TBI Biomarkers

| | | | UniProt | |
|---|---|---|---|---|
| | | | Primary | |
| Unique | | | Accession | SEQ ID |
| Protein | Abbreviated Name | Descriptor | Number | NO: |
| | CAND1 | CAND1_HUMAN | Q86VP6 | |
| | GLO2 | GLO2_HUMAN | Q16775 | |
| | ERAP1 | ERAP1_HUMAN | Q9NZ08 | |
| | SYYC | SYYC_HUMAN | P54577 | |
| | C1RL | C1RL_HUMAN | Q9NZP8 | |
| | ATPG | ATPG_HUMAN | P36542 | |
| Protein | Peptide Sequence | | | |
| P36542 | AGVAGLSAWTLQPQWIQVR | | | 1320 |
| P36542 | IYGLGSLALYEK | | | 1321 |
| P36542 | HLLIGVSSDR | | | 1322 |
| P36542 | SEVATLTAAGK | | | 1323 |
| P36542 | THSDQFLVAFK | | | 1324 |
| P36542 | SVISYK | | | 1325 |
| P36542 | ESTTSEQSAR | | | 1326 |
| P36542 | LTLTFNR | | | 1327 |
| P36542 | QAVITK | | | 1328 |
| P36542 | ELIEIISGAAALD | | | 1329 |
| P54577 | LHLITR | | | 1330 |
| P54577 | NLQEVLGEEK | | | 1331 |
| P54577 | IADFLK | | | 1332 |
| P54577 | APWELLELR | | | 1333 |
| P54577 | VSYYENVIK | | | 1334 |
| P54577 | GTDYQLSK | | | 1335 |
| P54577 | EYTLDVYR | | | 1336 |
| P54577 | LSSVVTQHDSK | | | 1337 |
| P54577 | AGAEVVK | | | 1338 |
| P54577 | QVEHPLLSGLLYPGLQALDEEYLK | | | 1339 |
| P54577 | VDAQFGGIDQR | | | 1340 |
| P54577 | IFTFAEK | | | 1341 |
| P54577 | YLPALGYSK | | | 1342 |
| P54577 | HVLFPLK | | | 1343 |
| P54577 | SEFVILR | | | 1344 |
| P54577 | TYTAYVDLEK | | | 1345 |
| P54577 | DFAAEVVHPGDLK | | | 1346 |
| P54577 | NSVEVALNK | | | 1347 |
| P54577 | LLDPIR | | | 1348 |
| P54577 | FNTPALK | | | 1349 |
| P54577 | LASAAYPDPSK | | | 1350 |
| P54577 | NSEPEEVIPSR | | | 1351 |
| P54577 | IITVEK | | | 1352 |
| P54577 | HPDADSLYVEK | | | 1353 |
| P54577 | IDVGEAEPR | | | 1354 |
| P54577 | TVVSGLVQFVPK | | | 1355 |
| P54577 | QVEPLDPPAGSAPGEHVFVK | | | 1356 |
| P54577 | GQPDEELKPK | | | 1357 |
| P54577 | LQADFK | | | 1358 |
| Q16775 | EAAIVDPVQPQK | | | 1359 |
| Q16775 | LTTVLTTHHHWDHAGGNEK | | | 1360 |
| Q16775 | LESGLK | | | 1361 |
| Q16775 | VYGGDDR | | | 1362 |
| Q16775 | IGALTHK | | | 1363 |
| Q16775 | ITHLSTLQVGSLNVK | | | 1364 |
| Q16775 | ALLEVLGR | | | 1365 |
| Q16775 | HVEPGNAAIR | | | 1366 |
| Q86VP6 | LDDDSER | | | 1367 |
| Q86VP6 | DISSIGLK | | | 1368 |
| Q86VP6 | LTSAIAK | | | 1369 |
| Q86VP6 | IGEYLEK | | | 1370 |
| Q86VP6 | TVSPALISR | | | 1371 |
| Q86VP6 | ADVFHAYLSLLK | | | 1372 |
| Q86VP6 | ITSEALLVTQQLVK | | | 1373 |
| Q86VP6 | ALTLIAGSPLK | | | 1374 |
| Q86VP6 | IDLRPVLGEGVPILASFLR | | | 1375 |
| Q86VP6 | LGTLSALDILIK | | | 1376 |
| Q86VP6 | VYPSSLSK | | | 1377 |
| Q86VP6 | ISGSILNELIGLVR | | | 1378 |
| Q86VP6 | QSYYSIAK | | | 1379 |
| Q86VP6 | EGPAVVGQFIQDVK | | | 1380 |
| Q86VP6 | STDSIR | | | 1381 |
| Q86VP6 | LLALLSLGEVGHHIDLSGQLELK | | | 1382 |
| Q86VP6 | SVILEAFSSPSEEVK | | | 1383 |
| Q86VP6 | QYLLLHSLK | | | 1384 |
| Q86VP6 | EIISSASVVGLKPYVENIWALLLK | | | 1385 |
| Q86VP6 | LTLIDPETLLPR | | | 1386 |
| Q86VP6 | GYLISGSSYAR | | | 1387 |
| Q86VP6 | SSVVTAVK | | | 1388 |
| Q86VP6 | FTISDHPQPIDPLLK | | | 1389 |
| Q86VP6 | TLEDPDLNVR | | | 1390 |
| Q86VP6 | VALVTFNSAAHNKPSLIR | | | 1391 |
| Q86VP6 | DLLDTVLPHLYNETK | | | 1392 |
| Q86VP6 | HTVDDGLDIR | | | 1393 |
| Q86VP6 | LDIFEFLNHVEDGLK | | | 1394 |
| Q86VP6 | DHYDIK | | | 1395 |
| Q86VP6 | LVEPLR | | | 1396 |
| Q86VP6 | AVAALLTIPEAEK | | | 1397 |
| Q86VP6 | VIRPLDQPSSFDATPYIK | | | 1398 |
| Q86VP6 | AADIDQEVK | | | 1399 |
| Q9NZ08 | SDGTPFPWNK | | | 1400 |
| Q9NZ08 | VEITASQPTSTIILHSHHLQISR | | | 1401 |
| Q9NZ08 | LSEEPLQVLEHPR | | | 1402 |
| Q9NZ08 | ILASTQFEPTAAR | | | 1403 |
| Q9NZ08 | ASFSIK | | | 1404 |
| Q9NZ08 | SVTVAEGLIEDHFDVTVK | | | 1405 |
| Q9NZ08 | VSVYAVPDK | | | 1406 |
| Q9NZ08 | ESALLFDAEK | | | 1407 |
| Q9NZ08 | VGDYFFGK | | | 1408 |
| Q9NZ08 | EYLSADAFK | | | 1409 |
| Q9NZ08 | SGIVQYLQK | | | 1410 |
| Q9NZ08 | SQHSSSSSHWHQEGVDVK | | | 1411 |
| Q9NZ08 | GFPLITITVR | | | 1412 |
| Q9NZ08 | GSDGAPDTGYLWHVPLTFITSK | | | 1413 |
| Q9NZ08 | TDVLILPEEVEWIK | | | 1414 |
| Q9NZ08 | GTHTAVSSNDR | | | 1415 |
| Q9NZ08 | ASLINNAFQLVSIGK | | | 1416 |
| Q9NZ08 | ALDLSLYLK | | | 1417 |
| Q9NZ08 | QTWTDEGSVSER | | | 1418 |
| Q9NZ08 | YQFSLSSTEK | | | 1419 |
| Q9NZ08 | LQWLLDESFK | | | 1420 |
| Q9NZ08 | TQEFPQILTLIGR | | | 1421 |
| Q9NZ08 | NPVGYPLAWQFLR | | | 1422 |
| Q9NZ08 | GFFSSLK | | | 1423 |
| Q9NZ08 | ENGSQLR | | | 1424 |
| Q9NZ08 | VWLQSEK | | | 1425 |
| Q9NZP8 | GSVLLAQELPQQLTSPGYPEPYGK | | | 1426 |
| Q9NZP8 | GQESSTDIK | | | 1427 |
| Q9NZP8 | APEGFAVR | | | 1428 |
| Q9NZP8 | EFVSSGR | | | 1429 |
| Q9NZP8 | TQPSSENK | | | 1430 |
| Q9NZP8 | TAHLHK | | | 1431 |
| Q9NZP8 | GFLALYQTVAVNYSQPISEASR | | | 1432 |
| Q9NZP8 | GSEAINAPGDNPAK | | | 1433 |
| Q9NZP8 | LGNFPWQAFTSIHGR | | | 1434 |
| Q9NZP8 | WILTAAHTIYPK | | | 1435 |
| Q9NZP8 | LGNHPVHR | | | 1436 |
| Q9NZP8 | VVVHPDYR | | | 1437 |
| Q9NZP8 | VLSYVDWIK | | | 1438 |

TABLE 21
Rule-Out TBI Biomarkers

| | | | UniProt | |
|---|---|---|---|---|
| | | | Primary | |
| Unique | | | Accession | SEQ ID |
| Protein | Abbreviated Name | Descriptor | Number | NO: |
| | ACTBL | ACTBL_HUMAN | Q562R1 | |
| | ALDH2 | ALDH2_HUMAN | P05091 | |
| | ANXA5 | ANXA5_HUMAN | P08758 | |
| | CAMP | CAMP_HUMAN | P49913 | |
| | CPNE3 | CPNE3_HUMAN | O75131 | |
| | CRAC1 | CRAC1_HUMAN | Q9NQ79 | |
| | CYTC | CYTC_HUMAN | P01034 | |
| | DNPEP | DNPEP_HUMAN | Q9ULA0 | |
| | EIF31 | EIF3I_HUMAN | Q13347 | |
| | GSHB | GSHB_HUMAN | P48637 | |
| | ICAM1 | ICAM1_HUMAN | P05362 | |
| | HV323 | HV323_HUMAN | P01764 | |
| | HNRPD | HNRPD_HUMAN | Q14103 | |
| | KVD33 | KVD33_HUMAN | P01593 | |
| | FA9 | FA9_HUMAN | P00740 | |
| | FHR4 | FHR4_HUMAN | Q92496 | |
| | FRPD1 | FRPD1_HUMAN | Q5SYB0 | |
| | HS9013 | HS90B_HUMAN | P08238 | |
| | MA2A1 | MA2A1_HUMAN | Q16706 | |
| | PCYOX | PCYOX_HUMAN | Q9UHG3 | |
| | PNPH | PNPH_HUMAN | P00491 | |
| | PROC | PROC_HUMAN | P04070 | |
| | RL3 | RL3_HUMAN | P39023 | |
| | SH3L3 | SH3L3_HUMAN | Q9H299 | |
| | SRRM2 | SRRM2_HUMAN | Q9UQ35 | |
| | TBB1 | TBB1_HUMAN | Q9H4B7 | |
| | TENA | TENA_HUMAN | P24821 | |
| | TRAP1 | TRAP1_HUMAN | Q12931 | |
| Protein | Peptide Sequence | | | |
| O75131 | TFIIDYYFEVVQK | | | 1439 |
| O75131 | FGVYDIDNK | | | 1441 |
| O75131 | TGRPAGK | | | 1442 |
| O75131 | GSITISAEEIK | | | 1443 |
| O75131 | SDPYLEFHK | | | 1444 |
| O75131 | NNLNPVWRPFK | | | 1445 |
| O75131 | NSGVISVK | | | 1446 |
| O75131 | LYGPTNFSPIINHVAR | | | 1447 |
| O75131 | QAIVNASR | | | 1448 |
| O75131 | SPLGEVAIR | | | 1449 |
| O75131 | QFQNAPK | | | 1450 |
| P00491 | NTAEWLLSHTK | | | 1451 |
| P00491 | LTQAQIFDYGEIPNFPR | | | 1452 |
| P00491 | STVPGHAGR | | | 1453 |
| P00491 | LVFGFLNGR | | | 1454 |
| P00491 | VFHLLGVDTLVVTNAAGGLNPK | | | 1455 |
| P00491 | DHINLPGFSGQNPLR | | | 1456 |
| P00491 | GPNDER | | | 1457 |
| P00491 | ALSTWK | | | 1458 |
| P00491 | VFGFSLITNK | | | 1459 |
| P00491 | ANHEEVLAAGK | | | 1460 |
| P00740 | ILNRPK | | | 1461 |
| P00740 | LEEFVQGNLER | | | 1462 |
| P00740 | EVFENTER | | | 1463 |
| P00740 | TTEFWK | | | 1464 |
| P00740 | NSADNK | | | 1465 |
| P00740 | LAENQK | | | 1466 |
| P00740 | VSVSQTSK | | | 1467 |
| P00740 | VVGGEDAKPGQFPWQVVLNGK | | | 1468 |
| P00740 | ITVVAGEHNIEETEHTEQK | | | 1469 |
| P00740 | IIPHHNYNAAINK | | | 1470 |
| P00740 | EYTNIFLK | | | 1471 |
| P00740 | FGSGYVSGWGR | | | 1472 |
| P00740 | SALVLQYLR | | | 1473 |
| P00740 | VPLVDR | | | 1474 |
| P01034 | ALDFAVGEYNK | | | 1475 |
| P01034 | QIVAGVNYFLDVELGR | | | 1476 |
| P01593 | VPAQLLGLLLLWLSGAR | | | 1477 |
| P01593 | LLIYDASNLETGVPSR | | | 1478 |
| P01764 | GLEWVSAISGSGGSTYYADSVK | | | 1479 |
| P04070 | AHQVLR | | | 1480 |
| P04070 | ANSFLEELR | | | 1481 |
| P04070 | EIFQNVDDTLAFWSK | | | 1482 |
| P04070 | SGWEGR | | | 1483 |
| P04070 | DTEDQEDQVDPR | | | 1484 |
| P04070 | GDSPWQVVLLDSK | | | 1485 |
| P04070 | LGEYDLR | | | 1486 |
| P04070 | WELDLDIK | | | 1487 |
| P04070 | EVFVHPNYSK | | | 1488 |
| P04070 | ELNQAGQETLVTGWGYHSSR | | | 1489 |
| P04070 | TFVLNFIK | | | 1490 |
| P04070 | YLDWIHGHIR | | | 1491 |
| P05091 | TIPIDGDFFSYTR | | | 1492 |
| P05091 | VAEQTPLTALYVANLIK | | | 1493 |
| P05091 | VAFTGSTEIGR | | | 1494 |
| P05091 | VIQVAAGSSNLK | | | 1495 |
| P05091 | TFVQEDIYDEFVER | | | 1496 |
| P05091 | VVGNPFDSK | | | 1497 |
| P05091 | TEQGPQVDETQFK | | | 1498 |
| P05091 | ILGYINTGK | | | 1499 |
| P05091 | TIEEVVGR | | | 1500 |
| P05091 | ANNSTYGLAAAVFTK | | | 1501 |
| P05091 | ELGEYGLQAYTEVK | | | 1502 |
| P05091 | AAFQLGSPWR | | | 1503 |
| P05362 | LLGIETPLPK | | | 1504 |
| P05362 | ELLLPGNNR | | | 1505 |
| P05362 | TFLTVYWTPER | | | 1506 |
| P05362 | VELAPLPSWQPVGK | | | 1507 |
| P05362 | ANLTVVLLR | | | 1508 |
| P05362 | EPAVGEPAEVTTTVLVR | | | 1509 |
| P05362 | LNPTVTYGNDSFSAK | | | 1510 |
| P05362 | ASVSVTAEDEGTQR | | | 1511 |
| P05362 | VTLNGVPAQPLGPR | | | 1512 |
| P05362 | AQLLLK | | | 1513 |
| P05362 | ATPEDNGR | | | 1514 |
| P05362 | VLYGPR | | | 1515 |
| P05362 | DGTFPLPIGESVTVTR | | | 1516 |
| P05362 | STQGEVTR | | | 1517 |
| P05362 | VTVNVLSPR | | | 1518 |
| P08238 | VVVITK | | | 1519 |
| P08238 | NPDDITQEEYGEFYK | | | 1520 |
| P08238 | ALLFIPR | | | 1521 |
| P08238 | FYEAFSK | | | 1522 |
| P08238 | LGIHEDSTNR | | | 1523 |
| P08238 | HLEINPDHPIVETLR | | | 1524 |
| P08758 | GTVTDFPGFDER | | | 1525 |
| P08758 | ADAETLR | | | 1526 |
| P08758 | GLGTDEESILTLLTSR | | | 1527 |
| P08758 | QEISAAFK | | | 1528 |
| P08758 | DLLDDLK | | | 1529 |
| P08758 | SELTGK | | | 1530 |
| P08758 | LYDAYELK | | | 1531 |
| P08758 | GAGTNEK | | | 1532 |
| P08758 | VLTEIIASR | | | 1533 |
| P08758 | QVYEEEYGSSLEDDVVGDTSGY | | | 1534 |
| | YQR | | | |
| P08758 | DPDAGIDEAQVEQDAQALFQA | | | 1535 |
| | GELK | | | |
| P08758 | WGTDEEK | | | 1536 |
| P08758 | FITIFGTR | | | 1537 |
| P08758 | ETSGNLEQLLLAVVK | | | 1538 |
| P08758 | GAGTDDHTLIR | | | 1539 |
| P08758 | SEIDLFNIR | | | 1540 |
| P08758 | GDTSGDYK | | | 1541 |
| P24821 | QSGVNATLPEENQPVVFNHVY | | | 1542 |
| | NIK | | | |
| P24821 | LEELENLVSSLR | | | 1543 |
| P24821 | VPGDQTSTIIQELEPGVEYFIR | | | 1544 |
| P24821 | VFAILENK | | | 1545 |
| P24821 | SIPVSAR | | | 1546 |
| P24821 | VATYLPAPEGLK | | | 1547 |
| P24821 | ETSVEVEWDPLDIAFETWEIIFR | | | 1548 |
| P24821 | EDEGEITK | | | 1549 |
| P24821 | RPETSYR | | | 1550 |
| P24821 | QTGLAPGQEYEISLHIVK | | | 1551 |
| P24821 | LDAPSQIEVK | | | 1552 |
| P24821 | ETFTTGLDAPR | | | 1553 |
| P24821 | VSQTDNSITLEWR | | | 1554 |
| P24821 | AAIDSYR | | | 1555 |
| P24821 | YAPISGGDHAEVDVPK | | | 1556 |
| P24821 | SQQATTK | | | 1557 |
| P24821 | TTLTGLRPGTEYGIGVSAVK | | | 1558 |
| P24821 | ESNPATINAATELDTPK | | | 1559 |
| P24821 | DLQVSETAETSLTLLWK | | | 1560 |
| P24821 | LNYSLPTGQWVGVQLPR | | | 1561 |
| P24821 | NTTSYVLR | | | 1562 |
| P24821 | GLEPGQEYNVLLTAEK | | | 1563 |
| P24821 | ASTEQAPELENLTVTEVGWDGLR | | | 1564 |
| P24821 | LNWTAADQAYEHFIIQVQEANK | | | 1565 |
| P24821 | NLTVPGSLR | | | 1566 |
| P24821 | AVDIPGLK | | | 1567 |
| P24821 | AATPYTVSIYGVIQGYR | | | 1568 |
| P24821 | STDLPGLK | | | 1569 |
| P24821 | AATHYTITIR | | | 1570 |
| P24821 | AGTPYTVTLHGEVR | | | 1571 |
| P24821 | LNWTAADNAYEHFVIQVQEVNK | | | 1572 |
| P24821 | VEAAQNLTLPGSLR | | | 1573 |
| P24821 | AVDIPGLEAATPYR | | | 1574 |
| P24821 | VSIYGVIR | | | 1575 |
| P24821 | TPVLSAEASTAK | | | 1576 |
| P24821 | LLETVEYNISGAER | | | 1577 |
| P24821 | TAHISGLPPSTDFIVYLSGLAPSIR | | | 1578 |
| P24821 | LLDPQEFTLSGTQR | | | 1579 |
| P24821 | AEIVTEAEPEVDNLLVSDATPD | | | 1580 |
| | GFR | | | |
| P24821 | LSWTADEGVFDNFVLK | | | 1581 |
| P24821 | QSEPLEITLLAPER | | | 1582 |
| P24821 | EATEYEIELYGISK | | | 1583 |
| P24821 | EVIFSDITENSATVSWR | | | 1584 |
| P24821 | APTAQVESFR | | | 1585 |
| P24821 | WQPAIATVDSYVISYTGEK | | | 1586 |
| P24821 | VPEITR | | | 1587 |
| P24821 | TVSGNTVEYALTDLEPATEYTLR | | | 1588 |
| P24821 | SSTITAK | | | 1589 |
| P24821 | FTTDLDSPR | | | 1590 |
| P24821 | DLTATEVQSETALLTWRPPR | | | 1591 |
| P24821 | ASVTGYLLVYESVDGTVK | | | 1592 |
| P24821 | EVIVGPDTTSYSLADLSPSTHYT | | | 1593 |
| | AK | | | |
| P24821 | IQALNGPLR | | | 1594 |
| P24821 | ENFYQNWK | | | 1595 |
| P24821 | AYAAGFGDR | | | 1596 |
| P24821 | EEFWLGLDNLNK | | | 1597 |
| P24821 | ITAQGQYELR | | | 1598 |
| P24821 | DHGETAFAVYDK | | | 1599 |
| P24821 | FSVGDAK | | | 1600 |
| P24821 | SFSTFDK | | | 1601 |
| P24821 | GAFWYR | | | 1602 |
| P24821 | YGDNNHSQGVNWFHWK | | | 1603 |
| P39023 | HGSLGFLPR | | | 1604 |
| P39023 | DDPSKPVHLTAFLGYK | | | 1605 |
| P39023 | EVDRPGSK | | | 1606 |
| P39023 | WQDEDGK | | | 1607 |
| P39023 | VAFSVAR | | | 1608 |
| P39023 | IGQGYLIK | | | 1609 |
| P39023 | NNASTDYDLSDK | | | 1610 |
| P39023 | SLLVQTK | | | 1611 |
| P48637 | QQLEELAR | | | 1612 |
| P48637 | ALAEGVLLR | | | 1613 |
| P48637 | NIFDQR | | | 1614 |
| P48637 | AIENELLAR | | | 1615 |
| P48637 | NIHVIR | | | 1616 |
| P48637 | TFEDISEK | | | 1617 |
| P48637 | GSLDQDR | | | 1618 |
| P48637 | LFVDGQEIAVVYFR | | | 1619 |
| P48637 | QYSLQNWEAR | | | 1620 |
| P48637 | FVLKPQR | | | 1621 |
| P48637 | HVGHLLR | | | 1622 |
| P48637 | AIEHADGGVAAGVAVLDNPYPV | | | 1623 |
| P48637 | QDDFTAR | | | 1624 |
| P48637 | EGIAQTVFLGLNR | | | 1625 |
| P48637 | SADGSPALK | | | 1626 |
| P48637 | QIEINTISASFGGLASR | | | 1627 |
| P48637 | TPAVHR | | | 1628 |
| P48637 | HVLSVLSK | | | 1629 |
| P48637 | ILSNNPSK | | | 1630 |
| P48637 | GLALGIAK | | | 1631 |
| P48637 | AWELYGSPNALVLLIAQEK | | | 1632 |
| P49913 | DGHSLGR | | | 1633 |
| P49913 | AIDGINQR | | | 1634 |
| P49913 | SSDANLYR | | | 1635 |
| P49913 | FALLGDFFR | | | 1636 |
| Q12931 | ALLLWGR | | | 1637 |
| Q12931 | HEFQAETK | | | 1638 |
| Q12931 | LLDIVAR | | | 1639 |
| Q12931 | SLYSEK | | | 1640 |
| Q12931 | ELISNASDALEK | | | 1641 |
| Q12931 | AFLDALQNQAEASSK | | | 1642 |
| Q12931 | IIIHLK | | | 1643 |
| Q12931 | EFSSEAR | | | 1644 |
| Q12931 | YSNFVSFPLYLNGR | | | 1645 |
| Q12931 | EWQHEEFYR | | | 1646 |
| Q12931 | YVAQAHDKPR | | | 1647 |
| Q12931 | YTLHYK | | | 1648 |
| Q12931 | TDAPLNIR | | | 1649 |
| Q12931 | ELGSSVALYSR | | | 1650 |
| Q12931 | VLIQTK | | | 1651 |
| Q12931 | ATDILPK | | | 1652 |
| Q12931 | GVVDSEDIPLNLSR | | | 1653 |
| Q12931 | ELLQESALIR | | | 1654 |
| Q12931 | FFIDQSK | | | 1655 |
| Q12931 | EGIVTATEQEVK | | | 1656 |
| Q12931 | YESSALPSGQLTSLSEYASR | | | 1657 |
| Q12931 | LISVETDIVVDHYK | | | 1658 |
| Q12931 | NVLGSR | | | 1659 |
| Q12931 | AQLLQPTLEINPR | | | 1660 |
| Q12931 | LNELLVK | | | 1661 |
| Q12931 | TTAQLGPR | | | 1662 |
| Q12931 | NPAWSLQAGR | | | 1663 |
| Q12931 | LFSTQTAEDK | | | 1664 |
| Q12931 | EEPLHSIISSTESVQGSTSK | | | 1665 |
| Q13347 | SITQIK | | | 1666 |
| Q13347 | EGDLLFTVAK | | | 1667 |
| Q13347 | DPIVNVWYSVNGER | | | 1668 |
| Q13347 | TNSAVR | | | 1669 |
| Q13347 | SGEVLVNVK | | | 1670 |
| Q13347 | QINDIQLSR | | | 1671 |
| Q13347 | LFDSTTLEHQK | | | 1672 |
| Q13347 | FFHLAFEEEFGR | | | 1673 |
| Q13347 | GHFGPINSVAFHPDGK | | | 1674 |
| Q13347 | SYSSGGEDGYVR | | | 1675 |
| Q13347 | IHYFDPQYFEFEFEA | | | 1676 |
| Q14103 | NEEDEGHSNSSPR | | | 1677 |
| Q14103 | HSEAATAQR | | | 1678 |
| Q14103 | LDPITGR | | | 1679 |
| Q14103 | ESESVDK | | | 1680 |
| Q14103 | IFVGGLSPDTPEEK | | | 1681 |
| Q14103 | YHNVGLSK | | | 1682 |
| Q14103 | EQYQQQQQWGSR | | | 1683 |
| Q14103 | GGHQNSYKPY | | | 1684 |
| Q16706 | GHLDYPR | | | 1685 |
| Q16706 | IDHLER | | | 1686 |
| Q16706 | LLAENNEIISNIR | | | 1687 |
| Q16706 | DSVINLSESVEDGPK | | | 1688 |
| Q16706 | TFNDYFR | | | 1689 |
| Q16706 | FIWSEISYLSK | | | 1690 |
| Q16706 | WWDIIDIQK | | | 1691 |
| Q16706 | VHYAVK | | | 1692 |
| Q16706 | HFALHK | | | 1693 |
| Q16706 | TLEFFWR | | | 1694 |
| Q16706 | VLLAPLGDDFR | | | 1695 |
| Q16706 | IQFGTLSDFFDALDK | | | 1696 |
| Q16706 | ADETQR | | | 1697 |
| Q16706 | DDHYWSGYFTSRPFYK | | | 1698 |
| Q16706 | AAEILYYFALR | | | 1699 |
| Q16706 | FLSSSLYTALTEAR | | | 1700 |
| Q16706 | NLGLFQHHDAITGTAK | | | 1701 |
| Q16706 | DWVVVDYGTR | | | 1702 |
| Q16706 | IIGNSAFLLILK | | | 1703 |
| Q16706 | SQDSLPQK | | | 1704 |
| Q16706 | YLVVYNPLEQDR | | | 1705 |
| Q16706 | AHIPPLGLK | | | 1706 |
| Q16706 | ILESASSNSHLADYVLYK | | | 1707 |
| Q16706 | VEDSGIFTIK | | | 1708 |
| Q16706 | HHEVNVQFSWYGTTIK | | | 1709 |
| Q16706 | SGAYLFLPDGNAKPYVYTTPPFVR | | | 1710 |
| Q16706 | LYHIQGIEGQSVEVSNIVDIR | | | 1711 |
| Q16706 | ISSDIK | | | 1712 |
| Q16706 | FYTDLNGYQIQPR | | | 1713 |
| Q16706 | GLEQGIQDNK | | | 1714 |
| Q16706 | ITANLFR | | | 1715 |
| Q16706 | SAVNTEEEK | | | 1716 |
| Q16706 | VGNGHSNEAALILHR | | | 1717 |
| Q562R1 | AGFGGDDAPR | | | 1718 |
| Q562R1 | IWYHTFYNELR | | | 1719 |
| Q562R1 | VAPDEHPILLTEAPLNPK | | | 1720 |
| Q562R1 | GYNFTTTAER | | | 1721 |
| Q562R1 | AAASSSPER | | | 1722 |
| Q562R1 | QEYDEAGPPIVHR | | | 1723 |
| Q5SYB0 | SSFLTEEK | | | 1724 |
| Q5SYB0 | LYLENGQTK | | | 1725 |
| Q5SYB0 | FEANTTVK | | | 1726 |
| Q5SYB0 | DIILTVK | | | 1727 |
| Q5SYB0 | SIEYFALALEEQYSISR | | | 1728 |
| Q5SYB0 | LHLLHEEELIQQVVER | | | 1729 |
| Q5SYB0 | EESHDYR | | | 1730 |
| Q5SYB0 | DPLDLLK | | | 1731 |
| Q5SYB0 | LAALHIQER | | | 1732 |
| Q5SYB0 | DWGIENFISPTLLR | | | 1733 |
| Q5SYB0 | NQNLLEPR | | | 1734 |
| Q5SYB0 | QLISAAQLR | | | 1735 |
| Q5SYB0 | LNYLQILGELK | | | 1736 |
| Q5SYB0 | ESYIALLVGAK | | | 1737 |
| Q5SYB0 | YGISQVINSK | | | 1738 |
| Q5SYB0 | VELTEESEK | | | 1739 |
| Q5SYB0 | VYLQDVK | | | 1740 |
| Q5SYB0 | VLTLLLESNSAK | | | 1741 |
| Q5SYB0 | LLVDPVTSIFLWPGNK | | | 1742 |
| Q5SYB0 | VSAEEGYESR | | | 1743 |
| Q5SYB0 | EEQPPGNSPTPEVAR | | | 1744 |
| Q5SYB0 | STFFHFGSPGLAESIDSDSQEER | | | 1745 |
| Q5SYB0 | TEFSESAALETFGWAPELSTVR | | | 1746 |
| Q5SYB0 | VAAADGPAR | | | 1747 |
| Q5SYB0 | NPTQTLIPVR | | | 1748 |
| Q5SYB0 | AVDILR | | | 1749 |
| Q5SYB0 | EAEDSLSITVVR | | | 1750 |
| Q5SYB0 | LTPPGPPSGPR | | | 1751 |
| Q5SYB0 | DVSTAEPSATSLQNK | | | 1752 |
| Q5SYB0 | ALGLLAPLR | | | 1753 |
| Q5SYB0 | STNPASR | | | 1754 |
| Q5SYB0 | SVIDSR | | | 1755 |
| Q5SYB0 | VSSISAIR | | | 1756 |
| Q5SYB0 | IDPNNK | | | 1757 |
| Q5SYB0 | EPTIEHGDSSFSLSSGDPNPDR | | | 1758 |
| Q5SYB0 | YTEPLLSPR | | | 1759 |
| Q5SYB0 | IASIPTK | | | 1760 |
| Q5SYB0 | EEPQGQLSLER | | | 1761 |
| Q5SYB0 | NGTNVFQEESR | | | 1762 |
| Q5SYB0 | DSGDSPGDVSNNVSQTLDISSP | | | 1763 |
| | AGK | | | |
| Q5SYB0 | IVTSLSLDAPVTGTEQIPPHPPR | | | 1764 |
| Q5SYB0 | DPQGQSR | | | 1765 |
| Q5SYB0 | LFVELDLDPDFFLGK | | | 1766 |
| Q5SYB0 | QTVSPAVPPEGIK | | | 1767 |
| Q5SYB0 | AEAPNHVTGQDIAPR | | | 1768 |
| Q5SYB0 | GPQPETEEEDR | | | 1769 |
| Q5SYB0 | EAAGNLR | | | 1770 |
| Q5SYB0 | DVVYTYHQFIEAAK | | | 1771 |
| Q5SYB0 | GYHDLSVK | | | 1772 |
| Q92496 | VYLPWSR | | | 1773 |
| Q9H299 | VYSTSVTGSR | | | 1774 |
| Q9H299 | SQQSEVTR | | | 1775 |
| Q9H299 | IQYQLVDISQDNALR | | | 1776 |
| Q9H4B7 | GASALQLER | | | 1777 |
| Q9H4B7 | ISVYYNEAYGR | | | 1778 |
| Q9H4B7 | LGALFQPDSFVHGNSGAGNN | | | 1779 |
| | WAK | | | |
| Q9H4B7 | GHYTEGAELIENVLEVVR | | | 1780 |
| Q9H4B7 | EVDQQLLSVQTR | | | 1781 |
| Q9NQ79 | LVNIAVDER | | | 1782 |
| Q9NQ79 | SSPYYALR | | | 1783 |
| Q9NQ79 | EEIYFLNTNNAFSGVATYTDK | | | 1784 |
| Q9NQ79 | WEDILSDEVNVAR | | | 1785 |
| Q9NQ79 | GVASLFAGR | | | 1786 |
| Q9NQ79 | GILALR | | | 1787 |
| Q9NQ79 | DVAAEAGVSK | | | 1788 |
| Q9NQ79 | GDGTFVDAAASAGVDDPHQH | | | 1789 |
| | GR | | | |
| Q9NQ79 | GVALADFNR | | | 1790 |
| Q9NQ79 | VDIVYGNWNGPHR | | | 1791 |
| Q9NQ79 | DIASPK | | | 1792 |
| Q9NQ79 | TVITADFDNDQELEIFFNNIAYR | | | 1793 |
| Q9NQ79 | SSSANR | | | 1794 |
| Q9NQ79 | EHGDPLIEELNPGDALEPEGR | | | 1795 |
| Q9NQ79 | GNQGFNNNWLR | | | 1796 |
| Q9NQ79 | FGAFAR | | | 1797 |
| Q9NQ79 | VVLYTK | | | 1798 |
| Q9NQ79 | SGAHLR | | | 1799 |
| Q9NQ79 | DEASSVEVTWPDGK | | | 1800 |
| Q9UHG3 | YGFQSLR | | | 1801 |
| Q9UHG3 | YQSHDYAFSSVEK | | | 1802 |
| Q9UHG3 | TLLETLQK | | | 1803 |
| Q9UHG3 | AGFSEK | | | 1804 |
| Q9UHG3 | YTGNPTK | | | 1805 |
| Q9UHG3 | SDFYDIVLVATPLNR | | | 1806 |
| Q9UHG3 | GELNTSIFSSRPIDK | | | 1807 |
| Q9UHG3 | EDPEPSTDGTYVWK | | | 1808 |
| Q9UHG3 | IFSQETLTK | | | 1809 |
| Q9UHG3 | LFLSYDYAVK | | | 1810 |
| Q9UHG3 | KPWLAYPHYKPPEK | | | 1811 |
| Q9UHG3 | IAIIGAGIGGTSAAYYLR | | | 1812 |
| Q9UHG3 | IDLFER | | | 1813 |
| Q9UHG3 | EEVGGR | | | 1814 |
| Q9ULA0 | EAVQTAAK | | | 1815 |
| Q9ULA0 | LLQAGFSELK | | | 1816 |
| Q9ULA0 | WNIKPESK | | | 1817 |
| Q9ULA0 | SQVGFQQVGVETYGGGIWST | | | 1818 |
| | WFDR | | | |
| Q9ULA0 | DLTLAGR | | | 1819 |
| Q9ULA0 | LEQQLVHVERPILR | | | 1820 |
| Q9ULA0 | IPHLAIHLQR | | | 1821 |
| Q9ULA0 | GTPEPGPLNAVDER | | | 1822 |
| Q9ULA0 | HEENHRPLFHK | | | 1823 |
| Q9ULA0 | YASNAVSEALIR | | | 1824 |
| Q9ULA0 | GFFELFPSLSHNLLVD | | | 1825 |
| Q9UQ35 | GSGTNGYVQR | | | 1826 |
| Q9UQ35 | NLSLVR | | | 1827 |
| Q9UQ35 | GERPDYK | | | 1828 |
| Q9UQ35 | LEAALVK | | | 1829 |
| Q9UQ35 | RPNPDILDHER | | | 1830 |
| Q9UQ35 | DVNPGGK | | | 1831 |
| Q9UQ35 | EETPGQRPAVTETHQLAELNEK | | | 1832 |
| Q9UQ35 | AAFGISDSYVDGSSFDPQR | | | 1833 |
| Q9UQ35 | QPAPEPPKPYSLVR | | | 1834 |
| Q9UQ35 | STTPAPK | | | 1835 |
| Q9UQ35 | STSADSASSSDTSR | | | 1836 |
| Q9UQ35 | THTTALAGR | | | 1837 |
| Q9UQ35 | SPSPASGR | | | 1838 |
| Q9UQ35 | QPSSPYEDK | | | 1839 |
| Q9UQ35 | SATRPSPSPER | | | 1840 |
| Q9UQ35 | SSTGPEPPAPTPLLAER | | | 1841 |
| Q9UQ35 | LPQSSSSESSPPSPQPTK | | | 1842 |
| Q9UQ35 | HASSSPESPKPAPAPGSHR | | | 1843 |
| Q9UQ35 | EISSSPTSK | | | 1844 |
| Q9UQ35 | SHSHTPSR | | | 1845 |
| Q9UQ35 | SPATAK | | | 1846 |
| Q9UQ35 | SPQRPGWSR | | | 1847 |
| Q9UQ35 | SGSSQPK | | | 1848 |
| Q9UQ35 | SSSSPPPK | | | 1849 |
| Q9UQ35 | QSHSSSSPHPK | | | 1850 |
| Q9UQ35 | SGTPPR | | | 1851 |
| Q9UQ35 | QGSITSPQANEQSVTPQR | | | 1852 |
| Q9UQ35 | AIISPR | | | 1853 |
| Q9UQ35 | SHSGSSSPSPSR | | | 1854 |
| Q9UQ35 | YSHSGSSSPDTK | | | 1855 |
| Q9UQ35 | VKPETPPR | | | 1856 |
| Q9UQ35 | QSHSGSISPYPK | | | 1857 |
| Q9UQ35 | AQTPPGPSLSGSK | | | 1858 |
| Q9UQ35 | SSTPPGESYFGVSSLQLK | | | 1859 |
| Q9UQ35 | GQSQTSPDHR | | | 1860 |
| Q9UQ35 | SDTSSPEVR | | | 1861 |
| Q9UQ35 | QSHSESPSLQSK | | | 1862 |
| Q9UQ35 | SQTSPK | | | 1863 |
| Q9UQ35 | SSSPVTELASR | | | 1864 |
| Q9UQ35 | FQSDSSSYPTVDSNSLLGQSR | | | 1865 |
| Q9UQ35 | LETAESK | | | 1866 |
| Q9UQ35 | FSPFPVQDRPESSLVFK | | | 1867 |
| Q9UQ35 | SGAGSSPETK | | | 1868 |
| Q9UQ35 | SEEPAGQILSHLSSELK | | | 1869 |
| Q9UQ35 | ELSNSPLR | | | 1870 |
| Q9UQ35 | ENSFGSPLEFR | | | 1871 |
| Q9UQ35 | SSGHSSSELSPDAVEK | | | 1872 |
| Q9UQ35 | SGSSPGLR | | | 1873 |
| Q9UQ35 | DGSGTPSR | | | 1874 |
| Q9UQ35 | ALPQTPRPR | | | 1875 |
| Q9UQ35 | SPSSPELNNK | | | 1876 |
| Q9UQ35 | SGSESSVDQK | | | 1877 |
| Q9UQ35 | TPLGQR | | | 1878 |
| Q9UQ35 | SGSSQELDVKPSASPQER | | | 1879 |
| Q9UQ35 | SESDSSPDSK | | | 1880 |
| Q9UQ35 | SGSSPEVDSK | | | 1881 |
| Q9UQ35 | SGSSPEVK | | | 1882 |
| Q9UQ35 | AQSGSDSSPEPK | | | 1883 |
| Q9UQ35 | GPSPEGSSSTESSPEHPPK | | | 1884 |
| Q9UQ35 | SSPELTR | | | 1885 |
| Q9UQ35 | SASSSPETR | | | 1886 |
| Q9UQ35 | SPSVSSPEPAEK | | | 1887 |
| Q9UQ35 | SPSPKPR | | | 1888 |
| Q9UQ35 | SGSSQSTSR | | | 1889 |
| Q9UQ35 | GGSGYHSR | | | 1890 |
| Q9UQ35 | TSPAPWK | | | 1891 |
| Q9UQ35 | ASPATHR | | | 1892 |
| Q9UQ35 | TPLISR | | | 1893 |
| Q9UQ35 | TPPVTR | | | 1894 |
| Q9UQ35 | TSPITR | | | 1895 |
| Q9UQ35 | SPVPSAFSDQSR | | | 1896 |
| Q9UQ35 | VPSPTPAPK | | | 1897 |
| Q9UQ35 | EGRPPEPTPAK | | | 1898 |
| Q9UQ35 | KPPPGER | | | 1899 |
| Q9UQ35 | KPIDSLR | | | 1900 |
| Q9UQ35 | SLSYSPVER | | | 1901 |
| Q9UQ35 | RPSPQPSPR | | | 1902 |
| Q9UQ35 | DQQSSSSER | | | 1903 |
| Q9UQ35 | TPPAIR | | | 1904 |
| Q9UQ35 | SPLAIR | | | 1905 |
| Q9UQ35 | SPPAIR | | | 1906 |
| Q9UQ35 | SASGSSSDR | | | 1907 |
| Q9UQ35 | SATPPATR | | | 1908 |
| Q9UQ35 | NHSGSR | | | 1909 |
| Q9UQ35 | TPPVALNSSR | | | 1910 |
| Q9UQ35 | IPDHQR | | | 1911 |
| Q9UQ35 | TSVPENHAQSR | | | 1912 |
| Q9UQ35 | TAPAANLASR | | | 1913 |
| Q9UQ35 | TPAIPTAVNLADSR | | | 1914 |
| Q9UQ35 | TAVAPSAVNLADPR | | | 1915 |
| Q9UQ35 | TPTAPAVNLAGAR | | | 1916 |
| Q9UQ35 | TPQAPASANLVGPR | | | 1917 |
| Q9UQ35 | SAHATAPVNIAGSR | | | 1918 |
| Q9UQ35 | TAAALAPASLTSAR | | | 1919 |
| Q9UQ35 | VPLSAYER | | | 1920 |
| Q9UQ35 | TSPPLLDR | | | 1921 |
| Q9UQ35 | TPPSAPSQSR | | | 1922 |
| Q9UQ35 | APSPSSR | | | 1923 |

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A method of measuring or detecting at least one biomarker, the method comprising: testing a sample obtained from a subject; and measuring or detecting at least one biomarker or fragment thereof selected from the group consisting of AL9A1, ATPG, C1RL, CAND1, EPIPL, GLO2, IGHA2, PZP, SYTC, SYYC, or any combinations thereof in the sample; and/or measuring or detecting at least one biomarker of fragment thereof selected from the group consisting of ABHEB, AL9A1, DNM1L, FCN2, INF2, K22E, M3K5, NCOR1, SBSN, SYEP, TPP2, or any combinations thereof in the sample; wherein the measurement or detection of the at least one biomarker indicates that the subject has sustained or may have sustained a traumatic brain injury (TBI).

Clause 2. The method of clause 1, wherein the at least one biomarker or fragment thereof is selected from the group consisting of AL9A1, ATPG, C1RL, EPIPL, IGHA2, PZP, SYTC, SYYC, or any combinations thereof; and wherein the at least one biomarker is not detectable in a sample obtained from a healthy subject.

Clause 3. The method of either clause 1 or clause 2, wherein the at least one biomarker or fragment thereof is selected from the group consisting of ATPG, C1RL, SYYC, or any combinations thereof.

Clause 4. The method of clause 1, wherein the at least one biomarker or fragment thereof is selected from the group consisting of CAND1, GLO2, or the combination thereof, wherein levels of CAND1 and GLO2 are higher compared to levels of CAND1 and GLO2 in a sample obtained from a healthy subject.

Clause 5. The method of clause 1, wherein the at least one biomarker or fragment thereof is selected from the group consisting of ABHEB, AL9A1, DNM1L, or combinations thereof; wherein levels of ABHEB are higher compared to levels of ABHEB in a sample obtained from a healthy subject; and/or wherein levels of AL9A1 and DNM1L are lower compared to levels of AL9A1 and DNM1L in a sample obtained from a healthy subject.

Clause 6. The method of clause 1, wherein the at least one biomarker or fragment thereof is selected from the group consisting of M3K5, SBSN, SYEP, or combinations thereof; and wherein the measurement or detection of the at least one biomarker indicates that the subject has sustained or may have sustained a mild TBI.

Clause 7. The method of clause 1 or clause 6, wherein levels of SBSN and SYEP are higher compared to levels of SBSN and SYEP in a sample obtained from a healthy subject; and/or wherein levels of M3K5 are lower compared to levels of M3K5 in a sample obtained from a healthy subject.

Clause 8. The method of clause 1, wherein the at least one biomarker or fragment thereof is selected from the group consisting of INF2, SYEP, or combinations thereof.

Clause 9. The method of clause 1, wherein the at least one biomarker or fragment thereof further comprises at least one biomarker selected from the group consisting of ANXA6, ERAP1, EZRI, FA5, G6PI, MYLK, SAMP, or combinations thereof.

Clause 10. A method of measuring or detecting at least one biomarker, the method comprising: testing a sample obtained from a subject; and measuring or detecting at least one biomarker or fragment thereof selected from the group consisting of 1433G, ACK1, ACY1, AKA12, ARGI1, CADH5, CLH1, COPG2, DPOD2, DSG2, HV307, IQGA2, K1C14, K1C19, KV105, LAMC1, MDHM, NQO2, PERM, PLST, PNCB, PTPRC, SEPT7, SYRC, TRXR2, TXNL1, UGGG1, WDR1 or any combinations thereof in the sample; wherein the measurement or detection of the at least one biomarker indicates that the subject has sustained or may have sustained a mild traumatic brain injury (mTBI).

Clause 11. The method of clause 10, wherein the at least one biomarker is not detectable in a sample obtained from a healthy subject.

Clause 12. The method of clause 10, wherein the at least one biomarker is not detectable in a sample obtained from a subject that has sustained a severe TBI (sTBI).

Clause 13. The method of any of clauses 10 to 12, wherein the at least one biomarker or fragment thereof is selected from the group consisting of ACK1, ACY1, PLST, PNCB, PTPRC, UGGG1, or any combinations thereof in the sample; wherein the measurement or detection of the at least one biomarker indicates that the subject has sustained or may have sustained an mTBI of subclass 1.

Clause 14. The method of any of clauses 10 to 12, wherein the at least one biomarker or fragment thereof is selected from the group consisting of AKA12, HV307, PERM, KV105, NQO2, SEPT7, SYRC, TRXR2, or any combinations thereof in the sample; wherein the measurement or detection of the at least one biomarker indicates that the subject has sustained or may have sustained an mTBI of subclass 2.

Clause 15. The method of any of clauses 10 to 12, wherein the at least one biomarker or fragment thereof is selected from the group consisting of 1433B, ARGI1, CADH5, CLH1, COPG2, DPOD2, DSG2, IQGA2, K1C14, LAMC1, MDHM, TXNL1, or any combinations thereof in the sample; wherein the measurement or detection of the at least one biomarker indicates that the subject has sustained or may have sustained an mTBI of subclass 3.

Clause 16. The method of clause 10, wherein the at least one biomarker or fragment thereof further comprises at least one biomarker selected from the group consisting of AHNK, AL1A1, AMPN, CAPZB, CATD, CLIP2, CMGA, FSCN1, GMPR2, GRP78, GSH1, IDHC, K1C20, KRR1, MBL2, NTF2, PCBP2, PGK1, SAA1, TFR1, or combinations thereof.

Clause 17. A biomarker panel for determining traumatic brain injury (TBI) status of a subject, the panel comprising at least one of the following biomarkers: TPP2, CAND1, NCOR1, K22E, AL9A1, ABHEB, DNM1L, INF2, or any combinations thereof; wherein measurement or detection of the at least one biomarker indicates that the subject has sustained or may have sustained a mild TBI of subclass 4.

Clause 18. The biomarker panel of clause 17, wherein levels of CAND1, NCOR1, K22E, ABHEB, and DNM1L are higher compared to levels of CAND1, NCOR1, K22E, ABHEB, and DNM1L in a sample obtained from a healthy subject; and/or wherein levels of TPP2, AL9A1, and INF2 are lower compared to levels of TPP2, AL9A1, and INF2 in a sample obtained from a healthy subject.

Clause 19. A biomarker panel for determining traumatic brain injury (TBI) status of a subject, the panel comprising at least one of the following biomarkers: TPP2, NCOR1, HV103, INF2, IGHD, CK054, M3K5, ABHEB, AL9A1, DNM1L, or any combinations thereof; wherein measurement or detection of the at least one biomarker indicates that the subject has sustained or may have sustained a mild TBI of subclass 3.

Clause 20. The biomarker panel of clause 19, wherein levels of NCOR1, HV103, IGHD, ABHEB, and DNM1L are higher compared to levels of NCOR1, HV103, IGHD, ABHEB, and DNM1L in a sample obtained from a healthy subject; and/or wherein levels of TPP2, IGHD, CK054, M3K5, and AL9A1 are lower compared to levels of TPP2, IGHD, CK054, M3K5, and AL9A1 in a sample obtained from a healthy subject.

Clause 21. A biomarker panel for determining traumatic brain injury (TBI) status of a subject, the panel comprising at least one of the following biomarkers: NCOR1, TPP2, K22E, ABHEB, INF2, SBSN, AL9A1, MA2B2, or any combinations thereof; wherein measurement or detection of the at least one biomarker indicates that the subject has sustained or may have sustained a mild TBI of subclass 2.

Clause 22. The biomarker panel of clause 21, wherein levels of NCOR1, K22E, ABHEB, and SBSN are higher compared to levels of NCOR1, K22E, ABHEB, and SBSN in a sample obtained from a healthy subject; and/or wherein levels of TPP2, INF2, AL9A1, and MA2B2 are lower compared to levels of TPP2, INF2, AL9A1, and MA2B2 in a sample obtained from a healthy subject.

Clause 23. A biomarker panel for determining traumatic brain injury (TBI) status of a subject, the panel comprising at least one of the following biomarkers: K22E, DNM1L, DIAP1, ABHEB, PLOD1, SYEP, KV133, AL9A1, EPHB4, or any combinations thereof; wherein measurement or detection of the at least one biomarker indicates that the subject has sustained or may have sustained a mild TBI of subclass 1.

Clause 24. The biomarker panel of clause 23, wherein levels of K22E, DNM1L, DIAP1, ABHEB, PLOD1, SYEP, and EPHB4 are higher compared to levels of K22E, DNM1L, DIAP1, ABHEB, PLOD1, SYEP, and EPHB4 in a sample obtained from a healthy subject; and/or wherein levels of KV133 and AL9A1 are lower compared to levels of KV133 and AL9A1 in a sample obtained from a healthy subject.

Clause 25. The method of clause 1, wherein the at least one biomarker or fragment thereof further comprises at least one biomarker selected from the group consisting of MYLK, SAMP, or a combination thereof.

Clause 26. A biomarker panel for determining traumatic brain injury (TBI) status of a subject, the panel comprising at least one of the following biomarkers: CAND1, NCOR1, K22E, ABHEB, DNM1L, SBSN, GLO2, SYEP, or any combinations thereof; wherein measurement or detection of higher levels of the at least one biomarker in the subject as compared to levels of the at least one biomarkers in a healthy subject indicates that the subject has sustained or may have sustained a mild TBI of subclass 4.

Clause 27. A biomarker panel for determining traumatic brain injury (TBI) status of a subject, the panel comprising at least one of the following biomarkers: NCOR1, HV103, IGHD, ABHEB, DNM1L, ALBU, THIM, IGHA2, KV139, and, or any combinations thereof; wherein measurement or detection of higher levels of the at least one biomarker in the subject as compared to levels of the at least one biomarkers in a healthy subject indicates that the subject has sustained or may have sustained a mild TBI of subclass 3.

Clause 28. A biomarker panel for determining traumatic brain injury (TBI) status of a subject, the panel comprising at least one of the following biomarkers: NCOR1, K22E, ABHEB, SBSN, DNM1L, DIAP1, DYL1, PSA, EPHB4, or any combinations thereof; wherein measurement or detection of higher levels of the at least one biomarker in the subject as compared to levels of the at least one biomarkers in a healthy subject indicates that the subject has sustained or may have sustained a mild TBI of subclass 2.

Clause 29. A biomarker panel for determining traumatic brain injury (TBI) status of a subject, the panel comprising at least one of the following biomarkers: K22E, DNM1L, DIAP1, ABHEB, PLOD1, SYEP, EPHB4, FBLN3, or any combinations thereof; wherein measurement or detection of higher levels of the at least one biomarker in the subject as compared to levels of the at least one biomarkers in a healthy subject indicates that the subject has sustained or may have sustained a mild TBI of subclass 1.

Clause 30. The method of clause 10, wherein the at least one biomarker or fragment thereof further comprises at least one biomarker selected from the group consisting of ANXA6, MASP2, MYLK, SAMP, or a combination thereof.

Clause 31. A biomarker panel for determining that a subject has not sustained a traumatic brain injury (TBI), the panel comprising at least one of the following biomarkers: ACTBL, ALDH2, ANXA5, CAMP, CPNE3, CRAC1, CYTC, DNPEP, EIF3I, GSHB, ICAM1, HV323, HNRPD, KVD33, FA9, FHR4, FRPD1, HS90B, MA2A1, PCYOX, PNPH, PROC, RL3, SH3L3, SRRM2, TBB1, TENA, TRAP1 or any combinations thereof; wherein measurement or detection of the at least one biomarker in the subject indicates that the subject has not sustained a TBI.

Clause 32. A method of measuring or detecting at least one biomarker, the method comprising: obtaining a sample from a subject after an actual or suspected head injury; and measuring or detecting at least one biomarker or fragment thereof selected from the group consisting of AL9A1, ATPG, C1RL, CAND1, EPIPL, GLO2, IGHA2, PZP, SYTC, SYYC, or any combinations thereof in the sample; and/or measuring or detecting at least one biomarker of fragment thereof selected from the group consisting of ABHEB, AL9A1, DNM1L, FCN2, INF2, K22E, M3K5, NCOR1, SBSN, SYEP, TPP2, or any combinations thereof in the sample.

Clause 33. The method according to clause 32, wherein the measurement or detection of the at least one biomarker indicates that the subject has sustained a traumatic brain injury (TBI).

Clause 34. The method according to clause 32 or clause 33, wherein the at least one biomarker or fragment thereof is selected from the group consisting of AL9A1, ATPG, C1RL, EPIPL, IGHA2, PZP, SYTC, SYYC, or any combinations thereof; and wherein the at least one biomarker is not detectable in a sample obtained from a healthy subject.

Clause 35. The method according to any of clauses 32 to 34, wherein the at least one biomarker or fragment thereof is selected from the group consisting of ATPG, C1RL, SYYC, or any combinations thereof.

Clause 36. The method according to clause 32 or clause 33, wherein the at least one biomarker or fragment thereof is selected from the group consisting of CAND1, GLO2, or the combination thereof; and wherein levels of CAND1 and GLO2 are higher compared to levels of CAND1 and GLO2 in a sample obtained from a healthy subject.

Clause 37. The method according to clause 32 or clause 33, wherein the at least one biomarker or fragment thereof is selected from the group consisting of ABHEB, AL9A1, DNM1L, or combinations thereof; wherein levels of ABHEB are higher compared to levels of ABHEB in a sample obtained from a healthy subject; and/or wherein levels of AL9A1 and DNM1L are lower compared to levels of AL9A1 and DNM1L in a sample obtained from a healthy subject.

Clause 38. The method according to clause 32 or clause 33, wherein the at least one biomarker or fragment thereof is selected from the group consisting of M3K5, SBSN, SYEP, or combinations thereof; wherein levels of SBSN and SYEP are higher compared to levels of SBSN and SYEP in a sample obtained from a healthy subject; and/or wherein levels of M3K5 are lower compared to levels of M3K5 in a sample obtained from a healthy subject.

Clause 39. The method according to clause 32 or clause 33, wherein the at least one biomarker or fragment thereof is selected from the group consisting of INF2, SYEP, or combinations thereof.

Clause 40. The method according to any of clauses 32 to 39, wherein the at least one biomarker or fragment thereof further comprises at least one biomarker selected from the group consisting of ANXA6, ERAP1, EZRI, FA5, G6PI, MYLK, SAMP, or combinations thereof.

Clause 41. The method according to any of clauses 32 to 39, wherein the at least one biomarker or fragment thereof further comprises at least one biomarker selected from the group consisting of MYLK, SAMP, or a combination thereof.

Clause 42. A method of measuring or detecting at least one biomarker, the method comprising: obtaining a sample from a subject after an actual or suspected head injury; and measuring or detecting at least one biomarker or fragment thereof selected from the group consisting of 1433G, ACK1, ACY1, AKA12, ARGI1, CADH5, CLH1, COPG2, DPOD2, DSG2, HV319, IQGA2, K1C14, K1C19, KV105, LAMC1, MDHM, NQO2, PERM, PLST, PNCB, PTPRC, SEPT7, SYRC, TRXR2, TXNL1, UGGG1, WDR1 or any combinations thereof in the sample.

Clause 43. The method according to clause 42, wherein the measurement or detection of the at least one biomarker indicates that the subject has sustained a traumatic brain injury (TBI).

Clause 44. The method according to clause 42 or clause 43, wherein the at least one biomarker is not detectable in a sample obtained from a healthy subject.

Clause 45. The method according to any of clauses 42 to 44, wherein the at least one biomarker is not detectable in a sample obtained from a subject that has sustained a severe TBI.

Clause 46. The method according to any of clauses 42 to 44, wherein the at least one biomarker or fragment thereof is selected from the group consisting of ACK1, ACY1, PLST, PNCB, PTPRC, UGGG1, or any combinations thereof in the sample; wherein the measurement or detection of the at least one biomarker indicates that the subject has sustained a mild TBI of subclass 1.

Clause 47. The method according to any of clauses 42 to 44, wherein the at least one biomarker or fragment thereof is selected from the group consisting of AKA12, HV319, PERM, KV105, NQO2, SEPT7, SYRC, TRXR2, or any combinations thereof in the sample; wherein the measurement or detection of the at least one biomarker indicates that the subject has sustained a mild TBI of subclass 2.

Clause 48. The method according to any of clauses 42 to 44, wherein the at least one biomarker or fragment thereof is selected from the group consisting of 1433B, ARGI1, CADH5, CLH1, COPG2, DPOD2, DSG2, IQGA2, K1C14, LAMC1, MDHM, TXNL1, or any combinations thereof in the sample; wherein the measurement or detection of the at least one biomarker indicates that the subject has sustained a mild TBI of subclass 3.

Clause 49. The method according to any of clauses 42 to 48, wherein the at least one biomarker or fragment thereof further comprises at least one biomarker selected from the group consisting of AHNK, AL1A1, AMPN, CAPZB, CATD, CLIP2, CMGA, FSCN1, GMPR2, GRP78, GSH1, IDHC, K1C20, KRR1, MBL2, NTF2, PCBP2, PGK1, SAA1, TFR1, or combinations thereof.

Clause 50. The method according to any of clauses 42 to 48, wherein the at least one biomarker or fragment thereof further comprises at least one biomarker selected from the group consisting of ANXA6, MASP2, MYLK, SAMP, or a combination thereof.

Clause 51. A biomarker panel for determining traumatic brain injury (TBI) status of a subject, the panel comprising at least one of the following biomarkers: TPP2, CAND1, NCOR1, K22E, AL9A1, ABHEB, DNM1L, INF2, or any combinations thereof; wherein measurement or detection of the at least one biomarker indicates that the subject has sustained a mild TBI of subclass 4.

Clause 52. The biomarker panel according to clause 51, wherein levels of CAND1, NCOR1, K22E, ABHEB, and DNM1L are higher compared to levels of CAND1, NCOR1, K22E, ABHEB, and DNM1L in a sample obtained from a healthy subject; and/or wherein levels of TPP2, AL9A1, and INF2 are lower compared to levels of TPP2, AL9A1, and INF2 in a sample obtained from a healthy subject.

Clause 53. A biomarker panel for determining traumatic brain injury (TBI) status of a subject, the panel comprising at least one of the following biomarkers: TPP2, NCOR1, HV103, INF2, IGHD, CK054, M3K5, ABHEB, AL9A1, DNM1L, or any combinations thereof; wherein measurement or detection of the at least one biomarker indicates that the subject has sustained a mild TBI of subclass 3.

Clause 54. The biomarker panel according to clause 53, wherein levels of NCOR1, HV103, IGHD, ABHEB, and DNM1L are higher compared to levels of NCOR1, HV103, IGHD, ABHEB, and DNM1L in a sample obtained from a healthy subject; and/or wherein levels of TPP2, IGHD, CK054, M3K5, and AL9A1 are lower compared to levels of TPP2, IGHD, CK054, M3K5, and AL9A1 in a sample obtained from a healthy subject.

Clause 55. A biomarker panel for determining traumatic brain injury (TBI) status of a subject, the panel comprising at least one of the following biomarkers: NCOR1, TPP2, K22E, ABHEB, INF2, SBSN, AL9A1, MA2B2, or any combinations thereof; wherein measurement or detection of the at least one biomarker indicates that the subject has sustained a mild TBI of subclass 2.

Clause 56. The biomarker panel according to clause 55, wherein levels of NCOR1, K22E, ABHEB, and SBSN are higher compared to levels of NCOR1, K22E, ABHEB, and SBSN in a sample obtained from a healthy subject; and/or wherein levels of TPP2, INF2, AL9A1, and MA2B2 are lower compared to levels of TPP2, INF2, AL9A1, and MA2B2 in a sample obtained from a healthy subject.

Clause 57. A biomarker panel for determining traumatic brain injury (TBI) status of a subject, the panel comprising at least one of the following biomarkers: K22E, DNM1L, DIAP1, ABHEB, PLOD1, SYEP, KV102, AL9A1, EPHB4, or any combinations thereof; wherein measurement or detection of the at least one biomarker indicates that the subject has sustained a mild TBI of subclass 1.

Clause 58. The biomarker panel according to clause 57, wherein levels of K22E, DNM1L, DIAP1, ABHEB, PLOD1, SYEP, and EPHB4 are higher compared to levels of K22E, DNM1L, DIAP1, ABHEB, PLOD1, SYEP, and EPHB4 in a sample obtained from a healthy subject; and/or wherein levels of KV102 and AL9A1 are lower compared to levels of KV102 and AL9A1 in a sample obtained from a healthy subject.

Clause 59. A biomarker panel for determining traumatic brain injury (TBI) status of a subject, the panel comprising at least one of the following biomarkers: CAND1, NCOR1, K22E, ABHEB, DNM1L, SBSN, GLO2, SYEP, or any combinations thereof; wherein measurement or detection of higher levels of the at least one biomarker in the subject as compared to levels of the at least one biomarkers in a healthy subject indicates that the subject has sustained a mild TBI of subclass 4.

Clause 60. A biomarker panel for determining traumatic brain injury (TBI) status of a subject, the panel comprising at least one of the following biomarkers: NCOR1, HV103, IGHD, ABHEB, DNM1L, ALBU, THIM, IGHA2, KV139, and, or any combinations thereof; wherein measurement or detection of higher levels of the at least one biomarker in the subject as compared to levels of the at least one biomarkers in a healthy subject indicates that the subject has sustained a mild TBI of subclass 3.

Clause 61. A biomarker panel for determining traumatic brain injury (TBI) status of a subject, the panel comprising at least one of the following biomarkers: NCOR1, K22E, ABHEB, SBSN, DNM1L, DIAP1, DYL1, PSA, EPHB4, or any combinations thereof; wherein measurement or detection of higher levels of the at least one biomarker in the subject as compared to levels of the at least one biomarkers in a healthy subject indicates that the subject has sustained a mild TBI of subclass 2.

Clause 62. A biomarker panel for determining traumatic brain injury (TBI) status of a subject, the panel comprising at least one of the following biomarkers: K22E, DNM1L, DIAP1, ABHEB, PLOD1, SYEP, EPHB4, FBLN3, or any combinations thereof; wherein measurement or detection of higher levels of the at least one biomarker in the subject as compared to levels of the at least one biomarkers in a healthy subject indicates that the subject has sustained a mild TBI of subclass 1.

Clause 63. A biomarker panel for determining that a subject has not sustained a traumatic brain injury (TBI), the panel comprising at least one of the following biomarkers: ACTBL, ALDH2, ANXA5, CAMP, CPNE3, CRAC1, CYTC, DNPEP, EIF3I, GSHB, ICAM1, HV303, HNRPD, KV121, FA9, FHR4, FRPD1, HS90B, MA2A1, PCYOX, PNPH, PROC, RL3, SH3L3, SRRM2, TBB1, TENA, TRAP1 or any combinations thereof; wherein measurement or detection of the at least one biomarker in the subject indicates that the subject has not sustained a TBI.

Clause 64. A method of measuring or detecting at least one biomarker, the method comprising: obtaining a sample from a subject after an actual or suspected head injury; and measuring or detecting at least one peptide of at least one biomarker or fragment thereof selected from the group consisting of PZP, EPIPL, IGHA2, AL9A1, G6PI, SYTC, EZRI, FA5, or any combinations thereof in the sample; wherein the at least one peptide of the at least one biomarker is selected from the group consisting of SEQ ID NOs: 4-260.

Clause 65. Use of at least one peptide selected from the group consisting of SEQ ID NOs: 4-260 to isolate or identify at least one biomarker or fragment thereof selected from the group consisting of PZP, EPIPL, IGHA2, AL9A1, G6PI, SYTC, EZRI, FA5, or any combinations thereof.

Clause 66. A method of measuring or detecting at least one biomarker, the method comprising: obtaining a sample from a subject after an actual or suspected head injury; and measuring or detecting at least one peptide of at least one biomarker or fragment thereof selected from the group consisting of NCOR1, ABHEB, ANXA6, DNM1L, FCN2, HBA, MYLK, SBSN, AHNK, K22E, or any combinations thereof in the sample; wherein the at least one peptide of the at least one biomarker is selected from the group consisting of SEQ ID NOs: 261-698.

Clause 67. Use of at least one peptide selected from the group consisting of SEQ ID NOs: 261-698 to isolate or identify at least one biomarker or fragment thereof selected from the group consisting of NCOR1, ABHEB, ANXA6, DNM1L, FCN2, HBA, MYLK, SBSN, AHNK, K22E, or any combinations thereof.

Clause 68. A method of measuring or detecting at least one biomarker, the method comprising: obtaining a sample from a subject after an actual or suspected head injury; and measuring or detecting at least one peptide of at least one biomarker or fragment thereof selected from the group consisting of CAND1, SYEP, GLO2, DIAP1, DYL1, PSA, EPHB4, PLOD1, FBLN3, HV103, IGHD, THIM, IGHA2, KV105, MASP2, or any combinations thereof in the sample; wherein the at least one peptide of the at least one biomarker is selected from the group consisting of SEQ ID NOs: 699-926.

Clause 69. Use of at least one peptide selected from the group consisting of SEQ ID NOs: 699-926 to isolate or identify at least one biomarker or fragment thereof selected from the group consisting of CAND1, SYEP, GLO2, DIAP1, DYL1, PSA, EPHB4, PLOD1, FBLN3, HV103, IGHD, THIM, IGHA2, KV105, MASP2, or any combinations thereof.

Clause 70. A method of measuring or detecting at least one biomarker, the method comprising: obtaining a sample from a subject after an actual or suspected head injury; and measuring or detecting at least one peptide of at least one biomarker or fragment thereof selected from the group consisting of SAMP, 1433B, FIBB, RS28, TRFE, or any combinations thereof in the sample; wherein the at least one peptide of the at least one biomarker is selected from the group consisting of SEQ ID NOs: 927-968.

Clause 71. Use of at least one peptide selected from the group consisting of SEQ ID NOs: 927-968 to isolate or identify at least one biomarker or fragment thereof selected from the group consisting of SAMP, 1433B, FIBB, RS28, TRFE, or any combinations thereof.

Clause 72. A method of measuring or detecting at least one biomarker, the method comprising: obtaining a sample from a subject after an actual or suspected head injury; and measuring or detecting at least one peptide of at least one biomarker or fragment thereof selected from the group consisting of DYL1, FBLN3, PSA, HV103, IGHD, or any combinations thereof in the sample; wherein the at least one peptide of the at least one biomarker is selected from the group consisting of SEQ ID NOs: 969-996.

Clause 73. Use of at least one peptide selected from the group consisting of SEQ ID NOs: 969-996 to isolate or identify at least one biomarker or fragment thereof selected from the group consisting of DYL1, FBLN3, PSA, HV103, IGHD, or any combinations thereof.

Clause 74. A method of measuring or detecting at least one biomarker, the method comprising: obtaining a sample from a subject after an actual or suspected head injury; and measuring or detecting at least one peptide of at least one biomarker or fragment thereof selected from the group consisting of FCN2, HBA, AHNK, or any combinations thereof in the sample; wherein the at least one peptide of the at least one biomarker is selected from the group consisting of SEQ ID NOs: 997-1182.

Clause 75. Use of at least one peptide selected from the group consisting of SEQ ID NOs: 997-1182 to isolate or identify at least one biomarker or fragment thereof selected from the group consisting of FCN2, HBA, AHNK, or any combinations thereof.

Clause 76. A method of measuring or detecting at least one biomarker, the method comprising: obtaining a sample from a subject after an actual or suspected head injury; and measuring or detecting at least one peptide of at least one biomarker or fragment thereof selected from the group consisting of HV103, IGHD, THIM, IGHA2, KV105, MASP2, DIAP1, PLOD1, EPHB4, FBLN3, DYL1, PSA, or any combinations thereof in the sample; wherein the at least one peptide of the at least one biomarker is selected from the group consisting of SEQ ID NOS: 1183-1319.

Clause 77. Use of at least one peptide selected from the group consisting of SEQ ID NOs: 1183-1319 to isolate or identify at least one biomarker or fragment thereof selected from the group consisting of HV103, IGHD, THIM, IGHA2, KV105, MASP2, DIAP1, PLOD1, EPHB4, FBLN3, DYL1, PSA, or any combinations thereof.

Clause 78. A method of measuring or detecting at least one biomarker, the method comprising: obtaining a sample from a subject after an actual or suspected head injury; and measuring or detecting at least one peptide of at least one biomarker or fragment thereof selected from the group consisting of CAND1, GLO2, ERAP1, SYYC, C1RL, ATPG, or any combinations thereof in the sample; wherein the at least one peptide of the at least one biomarker is selected from the group consisting of SEQ ID NOs: 1320-1438.

Clause 79. Use of at least one peptide selected from the group consisting of SEQ ID NOs: 1320-1438 to isolate or identify at least one biomarker or fragment thereof selected from the group consisting of CAND1, GLO2, ERAP1, SYYC, C1RL, ATPG, or any combinations thereof.

Clause 80. A method of measuring or detecting at least one biomarker, the method comprising: obtaining a sample from a subject after an actual or suspected head injury; and measuring or detecting at least one peptide of at least one biomarker or fragment thereof selected from the group consisting of ACTBL, ALDH2, ANXA5, CAMP, CPNE3, CRAC1, CYTC, DNPEP, EIF3I, GSHB, ICAM1, HV323, HNRPD, KVD33, FA9, FHR4, FRPD1, HS90B, MA2A1, PCYOX, PNPH, PROC, RL3, SH3L3, SRRM2, TBB1, TENA, TRAP1, or any combinations thereof in the sample; wherein the at least one peptide of the at least one biomarker is selected from the group consisting of SEQ ID NOs: 1439-1923.

Clause 81. Use of at least one peptide selected from the group consisting of SEQ ID NOs: 1439-1923 to isolate or identify at least one biomarker or fragment thereof selected from the group consisting of ACTBL, ALDH2, ANXA5, CAMP, CPNE3, CRAC1, CYTC, DNPEP, EIF3I, GSHB, ICAM1, HV323, HNRPD, KVD33, FA9, FHR4, FRPD1, HS90B, MA2A1, PCYOX, PNPH, PROC, RL3, SH3L3, SRRM2, TBB1, TENA, TRAP1, or any combinations thereof.

1.-32. (canceled)

33. A method comprising:
measuring or detecting GFAP in a sample obtained from a subject, wherein: (a) the measurement or detection of GFAP indicates that the subject has sustained or may have sustained a mild traumatic brain (mTBI) injury of subclass 4, and (b) the levels of GFAP are higher compared to levels obtained from a healthy subject, and
treating the subject for a mTBI, wherein treating the subject for a mTBI comprises: treatment (i) with rest: (ii) by abstaining from physical activities: (iii) by avoiding light: (iv) by wearing protective eyewear when in light: (v) with one or more therapeutic agents selected from the group consisting of a medication for relief of a headache or migraine, and an anti-nausea medication, or combinations thereof; or (vi) with any combination of (i)-(v).

34. The method according to any of claim 33, wherein the subject is a human.

35. The method of claim 33, wherein the method further comprises measuring or detecting at least one of the following biomarkers or fragments thereof: TPP2, CAND1, NCOR1, K22E, AL9A1, ABHEB, DNM1L, INF2, or any combinations thereof in the sample.

36. The method according to claim 35, wherein:
levels of CAND1, NCOR1, K22E, ABHEB, and DNMI1L are higher compared to levels of CAND1, NCOR1, K22E, ABHEB, and DNMI1L in a sample obtained from a healthy subject;
levels of TTP2, AL9A1, and INF2 are lower compared to levels of TPP2, AL9A1, and INF2 in a sample obtained from a healthy subject, or
levels of CAND1, NCOR1, K22E, ABHEB, and DNMI1L are higher compared to levels of CAND1, NCOR1, K22E, ABHEB, and DNMI1L in a sample obtained from a healthy subject and levels of TTP2, AL9A1, and INF2 are lower compared to levels of TPP2, AL9A1, and INF2 in a sample obtained from a healthy subject.

37. The method of claim 33, wherein measuring or detecting a level of GFAP comprises performing an immunoassay, a clinical chemistry assay, a single molecule detection assay, a point-of-care assay, and/or mass spectroscopy.
